(19) United States
(12) Patent Application Publication
(10) Pub. No.: US 2021/0115040 A1
(43) Pub. Date: Apr. 22, 2021

(54) OGA INHIBITOR COMPOUNDS

(71) Applicant: Janssen Pharmaceutica NV, Beerse (BE)

(72) Inventors: José Manuel Bartolomé-Nebreda, Toledo (ES); Andrés Avelino Trabanco-Suárez, Olias del Rey (ES); Ana Isabel De Lucas Olivares, Bargas (ES); Gary John Tresadern, Toledo (ES); Susana Conde-Ceide, Toledo (ES)

(21) Appl. No.: 17/253,477

(22) PCT Filed: Jun. 20, 2019
(86) PCT No.: PCT/EP2019/066386

§ 371 Date: Dec. 17, 2020

(30) Foreign Application Priority Data
Jun. 20, 2018 (EP) 18382448.1

(51) Int. Cl. C07D 471/04; C07D 401/14; C07D 498/04; C07D 413/14; C07D 513/04; C07D 491/048; C07D 417/14

(52) U.S. Cl. CPC C07D 471/04; C07D 401/14; C07D 498/04; C07D 417/14; C07D 513/04; C07D 491/048; C07D 413/14

(57) ABSTRACT

The present invention relates to O-GlcNAc hydrolase (OGA) inhibitors. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### FIELD OF THE INVENTION

The present invention relates to O-GlcNAc hydrolase (OGA) inhibitors, having the structure shown in Formula (I)

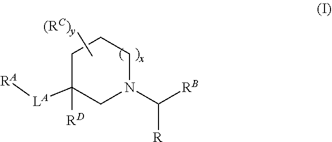

wherein the radicals are as defined in the specification. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### BACKGROUND OF THE INVENTION

O-GlcNAcylation is a reversible modification of proteins where N-acetyl-D-glucosamine residues are transferred to the hydroxyl groups of serine- and threonine residues yield O-GlcNAcylated proteins. More than 1000 of such target proteins have been identified both in the cytosol and nucleus of eukaryotes. The modification is thought to regulate a huge spectrum of cellular processes including transcription, cytoskeletal processes, cell cycle, proteasomal degradation, and receptor signalling.

O-GlcNAc transferase (OGT) and O-GlcNAc hydrolase (OGA) are the only two proteins described that add (OGT) or remove (OGA) O-GlcNAc from target proteins. OGA was initially purified in 1994 from spleen preparation and 1998 identified as antigen expressed by meningiomas and termed MGEA5, consists of 916 amino (102915 Dalton) as a monomer in the cytosolic compartment of cells. It is to be distinguished from ER- and Golgi-related glycosylation processes that are important for trafficking and secretion of proteins and different to OGA have an acidic pH optimum, whereas OGA display highest activity at neutral pH.

The OGA catalytic domain with its double aspartate catalytic center resides in the N-terminal part of the enzyme which is flanked by two flexible domains. The C-terminal part consists of a putative HAT (histone acetyl transferase domain) preceded by a stalk domain. It has yet still to be proven that the HAT-domain is catalytically active.

O-GlcNAcylated proteins as well as OGT and OGA themselves are particularly abundant in the brain and neurons suggesting this modification plays an important role in the central nervous system. Indeed, studies confirmed that O-GlcNAcylation represents a key regulatory mechanism contributing to neuronal communication, memory formation and neurodegenerative disease. Moreover, it has been shown that OGT is essential for embryogenesis in several animal models and ogt null mice are embryonic lethal. OGA is also indispensible for mammalian development. Two independent studies have shown that OGA homozygous null mice do not survive beyond 24-48 hours after birth. Oga deletion has led to defects in glycogen mobilization in pups and it caused genomic instability linked cell cycle arrest in MEFs derived from homozygous knockout embryos. The heterozygous animals survived to adulthood however they exhibited alterations in both transcription and metabolism.

It is known that perturbations in O-GlcNAc cycling impact chronic metabolic diseases such as diabetes, as well as cancer. Oga heterozygosity suppressed intestinal tumorigenesis in an Apc−/+ mouse cancer model and the Oga gene (MGEA5) is a documented human diabetes susceptibility locus.

In addition, O-GlcNAc-modifications have been identified on several proteins that are involved in the development and progression of neurodegenerative diseases and a correlation between variations of O-GlcNAc levels on the formation of neurofibrillary tangle (NFT) protein by Tau in Alzheimer's disease has been suggested. In addition, O-GlcNAcylation of alpha-synuclein in Parkinson's disease has been described.

In the central nervous system six splice variants of tau have been described. Tau is encoded on chromosome 17 and consists in its longest splice variant expressed in the central nervous system of 441 amino acids. These isoforms differ by two N-terminal inserts (exon 2 and 3) and exon 10 which lie within the microtubule binding domain. Exon 10 is of considerable interest in tauopathies as it harbours multiple mutations that render tau prone to aggregation as described below. Tau protein binds to and stabilizes the neuronal microtubule cytoskeleton which is important for regulation of the intracellular transport of organelles along the axonal compartments. Thus, tau plays an important role in the formation of axons and maintenance of their integrity. In addition, a role in the physiology of dendritic spines has been suggested as well.

Tau aggregation is either one of the underlying causes for a variety of so called tauopathies like PSP (progressive supranuclear palsy), Down's syndrome (DS), FTLD (frontotemporal lobe dementia), FTDP-17 (frontotemporal dementia with Parkinsonism-17), Pick's disease (PD), CBD (corticobasal degeneration), agryophilic grain disease (AGD), and AD (Alzheimer's disease). In addition, tau pathology accompanies additional neurodegenerative diseases like amyotrophic lateral sclerosis (ALS) or FTLD cause by C9ORF72 mutations. In these diseases, tau is post-translationally modified by excessive phosphorylation which is thought to detach tau from microtubules and makes it prone to aggregation. O-GlcNAcylation of tau regulates the extent of phosphorylation as serine or threonine residues carrying O-GlcNAc-residues are not amenable to phosphorylation. This effectively renders tau less prone to detaching from microtubules and reduces aggregation into neurotoxic tangles which ultimately lead to neurotoxicity and neuronal cell death. This mechanism may also reduce the cell-to-cell spreading of tau-aggregates released by neurons via along interconnected circuits in the brain which has recently been discussed to accelerate pathology in tau-related dementias. Indeed, hyperphosphorylated tau isolated from brains of AD-patients showed significantly reduced O-GlcNAcylation levels.

An OGA inhibitor administered to JNPL3 tau transgenic mice successfully reduced NFT formation and neuronal loss without apparent adverse effects. This observation has been confirmed in another rodent model of tauopathy where the expression of mutant tau found in FTD can be induced (tg4510). Dosing of a small molecule inhibitor of OGA was efficacious in reducing the formation of tau-aggregation and attenuated the cortical atrophy and ventricle enlargement.

Moreover, the O-GlcNAcylation of the amyloid precursor protein (APP) favours processing via the non-amyloidogenic route to produce soluble APP fragment and avoid cleavage that results in the AD associated amyloid-beta (Aβ) formation.

Maintaining O-GlcNAcylation of tau by inhibition of OGA represents a potential approach to decrease tau-phosphorylation and tau-aggregation in neurodegenerative diseases mentioned above thereby attenuating or stopping the progression of neurodegenerative tauopathy-diseases.

WO2012/117219 (Summit Corp. plc., published 7 Sep. 2012) describes N-[[5-(hydroxymethyl)pyrrolidin-2-yl]methyl]alkylamide and N-alkyl-2-[5-(hydroxymethyl)pyrrolidin-2-yl]acetamide derivatives as OGA inhibitors. WO2014/159234 (Merck Patent GMBH, published 2 Oct. 2014) discloses mainly 4-phenyl or benzyl-piperidine and piperazine compounds substituted at the 1-position with an acetamido-thiazolylmethyl or acetamidoxazolylmethyl substituent and the compound N-[5-[(3-phenyl-1-piperidyl)methyl]thiazol-2-yl]acetamide; WO2016/0300443 (Asceneuron S. A., published 3 Mar. 2016), WO2017/144633 and WO2017/0114639 (Asceneuron S. A., published 31 Aug. 2017) disclose 1,4-disubstituted piperidines or piperazines as OGA inhibitors; WO2017/144637 (Asceneuron S. A, published 31 Aug. 2017) discloses more particular 4-substituted 1-[1-(1,3-benzodioxol-5-yl)ethyl]-piperazine; 1-[1-(2,3-dihydrobenzofuran-5-yl)ethyl]-; 1-[1-(2,3-dihydrobenzofuran-6-yl)ethyl]-; and 1-[1-(2,3-dihydro-1,4-benzodioxin-6-yl)ethyl]-piperazine derivatives as OGA inhibitors; WO2017/106254 (Merck Sharp & Dohme Corp.) describes substituted N-[5-[(4-methylene-1-piperidyl)methyl]thiazol-2-yl]acetamide compounds as OGA inhibitors.

There is still a need for OGA inhibitor compounds with an advantageous balance of properties, for example with improved potency, good bioavailability, pharmacokinetics, and brain penetration, and/or better toxicity profile. It is accordingly an object of the present invention to provide compounds that overcome at least some of these problems.

### SUMMARY OF THE INVENTION

The present invention is directed to compounds of Formula (I)

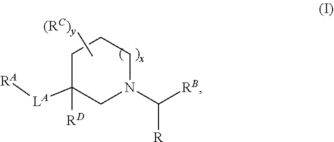

and the tautomers and the stereoisomeric forms thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;
L^{A} is selected from the group consisting of a covalent bond, —CH₂—, —O—, —OCH₂—, —CH₂O—, —NH—, —N(CH₃)—, —NHCH₂— and —CH₂NH—;
x represents 0 or 1;

### R is H or CH₃; and

R^{B} is an aromatic heterobicyclic radical selected from the group consisting of (b-1) to (b-12)

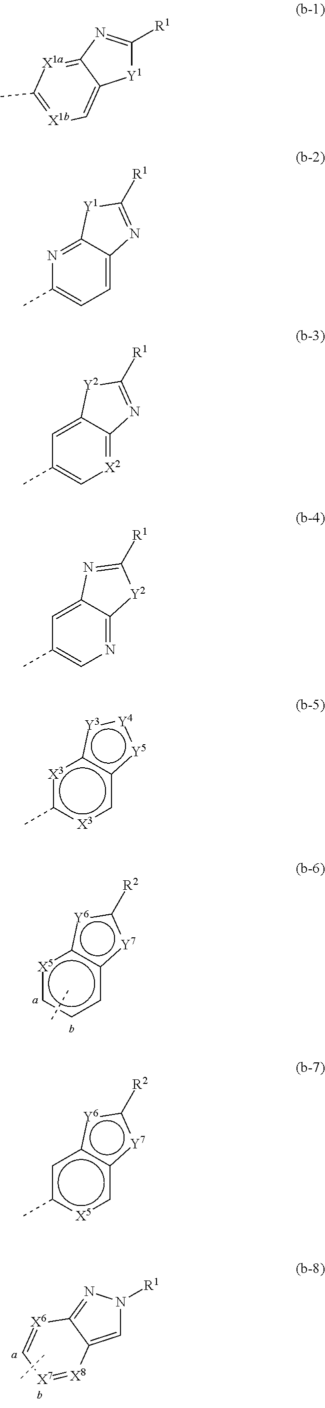

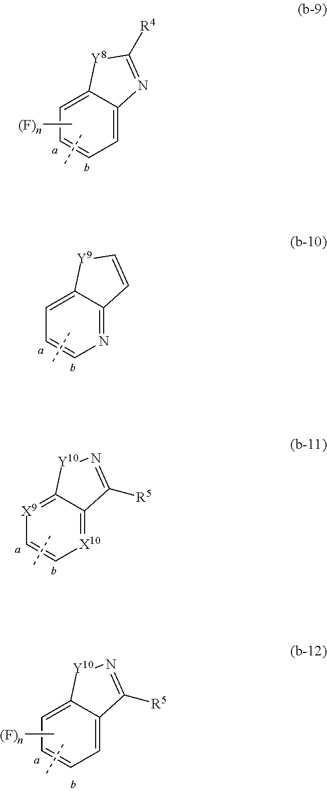

wherein
X^{1a} and X^{1b} each independently represents CH or N; and Y¹ represents O or S, with the proviso that at least one of X^{1a} and X^{1b} is CH, and when Y¹ is S, X^{1a} or X^{1b} is N;
X² represents CH or N; and Y² represents O or S;
X³ and X⁴ are each independently selected from N and CF; with the proviso that when X³ is N, X⁴ is CF and when X³ is CF, X⁴ is N;
one or two of Y³-Y⁵ is a heteroatom each independently selected from the group consisting of ═N—, >NH, >N(C₁₋₄alkyl), S and O, with the proviso that up to one of Y³—Y⁵ may be O or S when present; and the remaining Y³-Y⁵ are each independently selected from the group consisting of CH and C(C₁₋₄alkyl);
X⁵ represents CH or N;
one of Y⁶ or Y⁷ is ═N— and the other is >NH or >NCH₃;
X⁶, X⁷ and X⁸ each independently represent CH or N, with the proviso that up to one of them can be N and with the proviso that X⁷ is C when b is the point of attachment to CHR;
Y⁸ and Y⁹ are each independently selected from the group consisting of O, S, NH and NCH₃;
X⁹ and X^{th} each independently represent CH or N, with the proviso that at least one of them is CH;
a and b, when present, represent the point of attachment of the aromatic heterobicyclic radical R^{B} to CHR;
R¹, R², and R³ are each selected from C₁₋₄alkyl;
R⁴ and R⁵ are each selected from the group consisting of H and C₁₋₄alkyl;
Y¹⁰ represents O or S;
n represents 1 or 2;
R^{C} is selected from the group consisting of fluoro, methyl, hydroxy, methoxy, trifluoromethyl, and difluoromethyl;

R^{D} is selected from the group consisting of hydrogen, fluoro, methyl, hydroxy, methoxy, trifluoromethyl, and difluoromethyl; and

y represents 0, 1 or 2;
with the provisos that
a) R^{C} is not hydroxy or methoxy when present at the carbon atom adjacent to the nitrogen atom of the piperidinediyl or pyrrolidinediyl ring;
b) R^{C} or R^{D} cannot be selected simultaneously from hydroxy or methoxy when R^{C} is present at the carbon atom adjacent to C—R^{D};
c) R^{D} is not hydroxy or methoxy when L^{A} is —O—, —OCH₂—, —CH₂O—, —NH—, —N(CH₃)—, —NH(CH₂)— or —(CH₂)NH—;
   and the pharmaceutically acceptable salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of preventing or treating a disorder mediated by the inhibition of O-GlcNAc hydrolase (OGA), comprising administering to a subject in need thereof a therapeutically effective amount of any of the compounds or pharmaceutical compositions described above.

Further exemplifying the invention are methods of inhibiting OGA, comprising administering to a subject in need thereof a prophylactically or a therapeutically effective amount of any of the compounds or pharmaceutical compositions described above.

An example of the invention is a method of preventing or treating a disorder selected from a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of any of the compounds or pharmaceutical compositions described above.

Another example of the invention is any of the compounds described above for use in preventing or treating a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, in a subject in need thereof.

### DETAILED DESCRIPTION OF THE INVENTION

The present invention is directed to compounds of Formula (I), as defined herein before, and pharmaceutically acceptable addition salts and solvates thereof. The compounds of Formula (I) are inhibitors of O-GlcNAc hydrolase (OGA) and may be useful in the prevention or treatment of tauopathies, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or may be useful in the prevention or treatment of neurodegenerative diseases accompanied by a tau pathology, in particular a neurodegenerative disease selected from amyotrophic lateral sclerosis or frontotemporal lobe dementia caused by C9ORF72 mutations.

In a particular embodiment, the invention is directed to compounds of Formula (I) as referred to herein, and the tautomers and the stereoisomeric forms thereof, wherein R^{A} is a heteroaryl radical selected from the group consisting of pyridin-2-yl, pyridin-4-yl, and pyrimidin-4-yl, each of which may be optionally substituted with 1, 2 or 3 substituents each independently selected from the group consisting of halo; cyano, C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;

and the pharmaceutically acceptable salts and the solvates thereof.

In a further embodiment, the invention is directed to compounds of Formula (I) as referred to herein, and the tautomers and the stereoisomeric forms thereof, wherein R^{A} is a heteroaryl radical selected from the group consisting of pyridin-4-yl, and pyrimidin-4-yl, each of which may be optionally substituted with 1, 2 or 3 substituents each independently selected from the group consisting of halo; cyano, C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;

and the pharmaceutically acceptable salts and the solvates thereof.

In a further embodiment, the invention is directed to compounds of Formula (I) as referred to herein, and the tautomers and the stereoisomeric forms thereof, wherein R^{A} is a heteroaryl radical selected from the group consisting of pyridin-2-yl, pyridin-4-yl, and pyrimidin-4-yl, each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents;

and the pharmaceutically acceptable salts and the solvates thereof.

In a further embodiment, the invention is directed to compounds of Formula (I) as referred to herein, and the tautomers and the stereoisomeric forms thereof, wherein R^{A} is a heteroaryl radical selected from the group consisting of pyridin-2-yl and pyridin-4-yl, in particular pyridin-4-yl, each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents;

and the pharmaceutically acceptable salts and the solvates thereof.

In a further embodiment, the invention is directed to compounds of Formula (I) as referred to herein, and the tautomers and the stereoisomeric forms thereof, wherein R^{A} is a heteroaryl radical selected from the group consisting of pyridin-4-yl, and pyrimidin-4-yl, each of which may be optionally substituted with 1 or 2 substituents each independently selected from the group consisting of C₁₋₄ alkyl optionally substituted with 1, 2, or 3 independently selected halo substituents; and C₁₋₄alkyloxy optionally substituted with 1, 2, or 3 independently selected halo substituents;

and the pharmaceutically acceptable salts and the solvates thereof.

In an additional embodiment, the invention is directed to compounds of Formula (I) as referred to herein, and the tautomers and the stereoisomeric forms thereof, wherein L^{A} is selected from the group consisting of —CH₂—, —O—, —OCH₂—, —CH₂O—, —NH—, —N(CH₃)—, —NHCH₂— and —CH₂NH—.

In a further, embodiment, the invention is directed to compounds of Formula (I) as referred to herein, and the tautomers and the stereoisomeric forms thereof, wherein L^{A} is selected from the group consisting of a covalent bond, —CH₂—, —O—, —OCH₂— —CH₂O—, —NH—, —NHCH₂— and —CH₂NH—.

In an additional embodiment, the invention is directed to compounds of Formula (I) as referred to herein, and the tautomers and the stereoisomeric forms thereof, wherein L^{A} is selected from the group consisting of a covalent bond, —CH₂—, —O—, —OCH₂— and —NHCH₂—.

In a further embodiment, the invention is directed to compounds of Formula (I) as referred to herein, and the tautomers and the stereoisomeric forms thereof, wherein L^{A} is selected from the group consisting of a covalent bond, —CH₂—, —O—, —OCH₂—, and —CH₂O—.

In a further embodiment, the invention is directed to compounds of Formula (I) as referred to herein, and the tautomers and the stereoisomeric forms thereof, wherein L^{A} is selected from the group consisting of a covalent bond, —CH₂—, —O—, and —OCH₂—.

In an additional embodiment, the invention is directed to compounds of Formula (I) as referred to herein, and the tautomers and the stereoisomeric forms thereof, wherein L^{A} is selected from the group consisting of —CH₂—, —O—, —OCH₂— and —NHCH₂—.

In a further embodiment, the invention is directed to compounds of Formula (I) as referred to herein, and the tautomers and the stereoisomeric forms thereof, wherein L^{A} is selected from the group consisting of —CH₂—, —O—, and —OCH₂—.

In another embodiment, the invention is directed to compounds of Formula (I) as referred to herein, and the tautomers and the stereoisomeric forms thereof, wherein y is 0.

In another embodiment, the invention is directed to compounds of Formula (I) as referred to herein, and the tautomers and the stereoisomeric forms thereof, wherein R^{D} is H.

In a further embodiment, the invention is directed to compounds of Formula (I), as referred to herein, and the tautomers and the stereoisomeric forms thereof, wherein R^{B} is selected from the group consisting of (b-1), (b-2), (b-3), (b-4), (b-5), (b-6), (b-8), (b-9) and (b-10).

In another embodiment, the invention is directed to compounds of Formula (I), as referred to herein, and the tautomers and the stereoisomeric forms thereof, wherein R^{B} is selected from the group consisting of (b-1), (b-2), (b-3), (b-4), (b-5), and (b-9).

In another embodiment, the invention is directed to compounds of Formula (I), as referred to herein, and the tautomers and the stereoisomeric forms thereof, wherein R^{B} is selected from the group consisting of (b-1), (b-2), (b-5), and (b-9).

In a further embodiment, the invention is directed to compounds of Formula (I), and the tautomers and the stereoisomeric forms thereof, wherein R^{B} is selected from the group consisting of (b-5), (b-9) and (b-12), in particular (b-5) and (b-9).

In another embodiment, the invention is directed to compounds of Formula (I), as referred to herein, and the tautomers and the stereoisomeric forms thereof, wherein

R^{A} is a heteroaryl radical selected from the group consisting of pyridin-2-yl, pyridin-4-yl and pyrimidin-4-yl, each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents;
L^{A} is selected from the group consisting of a covalent bond, —CH₂—, —O—, —OCH₂—, —CH₂O—, —NH—, —NHCH₂— and —CH₂NH—;
x represents 0 or 1;

### R is H or CH₃; and

R^{B} is selected from the group consisting of (b-1), (b-2), (b-3), (b-4), (b-5), (b-6), (b-8), (b-9) and (b-10); wherein
X^{1a} and X^{1b} each independently represents CH or N; and Y¹ represents O or S, with the proviso that at least one of X^{1a} and X^{1b} is CH, and when Y¹ is S, X^{1a} or X^{1b} is N;
X² represents CH or N; and Y² represents O or S;
X³ is N and X⁴ CF;
one or two of Y³-Y⁵ each independently represent ═N—, >NH, or S, with the proviso that up to one of Y³—Y⁵ may be S when present; and the remaining Y³-Y⁵ are each independently selected from the group consisting of CH and C(C₁₋₄alkyl);
X⁵ represents CH or N;
one of Y⁶ or Y⁷ is ═N— and the other is >NH or >NCH₃;
X⁶, X⁷ and X⁸ each independently represent CH or N, with the proviso that up to one of them can be N and with the proviso that X⁷ is C when b is the point of attachment to CHR;
Y⁸ and Y⁹ are each O or S;
n is 1;

### R^{D} is H; and

y is 0;
and the pharmaceutically acceptable salts and the solvates thereof.

In a further embodiment, the invention is directed to compounds of Formula (I), as referred to herein, and the tautomers and the stereoisomeric forms thereof, wherein

R^{A} is a heteroaryl radical selected from the group consisting of pyridin-4-yl and pyrimidin-4-yl, each of which may be optionally substituted with 1, 2 or 3 substituents each independently selected from the group consisting of halo; C₁₋₄ alkyl optionally substituted with 1, 2, or 3 independently selected halo substituents; and C₁₋₄alkyloxy optionally substituted with 1, 2, or 3 independently selected halo substituents;
L^{A} is selected from the group consisting of a covalent bond, —CH₂—, —O—, —OCH₂—, and —CH₂O—;
x represents 0 or 1;

### R is H or CH₃; and

R^{B} is selected from the group consisting of (b-1), (b-2), (b-3), (b-4), (b-5), (b-6), (b-8), (b-9) and (b-10); wherein
X^{1a} and X^{1b} each independently represents CH or N; and Y¹ represents O or S, with the proviso that at least one of X^{1a} and X^{1b} is CH, and when Y¹ is S, X^{1a} or X^{1b} is N;
X² represents CH or N; and Y² represents O or S;
X³ is N and X⁴ CF;
one or two of Y³-Y⁵ each independently represent ═N— or S, with the proviso that up to one of Y³—Y⁵ may be S when present; and the remaining Y³-Y⁵ are each independently selected from the group consisting of CH and C(C₁₋₄alkyl);
X⁵ represents CH or N;
one of Y⁶ or Y⁷ is ═N— and the other is >NH or >NCH₃;
X⁶, X⁷ and X⁸ each independently represent CH or N, with the proviso that up to one of them can be N and with the proviso that X⁷ is C when b is the point of attachment to CHR;
Y⁸ and Y⁹ are each O;
n is 1;

### R^{D} is H; and

y is 0;
and the pharmaceutically acceptable salts and the solvates thereof.

In another embodiment, the invention is directed to compounds of Formula (I), as referred to herein, and the tautomers and the stereoisomeric forms thereof, wherein

R^{A} is a heteroaryl radical selected from the group consisting of pyridin-4-yl and pyrimidin-4-yl, each of which may be optionally substituted with 1, 2 or 3 substituents each independently selected from the group consisting of halo; C₁₋₄ alkyl optionally substituted with 1, 2, or 3 independently selected halo substituents; and C₁₋₄alkyloxy optionally substituted with 1, 2, or 3 independently selected halo substituents;
L^{A} is selected from the group consisting of a covalent bond, —CH₂—, —O—, and —OCH₂—;
x represents 0 or 1;

### R is H or CH₃; and

R^{B} is selected from the group consisting of (b-1), (b-2), (b-3), (b-4), (b-5), (b-6), (b-8), (b-9) and (b-10); wherein
X^{1a} and X^{1b} each independently represents CH or N; and Y¹ represents O or S, with the proviso that at least one of X^{1a} and X^{1b} is CH, and when Y¹ is S, X^{1a} or X^{1b} is N;
X² represents CH or N; and Y² represents O or S;
X³ is N and X⁴ CF;
one or two of Y³-Y⁵ each independently represent ═N— or S, with the proviso that up to one of Y³—Y⁵ may be S when present; and the remaining Y³-Y⁵ are each independently selected from the group consisting of CH and C(C₁₋₄alkyl);
X⁵ represents CH or N;
one of Y⁶ or Y⁷ is ═N— and the other is >NH or >NCH₃;
X⁶, X⁷ and X⁸ each independently represent CH or N, with the proviso that up to one of them can be N and with the proviso that X⁷ is C when b is the point of attachment to CHR;
Y⁸ and Y⁹ are each 0;
n is 1;

### R^{D} is H; and

y is 0;
and the pharmaceutically acceptable salts and the solvates thereof.

In a further embodiment, the invention is directed to compounds of Formula (I), as referred to herein, and the tautomers and the stereoisomeric forms thereof, wherein R^{B} is selected from the group consisting of

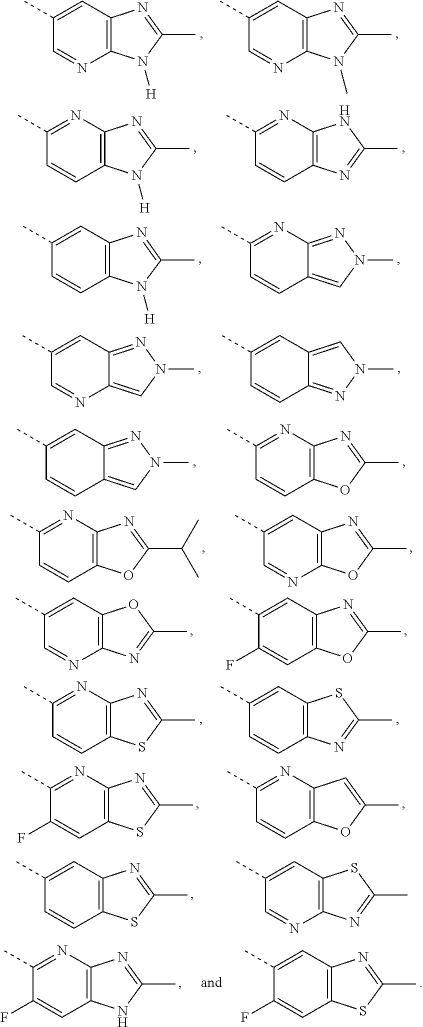

In a further embodiment, the invention is directed to compounds of Formula (I), as referred to herein, and the tautomers and the stereoisomeric forms thereof, wherein R^{B} is selected from the group consisting of

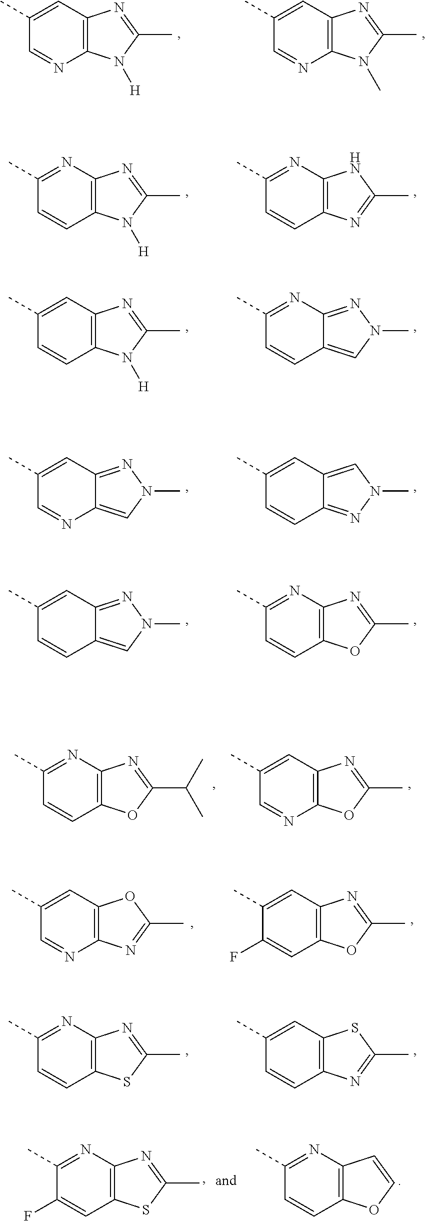

In yet another embodiment, the invention is directed to compounds of Formula (I), as referred to herein, and the tautomers and the stereoisomeric forms thereof, wherein R^{B} is selected from the group consisting of

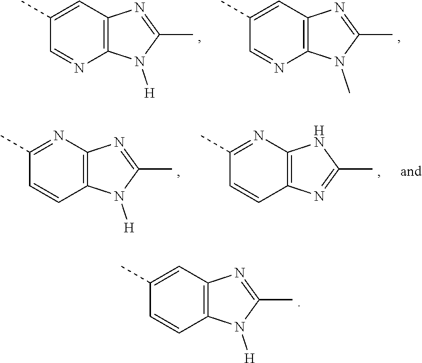

In a further embodiment, the invention is directed to compounds of Formula (I), as referred to herein, and the tautomers and the stereoisomeric forms thereof, wherein R^{B} is selected from the group consisting of

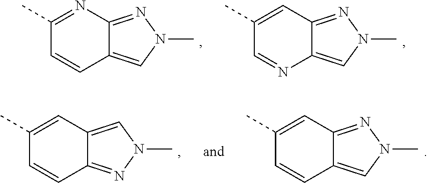

In a further embodiment, the invention is directed to compounds of Formula (I), as referred to herein, and the tautomers and the stereoisomeric forms thereof, wherein R^{B} is selected from the group consisting of

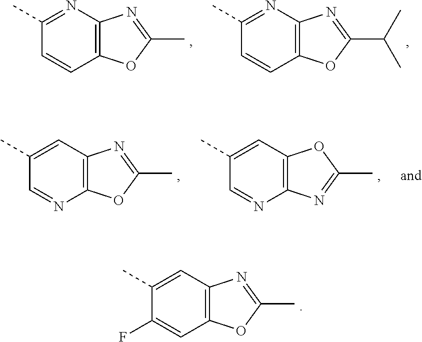

In a further embodiment, the invention is directed to compounds of Formula (I), as referred to herein, and the tautomers and the stereoisomeric forms thereof, wherein R^{B} is selected from the group consisting of

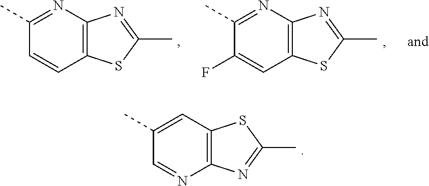

In a further embodiment, the invention is directed to compounds of Formula (I), as referred to herein, and the tautomers and the stereoisomeric forms thereof, wherein

R^{A} is pyridin-4-yl substituted with 1 or 2 substituents each independently selected from C₁₋₄alkyl;
L^{A} is selected from the group consisting of a covalent bond, —CH₂—, —O—, —OCH₂—, and —CH₂—, in particular selected from —CH₂—, —O—, —OCH₂—, and —CH₂O—;
x represents 0 or 1;

### R is CH₃; and

### R^{B} is

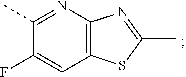

and the pharmaceutically acceptable salts and the solvates thereof.

### Definitions

“Halo” shall denote fluoro, chloro and bromo; “C₁₋₄alkyl” shall denote a straight or branched saturated alkyl group having 1, 2, 3 or 4 carbon atoms, respectively e.g. methyl, ethyl, 1-propyl, 2-propyl, butyl, 1-methyl-propyl, 2-methyl-1-propyl, 1,1-dimethylethyl, and the like; “C₁₋₄alkyloxy” shall denote an ether radical wherein C₁₋₄alkyl is as defined before. When reference is made to L^{A}, the definition is to be read from left to right, with the left part of the linker bound to R^{A} and the right part of the linker bound to the pyrrolidinediyl or piperidinediyl ring. Thus, when L^{A} is, for example, —O—CH₂—, then R^{A}-L^{A}- is R^{A}—O—CH₂—. When R^{C} is present more than once, where possible, it may be bound at the same carbon atom of the pyrrolidinediyl or piperidinediyl ring, and each instance may be different.

In general, whenever the term “substituted” is used in the present invention, it is meant, unless otherwise indicated or is clear from the context, to indicate that one or more hydrogens, in particular 1 to 3 hydrogens, preferably 1 or 2 hydrogens, more preferably 1 hydrogen, on the atom or radical indicated in the expression using “substituted” are replaced with a selection of substituents from the indicated group, provided that the normal valency is not exceeded, and that the substitution results in a chemically stable compound, i.e. a compound that is sufficiently robust to survive isolation to a useful degree of purity from a reaction mixture, and formulation into a therapeutic agent.

The term “subject” as used herein, refers to an animal, preferably a mammal, most preferably a human, who is or has been the object of treatment, observation or experiment. As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. The term “prophylactically effective amount” as used herein, means that amount of active compound or pharmaceutical agent that substantially reduces the potential for onset of the disease or disorder being prevented.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

### Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

### Experimental Procedure 1

The final compounds of Formulae (I-a), (I-b) or (I-c) can be prepared cleaving a protecting group in intermediate compounds of Formulae (IIa), (IIb) or (IIc) according to reaction scheme (1). In reaction scheme (1) all variables are defined as in Formula (I), and PG is a suitable protecting group of the nitrogen function such as, for example, 2-(trimethylsilyl)ethoxymethyl (SEM), tert-butoxycarbonyl (Boc), ethoxycarbonyl, benzyl, benzyloxycarbonyl (Cbz). Suitable methods for removing such protecting groups are widely known to the person skilled in the art and comprise but are not limited to: SEM deprotection: treatment with a protic acid, such as, for example, trifluoroacetic acid, in a reaction inert solvent, such as, for example, dichloromethane; Boc deprotection: treatment with a protic acid, such as, for example, trifluoroacetic acid, in a reaction inert solvent, such as, for example, dichloromethane; ethoxycarbonyl deprotection: treatment with a strong base, such as, for example, sodium hydroxide, in a reaction inert solvent such as for example wet tetrahydrofuran; benzyl deprotection: catalytic hydrogenation in the presence of a suitable catalyst, such as, for example, palladium on carbon, in a reaction inert solvent, such as, for example, ethanol; benzyloxycarbonyl deprotection: catalytic hydrogenation in the presence of a suitable catalyst, such as, for example, palladium on carbon, in a reaction inert solvent, such as, for example, ethanol. In reaction scheme (1) all variables are defined as in Formula (I). For simplicity only one of the two possible N-substituted regiosiomers on the imidazo ring is shown.

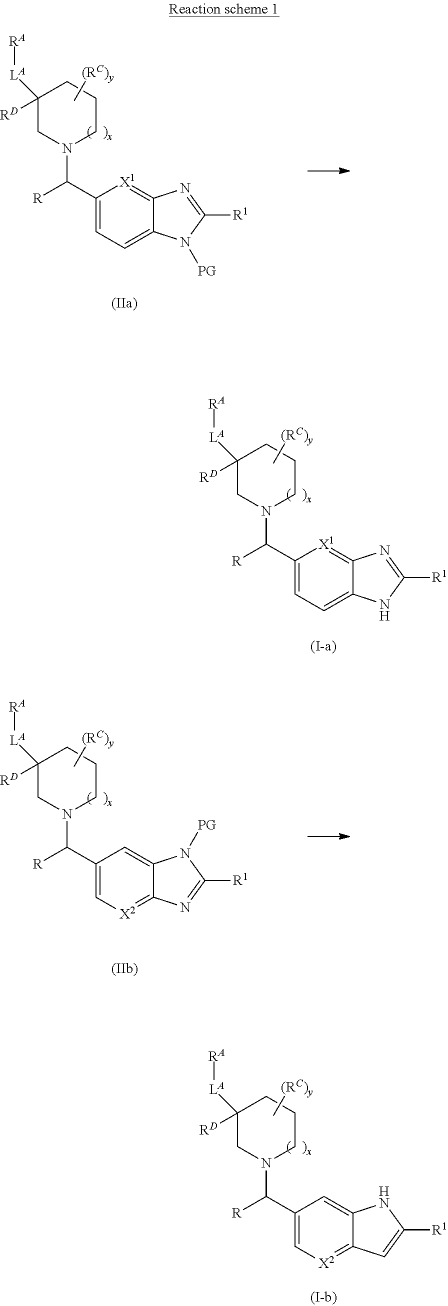

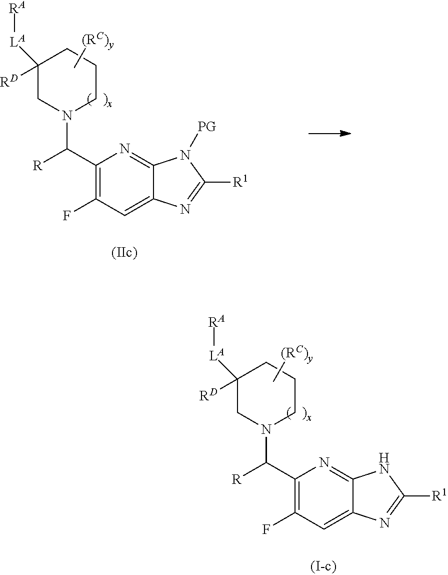

### Experimental Procedure 2

The final compounds of Formula (I-d) can be prepared by reacting an intermediate compound of Formula (III) with a compound of Formula (IV) according to reaction scheme (2). The reaction is performed in a suitable reaction-inert solvent, such as, for example, dichloromethane or 1,2-dichloroethane, a metal hydride, such as, for example sodium triacetoxyborohydride, sodium cyanoborohydride or sodium borohydride and may require the presence of a suitable base, such as, for example, triethylamine or diisopropylethylamine, and/or a Lewis acid, such as, for example titanium tetraisopropoxide or titanium tetrachloride, under thermal conditions, such as, 0° C. to 140° C., more in particular at 0° C., or at room temperature, or at 140° C., for example for 1 hour or 24 hours. In reaction scheme (2) all variables are defined as in Formula (I).

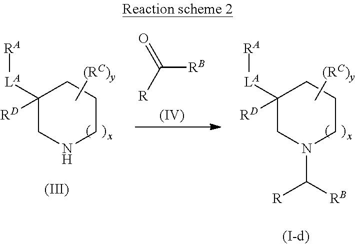

### Experimental Procedure 3

Additionally, final compounds of Formula (I-d) can be prepared by reacting an intermediate compound of Formula (III) with a compound of Formula (V) followed by reaction of the formed imine derivative with and intermediate compound of Formula (VI) according to reaction scheme (3). The reaction is performed in a suitable reaction-inert solvent, such as, for example, anhydrous dichloromethane, a Lewis acid, such as, for example titanium tetraisopropoxide or titanium tetrachloride, under thermal conditions, such as, 0° C. to room temperature, for example for 1 hour or 24 hours. In reaction scheme (3) all variables are defined as in Formula (I), and wherein halo is chloro, bromo or iodo.

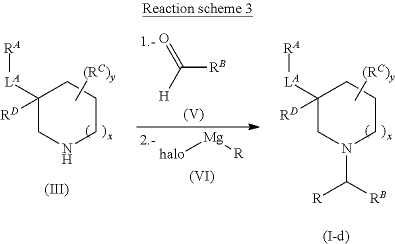

### Experimental Procedure 4

Additionally, final compounds of Formula (I-d) can be prepared by reacting an intermediate compound of Formula (III) with a compound of Formula (VII) according to reaction scheme (4). The reaction is performed in a suitable reaction-inert solvent, such as, for example, acetonitrile, a suitable base, such as, for example, triethylamine or diisopropylethylamine, under thermal conditions, such as, 0° C. to 75° C., in particular, at 0° C., or at room temperature, or at 75° C., for example for 1 hour or 24 hours. In reaction scheme (4) all variables are defined as in Formula (I), and wherein halo is chloro, bromo or iodo.

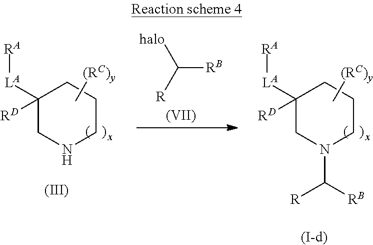

### Experimental Procedure 5

Additionally, final compounds of Formula (I), wherein L^{A} is —NH—CH₂—, herein referred to as (I-e), can be prepared by reacting an intermediate compound of Formula (VIII-a) with a compound of Formula (IX-a) according to reaction scheme (5). The reaction is performed in the presence of a palladium catalyst, such as, for example tris(dibenzylideneacetone)dipalladium(0), a ligand, such as, for example 2-dicyclohexylphosphino-2′-(N,N-dimethylamino)biphenyl, a base, such as, for example sodium tert-butoxide, a suitable reaction-inert solvent, such as, for example, anhydrous 1,4-dioxane, under thermal conditions, such as, at about 100° C., for example for 4 hour or 24 hours. In reaction scheme (5a) all variables are defined as in Formula (I), and wherein halo is chloro, bromo or iodo.

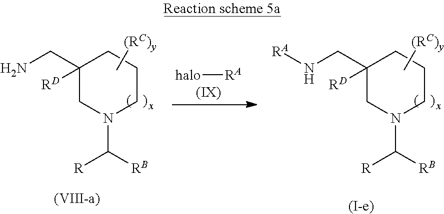

Final compounds of Formula (I), wherein L^{A} is —O—CH₂—, herein referred to as (I-f) can be prepared by “Mitsunobu reaction” of a hydroxy compound of Formula (VIII-b) and a hydroxy derivative of Formula (IX-b) according to reaction scheme (5b). The reaction is performed in a suitable reaction-inert solvent, such as, for example, toluene, a phosphine, such as, triphenylphosphine, a suitable coupling agent, such as, for example DIAD (CAS: 2446-83-5), under thermal conditions, such as, for example, at about 70° C., for example for 17 hours. In reaction scheme (5b) all variables are defined as in Formula (I).

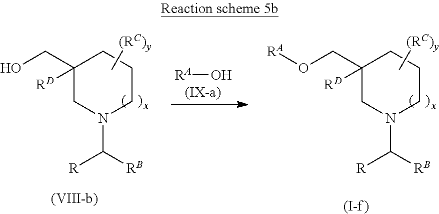

### Experimental Procedure 6

Intermediate compounds of Formulae (IIa), (IIb) or (IIc) can be prepared by reacting an intermediate compound of Formula (III) with a compound of Formulae (Xa), (Xb) or (Xc) followed by reaction of the formed imine derivative with and intermediate compound of Formula (VI) according to reaction scheme (6). The reaction is performed in a suitable reaction-inert solvent, such as, for example, anhydrous dichloromethane, a Lewis acid, such as, for example titanium tetraisopropoxide or titanium tetrachloride, under thermal conditions, such as, 0° C. to room temperature, for example for 1 hour or 24 hours. In reaction scheme (6) all variables are defined as in Formula (I), and wherein halo is chloro, bromo or iodo. For simplicity only one of the two possible N-substituted regiosiomers on the imidazo ring is shown.

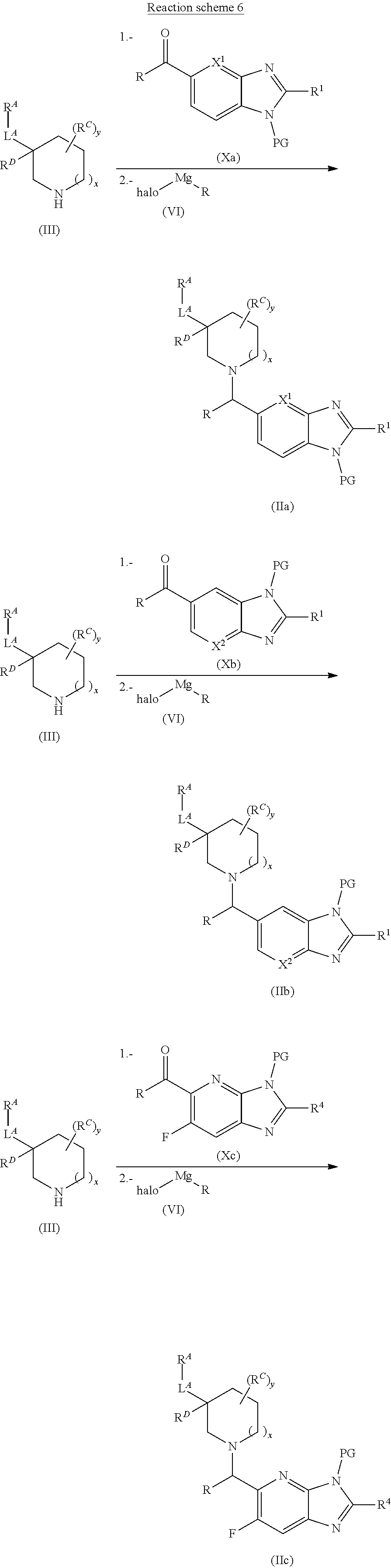

### Experimental Procedure 7

Intermediate compounds of Formula (III) can be prepared by cleaving a protecting group in an intermediate compound of Formula (XI) according to reaction scheme (7). In reaction scheme (7) all variables are defined as in Formula (I), and PG is a suitable protecting group of the nitrogen function such as, for example, tert-butoxycarbonyl (Boc), ethoxycarbonyl, benzyl, benzyloxycarbonyl (Cbz). Suitable methods for removing such protecting groups are widely known to the person skilled in the art and comprise but are not limited to: Boc deprotection: treatment with a protic acid, such as, for example, trifluoroacetic acid, in a reaction inert solvent, such as, for example, dichloromethane or with an acidic resin, such as for example, Amberlist® 15 hydrogen form in a reaction inert solvent such as methanol; ethoxycarbonyl deprotection: treatment with a strong base, such as, for example, sodium hydroxide, in a reaction inert solvent such as for example wet tetrahydrofuran; benzyl deprotection: catalytic hydrogenation in the presence of a suitable catalyst, such as, for example, palladium on carbon, in a reaction inert solvent, such as, for example, ethanol; benzyloxycarbonyl deprotection: catalytic hydrogenation in the presence of a suitable catalyst, such as, for example, palladium on carbon, in a reaction inert solvent, such as, for example, ethanol.

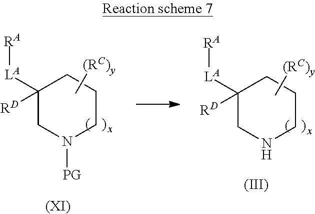

### Experimental Procedure 8

Intermediate compounds of Formula (XI) can be prepared by “Negishi coupling” reaction of a halo compound of Formula (IX) with an organozinc compound of Formula (XII-a) according to reaction scheme (8). The reaction is performed in a suitable reaction-inert solvent, such as, for example, tetrahydrofuran, and a suitable catalyst, such as, for example, Pd(OAc)₂, a suitable ligand for the transition metal, such as, for example, 2-dicyclohexylphosphino-2′,6′-diisopropoxybiphenyl [CAS: 787618-22-8], under thermal conditions, such as, for example, room temperature, for example for 1 hour. In reaction scheme (8) all variables are defined as in Formula (I), L^{A} is a bond or CH₂ and halo is preferably bromo or iodo. PG is defined as in Formula (XI).

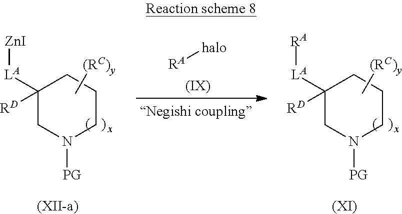

### Experimental Procedure 9

Intermediate compounds of Formula (XII) can be prepared by reaction of a halo compound of Formula (XIII) with zinc according to reaction scheme (9). The reaction is performed in a suitable reaction-inert solvent, such as, for example, tetrahydrofuran, and a suitable salt, such as, for example, lithium chloride, under thermal conditions, such as, for example, 40° C., for example in a continuous-flow reactor. In reaction scheme (9) all variables are defined as in Formula (I), L^{A} is a bond or CH₂ and halo is preferably iodo. PG is defined as in Formula (XI).

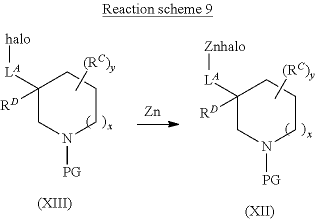

### Experimental Procedure 10

Intermediate compounds of Formula (XI-a) can be prepared by hydrogenation reaction of an alkene compound of Formula (XIV) according to reaction scheme (10). The reaction is performed in a suitable reaction-inert solvent, such as, for example, methanol, and a suitable catalyst, such as, for example, palladium on carbon, and hydrogen, under thermal conditions, such as, for example, room temperature, for example for 3 hours. In reaction scheme (10) all variables are defined as in Formula (I) and PG is defined as in Formula (XI).

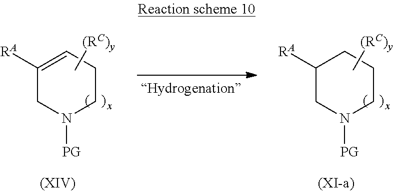

### Experimental Procedure 11

Intermediate compounds of Formula (XIV) can be prepared by “Suzuki coupling” reaction of an alkene compound of Formula (XV) and a halo derivative of Formula (IX) according to reaction scheme (11). The reaction is performed in a suitable reaction-inert solvent, such as, for example, 1,4-dioxane, and a suitable catalyst, such as, for example, tetrakis(triphenylphosphine)palladium(0), a suitable base, such as, for example, NaHCO₃ (aq. sat. soltn.), under thermal conditions, such as, for example, 130° C., for example for 30 min under microwave irradiation. In reaction scheme (11) all variables are defined as in Formula (I), halo is preferably bromo or iodo, L^{A} is a bond, and PG is defined as in Formula (XI).

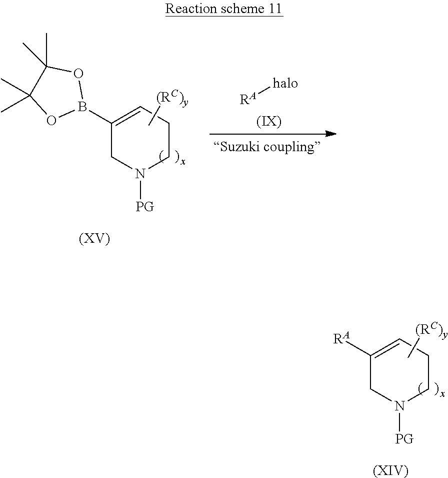

### Experimental Procedure 12

Intermediate compounds of Formula (XI-b) can be prepared by reaction of a hydroxy compound of Formula (XVI) and a halo derivative of Formula (IX) according to reaction scheme (12). The reaction is performed in a suitable reaction-inert solvent, such as, for example, dimethylformamide or dimethylsulfoxide, and a suitable base, such as, sodium hydride or potassium tert-butoxide, under thermal conditions, such as, for example, 50° C., for example for 48 hours. In reaction scheme (12) all variables are defined as in Formula (I), L^{A′} is a bond or CH₂ and halo is preferably chloro, bromo or fluoro. PG is defined as in Formula (XI).

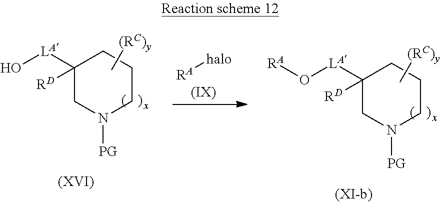

### Experimental Procedure 13

Alternatively, intermediate compounds of Formula (XI-c) can be prepared by “Mitsunobu reaction” of a hydroxy compound of Formula (XVI) and a hydroxy derivative of Formula (IX-a) according to reaction scheme (13). The reaction is performed in a suitable reaction-inert solvent, such as, for example, toluene, a phosphine, such as, triphenylphosphine, a suitable coupling agent, such as, for example DIAD (CAS: 2446-83-5), under thermal conditions, such as, for example, 70° C., for example for 17 hours. In reaction scheme (13) all variables are defined as in Formula (I), L^{A} is a bond or CH₂ and halo is preferably chloro, bromo or fluoro. PG is defined as in Formula (XI).

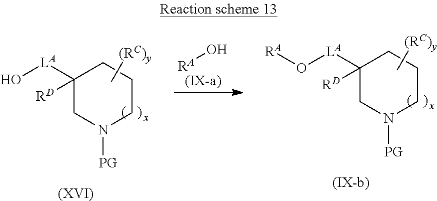

### Experimental Procedure 14

Intermediate compounds of Formula (VIII-b) can be prepared by deprotecting the alcohol group in an intermediate compound of Formula (XVII) according to reaction scheme (14). The reaction is performed in the presence of a fluoride source, such as, for example tetrabutylammonium fluoride, in a suitable reaction-inert solvent, such as, for example, dry tetrahydrofuran, under thermal conditions, such as, for example, room temperature, for example for 16 hours. In reaction scheme (14) all variables are defined as in Formula (I) and PG′ is selected from the group consisting of trimethylsilyl, tert-butyldimethylsilyl, triisopropylsilyl or tert-butyldiphenylsilyl.

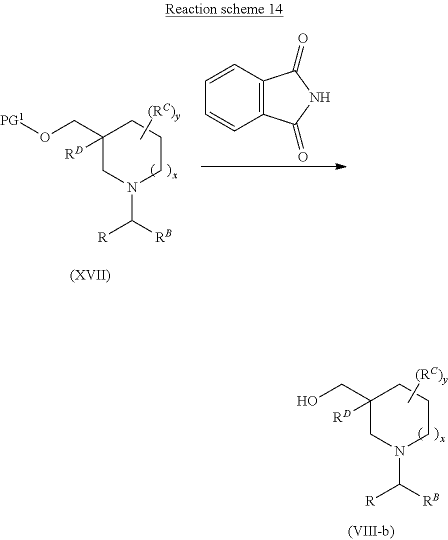

Intermediates of Formulae (IV), (V), (VI), (VII), (VIII-a), (VIII-b), (IX), (IX-a), (Xa), (Xb), (Xc), (XV), (XVI) and (XVII) are commercially available or can be prepared by known procedures to those skilled in the art.

### Pharmacology

The compounds of the present invention and the pharmaceutically acceptable compositions thereof inhibit O-GlcNAc hydrolase (OGA) and therefore may be useful in the treatment or prevention of diseases involving tau pathology, also known as tauopathies, and diseases with tau inclusions. Such diseases include, but are not limited to Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

As used herein, the term “treatment” is intended to refer to all processes, wherein there may be a slowing, interrupting, arresting or stopping of the progression of a disease or an alleviation of symptoms, but does not necessarily indicate a total elimination of all symptoms. As used herein, the term “prevention” is intended to refer to all processes, wherein there may be a slowing, interrupting, arresting or stopping of the onset of a disease.

The invention also relates to a compound according to the general Formula (I), a stereoisomeric form thereof or a pharmaceutically acceptable acid or base addition salt thereof, for use in the treatment or prevention of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

The invention also relates to a compound according to the general Formula (I), a stereoisomeric form thereof or a pharmaceutically acceptable acid or base addition salt thereof, for use in the treatment, prevention, amelioration, control or reduction of the risk of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Stráussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

In particular, the diseases or conditions may in particular be selected from a tauopathy, more in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or the diseases or conditions may in particular be neurodegenerative diseases accompanied by a tau pathology, more in particular a neurodegenerative disease selected from amyotrophic lateral sclerosis or frontotemporal lobe dementia caused by C9ORF72 mutations.

Preclinical States in Alzheimer's and Tauopathy Diseases:

In recent years the United States (US) National Institute for Aging and the International Working Group have proposed guidelines to better define the preclinical (asymptomatic) stages of AD (Dubois B, et al. Lancet Neurol. 2014; 13:614-629; Sperling, R A, et al. Alzheimers Dement. 2011; 7:280-292). Hypothetical models postulate that Aβ accumulation and tau-aggregation begins many years before the onset of overt clinical impairment. The key risk factors for elevated amyloid accumulation, tau-aggregation and development of AD are age (ie, 65 years or older), APOE genotype, and family history. Approximately one third of clinically normal older individuals over 75 years of age demonstrate evidence of AP or tau accumulation on PET amyloid and tau imaging studies, the latter being less advanced currently. In addition, reduced Abeta-levels in CSF measurements are observed, whereas levels of non-modified as well as phosphorylated tau are elevated in CSF. Similar findings are seen in large autopsy studies and it has been shown that tau aggregates are detected in the brain as early as 20 years of age and younger. Amyloid-positive (Aβ+) clinically normal individuals consistently demonstrate evidence of an “AD-like endophenotype” on other biomarkers, including disrupted functional network activity in both functional magnetic resonance imaging (MRI) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases Aβ production or the aggregation of tau is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease or tauopathies. Cognitively normal individuals with amyloid beta or tau aggregation evident on PET scans, or changes in CSF Abeta, tau and phospho-tau are defined as being in an “asymptomatic at risk state for Alzheimer's disease (AR-AD)” or in a “asymptomatic state of tauopathy”. Individuals with a fully penetrant dominant autosomal mutation for familial Alzheimer's disease are said to have “presymptomatic Alzheimer's disease”. Dominant autosomal mutations within the tau-protein have been described for multiple forms of tauopathies as well.

Thus, in an embodiment, the invention also relates to a compound according to the general Formula (I), a stereoisomeric form thereof or a pharmaceutically acceptable acid or base addition salt thereof, for use in control or reduction of the risk of preclinical Alzheimer's disease, prodromal Alzheimer's disease, or tau-related neurodegeneration as observed in different forms of tauopathies.

As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a prophylactically or a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a prophylactically or a therapeutically effective amount of a compound according to the invention to a subject in need thereof.

The invention also relates to a method for modulating O-GlcNAc hydrolase (OGA) activity, comprising administering to a subject in need thereof, a prophylactically or a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent any of the disorders mentioned above or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agent in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™) of the American Psychiatric Association utilizes terms such as neurocognitive disorders (NCDs) (both major and mild), in particular, neurocognitive disorders due to Alzheimer's disease. Such terms may be used as an alternative nomenclature for some of the diseases or conditions referred to herein by the skilled person.

### Pharmaceutical Compositions

The present invention also provides compositions for preventing or treating diseases in which inhibition of O-GlcNAc hydrolase (OGA) is beneficial, such as Alzheimer's disease, progressive supranuclear palsy, Down's syndrome, frontotemporal lobe dementia, frontotemporal dementia with Parkinsonism-17, Pick's disease, corticobasal degeneration, agryophilic grain disease, amyotrophic lateral sclerosis or frontotemporal lobe dementia caused by C9ORF72 mutations, said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The invention also provides a kit comprising a compound according to the invention, prescribing information also known as “leaflet”, a blister package or bottle, and a container. Furthermore, the invention provides a kit comprising a pharmaceutical composition according to the invention, prescribing information also known as “leaflet”, a blister package or bottle, and a container. The prescribing information preferably includes advice or instructions to a patient regarding the administration of the compound or the pharmaceutical composition according to the invention. In particular, the prescribing information includes advice or instruction to a patient regarding the administration of said compound or pharmaceutical composition according to the invention, on how the compound or the pharmaceutical composition according to the invention is to be used, for the prevention and/or treatment of a tauopathy in a subject in need thereof. Thus, in an embodiment, the invention provides a kit of parts comprising a compound of Formula (I) or a stereoisomeric for thereof, or a pharmaceutically acceptable salt or a solvate thereof, or a pharmaceutical composition comprising said compound, and instructions for preventing or treating a tauopathy. The kit referred to herein can be, in particular, a pharmaceutical package suitable for commercial sale.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### Experimental Part

Hereinafter, the term “m.p.” means melting point, “min” means minutes, “ACN”, “MeCN” or “CH₃CN” mean acetonitrile, “aq.” means aqueous, “DMF” means dimethylformamide, “r.t.” or “rt” means room temperature, “rac” or “RS” means racemic, “sat.” means saturated, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “iPrOH” means isopropyl alcohol, “RP” means reversed phase, “t” means retention time (in minutes), “[M+H]⁺” means the protonated mass of the free base of the compound, “wt” means weight, “THF” means tetrahydrofuran, “EtOAc” means ethyl acetate, “DCM” means dichloromethane, “DIPEA” means N,N-diisopropylethylamine, “MeOH” means methanol, “sat” means saturated, “soltn” or “sol.” means solution, “EtOH” means ethanol, and “NMP” means N-methylpyrrolidone, “Pd(PPh₃)₄” means tetrakis(triphenylphosphine)palladium(0), “Pd(PPh₃)₂Cl₂” means bis(triphenylphosphine)palladium(II) dichloride, “Pd(t-Bu₃P)₂” means bis(di-tert-butylphosphine)palladium(0), “PdCl₂(dppf)” means [1,1′-bis(diphenylphosphino)ferrocene]dichloropalladium(II), “DavePhos” means 2-dicyclohexylphosphino-2′-(N,N-dimethylamino)biphenyl, “tBuXPhos” means 2-di-tert-butylphosphino-2′,4′,6′-triisopropylbiphenyl and “Pd₂(dba)₃” means tris(dibenzylideneacetone)dipalladium(0).

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centers has been designated as “R*” or “S*” when the absolute stereochemistry is undetermined although the compound itself has been isolated as a single stereoisomer and is enantiomerically/diastereomerically pure. The enantiomeric excess of compounds reported herein was determined by analysis of the racemic mixture by supercritical fluid chromatography (SFC) followed by SFC comparison of the separated enantiomer(s).

Flow chemistry reactions were performed in a Vapourtec R2+R4 unit using standard reactors provided by the vendor.

Microwave assisted reactions were performed in a single-mode reactor: Initiator™ Sixty EXP microwave reactor (Biotage AB), or in a multimode reactor: MicroSYNTH Labstation (Milestone, Inc.).

Thin layer chromatography (TLC) was carried out on silica gel 60 F254 plates (Merck) using reagent grade solvents. Open column chromatography was performed on silica gel, particle size 60 Å, mesh=230-400 (Merck) using standard techniques. Automated flash column chromatography was performed using ready-to-connect cartridges, on irregular silica gel, particle size 15-40 μm (normal phase disposable flash columns) on different flash systems: either a SPOT or LAFLASH systems from Armen Instrument, or PuriFlash® 430evo systems from Interchim, or 971-FP systems from Agilent, or Isolera 1SV systems from Biotage.

### Preparation of Intermediates I-1a, 1b, 1c, 1d and 1e

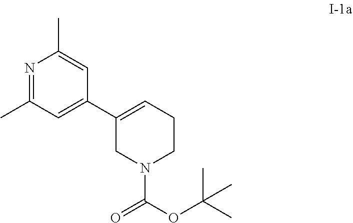 [SMILES: Cc1cc(C2=CCCN(C(=O)OC(C)(C)C)C2)cc(C)n1]

A mixture of 4-chloro-2,6-dimethylpyridine (CAS: 3512-75-2; 2 g, 14.1 mmol), tert-butyl-3-(4,4,5,5-tetramethyl-1,3,2-dioxaborolan-2-yl)-5,6-dihydropyridine-1(2H)-carboxylate (CAS: 1251537-34-4; 4.8 g, 15.5 mmol) and Pd(PPh₃)₄ (CAS: 14221-01-3; 0.98 g, 0.85 mmol) in a deoxygenated mixture of a saturated solution of NaHCO₃ (3 mL) and 1,4-dioxane (24 mL) was stirred in a sealed tube at 130° C. for 30 min under N₂. Then, the mixture was treated with water and extracted with DCM. The organic layer was separated, dried (Na₂SO₄), filtered and the solvents were evaporated in vacuo. The crude product was purified by flash column chromatography (silica; EtOAc in heptane 0/100 to 100/0). The desired fractions were collected and concentrated in vacuo to afford intermediate 1a as a colorless oil (3.8 g, 93%).

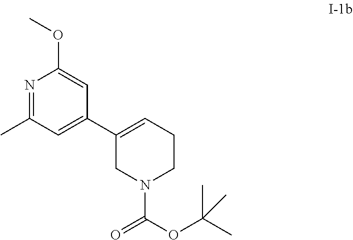 [SMILES: COc1cc(C2=CCCN(C(=O)OC(C)(C)C)C2)cc(C)n1]

Intermediate 1b was prepared following an analogous procedure to the one described for the synthesis of intermediate 1a using 4-bromo-2-methoxy-6-methylpyridine (CAS: 1083169-00-9) as starting material.

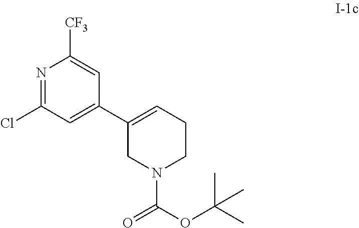 [SMILES: CC(C)(C)OC(=O)N1CCC=C(c2cc(Cl)nc(C(F)(F)F)c2)C1]

trans-Bis(dicyclohexylamine)palladium(II) acetate (DAPcy, CAS: 628339-96-8; 0.114 g, 0.20 mmol) was added to a stirred mixture of 2-chloro-4-iodo-6-trifluoromethylpyridine (CAS: 205444-22-0; 3 g, 9.76 mmol), tert-butyl-3-(4,4,5,5-tetramethyl-1,3,2-dioxaborolan-2-yl)-5,6-dihydropyridine-1(2H)-carboxylate (CAS: 1251537-34-4; 3.62 g, 11.71 mmol) and K₃PO₄ (6.21 g, 29.27 mmol) in EtOH (24 mL) under N₂. The mixture was stirred at rt for 18 h and then filtered through Celite®. The Celite® pad was washed with EtOAc and the filtrate evaporated in vacuo. The crude product was purified by flash column chromatography (silica; EtOAc in heptane, gradient from 0/100 to 20/80). The desired fractions were collected and concentrated in vacuo to afford intermediate 1c as a colorless oil (3.8 g, 93%).

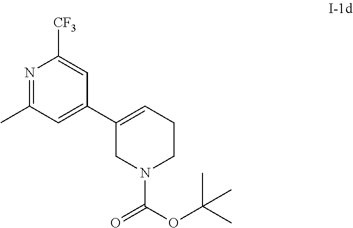 [SMILES: Cc1cc(C2=CCCN(C(=O)OC(C)(C)C)C2)cc(C(F)(F)F)n1]

Pd(OAc)₂ (CAS: 3375-31-3; 0.105 g, 0.47 mmol) and tricyclohexylphosphonium tetrafluoroborate (CAS: 58656-04-5; 0.345 g, 0.94 mmol) were added to a stirred mixture of intermediate 1c (3.4 g, 9.37 mmol), trimethylboroxine (CAS: 823-96-1; 2.36 mL, 16.87 mmol) and K₂CO₃ (2.59 g, 18.74 mmol) in deoxygenated 1,4-dioxane (35 mL) under N₂. The mixture was stirred at 100° C. for 2 h. After cooling to rt, the mixture was washed with H₂O and extracted with DCM. The organic layer was separated, dried (MgSO₄), filtered and the solvents were evaporated in vacuo. The crude product was purified by flash column chromatography (silica; EtOAc in heptane, gradient from 0/100 to 15/85). The desired fractions were collected and concentrated in vacuo to yield intermediate 1d as a pale-yellow oil that crystallized upon standing (2.8 g, 87%).

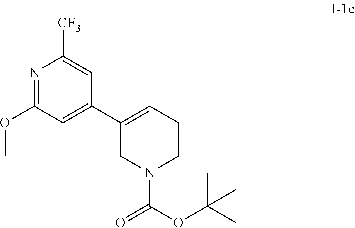 [SMILES: COc1cc(C2=CCCN(C(=O)OC(C)(C)C)C2)cc(C(F)(F)F)n1]

A 25% solution of sodium methoxide in MeOH (2.14 mL, 9.37 mmol) was added to a stirred solution of intermediate 1c (3.4 g, 9.37 mmol) in MeOH (50 mL). The mixture was stirred at rt for 16 h. Then water was added and the desired product was extracted with DCM. The organic layer was separated, dried (Na₂SO₄), filtered and the solvents were evaporated in vacuo. The crude product was purified by flash column chromatography (silica; DCM in heptane, gradient from 20/80 to 100/0). The desired fractions were collected and concentrated in vacuo to yield intermediate 1e as a colorless oil (3.1 g, 92%).

### Preparation of Intermediates I-2a, 2aR, 2aS, 2b, 2c and 2d

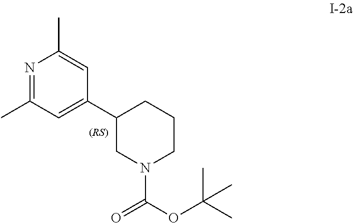 [SMILES: Cc1cc(C2CCCN(C(=O)OC(C)(C)C)C2)cc(C)n1]

A solution of intermediate 1a (3.8 g, 13.18 mmol) in EtOH (250 mL) was hydrogenated in a H-cube (Pd/C 10%, rt, full H₂, 1 ml/min). The solvent was evaporated in vacuo to yield intermediate 2a as a colorless oil that was used in the next step without further purification (2.7 g, 71%).

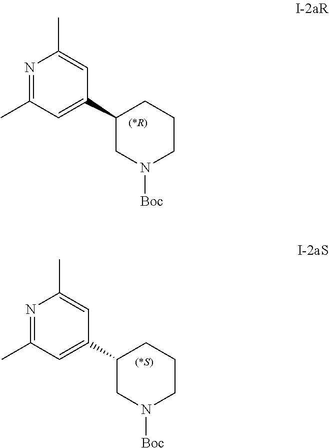 [SMILES: Cc1cc([C@H]2CCCN(C(=O)OC(C)(C)C)C2)cc(C)n1 ; Cc1cc([C@@H]2CCCN(C(=O)OC(C)(C)C)C2)cc(C)n1]

Pd/C (10% purity, 1.18 g, 1.11 mmol) was added to a stirred solution of intermediate 1a (3.20 g, 11.1 mmol) in EtOH (64.1 mL). The reaction mixture was hydrogenated (atmospheric pressure) at room temperature for 16 h. The mixture was filtered through a pad of Celite® and washed with MeOH. The filtrate was concentrated in vacuo. The residue was purified by flash column chromatography (Sift, EtOAc in heptane, gradient from 100:0 to 20:80) to afford intermediate 2a (3.10 g, 96%). A second purification was performed via chiral SFC (stationary phase: CHIRALPAK IC 5 μm 250*30 mm, mobile phase: 65% CO₂, 35% i-PrOH (0.3% i-PrNH₂) to afford intermediate 2aR (1.3 g, 40%) and intermediate 2aS (1.44 g, 45%).

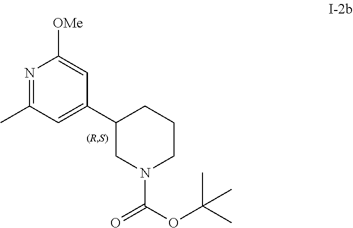 [SMILES: COc1cc(C2CCCN(C(=O)OC(C)(C)C)C2)cc(C)n1]

Intermediate 2b was prepared following an analogous procedure to the one described for the synthesis of intermediate 2a using intermediate 1b as starting material.

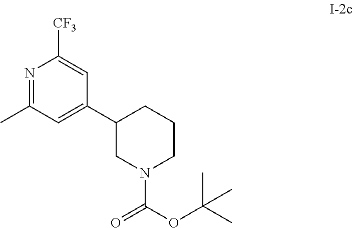 [SMILES: Cc1cc(C2CCCN(C(=O)OC(C)(C)C)C2)cc(C(F)(F)F)n1]

Intermediate 2c was prepared following an analogous procedure to the one described for the synthesis of intermediate 2a using intermediate 1d as starting material.

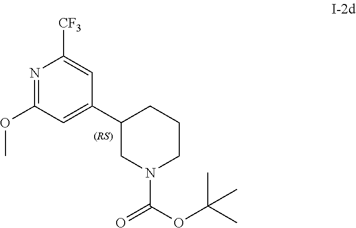 [SMILES: COc1cc(C2CCCN(C(=O)OC(C)(C)C)C2)cc(C(F)(F)F)n1]

Intermediate 2d was prepared following an analogous procedure to the one described for the synthesis of intermediate 2a using intermediate 1e as starting material.

### Preparation of Intermediates I-3a, I-3aR, 3b, 3c and 3d

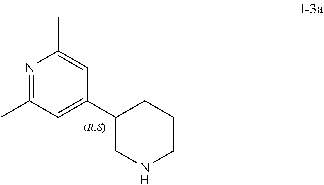

Amberlyst® 15 hydrogen form, strongly acidic, cation exchanger resin (CAS: 39389-20-3; 4 meq/g, 9.3 g) was added to a solution of intermediate 2a (2.7 g, 9.30 mmol) in MeOH (47 mL). The mixture was shaken in a solid phase reactor at rt for 16 h. The resin was washed with MeOH (filtrate discarded) and then with a 7N solution of NH₃ in MeOH. The filtrate was concentrated in vacuo to yield intermediate 3a as an orange oil (1.2 g, 68%).

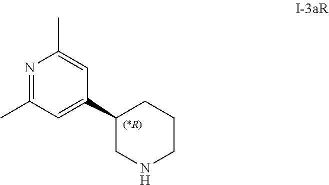

A solution of intermediate 2aR (1.30 g, 4.48 mmol) in MeOH (34.4 mL) was added to a closed reactor containing Amberlyst®15 hydrogen form (CAS: 39389-20-3; 4.76 g, 22.4 mmol). The reaction mixture was shaken in a solid phase reactor at room temperature for 16 h. The resin was washed with MeOH (the fraction was discarded). NH₃ (7N in MeOH) (34 mL) was added and the mixture was shaken in the solid phase reactor for 2 h. The resin was filtered and was washed with NH₃ (7N in MeOH) (3×34 mL; 30 min shaken). The filtrates were concentrated in vacuo to afford intermediate 3aR (820 mg, 96%).

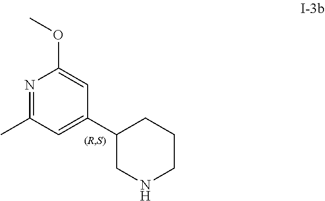

Intermediate 3b was prepared following an analogous procedure to the one described for the synthesis of intermediate 3a using intermediate 2b as starting material. Intermediate 3b was purified by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN).

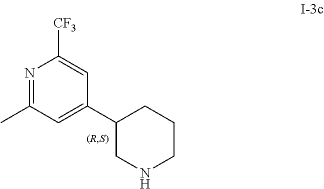 [SMILES: Cc1cc(C2CCCNC2)cc(C(F)(F)F)n1]

Intermediate 3c was prepared following an analogous procedure to the one described for the synthesis of intermediate 3a using intermediate 2c as starting material. Intermediate 3c was purified by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN).

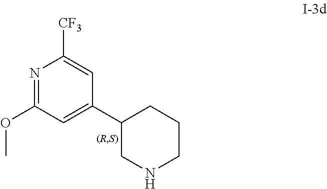 [SMILES: COc1cc(C2CCCNC2)cc(C(F)(F)F)n1]

Intermediate 3d was prepared following an analogous procedure to the one described for the synthesis of intermediate 3a using intermediate 2d as starting material. Intermediate 3b was purified by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN).

### Preparation of Intermediates I-4a, 4b, 4c, 4d and 4e

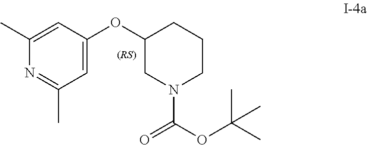 [SMILES: Cc1cc(OC2CCCN(C(=O)OC(C)(C)C)C2)cc(C)n1]

Sodium hydride (CAS: 7646-69-7; 60% dispersion in mineral oil, 0.30 g, 7.45 mmol) was added to a stirred solution of 1-Boc-3-hydroxypiperidine (CAS: 85275-45-2; 1.5 g, 7.45 mmol) in DMF (6 mL) at 0° C. and the mixture was stirred for 30 min. The mixture was allowed to warm to rt and a solution of 2,6-dimethyl-4-chloropyridine (CAS: 3512-75-2; 0.95 mL g, 7.45 mmol) in DMF (1 mL) was added dropwise. The mixture was stirred at rt for 16 h and then at 60° C. for 6 h. After cooling to rt, water was added and the mixture was extracted with EtOAc. The organic layer was dried (Na₂SO₄), filtered and concentrated in vacuo. The residue was purified by flash chromatography (silica; EtOAc in heptane, gradient from 0/100 to 30/70). The desired fractions were collected and concentrated in vacuo to yield intermediate 4a as a colorless oil (0.42 g, 18%).

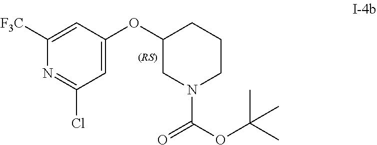 [SMILES: CC(C)(C)OC(=O)N1CCCC(Oc2cc(Cl)nc(C(F)(F)F)c2)C1]

Sodium hydride (CAS: 7646-69-7; 60% dispersion in mineral oil, 0.50 g, 12.42 mmol) was added to a stirred solution of 1-Boc-3-hydroxypiperidine (CAS: 85275-45-2; 2.5 g, 12.42 mmol) in DMF (14 mL) at −40° C. The mixture was stirred at −40° C. for 30 min and then a solution of 2-chloro-4-iodo-6-trifluoromethylpyridine (CAS: 205444-22-0; 3.82 g, 12.42 mmol) in DMF (4 mL) was added dropwise. The mixture was allowed to warm to rt and then was stirred for 16 h. Then the mixture was diluted with EtOAc and washed with water and brine. The organic layer was dried (Na₂SO₄), filtered and concentrated in vacuo. The residue was purified by flash chromatography (silica; EtOAc in heptane, gradient from 0/100 to 50/50). The desired fractions were collected and concentrated in vacuo to yield intermediate 4d as a light-yellow oil (2.8 g, 59%).

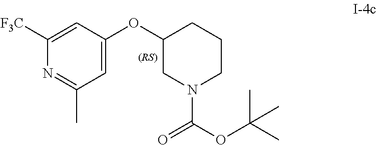 [SMILES: Cc1cc(OC2CCCN(C(=O)OC(C)(C)C)C2)cc(C(F)(F)F)n1]

Intermediate 4c was prepared following an analogous procedure to the one described for the synthesis of intermediate 1d using intermediate 4b as starting material.

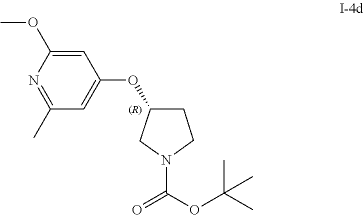

Sodium hydride (CAS: 7646-69-7; 60% dispersion in mineral oil, 0.32 g, 8.01 mmol) was added to a stirred solution of (3R)-1-(Boc)-3-hydroxypyrrolidine (CAS: 109431-87-0; 1.5 g, 8.01 mmol) in DMF (6.4 mL) at 0° C. and the mixture was stirred for 30 min. Then the mixture was allowed to warm to rt and a solution of 4-bromo-2-methoxy-6-methylpyridine (CAS: 1083169-00-9; 1.48 mL, 8.01 mmol) was added dropwise. The mixture was stirred at 60° C. for 16 h. After cooling to rt, water was added and the mixture was extracted with EtOAc. The organic layer was dried (Na₂SO₄), filtered and concentrated in vacuo. The residue was purified by flash chromatography (silica; EtOAc in heptane, gradient from 0/100 to 30/70). The desired fractions were collected and concentrated in vacuo to yield intermediate 4d as a colorless oil (1.67 g, 67%).

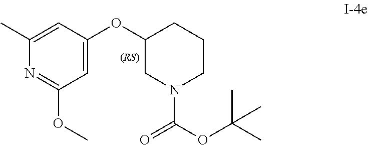 [SMILES: COc1cc(OC2CCCN(C(=O)OC(C)(C)C)C2)cc(C)n1]

Intermediate 4c was prepared following an analogous procedure to the one described for the synthesis of intermediate 4a using 4-bromo-2-methoxy-6-methylpyridine (CAS: 1083169-00-9) as starting material.

### Preparation of Intermediates I-5a, 5b, 5c and 5d

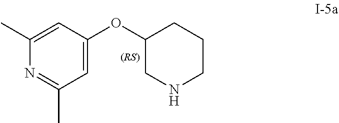

Intermediate 5a was prepared following an analogous procedure to the one described for the synthesis of intermediate 3a using intermediate 4a as starting material.

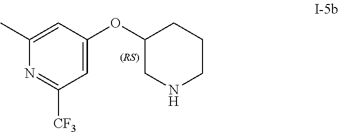 [SMILES: Cc1cc(OC2CCCNC2)cc(C(F)(F)F)n1]

Intermediate 5b was prepared following an analogous procedure to the one described for the synthesis of intermediate 3a using intermediate 4c as starting material.

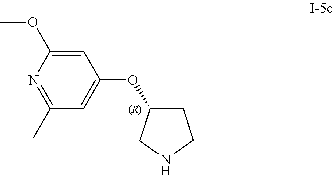

Intermediate 5c was prepared following an analogous procedure to the one described for the synthesis of intermediate 3a using intermediate 4d as starting material.

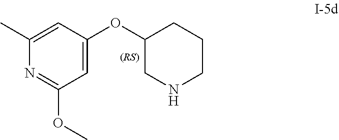 [SMILES: COc1cc(OC2CCCNC2)cc(C)n1]

Intermediate 5d was prepared following an analogous procedure to the one described for the synthesis of intermediate 3a using intermediate 4e as starting material.

### Preparation of Intermediates I-6a, 6b and 6c

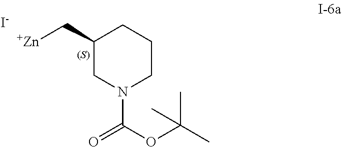

A solution of (3S)-1-Boc-3-iodomethylpiperidine (CAS: 384829-99-6; 35 g, 107.6 mmol) in a 0.5 M solution of LiCl in THF (192.5 mL, 96.3 mmol) was pumped through a column containing activated Zn (9.35 g, 143.0 mmol) at 40° C. with flow of 1 mL/min. The outcome solution was collected under N₂ atmosphere to yield intermediate 6a as a clear light-brown solution that was used without any further manipulation.

For the above reaction Zn was activated as follows: A solution of TMSCl (2.5 mL) and 1-bromo-2-choroethane (0.3 mL) in THF (10 mL) was passed through the column containing Zn at a flow of 1 mL/min.

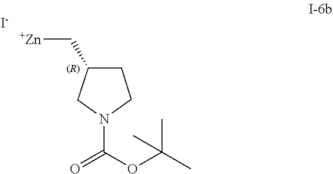

A solution of (3R)-1-Boc-3-iodomethylpyrrolidine (CAS: 1187932-69-9; 10.1 g, 32.4 mmol) in THF (65 mL) was pumped through a column containing activated Zn (30 g, 458.8 mmol) at 40° C. with a flow of 1 mL/min. The outcome solution was collected under N₂ atmosphere to yield intermediate 6b as a clear solution that was used without any further manipulation.

For the above reaction Zn was activated as follows: A solution of TMSCl (2 mL) and 1-bromo-2-choroethane (1.2 mL) in THF (20 mL) was passed through the column containing Zn at a flow of 1 mL/min.

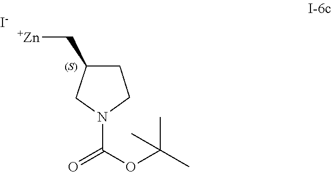

Intermediate 6c was prepared following an analogous procedure to the one described for the synthesis of intermediate 6b using (3S)-1-Boc-3-iodomethylpyrrolidine (CAS: 224168-68-7) as starting material.

### Preparation of Intermediates I-7a, 7b, 7c, 7d, 7e, 7f, 7g, 7h, 7i, 7j and 7k

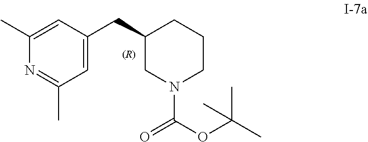

N,N,N′,N′-Tetramethylethylenediamine (CAS: 110-18-9; 11.97 mL, 79.8 mmol), 4-bromo-2,6-dimethylpyridine (CAS: 5093-70-9; 13.50 g, 72.55 mmol) and Pd(PPh₃)₂Cl₂ (1.02 g, 1.45 mmol) were added to a stirred 0.38 M solution of intermediate 6a in THF (210 mL, 79.8 mmol) in a 400 mL EasyMax® reactor equipped with an overhead stirrer and a temperature probe at rt. The mixture was degassed with N₂ and then stirred at 65° C. (internal temperature) for 16 h. After cooling to 20° C., a mixture of a 32% solution of NH₃ (50 mL) and a saturated solution of NH₄Cl (50 mL) were added. The mixture was diluted with water (100 mL) and EtOAc (200 mL) and filtered through a Celite® pad. The organic layer was separated, washed with brine, dried (MgSO4), filtered and the solvents evaporated in vacuo. The crude product was purified by flash column chromatography (silica, EtOAc in heptane, gradient from 0/100 to 50/50). The desired fractions were collected and concentrated in vacuo to yield intermediate 7a as an orange oil (18.5 g, 84% yield).

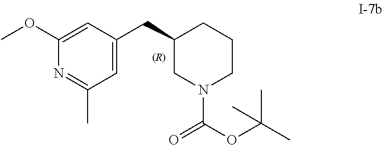

A 0.36 M solution of intermediate 6a in THF (42 mL, 15.12 mmol) was added to a stirred mixture of 4-bromo-2-methoxy-6-methylpyridine (CAS: 1083169-00-9; 2.98 g, 14.75 mmol) and Pd(t-Bu₃P)₂ (0.22 g, 0.31 mmol) at rt under N₂. The mixture was stirred at reflux for 16 h. After cooling to rt a (1:1) mixture of a 32% solution of NH₃ (50 mL) and a saturated solution of NH₄Cl (50 mL) was added. The mixture was extracted with EtOAc (200 mL). The organic layer was separated, dried (MgSO₄), filtered and the solvents were evaporated in vacuo. The crude product was purified by flash column chromatography (silica, EtOAc in heptane, gradient from 0/100 to 50/50). The desired fractions were collected and concentrated in vacuo to yield intermediate 7b as a colorless oil (4.34 g, 91%).

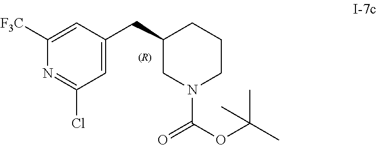

Intermediate 7c was prepared following an analogous procedure to the one described for the synthesis of intermediate 7b using 2-chloro-4-iodo-6-trifluoromethylpyridine (CAS: 205444-22-0) as starting material and stirring the reaction mixture at rt for 1 h.

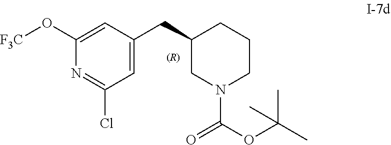

Intermediate 7d was prepared following an analogous procedure to the one described for the synthesis of intermediate 7b using 2-chloro-4-iodo-6-trifluoromethoxypyridine (CAS: 1221171-96-5; prepared according to Eur. J. Org. Chem. 2010, 6043-6066) as starting material and stirring the reaction mixture at 65° C. for 3 h.

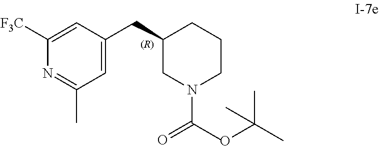

Intermediate 7e was prepared following an analogous procedure to the one described for the synthesis of intermediate 1d using intermediate 7c as starting material.

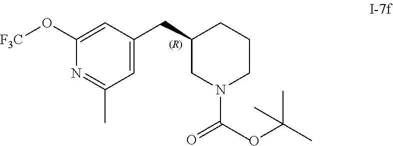

Intermediate 7f was prepared following an analogous procedure to the one described for the synthesis of intermediate 1d using intermediate 7d as starting material.

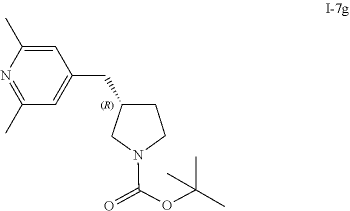

A 0.32 M solution of intermediate 6b (34 mL, 10.88 mmol), N,N,N′,N′-tetramethylethylenediamine (CAS: 110-18-9; 1.63 mL, 10.88 mmol) and Pd(PPh₃)₂Cl₂ (0.42 g, 0.59 mmol) were added to stirred 4-bromo-2,6-dimethylpyridine (CAS: 5093-70-9; 1.84 g, 9.89 mmol) at rt under N₂. The mixture was stirred at 60° C. for 1 h. After cooling to rt, a 1:1 mixture of a 32% solution of NH₃ and a saturated solution of NH₄Cl was added. The mixture was extracted with EtOAc. The organic layer was separated, washed with brine, dried (MgSO₄), filtered and the solvents were evaporated in vacuo. The crude product was purified by flash column chromatography (silica, EtOAc in heptane, gradient from 30/70 to 80/20). The desired fractions were collected and concentrated in vacuo to yield intermediate 7g as an oil (2.5 g, 87% yield).

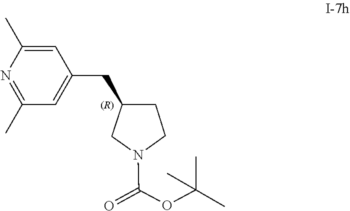

N,N,N′,N′-Tetramethylethylenediamine (CAS: 110-18-9; 4.40 mL, 29.3 mmol), 4-bromo-2,6-dimethylpyrimidine (CAS: 5093-70-9; 4.20 g, 26.4 mmol) and Pd(PPh₃)₂Cl₂ (0.45 g, 0.64 mmol) were added to a stirred 0.35 M solution of intermediate 6c in THF (83 mL, 29.4 mmol) at rt under N₂. The mixture was stirred at reflux for 16 h. After cooling to rt, a 1:1 mixture of a 32% solution of NH₃ and a saturated solution of NH₄Cl was added. The mixture was extracted with EtOAc. The organic layer was separated, dried (MgSO₄), filtered and the solvents were evaporated in vacuo. The crude product was purified by flash column chromatography (silica, EtOAc in heptane, gradient from 0/100 to 100/50). The desired fractions were collected and concentrated in vacuo to yield intermediate 7h as an orange oil (9.07 g, 92% yield).

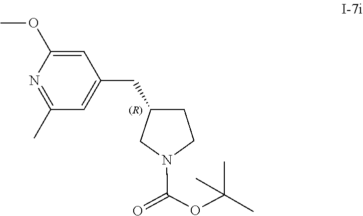

Intermediate 7i was prepared following an analogous procedure to the one described for the synthesis of intermediate 7h using intermediate 6c and 4-bromo-2-methoxy-6-methylpyridine (CAS: 1083169-00-9) as starting materials.

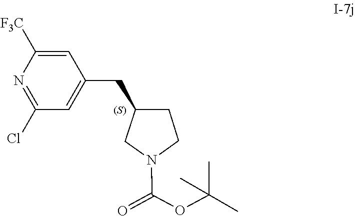

A 0.42 M solution of intermediate 6b (34 mL, 14.3 mmol) was added to a stirred mixture of 2-chloro-4-iodo-6-trifluoromethylpyridine (CAS: 205444-22-0; 4.0 g, 13.01 mmol) and Pd(t-Bu₃P)₂ (0.33 g, 0.65 mmol) at rt under N₂. The mixture was stirred at rt for 1 h and then a 1:1 mixture of a 32% solution of NH₃ and a saturated solution of NH₄Cl was added. The mixture was extracted with EtOAc. The organic layer was separated, dried (Na₂SO₄), filtered and the solvents were evaporated in vacuo. The crude product was purified by flash column chromatography (silica, EtOAc in heptane, gradient from 0/100 to 20/80). The desired fractions were collected and concentrated in vacuo to yield intermediate 7j as a pale-yellow oil (2.50 g, 39%).

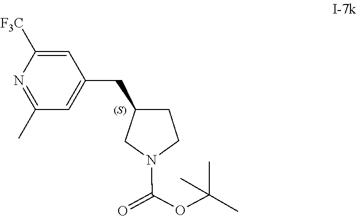

Intermediate 7k was prepared following an analogous procedure to the one described for the synthesis of intermediate 1d using intermediate 7j as starting material.

### Preparation of Intermediates I-8a, 8b, 8c, 8d, 8e, 8f, 8g and 8h

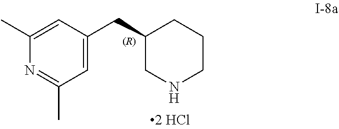

A 4M HCl solution in 1,4-dioxane (CAS: 7647-01-0; 148.4 mL, 593.71 mmol) was added to a stirred solution of intermediate 7a in 2-methyltetrahydrofuran (180.7 mL) at 0° C. under N₂. The mixture was stirred at 0° C. for 30 min and then allowed to warm to 20° C. After 1 h at 20° C., the mixture was warmed to 50° C. and stirred for a further 2 h. The solid formed was filtered off, washed with 2-methyltetrahydrofuran and dried under vacuum at 50° C. for 16 h to yield intermediate 8a.2HCl as a light-yellow solid (15.8, 96%).

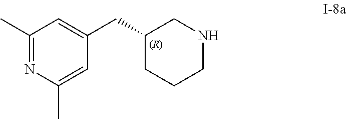

HCl (4M in 1,4-dioxane, 5.5 mL, 22.0 mmol) was added to intermediate 7a (670 mg, 2.20 mmol) at 0° C. and the reaction mixture was warmed to room temperature. The reaction mixture was stirred for 3 days and concentrated to dryness in vacuo. The residue was purified by ion exchange chromatography (ISOLUTE SCX-2, MeOH and then 7N solution of NH₃ in MeOH) to afford intermediate 8a (425 mg, 99%).

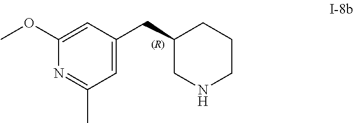

Intermediate 8a was prepared following an analogous procedure to the one described for the synthesis of intermediate 3a using intermediate 7b as starting material.

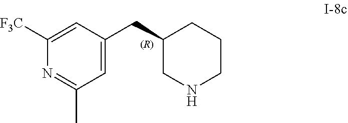

Intermediate 8a was prepared following an analogous procedure to the one described for the synthesis of intermediate 3a using intermediate 7d as starting material.

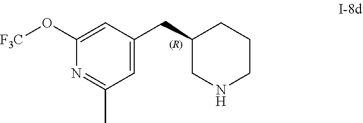

Intermediate 8d was prepared following an analogous procedure to the one described for the synthesis of intermediate 3a using intermediate 7f as starting material.

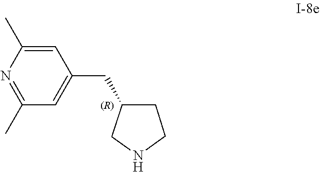

Intermediate 8e was prepared following an analogous procedure to the one described for the synthesis of intermediate 3a using intermediate 7g as starting material.

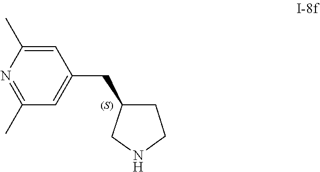

Intermediate 8f was prepared following an analogous procedure to the one described for the synthesis of intermediate 3a using intermediate 7h as starting material.

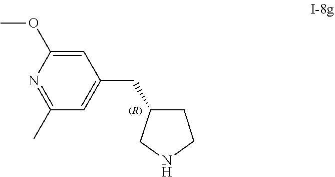

Intermediate 8g was prepared following an analogous procedure to the one described for the synthesis of intermediate 3a using intermediate 7i as starting material.

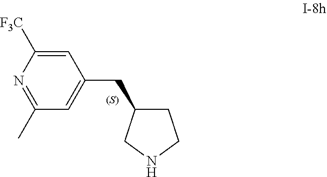

Intermediate 8h was prepared following an analogous procedure to the one described for the synthesis of intermediate 3a using intermediate 7k as starting material.

### Preparation of Intermediates I-9a, 9b, 9c, 9d, 9e and 9f

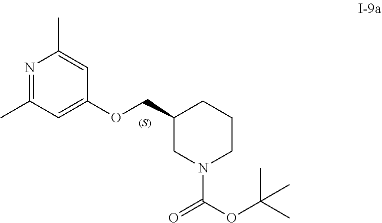

Sodium hydride (CAS: 7646-69-7; 60% dispersion in mineral oil, 0.46 g, 11.61 mmol) was added to a stirred solution of (3S)-1-Boc-3-hydroxymethylpiperidine (CAS: 140695-84-7; 2.5 g, 11.61 mmol) in DMF (10.3 mL) at 0° C. The mixture was stirred at 0° C. for 30 min and then a solution of 4-chloro-2,6-dimethylpyridine (CAS: 3512-75-2; 1.48 mL, 11.61 mmol) in DMF (1.3 mL) was added dropwise. The mixture was stirred at 60° C. for 16 h and then the solvent was evaporated. The residue was diluted with water and extracted with EtOAc. The organic layer was dried (Na₂SO₄), filtered and evaporated in vacuo. The residue was purified by flash column chromatography (SiO₂; EtOAc in heptane, gradient from 0/100 to 30/70). The desired fractions were collected and concentrated in vacuo to yield intermediate 9a as a colorless oil (2.37 g, 64%).

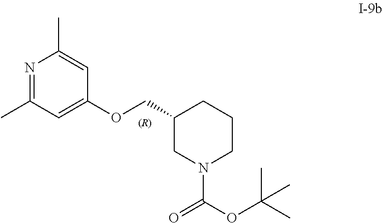

Intermediate 9b was prepared following an analogous procedure to the one described for the synthesis of intermediate 9a using (3R)-1-Boc-3-hydroxymethylpiperidine (CAS: 116574-71-1) as starting material.

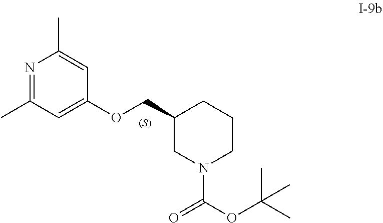

Intermediate 9c was prepared following an analogous procedure to the one described for the synthesis of intermediate 9a using 4-chloro-2,6-pyrimidine (CAS: 4472-45-1) as starting material.

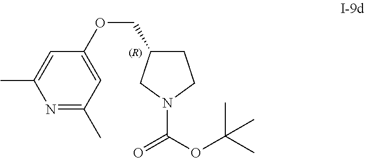

Sodium hydride (CAS: 7646-69-7; 60% dispersion in mineral oil, 0.24 g, 9.96 mmol) was added to a stirred solution of (3R)-1-Boc-3-hydroxymethylpyrrolidine (CAS: 138108-72-2; 1.0 g, 4.97 mmol) in DMF (10 mL) at 0° C. under N₂. The mixture was stirred at 0° C. for 30 min and then 4-chloro-2,6-dimethylpyridine (CAS: 3512-75-2; 0.70 mL, 5.46 mmol) was added dropwise. The mixture was stirred at 0° C. for 1 h and then at 80° C. for 20 h. After cooling to rt, a saturated solution of NH₄Cl was added and the mixture was extracted with EtOAc. The organic layer was separated, dried (MgSO₄), filtered and evaporated in vacuo. The residue was purified by flash column chromatography (SiO₂; EtOAc in heptane, gradient from 50/50 to 100/0). The desired fractions were collected and concentrated in vacuo to yield intermediate 9d as an oil (1.4 g, 92%).

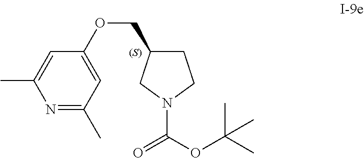

Intermediate 9e was prepared following an analogous procedure to the one described for the synthesis of intermediate 9d using (3S)-1-Boc-3-hydroxymethylpyrrolidine (CAS: 199174-24-8) as starting material.

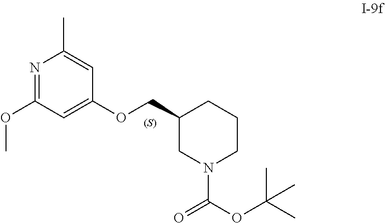

Intermediate 9f was prepared following an analogous procedure to the one described for the synthesis of intermediate 9a using 4-bromo-2-methoxy-6-methylpyridine (CAS: 1083169-00-9) as starting material.

### Preparation of Intermediates I-10a, 10b, 10c, 10d, 10e and 10f

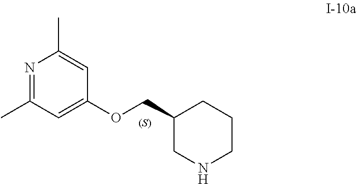

Intermediate 10a was prepared following an analogous procedure to the one described for the synthesis of intermediate 3a using intermediate 9a as starting material.

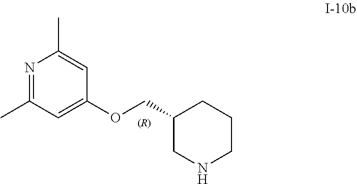

Intermediate 10b was prepared following an analogous procedure to the one described for the synthesis of intermediate 3a using intermediate 9b as starting material.

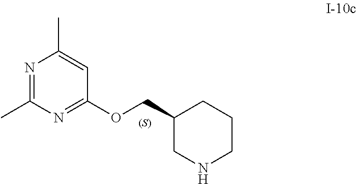

Intermediate 10c was prepared following an analogous procedure to the one described for the synthesis of intermediate 3a using intermediate 9c as starting material.

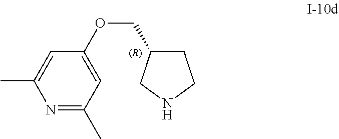

Intermediate 10d was prepared following an analogous procedure to the one described for the synthesis of intermediate 3a using intermediate 9d as starting material.

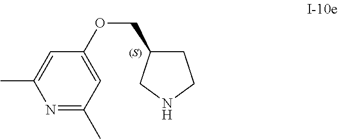

Intermediate 10e was prepared following an analogous procedure to the one described for the synthesis of intermediate 3a using intermediate 9e as starting material.

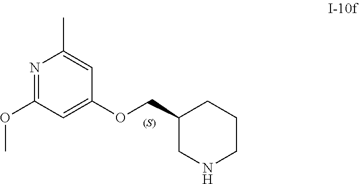

Intermediate 10f was prepared following an analogous procedure to the one described for the synthesis of intermediate 3a using intermediate 9f as starting material.

### Preparation of Intermediate 31

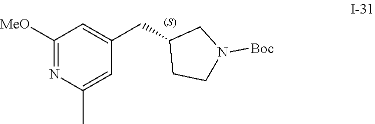

A solution of intermediate 6c (0.1M solution in THF, 66 mL, 6.6 mmol) was added to a solution of 4-bromo-2-methoxy-6-methylpyridine (CAS: 1083169-00-9; 1.21 g, 6.00 mmol) and Pd(t-Bu₃P)₂ (140 mg, 0.27 mmol). The reaction mixture was stirred at room temperature for 16 h. The mixture was treated with NH₄Cl (sat., aq.) and extracted with EtOAc. The organic layer was dried (Na₂SO₄), filtered and the solvent was evaporated in vacuo. The crude mixture was purified by flash column chromatography (SiO₂, EtOAc in heptane, gradient from 100:0 to 80:20) to afford intermediate 31 (1 g, 54%).

### Preparation of Intermediate 32

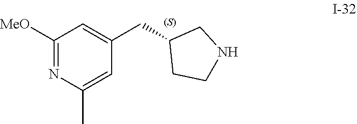

Amberlyst®15 hydrogen form (CAS: 39389-20-3; 4.11 mmol/g) was added to a solution of intermediate 31 (1.00 g, 3.26 mmol) in MeOH (16.6 mL). The reaction mixture was shaken for 18 h. The solvent was removed. The resin was washed few times with MeOH, then NH₃ (7N in MeOH) was added to the resin and shaken for 1 h. The solvent was removed and the resin was washed few times with NH₃ (7N in MeOH). The solvent was evaporated in vacuo to afford intermediate 32 (600 mg, 89%).

### Preparation of Intermediate 33

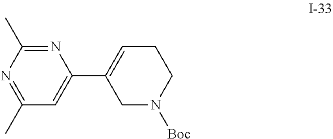 [SMILES: Cc1cc(C2=CCCN(C(=O)OC(C)(C)C)C2)nc(C)n1]

Pd(PPh₃)₄ (1.04 g, 0.90 mmol) was added to a stirred solution of 4-chloro-2,6-dimethylpyrimidine (CAS: 4472-45-1; 2.14 g, 14.9 mmol) and 5-(4,4,5,5-tetramethyl-[1,3,2]dioxaborolan-2-yl)-3,6-dihydro-2H-pyridine-1-carboxylic acid tert-butyl ester (CAS: 885693-20-9; 5.09 g, 16.5 mmol) in 1,4-dioxane (10 mL) in a sealed tube and under N₂ atmosphere. The reaction mixture was stirred at 130° C. for 30 min under microwave irradiation. The mixture was treated with water and extracted with EtOAc. The combined organic extracts were dried (Na₂SO₄), filtered and the solvents were evaporated in vacuo. The crude mixture was purified by flash column chromatography (SiO₂, EtOAc in heptane, gradient from 100/0 to 0/100) to afford intermediate 33 (4.12 g, 95%).

### Preparation of Intermediate 34

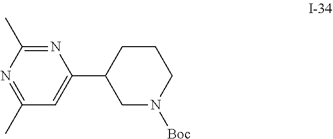 [SMILES: Cc1cc(C2CCCN(C(=O)OC(C)(C)C)C2)nc(C)n1]

Pd/C (10% purity, 1.51 g, 1.42 mmol) was added to a stirred solution of intermediate 33 (4.1 g, 14.2 mmol) in EtOH (82 mL) under N₂ atmosphere. The reaction mixture was hydrogenated (atmospheric pressure) at room temperature for 16 h. The mixture was filtered through a pad of Celite® and washed with MeOH. The filtrate was concentrated in vacuo to afford intermediate 34 (3.98 g, 96%).

### Preparation of Intermediate 35

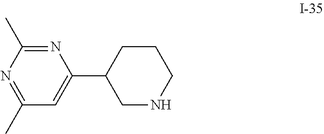

A solution of intermediate 34 (3.96 g, 13.6 mmol) in MeOH (105 mL) was added to a closed reactor containing Amberlyst®15 hydrogen form (CAS: 39389-20-3; 14.5 g, 67.9 mmol). The reaction mixture was shaken in a solid phase reactor at room temperature for 16 h. The resin was washed with MeOH (the fraction was discarded). NH₃ (7N in MeOH) (39 mL) was added and the mixture was shaken in the solid phase reactor for 2 h. The resin was filtered off and washed twice with NH₃ (7N in MeOH) (3×39 mL; 30 min shaken). The filtrates were combined and concentrated in vacuo to afford intermediate 35 (2.3 g, 88%).

### Preparation of Intermediate 36

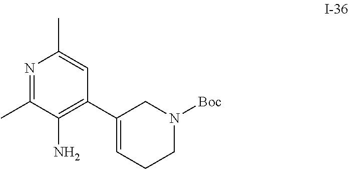 [SMILES: Cc1cc(C2=CCCN(C(=O)OC(C)(C)C)C2)c(N)c(C)n1]

Intermediate 36 was prepared following an analogous procedure to the one described for the synthesis of intermediate 33 using 4-chloro-2,6-dimethylpyridin-3-amine (CAS: 37652-11-2) and 5-(4,4,5,5-tetramethyl-[1,3,2]dioxaborolan-2-yl)-3,6-dihydro-2H-pyridine-1-carboxylic acid tert-butyl ester (CAS: 885693-20-9) as starting materials. The crude mixture was purified by flash column chromatography (SiO₂, EtOAc in DCM, gradient from 0/100 to 50/50) to afford intermediate 36 (2.38 g, 95%) as an oil.

### Preparation of Intermediate 37

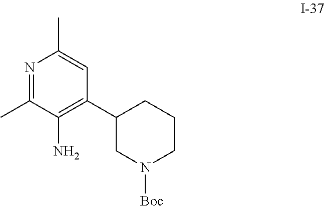 [SMILES: Cc1cc(C2CCCN(C(=O)OC(C)(C)C)C2)c(N)c(C)n1]

Intermediate 37 was prepared following an analogous procedure to the one described for the synthesis of intermediate 34 using intermediate 36 as starting material.

### Preparation of Intermediate 38

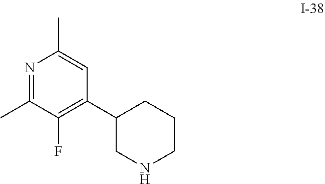

Nitrosyl tetrafluoroborate (2.29 g, 19.6 mmol) was added portion wise to a solution of intermediate 37 (2.00 g, 6.55 mmol) in anhydrous DCM (20 mL). The reaction mixture was stirred at room temperature for 18 h. The reaction was filtered. The filtrate was discarded, while the precipitate was dissolved in MeOH and passed thorough an Isolute SCX2 cartridge. The cartridge was washed with MeOH and the product was eluted with NH₃ in MeOH. The desired fractions were collected, and the solvents were concentrated in vacuo. The residue was purified by flash column chromatography (SiO₂, MeOH in DCM, gradient from 0/100 to 20/80). A second purification was performed by RP HPLC (stationary phase: C18 XBridge 30×100 mm 5 μm), mobile phase: NH₄HCO₃ (0.25% solution in water)/MeCN, gradient from 95/5 to 70/30) to afford intermediate 38 (310 mg, 23%).

### Preparation of Intermediate 39

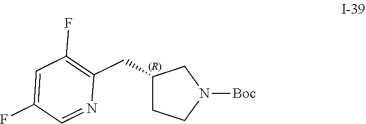

Intermediate 6c (0.38M in THF, 11 mL, 4.18 mmol), followed by TMEDA (0.63 mL) and Pd(PPh₃)₂Cl₂ (68 mg, 96.9 μmol) were added to 2-bromo-3,5-difluoropyridine [660425-16-1] (0.76 g, 3.92 mmol) in a sealed tube and under N₂ atmosphere. The reaction mixture was stirred at 65° C. for 16 h. The reaction was quenched with a 1:1 solution of NH₄Cl (sat.) and NH₃ (26% aq.) and extracted with EtOAc. The organic layer was separated, dried (MgSO₄), filtered and the solvent was evaporated in vacuo. The crude mixture was purified by flash column chromatography (SiO₂, EtOAc in heptane, gradient from 0/100 to 30/70) to afford intermediate 39 (715 mg, 61%).

### Preparation of Intermediate 40

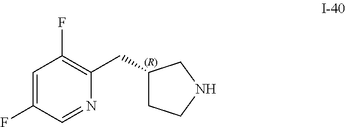

A solution of intermediate 38 (1.17 g, 3.91 mmol) in MeOH (19.6 mL) was added dropwise to Amberlyst®15 hydrogen form (CAS: 39389-20-3; 3.93 g, 18.5 mmol) in a solid phase reaction. Once the evolution of CO₂ stopped, the reaction mixture was shaken at room temperature for 2 days. The resin was washed with MeOH (fraction was discarded) and NH₃ (7N in MeOH). The filtrate was concentrated in vacuo to afford intermediate 40 (0.698 g, 90%).

### Preparation of Intermediate 41

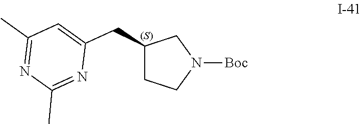

Intermediate 41 was prepared following an analogous procedure to the one described for intermediate 39 starting from 4-chloro-2,6-dimethylpyrimidine (CAS: 4472-45-1).

### Preparation of Intermediate 42

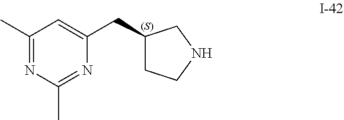

Intermediate 42 was prepared following an analogous procedure to the one described for intermediate 40 starting from intermediate 41.

### Preparation of Intermediate 43

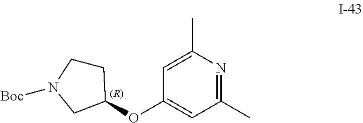

(R)-tert-Butyl 3-hydroxypyrrolidine-1-carboxylate (CAS: 109431-87-0; 1.50 g, 8.01 mmol) was stirred in DMF (3.2 mL) at room temperature. NaH (60% dispersion in mineral oil, 320 mg, 8.01 mmol) was added. A solution of 4-chloro-2,6-lutidine (CAS: 3512-75-2; 1.02 mL, 8.01 mmol) in DMF (3.22 mL) was added dropwise. The reaction mixture was stirred overnight at 60° C. The mixture was evaporated in vacuo. The residue was diluted with water and extracted with EtOAc. The organic layer was dried (Na₂SO₄), filtered and evaporated in vacuo. The crude mixture was purified by flash column chromatography (SiO₂, EtOAc in heptane, gradient from 0/100 to 90/10) to afford intermediate 43 (1.20 g, 51%).

### Preparation of Intermediate 44

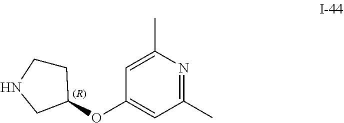

A solution of intermediate 43 (1.20 g, 4.10 mmol) in MeOH (31.6 mL) was added to a closed reactor containing Amberlyst®15 hydrogen form (CAS: 39389-20-3; 4.37 g, 20.5 mmol). The reaction mixture was shaken in a solid phase reactor at room temperature for 16 h. The resin was washed with MeOH (the fraction was discarded). NH₃ (7N in MeOH) (31.7 mL) was added and the mixture was shaken in the solid phase reactor for 2 h. The resin was filtered off and was washed with NH₃ (7N in MeOH) (2×31 mL; 30 min shaken). The filtrates were combined and concentrated in vacuo to afford intermediate 44 (710 mg, 90%).

### Preparation of Intermediate 45

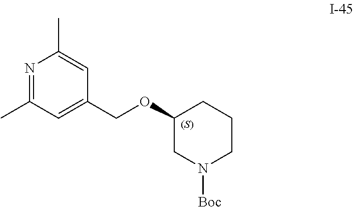

NaH (60% dispersion in mineral oil, 221 mg, 5.51 mmol) was added to a stirred solution of (S)-1-boc-3-hydroxypiperidine (CAS: 143900-44-1; 1.01 g, 5.01 mmol) in DMF (31 mL) at room temperature. The mixture was stirred for 15 min and 2,6-dimethyl-pyridin-4-ylmethyl chloride (CAS: 120739-87-9; 1.00 g, 5.01 mmol, 78% purity) was added. The reaction mixture was stirred at room temperature for 16 h.

NH₄Cl (sat., aq.) was added and the mixture was extracted with EtOAc. The organic layer was washed with brine (twice), dried (Na₂SO₄), filtered and concentrated in vacuo. The crude mixture was purified by flash column chromatography (SiO₂, EtOAc in heptane, gradient from 0/100 to 100/0) to afford intermediate 45 (1.19 g, 74%).

### Preparation of Intermediate 46

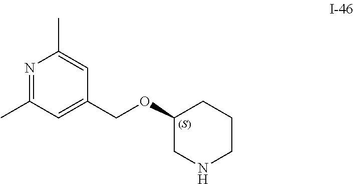

Intermediate 46 was prepared following an analogous procedure to the one described for the synthesis of intermediate 44 using intermediate 46 as starting material.

### Preparation of Intermediate 47

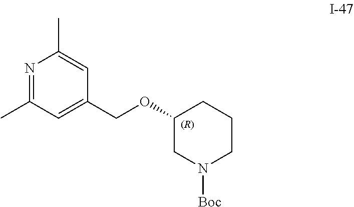

Intermediate 47 was prepared following an analogous procedure to the one described for intermediate 45 using (R)-1-boc-3-hydroxypiperidine (CAS: 143900-43-0) and 2,6-dimethyl-pyridin-4-ylmethyl chloride (CAS: 120739-87-9) as starting materials.

### Preparation of Intermediate 48

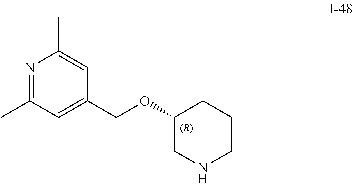

Intermediate 48 was prepared following an analogous procedure to the one described for the synthesis of intermediate 44 using intermediate 47 as starting material.

### Preparation of Intermediate 49

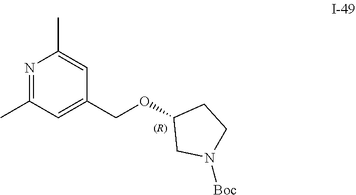

Intermediate 49 was prepared following an analogous procedure to the one described for intermediate 45 using (R)-1-boc-3-hydroxypyrrolidine (CAS: 109431-87-0) and 2,6-dimethyl-pyridin-4-ylmethyl chloride (CAS: 120739-87-9) as starting materials.

### Preparation of Intermediate 50

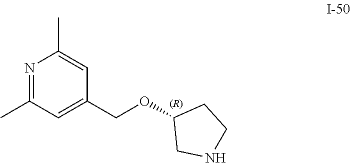

Intermediate 50 was prepared following an analogous procedure to the one described for the synthesis of intermediate 44 using intermediate 49 as starting material.

### Preparation of Intermediate 51

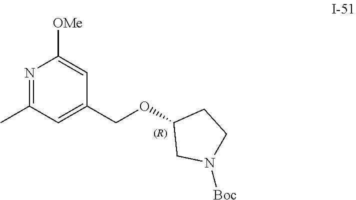

NaH (60% dispersion in mineral oil, 238 mg, 5.96 mmol) was added to a solution of (R)-3-hydroxymethyl-pyrrolidine-1-carboxylic acid tert-butyl ester (CAS: 138108-72-2; 1.00 g, 4.97 mmol) in DMF (10 mL) at 0° C. under N₂ atmosphere. The mixture was stirred at 0° C. for 15 min, and 4-bromo-2-methoxy-6-methylpyridine (CAS: 1083169-00-9; 1.15 g, 5.47 mmol) added dropwise. The reaction mixture was stirred at 0° C. for 1 h and then at 70° C. for 20 h. The reaction was quenched with NH₄Cl (sat., aq.) and extracted with heptane. The organic layer was dried (MgSO₄), filtered and evaporated in vacuo. The crude mixture was purified by flash column chromatography (SiO₂, EtOAc in heptane, gradient from 0/100 to 50/50) to afford intermediate 51 (970 mg, 61%).

### Preparation of Intermediate 52

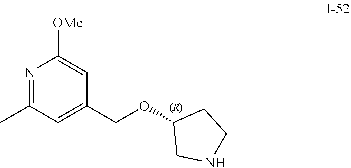

Intermediate 52 was prepared following an analogous procedure to the one described for the synthesis of intermediate 44 using intermediate 51 as starting material.

### Preparation of Intermediate 53

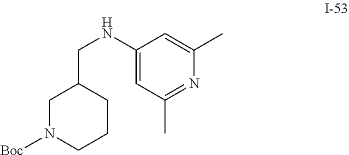 [SMILES: Cc1cc(NCC2CCCN([I-53])C2)cc(C)n1]

Pd₂dba₃ (187 mg, 0.20 mmol), DavePhos (166 mg, 0.41 mmol) and NaOt-Bu (1.57 g, 16.3 mmol) were added under N₂ atmosphere to a solution of 4-bromo-2,6-dimethylpyridine (CAS: 5093-70-9; 1.52 g, 8.17 mmol) in anhydrous 1,4-dioxane (40 mL) in a sealed tube. tert-Butyl 3-(aminomethyl)piperidine-1-carboxylate (CAS: 162167-97-7; 2.10 g, 9.80 mmol) was added at room temperature and the reaction mixture was stirred at 100° C. for 16 h. The mixture was diluted with EtOAc and NH₄Cl (aq., sat., 0.5 mL). The mixture was filtered over a pad of Celite® and the filtrate was concentrated in vacuo. The crude mixture was purified by flash column chromatography (SiO₂, MeOH in DCM, gradient from 0/100 to 50/50) to afford intermediate 53 (2.26 g, 82%).

### Preparation of Intermediate 54

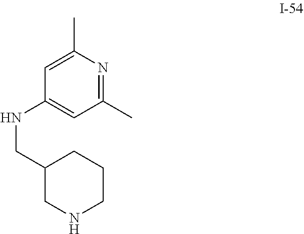 [SMILES: Cc1cc(NCC2CCCNC2)cc(C)n1]

HCl (4M in 1,4-dioxane, 25.6 mL, 103 mmol) was added dropwise to a stirred solution of intermediate 53 (2.23 g, 6.84 mmol) in MeOH (15.8 mL) at 0° C. The reaction mixture was stirred at room temperature for 16 h and the solvent was evaporated in vacuo. The crude mixture was purified by phase reverse ([25 mM NH₄HCO₃]/[MeCN/MeOH (1/1), gradient from 95/5 to 63/37). The desired fractions were collected and concentrated in vacuo. MeCN (3×10 mL) was added and the solvent was concentrated in vacuo to afford intermediate 53 (1.3 g, 87%).

### Preparation of Intermediate 55

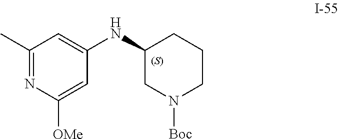

NaOt-Bu (119 mg, 1.24 mmol) was added to a stirred suspension of Pd₂dba₃ (22.7 mg, 24.7 μmop and tBuXPhos (31.5 mg, 74.2 μmol) in 1,4-dioxane (15 mL) in a sealed tube and under N₂ atmosphere at room temperature. The reaction mixture was stirred at 95° C. for 5 min, then a mixture of (S)-(+)-3-amino-1-boc-piperdine [625471-18-3] (129 mg, 0.64 mmol) and 4-bromo-2-methoxy-6-methylpyridine [1083169-00-9] (100 mg, 0.49 mmol) in 1,4-dioxane (5 mL) was added under N₂ atmosphere at 95° C. The reaction mixture was stirred at 100° C. for 30 min. The mixture was diluted with NaHCO₃ (sat., aq.) and extracted with EtOAc. The organic layer was dried (MgSO₄), filtered and the solvents were evaporated in vacuo. The crude mixture was purified by flash column chromatography (SiO₂, EtOAc in heptane, gradient from 5/95 to 100/0) to afford intermediate 55 (130 mg, 82%).

### Preparation of Intermediate 56

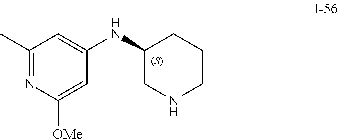

HCl (4M in 1,4-dioxane, 0.50 mL, 2.00 mmol) was added dropwise to intermediate 55 (130 mg, 0.40 mmol) at 0° C. The reaction mixture was stirred at room temperature for 16 h and the solvent was evaporated in vacuo. The residue was dissolved in MeOH (1 mL) and Amberlyst®A26 hydroxide form (CAS: 39339-85-0; 505 mg, 1.62 mmol) was added. The mixture was stirred at room temperature until pH was 7. The resin was removed by filtration and the solvents were evaporated in vacuo to afford intermediate 56 (85 mg, 95%).

### Preparation of Intermediate 57

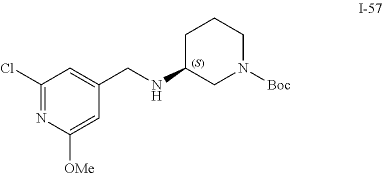

(S)-(+)-3-Amino-1-boc-piperidine (CAS: 625471-18-3; 117 mg, 0.58 mmol) and 2-methoxy-6-methylpyridine-4-carbaldehyde (CAS: 951795-43-0; 100 mg, 0.58 mmol) were dissolved in ACN (3 mL). The reaction mixture was stirred at room temperature for 30 min, and sodium triacetoxyborohydride (371 mg, 1.75 mmol) was added. The resulting mixture was stirred at room temperature for 16 h. The mixture was diluted with NaHCO₃ (sat., aq.) and DCM. The aqueous layer was extracted with DCM (twice). The combined organic layers were dried (MgSO₄), filtered and the solvents were evaporated in vacuo. The crude mixture was purified by flash column chromatography (SiO₂, EtOAc in heptane, gradient from 0/100 to 50/50) to afford intermediate 57 (161 mg, 77%).

### Preparation of Intermediate 58

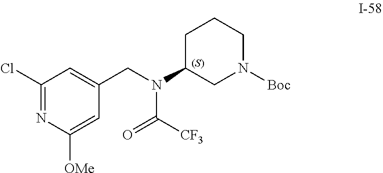

TFA (0.5 mL, 3.23 mmol) was added dropwise to a stirred mixture of intermediate 57 (1.00 g, 2.81 mmol) and DIPEA (0.64 mL, 3.65 mmol) in DCM (13 mL) under N₂ atmosphere at room temperature. The reaction mixture was stirred for 16 h. The reaction was quenched with HCl (1M) and extracted with DCM. The organic layer was washed with NaHCO₃ (sat., aq.) and brine, dried (MgSO₄), filtered and the solvents were evaporated in vacuo. The crude mixture was purified by flash column chromatography (SiO₂, EtOAc in heptane, gradient from 0/100 to 30/70) to afford intermediate 58 (1.1 g, 87%).

### Preparation of Intermediate 59

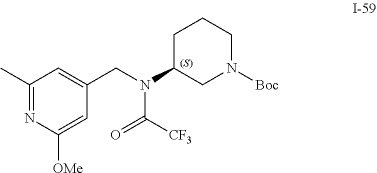

Intermediate 58 (900 mg, 1.99 mmol) and methylboronic acid [13061-96-6] (304 mg, 4.98 mmol) were added to a stirred solution of Na₂CO₃ (633 mg, 5.98 mmol) in 1,4-dioxane (4.98 mL) and H₂O (1.25 mL) under N₂ atmosphere. PdCl₂(dppf).DCM (81.3 mg, 99.6 μmop was added and the reaction mixture was stirred at 105° C. for 16 h. Additional amount of methylboronic acid (1.25 eq), PdCl₂(dppf).DCM (0.025 eq) and Na₂CO₃ (1.5 eq) were added under N₂ atmosphere. The reaction mixture was stirred at 105° C. for 16 h. The mixture was diluted with NaHCO₃ and extracted with EtOAc. The organic layer was dried (MgSO₄), filtered and the solvents were evaporated in vacuo. The crude mixture was purified by flash column chromatography (SiO₂, EtOAc in heptane, gradient from 0/100 to 20/80) to afford intermediate 59 (690 mg, 80%).

### Preparation of Intermediate 60

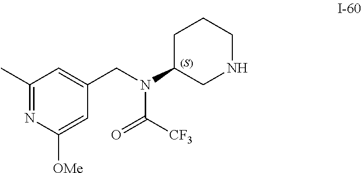

HCl (4M in 1,4-dioxane, 2.00 mL, 8.00 mmol) was added dropwise to intermediate 59 (690 mg, 1.60 mmol) at 0° C. The reaction mixture was stirred at room temperature for 16 h and the solvent was evaporated in vacuo. The crude mixture was purified by flash column chromatography (SiO₂, MeOH:NH₃ in DCM, gradient from 0/100 to 10/90). The desired fractions were collected and concentrated in vacuo to afford intermediate 60 (317 mg, 59%).

### Preparation of Intermediate 61

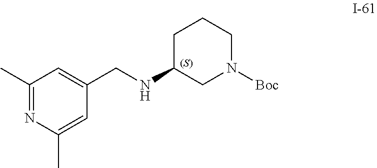

(S)-(+)-3-Amino-1-boc-piperidine (CAS: 625471-18-3; 449 mg, 2.24 mmol) and 2,6-dimethyl-4-pyridine carboxaldehyde (CAS: 18206-06-9; 303 mg, 2.24 mmol) were dissolved in DCM (10 mL). The reaction mixture was stirred at room temperature for 30 min and sodium triacetoxyborohydride (1.43 g, 6.73 mmol) was added. The resulting mixture was stirred at room temperature for 16 h. NaHCO₃ (sat., aq.) and DCM were added. The aqueous layer was extracted with DCM (twice). The combined organic extracts were dried (MgSO₄), filtered and the solvents were evaporated in vacuo. The crude mixture was purified by flash column chromatography (SiO₂, EtOAc in heptane, gradient from 0/100 to 50/50) to afford intermediate 61 (577 mg, 79%).

### Preparation of Intermediate 62

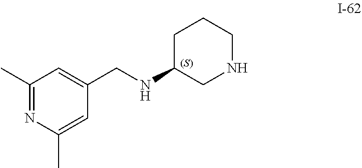

HCl (4M in 1,4-dioxane, 2.26 mL, 9.03 mmol) was added dropwise to intermediate 61 (577 mg, 1.81 mmol) at 0° C. The reaction mixture was stirred at room temperature for 16 h and the solvent was removed in vacuo. The crude mixture was purified by flash column chromatography (SiO₂, MeOH:NH₃ in DCM, gradient from 0/100 to 10/90) to afford intermediate 62 (320 mg, 80%).

### Preparation of Intermediates I-11a, 11b and 11c

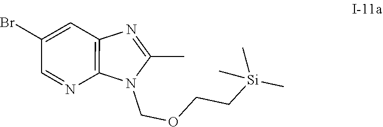 [SMILES: Cc1nc2cc(Br)cnc2n1COCC[Si](C)(C)C]

Sodium hydride (CAS: 7646-69-7; 60% dispersion in mineral oil, 3.80 g, 94.97 mmol) was added portionwise to a stirred solution of 6-bromo-2-methyl-1H-imidazo[4,5-b]pyridine (CAS: 42869-47-6; 10.0 g, 47.16 mmol) in DMF (100 mL) at 0° C. The mixture was stirred at 0° C. for 30 min and then 2-(trimethylsilyl)ethoxymethylchloride (CAS: 76513-69-4; 19.20 mL, 108.47 mmol) was added dropwise. The mixture was stirred at rt for 16 h and then it was diluted with a saturated NH₄Cl solution and extracted with EtOAc. The organic layer was separated, washed with brine, dried (Na₂SO₄), filtered and evaporated in vacuo. The residue was purified by flash column chromatography (SiO₂; EtOAc in heptane, gradient from 0/100 to 100/0). The desired fractions were collected and concentrated in vacuo to yield intermediate 11a as a pale-brown solid (8.07 g, 50%).

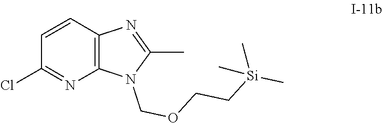 [SMILES: Cc1nc2ccc(Cl)nc2n1COCC[Si](C)(C)C]

Intermediate 11b was prepared following an analogous procedure to the one described for the synthesis of intermediate 1a using intermediate 5-chloro-2-methyl-3H-imidazo[4,5-b]pyridine (CAS: 40851-92-1) as starting material. Intermediate 11b was purified by flash column chromatography (SiO₂; MeOH in DCM, gradient from 0/100 to 5/95).

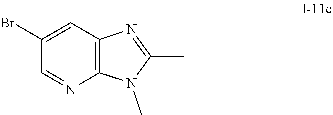

K₂CO₃ (3.05 g, 433.1 mmol) and methyl iodide (CAS: 74-88-4; 0.5 mL, 8.03 mmol) were added to a stirred solution of 6-bromo-2methyl-1H-imidazo[4,5-b]pyridine (CAS: 42869-47-6, 1.35 g, 6.37 mmol) in acetone (32 mL). The mixture was stirred at rt for 16 h and then water and EtOAc were added. The organic layer was separated, dried (Na₂SO₄), filtered and evaporated in vacuo. The residue was purified by flash column chromatography (SiO₂; 7N solution of NH₃ in MeOH in DCM, gradient from 0/100 to 10/90). The desired fractions were collected and concentrated in vacuo to yield intermediate 11c as a brown solid (0.835 g, 58%).

### Preparation of Intermediates I-12a and 12b

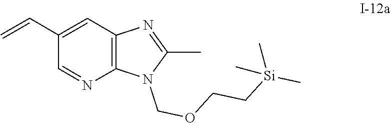 [SMILES: C=Cc1cnc2c(c1)nc(C)n2COCC[Si](C)(C)C]

Pd(PPh₃)₄ (CAS: 14221-01-3; 1.36 g, 1.18 mmol) was added to a stirred mixture of intermediate 11a (8.07 g, 23.57 mmol) and 4,4,5,5-tetramethyl-2-vinyl-1,3,2-dioxaborolane (CAS: 75927-49-0; 6.00 mL, 35.36 mmol) in a mixture of a saturated solution of K₂CO₃ (36.3 mL) and 1,4-dioxane (36.3 mL) at rt under N₂. The mixture was stirred at 95° C. for 16 h. Then a saturated solution of K₂CO₃ was added and the mixture was extracted with EtOAc. The organic layer was separated, washed with brine, dried (Na₂SO₄), filtered and evaporated in vacuo. The residue was purified by flash column chromatography (SiO₂; MeOH in DCM, gradient from 0/100 to 4/96). The desired fractions were collected and concentrated in vacuo to yield intermediate 12a as an orange oil that solidified upon standing (2.07 g, 28%).

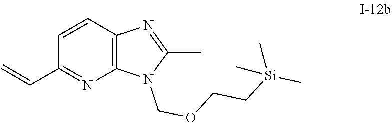 [SMILES: C=Cc1ccc2nc(C)n(COCC[Si](C)(C)C)c2n1]

Tributyl(vinyl)tin (CAS: 123-91-1; 0.82 mL, 2.83 mmol), 2,4-di-tert-butyl-4-methylphenol (CAS: 128-37-0; 0.24 g, 1.10 mmol) and Pd(PPh₃)₄ (CAS: 14221-01-3; 0.14 g, 0.12 mmol) were added to a stirred mixture of intermediate 11b (0.36 g, 1.20 mmol) in 1,4-dioxane (3.8 mL) in a sealed tube under N₂. The mixture was stirred at 100° C. for 16 h. After cooling to rt, the mixture was filtered off and the solid washed with EtOAc. The filtrate was evaporated in vacuo and the crude product was purified by flash column chromatography (SiO₂; MeOH in DCM, gradient from 0/100 to 5/95). The desired fractions were collected and concentrated in vacuo to yield intermediate 12b (0.25 g, 72%).

### Preparation of Intermediate I-13a

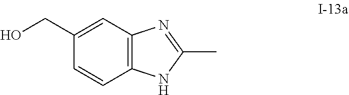

LiAlH₄ (CAS: 16853-85-3; 1M in THF, 2.8 mL, 2.77 mmol) was added dropwise to a stirred solution of 5-ethoxycarbonyl-2-methylbenzimidazole (CAS: 717-37-3; prepared according to Eur. J. Med. Chem. 2009, 1500-1508, 0.47 g, 2.31 mmol) in THF (14 mL) at 0° C. under N₂. The mixture was stirred at 0° C. for 5 min and then at rt for 2 h. Then the mixture was cooled down to 0° C. and more LiAlH₄ (1.4 mL, 1.39 mmol) was added. The mixture was stirred at 0° C. for 5 min and at rt for another 2 h. Then a saturated solution of Rochelle's salt in ice was added and the mixture was extracted with EtOAc. The organic layer was separated, washed with brine, dried (MgSO₄), filtered and the solvents were removed in vacuo. The residue was purified by flash column chromatography (SiO₂; 7N solution of NH₃ in MeOH in DCM, gradient from 0/100 to 10/90). The desired fractions were collected and concentrated in vacuo to yield intermediate 13a as a white solid (0.80 g, 21%).

### Preparation of Intermediates I-14a, 14b, 14c and 14d

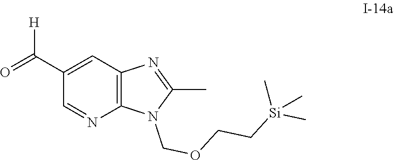 [SMILES: Cc1nc2cc(C=O)cnc2n1COCC[Si](C)(C)C]

Sudan III (CAS: 85-86-9; trace amount) was added to a stirred solution of intermediate 12a (4.3 g, 7.86 mmol) in a mixture of ACN (195.5 mL) and water (9.8 mL). The solution was cooled to 0° C. and a mixture of O₃/O₂ was passed through the flask until the red color dissipated. The reaction was purged with N₂ for 10 min. Then, the reaction was diluted with a saturated solution of sodium thiosulfate and extracted with EtOAc. The organic layer was separated, washed with brine, dried (Na₂SO₄), filtered and the solvents were removed in vacuo. The residue was purified by flash column chromatography (SiO₂; EtOAc in heptane, gradient from 0/100 to 60/40). The desired fractions were collected and concentrated in vacuo to yield intermediate 14a as white solid (2.39 g, 55%).

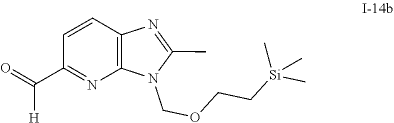 [SMILES: Cc1nc2ccc(C=O)nc2n1COCC[Si](C)(C)C]

Sodium periodate (CAS: 7790-28-5; 1.12 g, 5.25 mmol), osmium tetroxide (2.5% in tBuOH CAS: 20816-12-0; 0.18 mL, 0.013 mmol) and 2,6-dimethylpyridine (CAS: 108-48-5; 0.27 mL, 2.30 mmol) were added to a stirred solution of intermediate 12b (4.3 g, 7.86 mmol) in a mixture of 1,4-dioxane (8.0 mL) and water (2.66 mL) in a sealed tube under N₂. The mixture was stirred at rt for 17 h. Then, the reaction was diluted with water and extracted with EtOAc. The organic layer was separated, washed with brine, dried (MgSO₄), filtered and the solvents were removed in vacuo. The residue was purified by flash column chromatography (SiO₂; 7N solution of NH₃ in MeOH in DCM, gradient from 0/100 to 10/90). The desired fractions were collected and concentrated in vacuo to yield intermediate 14b as a yellow oil (0.13 g, 52%).

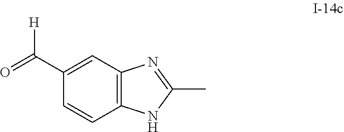

MnO₂ (0.50 g, 4.90 mmol) was added to a stirred suspension of intermediate 13a (0.080 g, 0.49 mmol) in 1,4-dioxane (3 mL) in a sealed tube under N₂. The mixture was stirred at 80° C. for 16 h. After cooling to rt, the mixture was filtered through a Celite® pad and the pad was washed with DCM. The filtrate was concentrated in vacuo to yield intermediate 14c as a white solid (0.047 g, 59%).

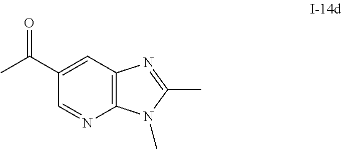

Tributyl(1-ethoxyvinyl)tin (CAS: 97674-02-7; 0.74 mL, 2.19 mmol) and Pd(PPh₃)₂Cl₂ (0.14 g, 0.19 mmol) were added to a stirred mixture of intermediate 11c (0.46 g, 2.03 mmol) in toluene (10 mL) in a sealed tube under N₂. The mixture was stirred at 80° C. for 16 h. After cooling to rt, a 1M HCl solution (4 mL) was added and the mixture was stirred at 80° C. for a further 5 h. After cooling to rt, the mixture was poured onto a stirred mixture of a saturated NaHCO₃ solution and ice and extracted with DCM. The organic layer was separated, washed with brine, dried (MgSO₄), filtered and the solvents were evaporated in vacuo. The residue was purified by flash column chromatography (SiO₂; 7N solution of NH₃ in MeOH in DCM, gradient from 0/100 to 5/95). The desired fractions were collected and concentrated in vacuo to yield intermediate 14d as a pale-orange solid (0.24 g, 63%).

### Preparation of Intermediates I-15a, 15b and 15c

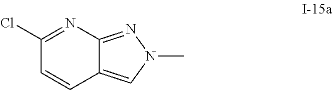

6-Chloro-1H-pyrazolo[4,3-b]pyridine (CAS: 63725-51-9; 0.35 g, 2.25 mmol) was added to a stirred solution of trimethyloxoniumtetrafluoroborate (CAS: 420-37-1; 1.35 g, 9.13 mmol) and DIPEA (1.93 mL, 11.23 mmol) in DCM (13.8 mL). The mixture was stirred at rt for 72 h and quenched with a saturated solution of NaHCO₃ and extracted with DCM. The organic layer was separated, dried (Na₂SO₄), filtered and the solvent was evaporated in vacuo. The residue was purified by flash column chromatography (SiO₂; EtOAc in heptane, gradient from 20/80 to 100/0). The desired fractions were collected and concentrated in vacuo to yield intermediate 15a as a white solid (0.27 g, 65%).

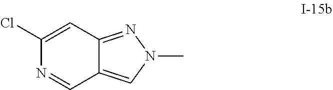

Intermediate 15b was prepared following an analogous procedure to the one described for the synthesis of intermediate 15a using 6-chloro-1H-pyrazolo[4,3-c]pyridine (CAS: 1206979-33-0) as starting material.

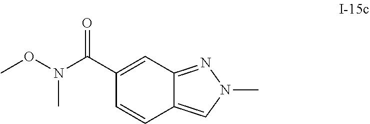

HATU (CAS: 148893-10-1; 2.70 g, 7.10 mmol), N,O-dimethylhydroxylamine hydrochloride (CAS: 6638-79-5, 067 g, 6.87 mmol) and Et₃N (2.50 mL, 17.99 mmol) were added to a stirred suspension of 2-methylindazole-6-carboxylic acid (CAS: 103141-74-8; 1 g, 5.68 mmol) in DMF (28 mL) at rt under N₂. The mixture was stirred at rt for 16 h and then water was added. The mixture was extracted with EtOAc and the organic layer was separated, washed with brine, dried (Na₂SO₄), filtered and the solvents evaporated in vacuo. The residue was purified by flash column chromatography (SiO₂; MeOH in DCM, gradient from 0/100 to 10/90). The desired fractions were collected and concentrated in vacuo to yield intermediate 15c as an orange oil which solidified upon standing (0.405 g, 33%).

### Preparation of Intermediates I-16a, 16b, 16c, 16d and 16e

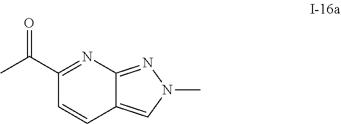

Pd(PPh₃)₄ (0.183 g, 0.16 mmol) was added to a stirred suspension of tributyl(1-ethoxyvinyl)tin (CAS: 97674-02-7; 0.80 mL, 2.37 mmol) and intermediate 15a (0.27 g, 1.58 mmol) in toluene (8.2 mL) in a sealed tube under N₂. The mixture was stirred at 100° C. for 16 h. After cooling to rt, a 2M HCl solution (2.37 mL) was added and the mixture was stirred at 80° C. for 1 h. After cooling to rt, the mixture was neutralized with a saturated NaHCO₃ solution addition and extracted with a 4:1 mixture of DCM and iPrOH. The organic layer was separated, dried (Na₂SO₄), filtered and the solvents were evaporated in vacuo. The residue was purified by flash column chromatography (SiO₂; 7N solution of NH₃ in MeOH in DCM, gradient from 0/100 to 5/95). The desired fractions were collected and concentrated in vacuo to yield intermediate 16a as a brown oil (0.097 g, 28%).

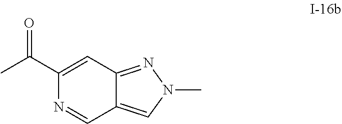

Intermediate 16b was prepared following an analogous procedure to the one described for the synthesis of intermediate 16a using intermediate 15b as starting material.

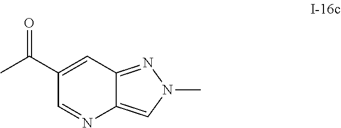

Intermediate 16c was prepared following an analogous procedure to the one described for the synthesis of intermediate 16a using 6-bromo-2-methyl-2H-pyrazolo[4,3-b]pyridine (CAS: 1897500-19-4) as starting material.

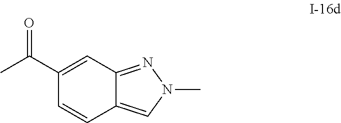

Intermediate 16d was prepared following an analogous procedure to the one described for the synthesis of intermediate 16a using 5-bromo-2-methyl-2H-indazole (CAS: 465529-56-0) as starting material.

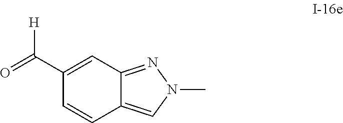

Diisobutylaluminium hydride (1M solution in THF, 2.5 mL, 2.5 mmol) was added dropwise to a stirred solution of intermediate 15c (0.4 g, 1.82 mmol) in 2-methyltetrahydrofurane (9.5 mL) at −78° C. under N₂. The mixture was stirred at −78° C. for 3 h and then diluted with EtOAc. Then sodium sulfate decahydrate was added and the mixture was stirred for 30 min. The mixture was filtered through a Celite® pad and the pad was washed with EtOAc. The filtrate was dried (Na₂SO₄) and the solvent was evaporated in vacuo. The residue was purified by flash column chromatography (SiO₂;

EtOAc in heptane, gradient from 0/100 to 100/0). The desired fractions were collected and concentrated in vacuo to yield intermediate 16e as a yellow solid (0.181 g, 62%).

### Preparation of Intermediates I-17a, 17b and 17c

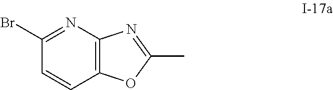

Triethyl orthoacetate (CAS: 78-39-7; 4.82 mL, 26.48 mmol) was added to a stirred mixture of 2-amino-6-bromopyridin-3-ol (CAS: 934758-27-7; 4.17 g, 22.06 mmol) and p-toluenesulfonic acid monohydrate (CAS: 104-15-4; 0.21 g, 1.10 mmol) in toluene (24.2 mL). The mixture was stirred at 130° C. for 1 h and then the solvent evaporated in vacuo. The residue was purified by flash column chromatography (SiO₂; EtOAc in heptane, gradient from 0/100 to 50/50). The desired fractions were collected and concentrated in vacuo to yield intermediate 17a as a yellow solid (0.27 g, 65%).

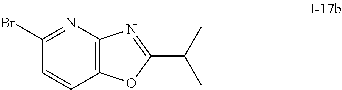

Intermediate 17b was prepared following an analogous procedure to the one described for the synthesis of intermediate 17a using triethyl orthoisobutyrate (CAS: 52698-46-1) as starting material.

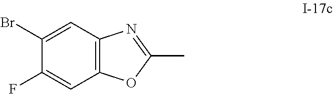

Intermediate 17c was prepared following an analogous procedure to the one described for the synthesis of intermediate 17a using 2-amino-4-bromo-5-fluorobenzene (CAS: 1016234-89-1) as starting material.

### Preparation of Intermediates I-18a, 18b, 18c, 18d and 18e

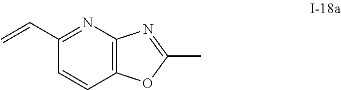

Pd(PPh₃)₄ (0.86 g, 0.75 mmol) was added to a stirred mixture of intermediate 17a (3.18 g, 14.93 mmol) and 4,4,5,5-tetramethyl-2-vinyl-1,3,2-dioxaborolane (CAS: 75927-49-0; 3.80 mL, 22.39 mmol) in a mixture of a saturated solution of K₂CO₃ (17.86 mL) and 1,4-dioxane (17.86 mL) at rt under N₂. The mixture was stirred at 95° C. for 16 h. Then water was added and the mixture was extracted with EtOAc. The organic layer was separated, washed with brine, dried (Na₂SO₄), filtered and evaporated in vacuo. The residue was purified by flash column chromatography (SiO₂; EtOAc in DCM, 0/100 to 50/50). The desired fractions were collected and concentrated in vacuo and the residue was purified again by flash column chromatography (SiO₂; EtOAc in heptane, gradient from 0/100 to 50/50). The desired fractions were collected and concentrated in vacuo to yield intermediate 18a as a light-yellow solid (1.39 g, 58%).

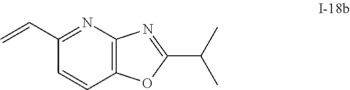

Intermediate 18b was prepared following an analogous procedure to the one described for the synthesis of intermediate 18a using intermediate 17b as starting material.

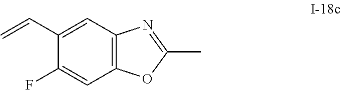

Intermediate 18c was prepared following an analogous procedure to the one described for the synthesis of intermediate 18a using intermediate 17c as starting material.

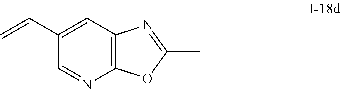

Tributyl(vinyl)tin (CAS: 123-91-1; 1.0 mL, 3.42 mmol), 2,4-di-tert-butyl-4-methylphenol (CAS: 128-37-0; 0.054 g, 0.25 mmol) and Pd(PPh₃)₄ (0.138 g, 0.12 mmol) was added to a stirred mixture of 6-bromo-2-methyloxazolo[5,4-b]pyridine (0.54 g, 2.54 mmol) in 1,4-dioxane (13 mL) in a sealed tube under N₂. The mixture was stirred at 100° C. for 18 h. After cooling to rt, the mixture was filtered through a Celite® pad and the pad was washed with EtOAc. The filtrate was evaporated in vacuo and the crude product was purified by flash column chromatography (SiO₂; EtOAc in heptane, gradient from 0/100 to 100/0). The desired fractions were collected and concentrated in vacuo to yield intermediate 18d (0.405 g, 99%).

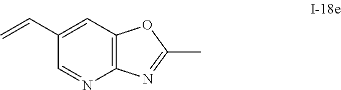

Intermediate 18e was prepared following an analogous procedure to the one described for the synthesis of intermediate 18d using 6-bromo-2-methyloxazolo[4,5-b]pyridine (CAS: 494747-09-0) as starting material.

### Preparation of Intermediates I-19a, 19b, 19c, 19d and 19e

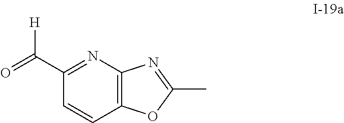

Sudan III (CAS: 85-86-9; trace amount) was added to a stirred solution of intermediate 18a (1.39 g, 8.68 mmol) in a mixture of ACN (114.2 mL) and water (5.7 mL). The solution was cooled to 0° C. and a mixture of O₃/O₂ was passed through the flask until the red color dissipated. The reaction was purged with N₂ for 10 min. Then, the reaction was diluted with a mixture of EtOAc and THF and extracted with a saturated solution of Na₂CO₃. The organic layer was separated, washed with brine, dried (Na₂SO₄), filtered and the solvents were removed in vacuo to yield intermediate 19a as beige solid (1.0 g, 71%).

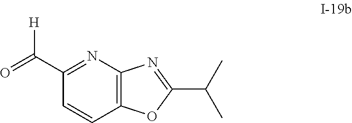

Intermediate 19b was prepared following an analogous procedure to the one described for the synthesis of intermediate 19a using intermediate 18b as starting material.

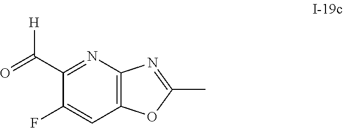

Intermediate 19c was prepared following an analogous procedure to the one described for the synthesis of intermediate 19a using intermediate 18c as starting material. Intermediate 19c was purified by flash column chromatography (SiO₂; EtOAc in DCM, gradient from 0/100 to 20/80).

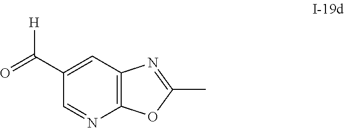

Sodium periodate (CAS: 7790-28-5; 1.19 g, 5.58 mmol) and osmium tetroxide (CAS: 20816-12-0; 2.5% in tBuOH, 0.18 mL, 0.013 mmol) were added to a stirred solution of intermediate 18d (0.40 g, 2.48 mmol) in a mixture of 1,4-dioxane (17.5 mL) and water (7.5 mL) under N₂. The mixture was stirred at rt for 2 h and then a saturated Na₂S₂O₃ solution was added. The mixture was extracted with EtOAc and the organic layer was separated, dried (MgSO₄), filtered and the solvents were removed in vacuo. The residue was purified by flash column chromatography (SiO₂; EtOAc in heptane, gradient from 0/100 to 100/0). The desired fractions were collected and concentrated in vacuo to yield intermediate 19d as a white solid (0.30 g, 75%).

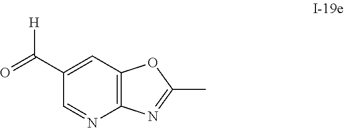

Intermediate 19e was prepared following an analogous procedure to the one described for the synthesis of intermediate 19b using intermediate 1e as starting material.

### Preparation of Intermediate I-20a

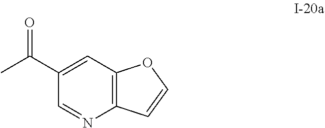

Tributyl(1-ethoxyvinyl)tin (CAS: 97674-02-7; 1.8 mL, 5.33 mmol) and PdCl₂(PPh₃)₂ (0.34 g, 0.49 mmol) were added to a stirred mixture of 6-bromofuro[3,2-b]pyridine (CAS: 935330-61-7, 0.96 g, 4.87 mmol) in toluene (25 mL) in a sealed tube under N₂. The mixture was stirred at 80° C. for 16 h. After cooling to rt, a 1M HCl solution (9.5 mL) was added and the mixture was stirred at 80° C. for a further 5 h. After cooling to rt, the mixture was poured onto a stirred mixture of a saturated NaHCO₃ solution and ice and extracted with DCM. The organic layer was separated, washed with brine, dried (MgSO₄), filtered and the solvents were evaporated in vacuo. The residue was purified by flash column chromatography (SiO₂; EtOAc in DCM, gradient from 0/100 to 50/50). The desired fractions were collected and concentrated in vacuo to yield intermediate 20a as a pale-orange solid (0.24 g, 63%).

### Preparation of Intermediates I-21a and 21b

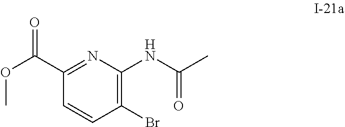

Acetic anhydride (CAS: 108-24-7; 13.2 g, 129.8 mmol) was added to a stirred mixture of methyl 6-amino-5-bromopyridine-2-carboxylate (CAS: 178876-82-9; 30 g, 129.8 mmol) in toluene (600 mL) under N₂. The mixture was stirred at 100° C. for 36 h and then the solvent evaporated in vacuo. The residue was purified by flash column chromatography (SiO₂; EtOAc in petroleum ether, gradient from 0/100 to 50/50). The desired fractions were collected and concentrated in vacuo to yield intermediate 21a as a white solid (14.0 g, 40%).

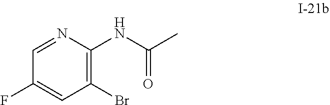

Intermediate 21b was prepared following an analogous procedure to the one described for the synthesis of intermediate 20a using 2-amino-3-bromo-5-fluoropyridine as starting material.

### Preparation of Intermediate I-22a

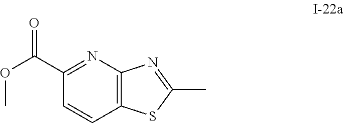

Phosphorus pentasulfide (CAS: 1314-80-3; 13.7 g, 61.5 mmol) was added to a suspension of intermediate 21a (14.0 g, 51.3 mmol) in THF (200 mL) under N₂. The mixture was stirred at 25° C. for 16 h and then at 70° C. for 48 h. Then the solvent was evaporated in vacuo and the residue purified by flash column chromatography (SiO₂; EtOAc in petroleum ether, gradient from 0/100 to 50/50). The desired fractions were collected and concentrated in vacuo to yield intermediate 22a as a yellow solid (7.5 g, 69%).

### Preparation of Intermediate I-23a

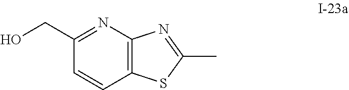

NaBH₄ (6.81 mL, 180.0 mmol) was added to a stirred suspension of intermediate 22a (7.55 g, 36.0 mmol) in THF (60 mL). The mixture was stirred at 25° C. for 5 h and then a saturated NH₄Cl solution (100 mL) was added. The mixture was extracted with DCM and the organic layer was separated, dried (Na₂SO₄), filtered and the solvents were evaporated in vacuo to yield intermediate 23a as a yellow solid (3.1 g, 51%).

### Preparation of Intermediate I-24a

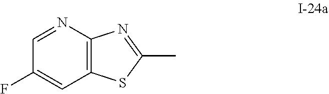

Phosphorus pentasulfide (1.70 g, 7.67 mmol) was added to a suspension of intermediate 21b (1.38 g, 5.90 mmol) in THF (32.2 mL). The mixture was stirred at rt for 16 h and an additional amount of phosphorus pentasulfide (0.39 g, 1.77 mmol) was added. The mixture was stirred at rt for another 16 h and then Cs₂CO₃ (3.08 g, 9.44 mmol) was added. The mixture was stirred at 70° C. for 16 h and then additional quantity of Cs₂CO₃ (3.08 g, 9.44 mmol) was added. The mixture was stirred at 70° C. for a further 16 h and then water was added. The mixture was extracted with EtOAc and the organic layer was separated, dried (Na₂SO₄), filtered and the solvents were evaporated in vacuo. The residue purified by flash column chromatography (SiO₂; EtOAc in heptane, gradient from 0/100 to 60/40). The desired fractions were collected and concentrated in vacuo to yield intermediate 24a as a pale-orange solid (0.78 g, 78%).

### Preparation of Intermediate I-25a

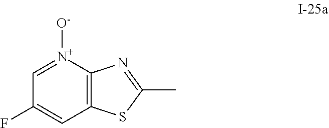

m-Chloroperbenzoic acid (CAS: 937-14-4; 1.13 g, 6.42 mmol) was added to a mixture of intermediate 24a (0.72 g, 4.28 mmol) in DCM (24 mL). The mixture was stirred at rt for 16 h and then more m-chloroperbenzoic acid (1.13 g, 6.42 mmol) was added. The mixture was stirred at rt for a further 3 d and then water was added and the mixture extracted with DCM. The organic layer was separated, dried (MgSO₄), filtered and the solvents were evaporated in vacuo. The residue was taken up into DCM and the solid formed was filtered off and discarded. The filtrate was evaporated in vacuo and the residue purified by flash column chromatography (SiO₂; MeOH in DCM, gradient from 0/100 to 10/90). The desired fractions were collected and concentrated in vacuo to yield intermediate 25a as a white solid (0.51 g, 65%).

### Preparation of Intermediate I-26a

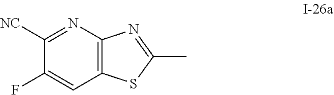

Trimethylsilyl cyanide (CAS: 7677-24-9; 0.54 mL, 4.34 mmol) was added to a mixture of intermediate 25a (0.40 g, 2.17 mmol) in ACN (5.9 mL). The mixture was stirred at 90° C. for 16 h. After cooling to rt water was added and the mixture extracted with EtOAc. The organic layer was separated, dried (MgSO₄), filtered and the solvents were evaporated in vacuo. The residue was purified by flash column chromatography (SiO₂; DCM). The desired fractions were collected and concentrated in vacuo to yield intermediate 26a as a white solid (0.22 g, 51%).

### Preparation of Intermediate I-27a

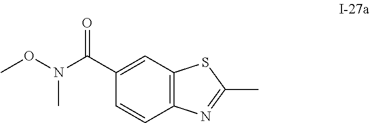

HATU (CAS: 148893-10-1; 2.36 g, 6.20 mmol) and DIPEA (2.88 mL, 16.53 mmol) and N,O-dimethylhydroxylamine hydrochloride (CAS: 6638-79-5; 0.613 g, 6.29 mmol) were added to a stirred solution of 2-methyl-1,3-benzothiazole-6-carboxylic acid (CAS: 6941-28-2; 1 g, 5.18 mmol) in DMF (25.9 mL). The mixture was stirred at rt for 16 h and then brine was added. The mixture was extracted with EtOAc and the organic layer was separated, dried (MgSO₄), filtered and the solvents were evaporated in vacuo. The residue was purified by flash column chromatography (SiO₂; EtOAc in heptane, gradient from 0/100 to 50/50). The desired fractions were collected and concentrated in vacuo to yield intermediate 27a as a colorless oil which solidified upon standing (1.3 g, 96%).

### Preparation of Intermediates I-28a, 28b and 28c

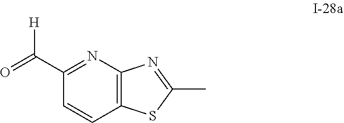

MnO₂ (CAS: 1313-13-9; 7.48 g, 86.0 mmol) was added to a stirred suspension of intermediate 23a (7.55 g, 36.0 mmol) in 1,4-dioxane (50 mL). The mixture was stirred at 80° C. for 16 h and then filtered through a Celite® pad. The filtrate was evaporated in vacuo and the residue was purified by flash column chromatography (SiO₂; EtOAc in petroleum ether, gradient from 0/100 to 50/50). The desired fractions were collected and concentrated in vacuo to yield intermediate 28a as a yellow solid (2.0 g, 65%).

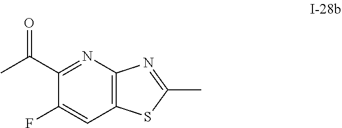

Methyl magnesium bromide (1.4M in THF/toluene, 0.85 mL, 1.19 mmol) was added to a mixture of intermediate 26a (0.12 g, 0.60 mmol) in toluene (5 mL). The mixture was stirred at rt for 16 h and then a saturated NH₄Cl solution was added. The mixture was extracted with EtOAc and the organic layer was separated, dried (Na₂SO₄), filtered and the solvents were evaporated in vacuo. The residue was purified by flash column chromatography (SiO₂; MeOH in DCM, gradient from 0/100 to 2/98). The desired fractions were collected and concentrated in vacuo to yield intermediate 28b as a yellow solid (0.020 g, 16%).

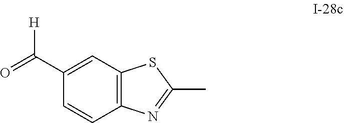

Diisobutylaluminium hydride (1M in DCM, 2.86 mL, 2.86 mmol) was added dropwise to a stirred solution of intermediate 27a (0.5 g, 1.90 mmol) in DCM (1.2 mL) at −30° C. under N₂. The mixture was stirred at −30° C. for 2 h and then sodium sulfate decahydrate was added and the mixture was stirred for 30 min. The mixture was filtered through a Celite® pad and the pad was washed with DCM. The filtrate was evaporated in vacuo. The residue was purified by flash column chromatography (SiO₂; EtOAc in heptane, gradient from 0/100 to 50/50). The desired fractions were collected and concentrated in vacuo to yield intermediate 28c as a pale-yellow solid (0.24 g, 71%).

### Preparation of Intermediate I-29a

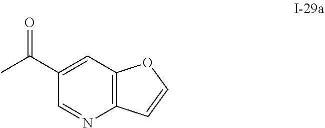

Tributyl(1-ethoxyvinyl)tin (CAS: 97674-02-7; 1.8 mL, 5.33 mmol) and Pd(PPh₃)₂Cl₂ (0.34 g, 0.49 mmol) were added to a stirred mixture of 6-bromofuro[3,2-b]pyridine (CAS: 935330-61-7, 0.96 g, 4.87 mmol) in toluene (25 mL) in a sealed tube under N₂. The mixture was stirred at 80° C. for 16 h. After cooling to rt, a 1M HCl solution (9.5 mL) was added and the mixture was stirred at 80° C. for a further 5 h. After cooling to rt, the mixture was poured onto a stirred mixture of a saturated NaHCO₃ solution and ice and extracted with DCM. The organic layer was separated, washed with brine, dried (MgSO₄), filtered and the solvents were evaporated in vacuo. The residue was purified by flash column chromatography (SiO₂; EtOAc in DCM, gradient from 0/100 to 50/50). The desired fractions were collected and concentrated in vacuo to yield intermediate 29a as a pale-orange solid (0.24 g, 63%).

### Preparation of Intermediate 63

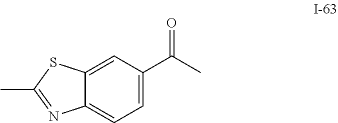

Methylmagnesium bromide (1.4M in THF/toluene, 3.6 mL, 5.04 mmol) was added dropwise to a stirred solution of intermediate 27a (991 mg, 4.19 mmol) in 2-MeTHF (20 mL) at 0° C. in a round-bottom flask and under N₂ atmosphere. The reaction mixture was stirred at 0° C. for 5 min and at room temperature for 2 h. The mixture was treated with NH₄Cl (sat., aq.) and extracted with EtOAc. The organic layer was dried (MgSO₄), filtered and the solvents were evaporated in vacuo. The crude mixture was purified by flash column chromatography (SiO₂, EtOAc in heptane, gradient from 0/100 to 100/0) to afford intermediate 63 (672 mg, 84%).

### Preparation of Intermediates 64 AND 11a

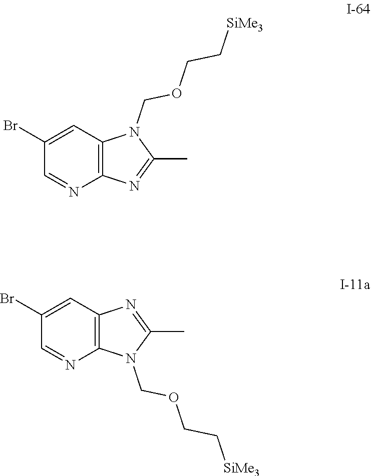 [SMILES: Cc1nc2ncc(Br)cc2n1COCC[Si](C)(C)C ; Cc1nc2cc(Br)cnc2n1COCC[Si](C)(C)C]

NaH (60% dispersion in mineral oil, 1.14 g, 28.5 mmol) was added portionwise to a solution of 6-bromo-2-methyl-1H-imidazolo[4,5-b]pyridine [42869-47-6] (3.00 g, 14.1 mmol) in DMF (30 mL) at 0° C. The mixture was stirred at room temperature for 30 min and 2-(trimethylsilyl)ethoxymethyl chloride (CAS: 76513-69-4; 4.51 mL, 25.5 mmol) was added at 0° C. The reaction mixture was stirred at room temperature for 16 h. The reaction was diluted with NH₄Cl and extracted with EtOAc. The organic layer was dried (MgSO₄), filtered and concentrated in vacuo. The crude mixture was purified by flash column chromatography (SiO₂, NH₃ (7N in MeOH) in DCM, gradient from 0/100 to 2/98) to afford intermediate 64 (873.3 mg, 18%) and intermediate 11a (219 mg, 4%) as well as a mixture of the 2 products (2.11 g).

### Preparation of Intermediate 65

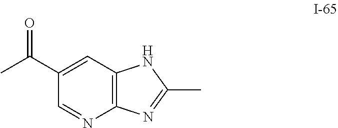

Tributyl(1-ethoxyvinyl)tin (CAS: 97674-02-7; 0.43 mL, 1.26 mmol) followed by PdCl₂(PPh₃)₂ (76.3 mg, 0.11 mmol) were added to a stirred deoxygenated solution of intermediate 64 (412 mg, 1.20 mmol) in toluene (5 mL) in a sealed tube and under N₂ atmosphere. The reaction mixture was stirred at 80° C. for 16 h. Then HCl (1M solution, 2.4 mL) was added and the mixture was stirred at 80° C. for 6 h. The mixture was added to a stirred solution of NaHCO₃ (sat., aq.) and ice and extracted with EtOAc. The organic layer was separated, dried (MgSO₄), filtered and the solvents were evaporated in vacuo. The crude mixture was purified by flash column chromatography (SiO₂, 7N solution of NH₃ in MeOH in DCM, gradient from 0/100 to 10/90) to afford intermediate 65 (56.7 mg, 27%).

### Preparation of Intermediate 66

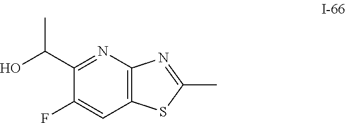

NaBH₄ (270 mg, 7.14 mmol) was added to a solution of intermediate I-28b (375 mg, 1.78 mmol) in EtOH (8.3 mL) at 0° C. The reaction mixture was stirred at room temperature for 10 min. Water was added and the mixture was extracted with DCM. The combined organic layers were dried (Na₂SO₄), filtered and concentrated in vacuo to afford intermediate 66 (335 mg) which was used in the next step.

### Preparation of Intermediate 67

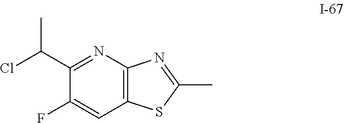

SOCl₂ (0.46 mL, 6.31 mmol) was added to a solution of intermediate 66 (335 mg, crude) in DCM (11 mL) at 0° C. The reaction mixture was stirred at room temperature for 12 h. Water was added and the mixture was extracted with DCM. The combined organic layers were dried (Na₂SO₄), filtered and evaporated in vacuo. The residue was co-evaporated with toluene (twice) and dried under vacuum to afford intermediate 67 (356 mg) which was used as such in the next step.

### Preparation of Intermediate 68

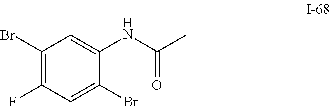

Acetic anhydride (0.35 mL, 3.72 mmol) was added to a solution of 2,5-dibromo-4-fluoroaniline (CAS: 172377-05-8; 1.00 g, 3.72 mmol) in toluene (5.6 mL). The reaction mixture was stirred at 100° C. for 2 days. The mixture was cooled down and the solid was filtered off and washed with Et₂O to afford intermediate 68 (0.97 g, 84%).

### Preparation of Intermediate 69

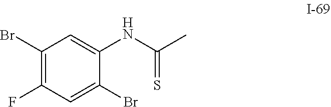

P₂S₅ (0.90 g, 4.06 mmol) was added to a suspension of intermediate 68 (0.97 g, 3.12 mmol) in THF (17 mL). The reaction mixture was stirred at room temperature for 16 h and Cs₂CO₃ (1.63 g, 4.99 mmol) was added. The mixture was stirred at 70° C. for 16 h. Water and NaOH (2N, aq.) were added and the mixture was extracted with EtOAc. The organic layer was dried (MgSO₄), filtered and the solvents were evaporated in vacuo. The crude mixture was purified by flash column chromatography (SiO₂, EtOAc in heptane, gradient from 0/100 to 80/20) to afford intermediate 69 (620 mg, 61%).

### Preparation of Intermediate 70

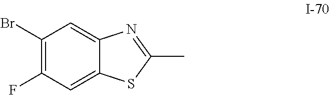

Intermediate 69 (620 mg, 1.90 mmol) was added to a suspension of NaH (60% dispersion in mineral oil, 91.0 mg, 2.28 mmol) in toluene (8.5 mL). The reaction mixture was stirred at room temperature for 2 h and DMF (1.7 mL) was added. The reaction mixture was stirred at 110° C. for 16 h. Brine was added and the mixture was extracted with EtOAc. The organic layer was dried (MgSO₄), filtered and the solvents were evaporated in vacuo to afford intermediate 70 (430 mg, 92%).

### Preparation of Intermediate 71

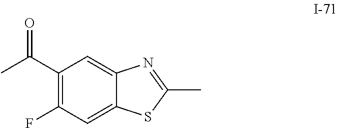

Tributyl(1-ethoxyvinyl)tin (CAS: 97674-02-7; 0.68 mL, 2.00 mmol) followed by Pd(PPh₃)₂Cl₂ (117 mg, 0.17 mmol) were added to a stirred solution of intermediate 70 (410 mg, 1.67 mmol) in toluene (8.2 mL) in a sealed tube and under N₂ atmosphere. The reaction mixture was stirred at 80° C. for 16 h and HCl (1N) was added. The mixture was stirred at 70° C. for 1 h. NaHCO₃ (sat., aq.) was added and the mixture was extracted with EtOAc. The organic layer was dried (Na₂SO₄), filtered and concentrated in vacuo. The crude mixture was purified by flash column chromatography (SiO₂, EtOAc in DCM, gradient from 0/100 to 30/70) to afford intermediate 71 (326 mg, 94%).

### Preparation of Intermediate 72

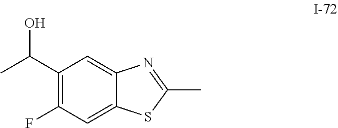

NaBH₄ (163 mg, 4.30 mmol) was added to a solution of intermediate 71 (225 mg, 1.08 mmol) in EtOH (5.0 mL) at 0° C. The reaction mixture was stirred at room temperature for 10 min. The mixture was diluted with water and extracted with DCM (3×80 mL). The combined organic layers were dried (Na₂SO₄), filtered and concentrated in vacuo to afford intermediate 72 (160 mg, 70%).

### Preparation of Intermediate 73

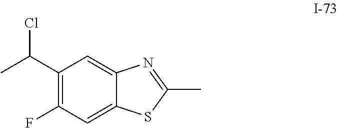

SOCl₂ (0.19 mL, 2.65 mmol) was added to a solution of intermediate 72 (140 mg, 0.66 mmol) in DCM (4.45 mL) at 0° C. The reaction mixture was stirred at room temperature for 12 h. The mixture was diluted with water (10 mL) and extracted with DCM (3×10 mL). The combined organic layers were dried (Na₂SO₄), filtered and evaporated in vacuo to afford intermediate 73 (170 mg) which was used as such in the next step.

### Preparation of Intermediate 74

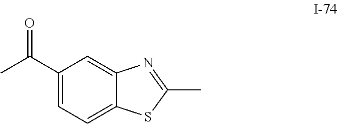

Tributyl(1-ethoxyvinyl)tin (CAS: 97674-02-7; 0.89 mL, 2.62 mmol) and Pd(PPh₃)₂Cl₂ (153 mg, 0.22 mmol) were added to a stirred solution of 6-bromo-2-methylthiazolo[5,4-b]pyridine (CAS: 886372-92-5; 500 mg, 2.18 mmol) in toluene (10.7 mL) in a sealed tube and under N₂ atmosphere. The reaction mixture was stirred at 80° C. for 16 h. HCl (1N) was added and the mixture was stirred at 70° C. for another 2 h. NaHCO₃ (sat., aq.) was added and the mixture was extracted with EtOAc. The organic layer was dried (Na₂SO₄), filtered and concentrated in vacuo. The crude mixture was purified by flash column chromatography (SiO₂, EtOAc in DCM, gradient from 0/100 to 30/70) to afford intermediate 74 (230 mg, 55%).

### Preparation of Intermediate 75

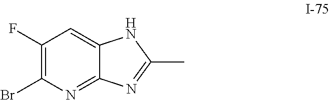

Bromine (0.51 mL, 9.92 mmol) was added to a solution of 6-fluoro-2-methyl-3H-imidazo[4,5-b]pyridine (CAS: 954218-00-9; 1.00 g, 6.62 mmol) and sodium acetate (1.36 g, 16.5 mmol) in acetic acid (10 mL). The reaction mixture was stirred at room temperature for 16 h and at 50° C. for 4 h. Additional amount of bromine (0.85 mL, 16.5 mmol) and sodium acetate (1.35 g, 16.5 mmol) were added and the reaction mixture was stirred at room temperature for 16 h and at 50° C. for 4 h. Additional quantity of bromine (0.51 mL, 9.92 mmol) was added and the reaction mixture was stirred at room temperature for another 16 h. Na₂S₂O₃ was added and the mixture was extracted with EtOAc. The organic layer was dried (Na₂SO₄), filtered and concentrated in vacuo. The crude mixture was combined with another fraction (0.33 mmol) and purified by flash column chromatography (SiO₂, MeOH in DCM, gradient from 0/100 to 6/94) to afford intermediate 75 (0.52 g, 33%).

### Preparation of Intermediate 76

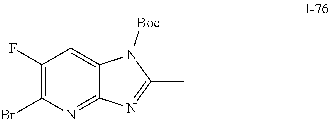 [SMILES: Cc1nc2nc(Br)c(F)cc2n1C(=O)OC(C)(C)C]

To a suspension of intermediate 75 (346 mg, 1.50 mmol) and DMAP (36.8 mg, 0.30 mmol) in THF (5.77 mL) was added dropwise bis(tert-butyl)dicarbonate (CAS: 24424-58-3; 657 mg, 3.00 mmol). The reaction mixture was stirred at room temperature for 16 h. NH₄Cl (sat., aq.) was added and the mixture was extracted with EtOAc. The organic layer was dried (MgSO₄), filtered and concentrated in vacuo to afford intermediate 76 (516 mg, 89%, 86% purity) which was used as such in the next step.

### Preparation of Intermediate 77

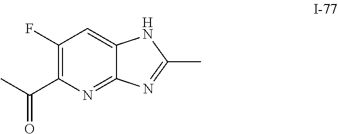

Tributyl(1-ethoxyvinyl)tin (CAS: 97674-02-7; 0.48 mL, 1.41 mmol) and Pd(PPh₃)₂Cl₂ (82.3 mg, 0.12 mmol) were added to a stirred solution of intermediate 76 (450 mg, 1.17 mmol) in toluene (9.0 mL) in a sealed tube and under N₂ atmosphere. The reaction mixture was stirred at 80° C. for 48 h. HCl (1M in H₂O, 7.5 mL, 7.5 mmol) was added and the mixture was stirred at room temperature for 16 h. NaHCO₃ (sat., aq.) was added and the mixture was extracted with EtOAc. The organic layer was dried (Na₂SO₄), filtered and concentrated in vacuo. The crude mixture was purified by flash column chromatography (SiO₂, MeOH in DCM, gradient from 0/100 to 10/90) to afford intermediate 77 (190 mg, 84%).

### Preparation of Intermediate 78

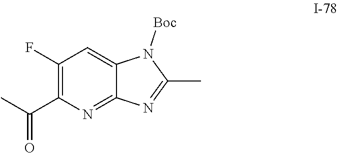 [SMILES: CC(=O)c1nc2nc(C)n(C(=O)OC(C)(C)C)c2cc1F]

To a suspension of intermediate 77 (122 mg, 0.63 mmol) and DMAP (15.4 mg, 0.13 mmol) in THF (2.4 mL) was added dropwise bis(tert-butyl)dicarbonate (275 mg, 1.26 mmol). The reaction mixture was stirred at room temperature for 16 h. NH₄Cl (sat., aq.) was added and the mixture was extracted with EtOAc. The organic layer was dried (MgSO₄), filtered and concentrated in vacuo. The crude mixture was purified by flash column chromatography (SiO₂, EtOAc in heptane, gradient from 0/100 to 40/60) to afford intermediate 78 (176 mg, 95%).

### Preparation of Intermediate 79

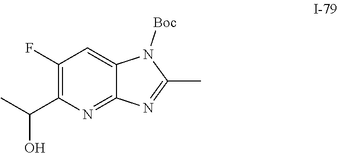 [SMILES: Cc1nc2nc(C(C)O)c(F)cc2n1C(=O)OC(C)(C)C]

Sodium methoxide (2.84 μL, 12.4 μmol) was added to a stirred suspension of intermediate 78 (150 mg, 0.51 mmol) in MeOH (2.0 mL) at 0° C. under N₂ atmosphere. NaBH₄ (19.3 mg, 0.51 mmol) was added portionwise at this temperature. The mixture was stirred for 45 min. Water was added and the mixture was extracted with EtOAc. The organic layer was dried (MgSO₄), filtered and concentrated in vacuo. The residue was dissolved in THF (2 mL), and Et₃N (70 μL, 0.5 mmol) and bis(tert-butyl)dicarbonate (CAS: 24424-58-3; 120 mg, 0.55 mmol) were added at 0° C. The reaction mixture was stirred at room temperature for 16 h and quenched with water. The mixture was extracted with DCM. The combined organic layers were dried (Na₂SO₄), filtered and evaporated in vacuo to afford intermediate 79 (140 mg, 93%).

### Preparation of Intermediate 80

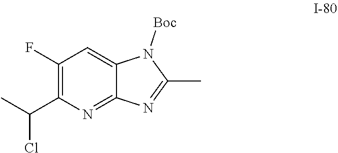 [SMILES: Cc1nc2nc(C(C)Cl)c(F)cc2n1C(=O)OC(C)(C)C]

SOCl₂ (84 μL, 1.15 mmol) was added dropwise to a mixture of intermediate 79 (85.0 mg, 0.29 mmol) and Et₃N (0.32 mL, 2.30 mmol) in DCM (1.9 mL) at 0° C. The reaction mixture was stirred at room temperature for 12 h. The reaction was cooled to 0° C. and water was carefully added. The mixture was extracted with DCM. The combined organic layers were dried (Na₂SO₄), filtered and evaporated in vacuo to afford intermediate 80 which was used as such in the next step.

### Preparation of Intermediate 85

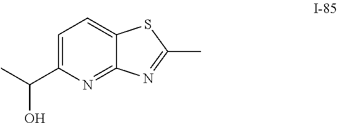

Methylmagnesium bromide (1.4M in THF and toluene, 1.31 mL, 1.83 mmol) was added over a solution of intermediate 28a (200 mg, 1.12 mmol) in anhydrous THF (11.2 mL) at 0° C. and under N₂ atmosphere. The reaction mixture was stirred from 0° C. to room temperature for 2 h, diluted with NH₄Cl (sat., aq.) and extracted with Et₂O. The organic layer was separated, dried (MgSO₄), filtered and the solvents were evaporated in vacuo. The crude product was purified by flash column chromatography (SiO₂, EtOAc in heptane, gradient from 20/80 to 100/0) to afford intermediate 85 (176 mg, 81%) as a yellow solid.

### Preparation of Intermediate 86

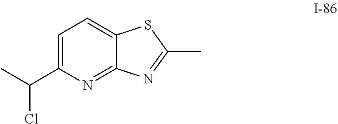

SOCl₂ (88.6 μL, 1.18 mmol) was added to a stirred solution of intermediate 85 in anhydrous DCM (9.1 mL) at 0° C. The reaction mixture was stirred from 0° C. to room temperature for 2 h and the solvent was evaporated in vacuo to yield intermediate 86 which was used as such in the next step.

### Preparation of Intermediate 87

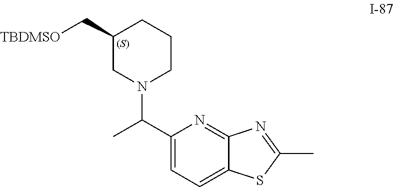

Intermediate I-28a (376 mg, 2.11 mmol) and Ti(Oi-Pr)₄ (CAS: 546-68-9; 1.87 mL, 6.33 mmol) were added to a solution of 3-((tert-butyldimethylsiloxyl)methyl)piperidine [876147-50-1] (508 mg, 2.22 mmol) in anhydrous THF (5.41 mL) at room temperature. The reaction mixture was stirred at room temperature for 18 h. The mixture was distilled and dried in vacuo. Anhydrous THF (5.41 mL) was added and the reaction was cooled to 0° C. Methylmagnesium bromide (1.4M in THF, 7.53 mL, 10.6 mmol) was added dropwise. The reaction mixture was stirred at 0° C. for 15 min and at room temperature for 15 h. NH₄Cl (sat., aq.) was added and the mixture was extracted with DCM (3 times). The combined organics extracts were dried (MgSO₄), filtered and concentrated in vacuo. The crude mixture was purified by flash column chromatography (SiO₂, heptane/EtOAc, 95:5 to 0:100) to afford intermediate 87 (635 mg, 74%).

### Preparation of Intermediate 88

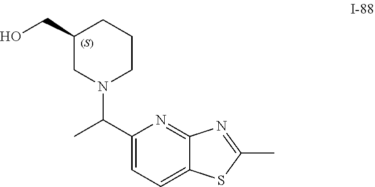

TBAF (875 mg, 3.13 mmol) was added to a stirred solution of intermediate I-87 (635 mg, 1.57 mmol) in THF (25 mL) at room temperature. The reaction mixture was stirred at room temperature for 3 h. The solvents were evaporated in vacuo. The crude mixture was purified by flash column chromatography (SiO₂, DCM:MeOH (10:1)/DCM, gradient from 0:100 to 10:90) to afford intermediate 88 (312 mg, 68%).

### Preparation of Intermediate 89

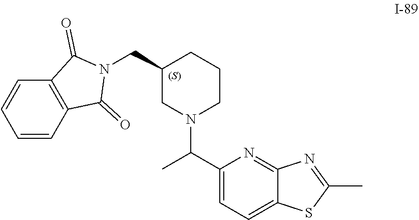

A solution of intermediate I-88 (312 mg, 1.07 mmol), phtalimide (173 mg, 1.18 mmol) and triphenylphosphine (421 mg, 1.61 mmol) in anhydrous THF (12.7 mL) was stirred under N₂ atmosphere. DIAD (318 mg, 1.61 mmol) was added and the reaction mixture was stirred at room temperature overnight. The mixture was diluted with water and extracted with EtOAc. The organic layer was dried (MgSO₄), filtered and the solvents were evaporated in vacuo. The crude mixture was purified by flash column chromatography (SiO₂, heptane/EtOAc, gradient from 100:0 to 0:100) to afford intermediate 89 (434 mg, 96%).

### Preparation of Intermediate 90

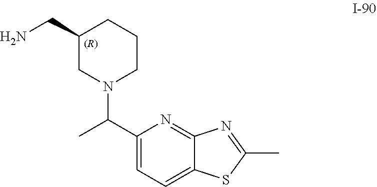

Hydrazine monohydrate (75.3 μL, 1.55 mmol) was added to a stirred solution of intermediate I-89 (434 mg, 1.03 mmol) in EtOH (12 mL). The reaction mixture was stirred at 80° C. for 2 h and room temperature for 15 h. The solvent was evaporated in vacuo. The crude mixture was dissolved in DCM and filtered. The filtrate was evaporated in vacuo. The crude mixture was purified by flash column chromatography (SiO₂, MeOH:NH₃ in DCM, gradient from 0:100 to 10:90) to afford intermediate 90 (137 mg, 46%).

### Preparation of Intermediates I-30a, 30b, 30c, 30d, 30e, 30f, 30g, 30h and 30i

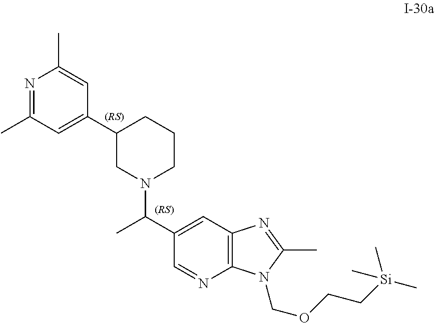

Titanium (IV) isopropoxide (CAS: 546-68-9; 0.23 mL, 0.79 mmol) was added to a stirred solution of intermediate 3a (0.1 g, 0.53 mmol) and intermediate 14a in DCM (1.81 mL) at rt under N₂. The mixture was stirred at rt for 16 h. Then mixture was cooled down to 0° C. and methylmagnesium bromide (1.4M in THF/toluene, 1.88 mL, 2.63 mmol) was added. The mixture was stirred at 0° C. for 15 min and then allowed to warm up to rt and stirred for a further 2 h. Then a saturated NH₄Cl solution and DCM were added and the mixture was filtered through a Celite® pad. The organic layer was separated, dried (Na₂SO₄), filtered and the solvents removed in vacuo. The residue was purified by flash column chromatography (SiO₂; EtOAc in DCM, gradient from 0/100 to 50/50). The desired fractions were collected and concentrated in vacuo to yield intermediate 30a as pale-yellow oil (0.214 g, 68%).

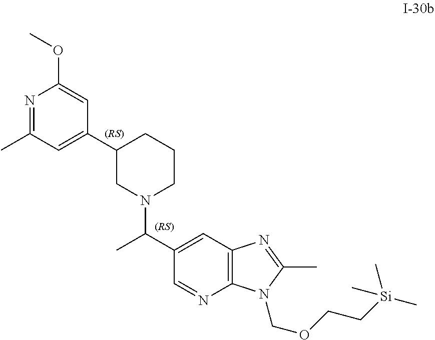

Intermediate 30b was prepared following an analogous procedure to the one described for the synthesis of intermediate 30a using intermediates 3b and 14a as starting materials.

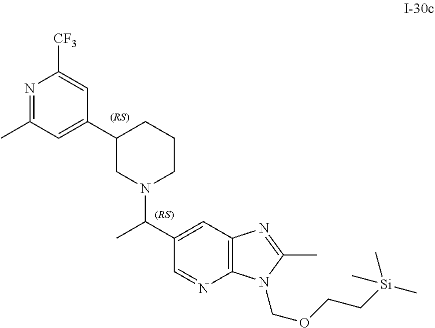

Intermediate 30c was prepared following an analogous procedure to the one described for the synthesis of intermediate 30a using intermediates 3c and 14a as starting materials.

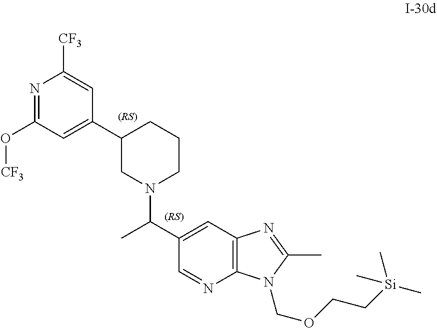 [SMILES: Cc1nc2cc(C(C)N3CCCC(c4cc(OC(F)(F)F)nc(C(F)(F)F)c4)C3)cnc2n1COCC[Si](C)(C)C]

Intermediate 30d was prepared following an analogous procedure to the one described for the synthesis of intermediate 30a using intermediates 3d and 14a as starting materials.

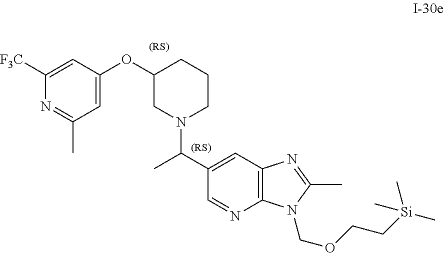 [SMILES: Cc1cc(OC2CCCN(C(C)c3cnc4c(c3)nc(C)n4COCC[Si](C)(C)C)C2)cc(C(F)(F)F)n1]

Titanium (IV) isopropoxide (CAS: 546-68-9; 0.205 mL, 0.69 mmol) was added to a stirred solution of intermediate 5b (0.12 g, 0.46 mmol) and intermediate 14a in DCM (1.81 mL) at rt under N₂. The mixture was stirred at 80° C. for 16 h. Then mixture was cooled down to rt and methylmagnesium bromide (1.4M in THF/toluene, 1.65 mL, 2.31 mmol) was added. The mixture was stirred at rt for 16 h and then a saturated NaHCO₃ solution was added. The mixture was extracted with DCM and the organic layer was separated, dried (MgSO₄), filtered and the solvents removed in vacuo. The residue was purified by flash column chromatography (SiO₂; 7N solution of NH₃ in MeOH in DCM, gradient from 0/100 to 10/90). The desired fractions were collected and concentrated in vacuo to yield intermediate 30e as a colorless oil (0.132 g, 52%).

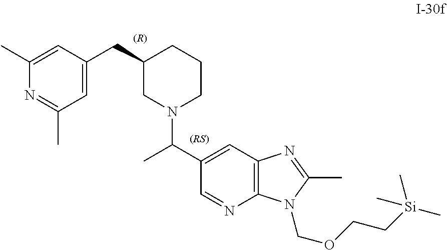

Intermediate 30f was prepared following an analogous procedure to the one described for the synthesis of intermediate 30a using intermediates 8a and 14a as starting materials.

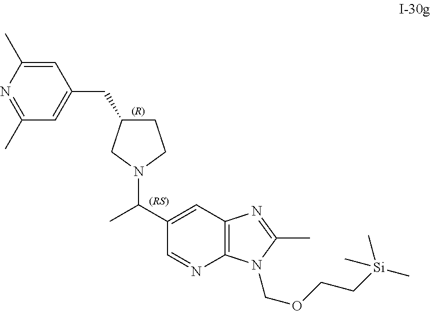

Intermediate 30g was prepared following an analogous procedure to the one described for the synthesis of intermediate 30a using intermediates 8e and 14a as starting materials.

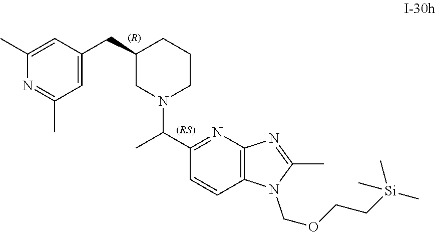

Titanium (IV) isopropoxide (CAS: 546-68-9; 0.205 mL, 0.69 mmol) and intermediate 14b (0.135 g, 0.47 mmol) were added to a stirred solution of intermediate 8a (0.063 g, 0.31 mmol) in DCM (1 mL) at rt under N₂. The mixture was stirred at rt for 16 h. Then the solvent was evaporated in vacuo and the residue was dissolved in DCM (1 mL). The mixture was cooled down to 0° C. and methylmagnesium bromide (1.4M in THF/toluene, 1.11 mL, 1.55 mmol) was added. The mixture was stirred at 0° C. for 15 min and at rt for 1.5 h and then a saturated NH₄Cl solution was added and the mixture was extracted with DCM. The organic layer was separated, dried (MgSO₄), filtered and the solvents evaporated in vacuo. The residue was purified by flash column chromatography (SiO₂; MeOH in DCM, gradient from 0/100 to 5/95). The desired fractions were collected and concentrated in vacuo to yield intermediate 30h as a yellow oil (0.132 g, 52%).

### Preparation of Intermediate 91

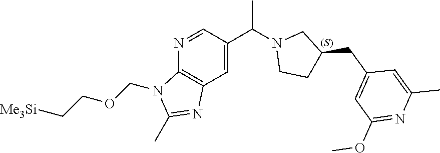

Intermediate 14a (109 mg, 0.37 mmol) and Ti(Oi-Pr)₄ (151 μL, 0.51 mmol) were added to a stirred solution of intermediate 32 (70.0 mg, 0.34 mmol) in DCM (2 mL) under N₂ atmosphere. The reaction mixture was stirred at room temperature for 16 h. The mixture was cooled to 0° C. and THF (1 mL) was added, followed by MeMgBr (1.4 M in THF/toluene, 1.2 mL, 1.70 mmol) dropwise. The reaction mixture was stirred at this temperature for 25 min and at room temperature for 2 h. The mixture was treated with NH₄Cl (sat., aq.) and water and extracted with DCM. The organic layer was dried (Na₂SO₄), filtered and the solvent was evaporated in vacuo. The crude mixture was purified by flash column chromatography (Sift, amino functionalized, MeOH in DCM, gradient from 0/100 to 4/96). The residue was purified by RP HPLC (stationary phase: C18 XBridge 30×100 mm 5 μm), mobile phase: NH₄HCO₃ (0.25% solution in water)/ACN, gradient from 47/53 to 30/70) to afford intermediate 91 (84 mg, 50%).

### Preparation of Intermediate 92

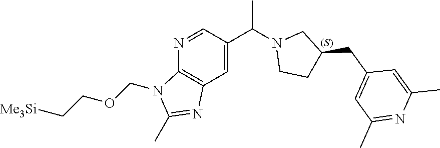

Intermediate 92 was prepared following an analogous procedure to the one described for intermediate 91 using intermediate 14a and intermediate 8f as starting materials. The crude mixture was purified by flash column chromatography (Sift, NH₃ (7M in MeOH)/DCM, gradient from 0/100 to 10/90) to afford intermediate 92 (170 mg, 67%).

### Preparation of Intermediate 93

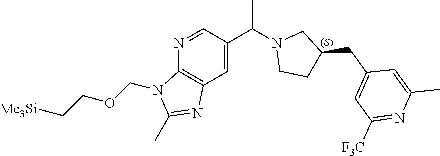

Intermediate 14a (80 mg, 0.33 mmol), intermediate 8h (105 mg, 0.36 mmol) and Ti(O-iPr)₄ (145 μL, 0.49 mmol) were dissolved in DCM (1.13 mL) at room temperature and under N₂ atmosphere. The reaction mixture was stirred at this temperature for 16 h. Then it was cooled to 0° C. and MeMgBr (1.4M in THF/toluene, 1.17 mL, 1.64 mmol) was added dropwise. The mixture was stirred at this temperature for 15 min and at room temperature for 1 h. The mixture was treated with NH₄Cl (sat., aq.) and diluted with DCM. The mixture was filtered through a pad of diatomaceus earth. The organic layer was separated, dried (MgSO₄), filtered and the solvents were evaporated in vacuo. The crude mixture was purified by flash column chromatography (SiO₂, EtOAc/MeOH, gradient from 100:0 to 90:10) to 10/90) afford intermediate 93 (110 mg, 63%).

### Preparation of Intermediate 94

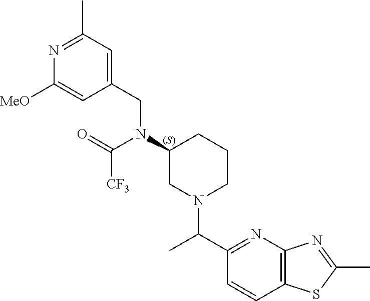

Intermediate 86 (112 mg, 0.53 mmol) was added to a solution of intermediate 60 (146 mg, 0.44 mmol) in ACN (5 mL) at room temperature. The reaction mixture was stirred at 75° C. for 48 h. The solvent was removed in vacuo and the crude mixture was purified by flash column chromatography (SiO₂, EtOAc in heptane, gradient from 20/80 to 100/0) to afford intermediate 94 (54.7 mg, 24%) as a yellow oil.

### Preparation of Intermediate 95

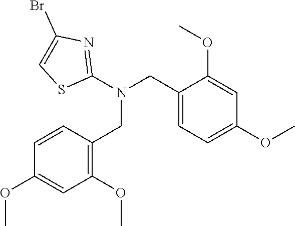 [SMILES: COc1ccc(CN(Cc2ccc(OC)cc2OC)c2nc(Br)cs2)c(OC)c1]

2,4-Dibromo-thiazole ([CAS 4175-77-3], 50 g, 205.83 mmol), N-[(2,4-dimethoxyphenyl)methyl]-2,4-dimethoxy-benzenemethanamine ([CAS 20781-23-1], 65.33 g, 205, 83 mmol) and Na₂CO₃ (65.51 g, 618 mmol) in CH₃CN (500 mL) was heated for 36 hours. The mixture was concentrated and dissolved in EtOAc (1000 mL). The mixture was washed with water (50 mL) and brine, dried over MgSO₄, and concentrated to give crude product, which was purified by column chromatography on silica gel (petroleum ether/EtOAc, from 100/0 to 70/30) to give intermediate 95 (70 g, 70%) as a yellow solid.

### Preparation of Intermediate 96

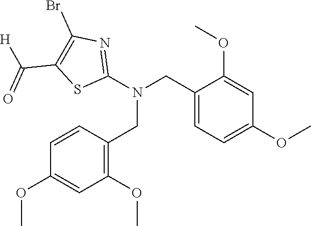 [SMILES: COc1ccc(CN(Cc2ccc(OC)cc2OC)c2nc(Br)c(C=O)s2)c(OC)c1]

To a solution of intermediate 95 (15 g, 31.29 mmol) in anhydrous THF (20 mL) was added dropwise LDA (34.42 mL, 34.42 mmol) at a rate so the temperature did not exceed −70° C. The resulting solution was stirred at −78° C. for 30 min. Then DMF (2.52 g, 34.42 mmol) was added dropwise as a solution in THF (20 mL) and the mixture was allowed to warm up to room temperature. The reaction was quenched with saturated NH₄Cl (30 mL). The mixture was extracted with EtOAc (2×50 mL). The combined organic layers were washed with brine, dried over MgSO₄, and concentrated. The crude was purified by flash chromatography on silica gel (petroleum ether/EtOAc, from 100/0 to 80/20) to yield intermediate 96 (8 g, 45%) as a light yellow solid.

### Preparation of Intermediate 97

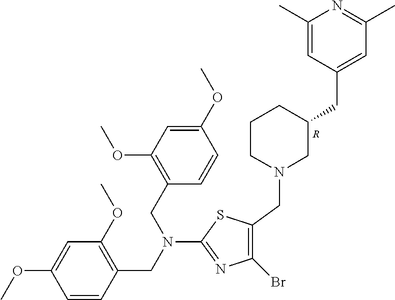

Intermediate 96 (2006.23 mg, 3.95 mmol) was added to intermediate (3R)-34 from WO2018/109202 (729 mg, 3.57 mmol) at RT. After 30 min, sodium triacetoxyborohydride (1512.43 mg, 7.14 mmol) was added to the mixture at RT and the RM was stirred for 48 h at RT. The crude was quenched with NH₃/H₂O and extracted with EtOAc. The organic layer was separated, dried (Na₂SO₄), filtered and the solvent was evaporated in vacuo. The residue was purified by automated flash chromatography (silica, 10% MeOH in DCM 0/100 to 5/95). Desired fractions were collected, concentrated under vacuo to yield intermediate 97 (1.1 g, 44%) as a sticky solid.

### Preparation of Intermediate 98

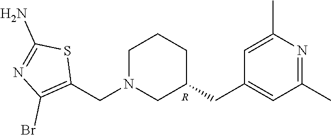

A mixture of intermediate 97 (1050 mg, 1.51 mmol) in TFA (26.25 mL) was stirred at RT under a nitrogen atmosphere for 1.5 h. The solvent was evaporated and the mixture was taken in water, basified with K₂CO₃ and extracted with DCM. The organic layer was dried over MgSO₄ and concentrated. The residue was purified on a column with silica gel, eluent DCM/MeOH (100/0 to 90/10). The pure fractions were evaporated, yielding intermediate 98 (521 mg, 87%) as a white solid.

### Preparation of Intermediate 99

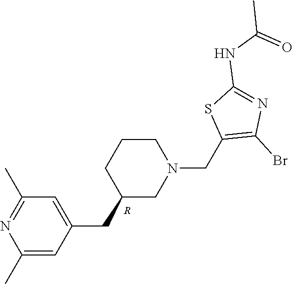

Acetic anhydride (7.75 mg, 0.076 mmol) was added dropwise to a solution of intermediate 98 (20 mg, 0.051 mmol) in 1,4-dioxane (15 mL) while stirring. After the addition was complete, the reaction was heated at 60° C. for 2 h, then at 110° C. for 4 h. The RM was evaporated, taken up in water/0.5 g NaHCO₃/DCM. The organic layer was separated, dried over MgSO₄ and concentrated. The residue was purified on a column with silica gel, eluent: DCM/MeOH (100/0 to 95/5). The pure fractions were concentrated, yielding intermediate 99 (135 mg, 41%) as a pale yellow foam.

### Preparation of [³H]-Ligand for Occupancy Study

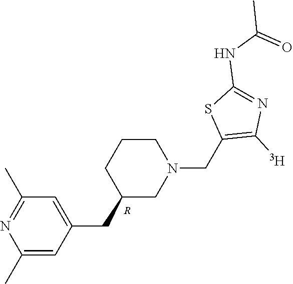

Compound 28 from WO2018/109202 was labelled with [³H] as follows: Intermediate 99 (4.10 mg, 9.38 μmol) and Palladium supported on Carbon (10%, 14.4 mg) were suspended in DMF (0.2 mL) and DIPEA (12 μL, 70.6 μmol) was added. The suspension was degassed three times and stirred under an atmosphere of Tritium gas (4.2 Ci, 525 mbar initial pressure) for 2 h 47 min at RT (end pressure was 311 mbar, no more consumption of gas was observed). The solvent was removed in vacuo, and labile tritium was exchanged by adding MeOH (0.3 mL), stirring the solution, and removing the solvent again under vacuo. This process was repeated twice. Finally, the well dried solid was extracted with EtOH (5 mL) and the suspension was filtered through a 0.2 μm nylon membrane (Macherey-Nagel Polyamide syringe filter CHROMAFIL®Xtra PA-20/25), obtaining a clear solution.

The radiochemical purity (RCP) of the crude material was determined to be 56% using the following HPLC system: Waters Atlantis T3, 5 μm, 4.6×250 mm; solvents A: water+0.05% TFA, B: acetonitrile+0.05% TFA; 0 min 0% B; 10 min 30% B; 10.2-14.5 min 95% B; 15 min 0% B; 254 nm; 1.0 mL/min; 30° C.

The crude was purified by HPLC: Waters Atlantis T3, 5 μm, 10×250 mm; solvents A: water+0.1% TFA; B: acetonitrile+0.1% TFA; 0 min 0% B, 15 min 45% B; 4.7 mL/min; 25° C. The target compound eluted at 9.5 min, and isolated from the HPLC solvent mixture by solid phase extraction. Therefore, the HPLC solution was neutralized with an aqueous solution of NaHCO₃ and the volume of the fractions were partially reduced at the rotary evaporator. Then the product was extracted with a Phenomenex StrataX cartridge (33 μm Polymeric Reversed Phase, 100 mg, 3 mL; 8B-S100-EB) which was eluted with EtOH (5 mL). The extracted product showed an RCP of >99% and the specific activity (SA) was determined to be 10.7 Ci/mmol (396 GBq/mmol, determined by MS). Two batches 250 μCi (9.25 MBq) in 0.25 mL EtOH (1 mCi/mL) and 38.8 mCi in 5 mL EtOH of [³H]-ligand were isolated.

### Preparation of the Final Compounds

### Preparation of Final Compound 1

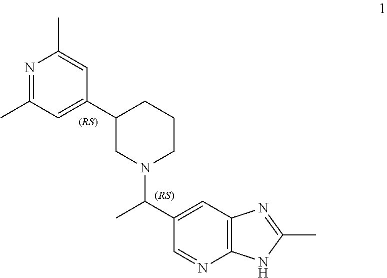

Trifluoroacetic acid (0.49 mL, 6.42 mmol) was added to a stirred solution of intermediate 30a (0.214 g, 0.36 mmol) in DCM. The mixture was stirred at rt for 16 h and then evaporated in vacuo. The residue was diluted with a saturated Na₂CO₃ solution and extracted with EtOAc. The organic layer was separated, dried (Na₂SO₄), filtered and the solvents were evaporated in vacuo. The residue was purified by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected and extracted with EtOAc. The organic layer was separated, dried (Na₂SO₄), filtered and the solvents were evaporated in vacuo. The residue was dissolved in MeOH and purified by ion exchange chromatography (ISOLUTE® SCX2 cartridge; MeOH and 7N solution of NH₃ in MeOH). The desired fractions were collected and evaporated in vacuo to yield compound 1 as a syrup which crystallized upon standing as a white solid (0.080 g, 64%).

### Preparation of Final Compound 2

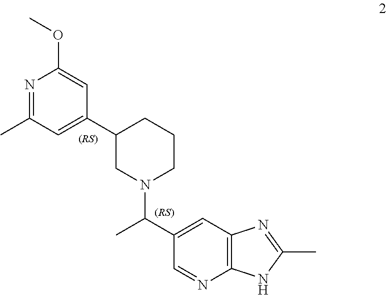

Compound 2 was prepared following an analogous procedure to the one described for the synthesis of compound 1 using intermediate 30b as starting material (0.065 g, 0.13 mmol). Compound 2 was purified by reverse phase HPLC (stationary phase: C18 XBridge 30×100 mm 5 μm, mobile phase: gradient from 80% NH₄HCO₃ 0.25% solution in water, 20% CH₃CN to 0% NH₄HCO₃ 0.25% solution in water, 100% CH₃CN) to yield compound 2 as a white solid (0.024 g, 50%).

### Preparation of Final Compound 3

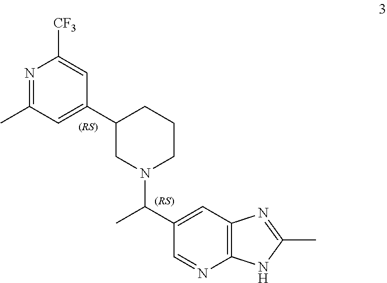

Compound 3 was prepared following an analogous procedure to the one described for the synthesis of compound 1 using intermediate 30c as starting material (0.055 g, 0.10 mmol). Compound 2 was purified by reverse phase HPLC (stationary phase: C18 XBridge 30×100 mm 5 μm), mobile phase: gradient from 80% NH₄HCO₃ 0.25% solution in water, 20% CH₃CN to 0% NH₄HCO₃ 0.25% solution in water, 100% CH₃CN) to yield compound 3 as a white solid (0.012 g, 29%).

### PREPARATION OF FINAL COMPOUNDS 4a and 4b

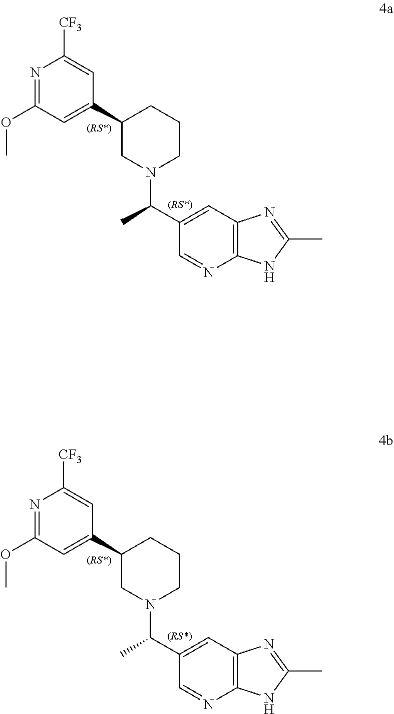 [SMILES: COc1cc([C@H]2CCCN([C@H](C)c3cnc4[nH]c(C)nc4c3)C2)cc(C(F)(F)F)n1 ; COc1cc([C@H]2CCCN([C@@H](C)c3cnc4[nH]c(C)nc4c3)C2)cc(C(F)(F)F)n1]

Compounds 4a and 4b were prepared following an analogous procedure to the one described for the synthesis of compound 1 using intermediate 30d as starting material (0.060 g, 0.11 mmol). The mixture containing compounds 4a and 4b was separated by reverse phase HPLC (stationary phase: C18 XBridge 30×100 mm 5 μm, mobile phase: gradient from 80% NH₄HCO₃ 0.25% solution in water, 20% CH₃CN to 0% NH₄HCO₃ 0.25% solution in water, 100% CH₃CN) to yield compound 4a as (0.009 g, 20%) and compound 4b as white solids (0.012 g, 26%).

### Preparation of Final Compound 5

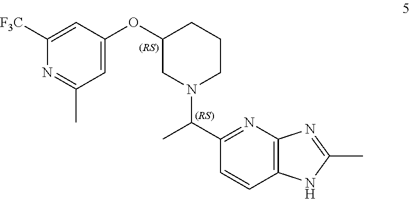 [SMILES: Cc1cc(OC2CCCN(C(C)c3ccc4[nH]c(C)nc4n3)C2)cc(C(F)(F)F)n1]

Compound 5 was prepared following an analogous procedure to the one described for the synthesis of compound 1 using intermediate 30e as starting material (0.132 g, 0.24 mmol). Compound 5 was purified by flash column chromatography (SiO₂; MeOH in DCM, gradient from 0/100 to 10/90) to yield compound 5 as a colorless oil (0.075 g, 74%).

### Preparation of Final Compounds 6a and 6b

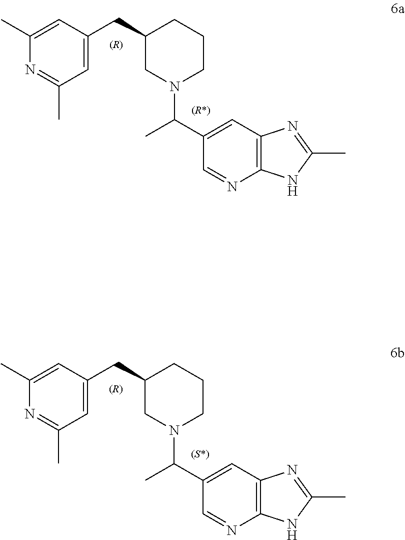

Compounds 6a and 6b were prepared following an analogous procedure to the one described for the synthesis of compound 1 using intermediate 30f as starting material (0.77 g, 0.11 mmol). The mixture of compounds 6a and 6b was purified by ion exchange chromatography (ISOLUTE® SCX2 cartridge; MeOH and 7N solution of NH₃ in MeOH). Compounds 6a and 6b were obtained by chiral SFC (stationary phase: Chiralpak IC 5 μm 250×21.2 mm, mobile phase: 60% CO₂, 40% (iPrOH/DCM 80/20 (0.3% iPrNH₂)). The fractions containing compound 6a were evaporated in vacuo and further purified by reverse phase HPLC (stationary phase: C18 XBridge 50×100 mm 5 μm, mobile phase: gradient from 84% NH₄HCO₃ 0.25% solution in water, 16% CH₃CN to 60% NH₄HCO₃ 0.25% solution in water, 40% CH₃CN) to yield 6a as a white solid (0.073 g, 13%). The fractions containing compound 6b were evaporated in vacuo and further purified by reverse phase HPLC (stationary phase: C18 XBridge 50×100 mm 5 μm, mobile phase: gradient from 84% NH₄HCO₃ 0.25% solution in water, 16% CH₃CN to 60% NH₄HCO₃ 0.25% solution in water, 40% CH₃CN) to yield 6b as a pale-yellow sticky solid (0.062 g, 11%).

### Preparation of Final Compound 7

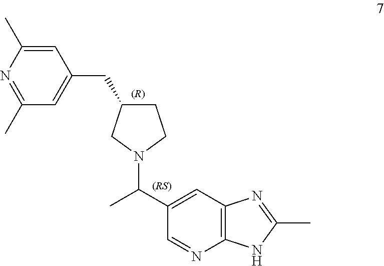

Compound 7 was prepared following an analogous procedure to the one described for the synthesis of compound 1 using intermediate 30g as starting material (0.181 g, 0.25 mmol). Compound 7 was purified by flash column chromatography (SiO₂; 7N solution of NH₃ in MeOH in DCM, gradient from 0/100 to 6/94) and by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN) to yield compound 7 as a pale-yellow foam (0.058 g, 67%).

### Preparation of Final Compound 8

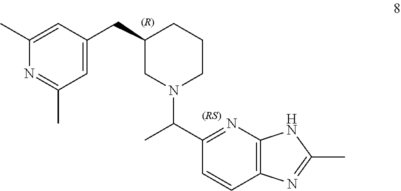

Compound 8 was prepared following an analogous procedure to the one described for the synthesis of compound 1 using intermediate 30h as starting material (0.095 g, 0.19 mmol). Compound 8 was purified by flash column chromatography (SiO₂; 7N solution of NH₃ in MeOH in DCM, gradient from 0/100 to 5/95), by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN) and triturated with DIPE to yield compound 8 as a beige sticky solid (0.037 g, 52%).

### Preparation of Final Compound 9

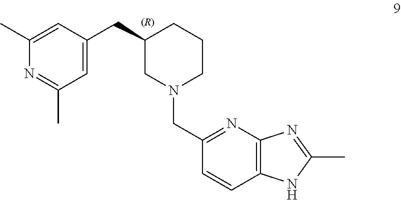

Intermediate 14a (0.079 g, 0.27 mmol) was added to a stirred solution of intermediate 8a (0.085 g, 0.31 mmol) and Et₃N (0.17 mL, 1.23 mmol) in DCM (2 mL). The mixture was stirred at rt for 30 min and then sodium triacetoxyborohydride (CAS: 56553-60-7, 0.179 g, 0.85 mmol) was added. The mixture was stirred at rt for 16 h and then a saturated NaHCO₃ solution was added. The mixture was extracted with DCM and the organic layer was separated, dried (MgSO₄), filtered and the solvents removed in vacuo. The residue was purified by flash column chromatography (SiO₂; 7N solution of NH₃ in MeOH in DCM, gradient from 0/100 to 10/90). The desired fractions were collected and concentrated in vacuo to yield compound 9 as a yellow oil (0.111 g, 85%).

### Preparation of Final Compound 10

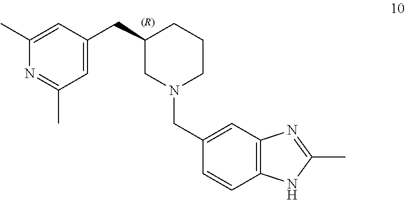

A solution of intermediate 8a (0.070 g, 0.26 mmol) and Et₃N (0.145 mL, 1.04 mmol) in DCM (1.3 mL) was added to intermediate 14c (0.085 g, 0.31 mmol) in a sealed tube under N₂. The mixture was stirred at rt for 30 min and then sodium triacetoxyborohydride (CAS: 56553-60-7, 0.179 g, 0.85 mmol) was added. The mixture was stirred at rt for 16 h and then a saturated NaHCO₃ solution was added. The mixture was extracted with DCM and the organic layer was separated, dried (MgSO₄), filtered and the solvents evaporated in vacuo. The residue was purified by flash column chromatography (SiO₂; MeOH in DCM, gradient from 0/100 to 5/95). The desired fractions were collected and concentrated in vacuo to yield compound 10 as a pale-yellow solid (0.062 g, 70%).

### Preparation of Final Compound 11

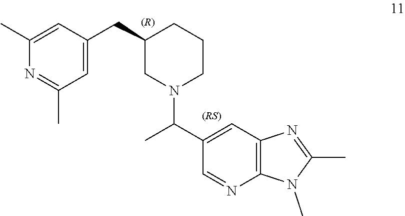

Et₃N (0.125 mL, 0.90 mmol), intermediate 14d (0.075 g, 0.40 mmol), titanium (IV) isopropoxide (CAS: 546-68-9; 0.110 mL, 0.37 mmol) and sodium cyanoborohydride (CAS: 25895-60-7; 0.050 g, 0.80 mmol) were added to a solution of intermediate 8a (0.110 g, 0.40 mmol) in 1,2-dichloroethane (1.5 mL) in a sealed tube under N₂. The mixture was stirred at 80° C. for 2 d and then a saturated NaHCO₃ solution was added. The mixture was extracted with DCM and the organic layer was separated, dried (MgSO₄), filtered and the solvents evaporated in vacuo. The residue was purified by flash column chromatography (amino functionalized SiO₂; EtOAc in heptane, gradient from 0/100 to 100/0), by flash column chromatography (SiO₂; 7N solution of NH₃ in MeOH in DCM, gradient from 0/100 to 5/95) and by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected and extracted with DCM and the organic layer was separated, dried (MgSO₄), filtered and the solvents were evaporated in vacuo. to yield compound 11 as a colorless oil (0.016 g, 11%).

### Preparation of Final Compound 12a, 12b and 12c

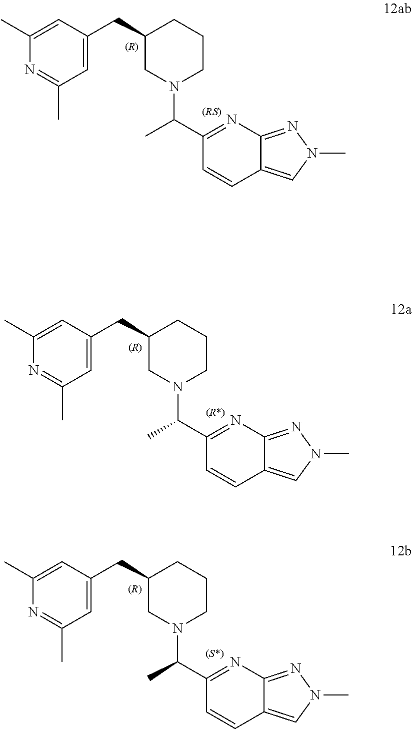

DIPEA (0.226 mL, 1.31 mmol) was added to a stirred suspension of intermediate 16a (0.097 g, 0.44 mmol) and intermediate 8a (0.158 g, 0.57 mmol) in DCM (1.34 mL). The mixture was stirred at rt for 5 min and then titanium (IV) isopropoxide (CAS: 546-68-9; 0.311 g, 1.09 mmol) and sodium cyanoborohydride (CAS: 25895-60-7; 0.068 g, 1.09 mmol) were added. The mixture was stirred at 80° C. for a further 1.5 h and then the solvent was evaporated in vacuo. The residue was purified by flash column chromatography (SiO₂; 7N solution of NH₃ in MeOH in DCM, 0/100 to 10/90) and by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN) to yield compound 12a as a yellow oil (0.036 g, 23%). A 0.03 g sample of compound 12ab was further purified by achiral SFC (stationary phase: Chiralcel OD-H 5 μm 250×21.2 mm, mobile phase: 85% CO₂, 15% (EtOH (0.3% iPrNH₂)). Desired fractions were collected and evaporated in vacuo to yield compound 12a (0.006 g, 4%) and compound 12b (0.015 g, 9%).

### Preparation of Final Compound 13

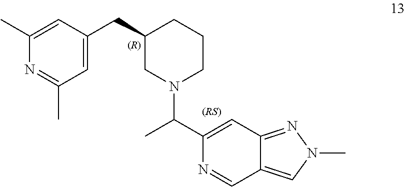

Compound 13 was prepared following an analogous procedure to the one described for the synthesis of compound 12a using intermediate 16b as starting material (0.089 g, 0.51 mmol). Compound 13 was purified by flash column chromatography (SiO₂; 7N solution of NH₃ in MeOH in DCM, gradient from 0/100 to 10/90), by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN) and by ion exchange chromatography (ISOLUTE® SCX2 cartridge; MeOH and 7N solution of NH₃ in MeOH) to yield compound 13 as a white solid (0.037 g, 52%).

### Preparation of Final Compound 14

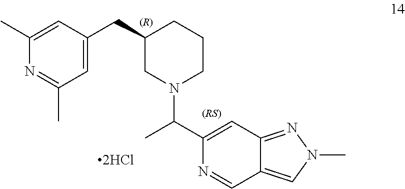

Compound 14 was prepared following an analogous procedure to the one described for the synthesis of compound 12a using intermediate 16c as starting material (0.089 g, 0.51 mmol) and Et₃N (0.150 mL, 1.08 mmol) instead of DIPEA. Compound 14 was purified by flash column chromatography (SiO₂; 7N solution of NH₃ in MeOH in DCM, 0/100 to 10/90), by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected and evaporated in vacuo and the residue was dissolved in MeOH (1 mL) and a 4M solution of HCl in 1,4-dioxane was added (0.5 mL, 2.0 mmol). The mixture was stirred at rt for 5 min and then the solvents were evaporated in vacuo to yield compound 14 as a white solid (0.065 g, 41%).

### Preparation of Final Compound 15

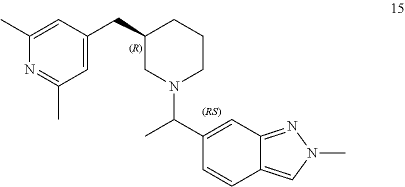

Compound 15 was prepared following an analogous procedure to the one described for the synthesis of compound 12a using intermediate 16d as starting material (0.089 g, 0.51 mmol) and Et₃N (0.150 mL, 1.08 mmol) instead of DIPEA. Compound 15 was purified by flash column chromatography (SiO₂; 7N solution of NH₃ in MeOH in DCM, 0/100 to 10/90) and by reverse phase HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN) to yield compound 15 as a pale-yellow oil (0.027 g, 23%).

### Preparation of Final Compound 16

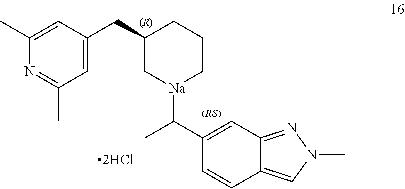

Compound 16 was prepared following an analogous procedure to the one described for the synthesis of compound 12a using 2-acetyl-2-methyl-2H-indazole as starting material (CAS: 1159511-29-1; 0.125 g, 0.72 mmol) and Et₃N (0.30 mL, 2.16 mmol) instead of DIPEA. Compound 16 was purified by flash column chromatography (SiO₂; 0.7N solution of NH₃ in MeOH in DCM, gradient from 0/100 to 100/0), by reverse phase HPLC (stationary phase: C18 XBridge 50×100 mm 5 μm, mobile phase: gradient from 90% NH₄HCO₃ 0.25% solution in water, 10% CH₃CN to 66% NH₄HCO₃ 0.25% solution in water, 34% CH₃CN). The desired fractions were collected and evaporated in vacuo and the residue was dissolved in MeOH (4 mL) and a 4M solution of HCl in 1,4-dioxane was added (0.2 mL, 2.39 mmol) in a sealed tube. The mixture was stirred at rt for 1 h and then the solvents were evaporated in vacuo to yield compound 16 as a yellow solid (0.030 g, 10%).

### Preparation of Final Compound 17

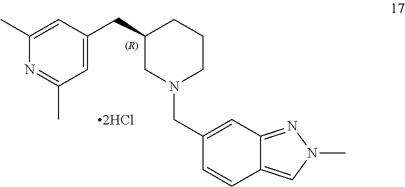

Intermediate 16e (0.085 g, 0.53 mmol) was added to a stirred solution of intermediate 8a (0.147 g, 0.53 mmol) and Et₃N (0.226 mL, 1.62 mmol) in anhydrous MeOH (1.75 mL). The mixture was stirred at rt for 16 h and the sodium triacetoxyborohydride (CAS: 56553-60-7; 0.168 g, 0.80 mmol) and the mixture was stirred at rt for a further 1 d. Then the solvent was evaporated in vacuo and the residue purified by flash column chromatography (SiO₂; 0.7N solution of NH₃ in MeOH in DCM, gradient from 0/100 to 100/0) and by reverse phase HPLC (stationary phase: C18 XBridge 30×100 mm 5 μm, mobile phase: gradient from 80% 10 mM NH₄HCO₃ pH 9 solution in water, 20% CH₃CN to 0% 10 mM NH₄HCO₃ pH 9 solution in water, 100% CH₃CN). The desired fractions were collected and evaporated in vacuo to yield the free base of compound 17 (0.092 g, 50%). A sample of the free base of compound 17 (0.078 g, 0.22 mmol) was dissolved in MeOH (1.09 mL) and a 37% solution of HCl was added (0.056 mL, 0.67 mmol) in a sealed tube. The mixture was stirred at rt for 1 h and then the solvents were evaporated in vacuo to yield compound 17 as a light-yellow solid (0.093 g, 99%).

### Preparation of Final Compound 18

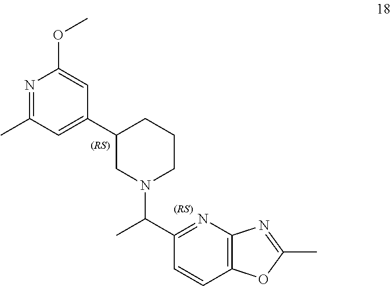

Intermediate 19a (0.094 g, 0.58 mmol) and titanium (IV) isopropoxide (CAS: 546-68-9; 0.213 mL, 0.73 mmol) were added to a stirred solution of intermediate 3b (0.10 g, 0.48 mmol) in DCM (2 mL) at rt under N₂. The mixture was stirred at rt for 16 h, cooled down to 0° C. and then methylmagnesium bromide (1.4M in THF/toluene, 1.73 mL, 2.42 mmol) was added. The mixture was stirred at 0° C. for 1 h and then a saturated NH₄Cl solution and DCM were added. The mixture was filtered through a Celite® pad. The filtrate was diluted with DCM and the organic layer was separated, dried (Na₂SO₄), filtered and the solvents evaporated in vacuo. The residue was purified by flash column chromatography (SiO₂; MeOH in EtOAc, gradient from 0/100 to 10/90). The desired fractions were collected and concentrated in vacuo and the residue was purified by reverse phase HPLC (stationary phase: C18 XBridge 30×100 mm 5 μm, mobile phase: gradient from 47% NH₄HCO₃ 0.25% solution in water, 53% CH₃CN to 30% NH₄HCO₃ 0.25% solution in water, 70% CH₃CN). The desired fractions were collected and evaporated in vacuo and the residue was dissolved in EtOAc and extracted with water. The organic layer was separated, dried (Na₂SO₄), filtered and the solvents evaporated in vacuo to yield compound 18 as a colorless film (0.074 g, 42%).

### Preparation of Final Compound 19

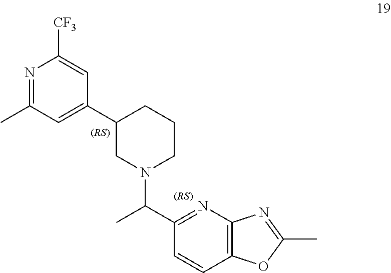

Compound 19 was prepared following an analogous procedure to the one described for the synthesis of compound 18 using intermediate 3c as starting material (0.080 g, 0.49 mmol). Compound 19 was purified by flash column chromatography (SiO₂; MeOH in EtOAc, gradient from 0/100 to 10/90) and by reverse phase HPLC (stationary phase: C18 XBridge 30×100 mm 5 μm, mobile phase: gradient from 47% NH₄HCO₃ 0.25% solution in water, 53% CH₃CN to 30% NH₄HCO₃ 0.25% solution in water, 70% CH₃CN). The desired fractions were collected and evaporated in vacuo to yield compound 19 as a colorless oil (0.090 g, 54%).

### Preparation of Final Compound 20

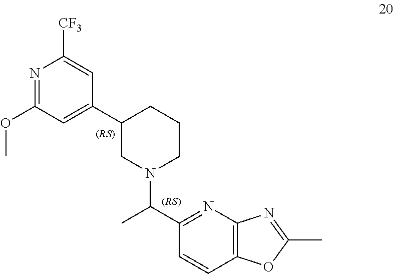 [SMILES: COc1cc(C2CCCN(C(C)c3ccc4oc(C)nc4n3)C2)cc(C(F)(F)F)n1]

Compound 20 was prepared following an analogous procedure to the one described for the synthesis of compound 18 using intermediate 3d as starting material (0.075 g, 0.46 mmol). Compound 19 was purified by flash column chromatography (SiO₂; MeOH in EtOAc, gradient from 0/100 to 10/90) and by reverse phase HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and the organic solvent evaporated in vacuo. EtOAc was added and the organic layer was separated, dried (Na₂SO₄), filtered and the solvents evaporated in vacuo to yield compound 20 as a colorless film (0.073 g, 45%).

### Preparation of Final Compounds 21ab and 21a

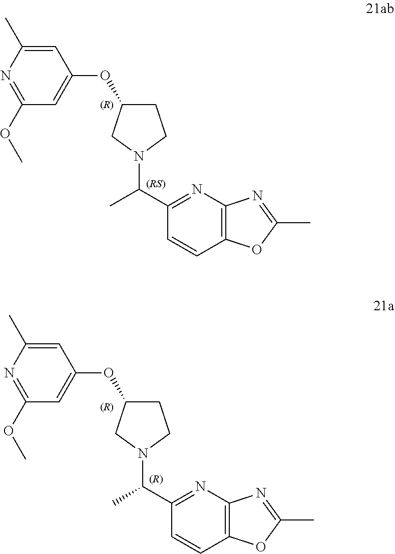

Intermediate 19a (0.082 g, 0.50 mmol) and titanium (IV) isopropoxide (CAS: 546-68-9; 0.213 mL, 0.73 mmol) were added to a stirred solution of intermediate 3b (0.10 g, 0.48 mmol) in DCM (1.5 mL) at rt under N₂. The mixture was stirred at rt for 16 h, cooled down to 0° C. and then methylmagnesium bromide (1.4M in THF/toluene, 1.72 mL, 2.40 mmol) was added. The mixture was stirred at 0° C. for 5 min and at rt for 2 h. Then a saturated NH₄Cl solution was added and the mixture extracted with DCM. The organic layer was separated, dried (MgSO₄), filtered and the solvents evaporated in vacuo. The residue was purified by flash column chromatography (SiO₂; 7N solution of NH₃ in MeOH in DCM, gradient from 0/100 to 10/90). The desired fractions were collected and concentrated in vacuo and the residue was purified by reverse phase HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and evaporated in vacuo and the residues were dissolved in EtOAc and extracted with a saturated NaHCO₃ solution. The organic layer was separated, dried (Na₂SO₄), filtered and the solvents were evaporated in vacuo to yield compound 21ab (mixture 38/62 of diasterosisomers, 0.020 g, 11%) and compound 21a (0.01 g, 6%) as brown oils.

### Preparation of Final Compound 22

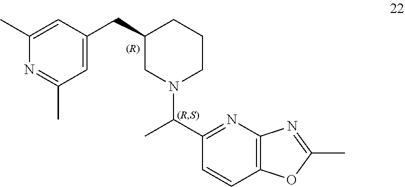

Compound 22 was prepared following an analogous procedure to the one described for the synthesis of compound 18 using intermediate 8a as starting material (0.090 g, 0.44 mmol). Compound 22 was purified by flash column chromatography (SiO₂; MeOH in DCM, gradient from 0/100 to 5/95) to yield compound 22 as a colorless film (0.072 g, 45%).

### Preparation of Final Compound 23a and 23b

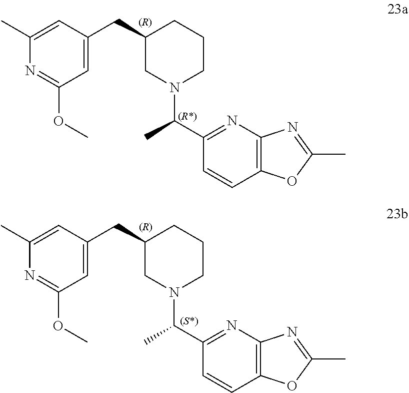

Compounds 23a and 23b were prepared following an analogous procedure to the one described for the synthesis of compound 18 using intermediate 8b as starting material (0.10 g, 0.45 mmol). The mixture of compounds 23a and 23b was purified by flash column chromatography (SiO₂; 7N solution of NH₃ in MeOH in DCM in DCM, gradient from 0/100 to 10/90) and the desired fractions were collected and evaporated in vacuo. Compounds 23a and 23b were separated by reverse phase HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and the solvents were evaporated in vacuo to yield compound 23a after drying under vacuum at 50° C. for 16 h (0.039 g, 22%), and compound 23b (0.008 g, 5%) as colorless oils.

### Preparation of Final Compound 24ab, 24a and 24b

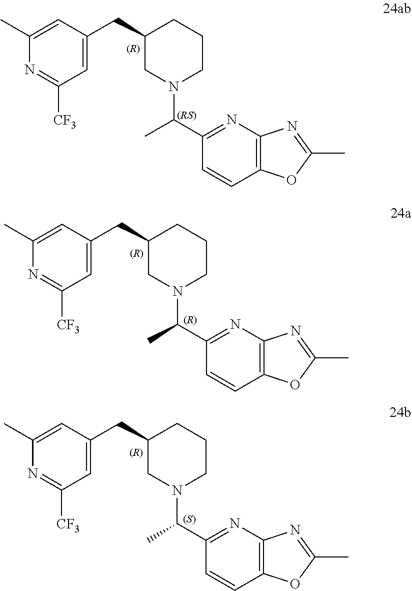

Compounds 24ab, 24a and 24b were prepared following an analogous procedure to the one described for the synthesis of compound 18 using intermediate 8c as starting material (0.10 g, 0.45 mmol). Compound 24ab was purified by flash column chromatography (SiO₂; 7N solution of NH₃ in MeOH in DCM in DCM, gradient from 0/100 to 10/90) and the desired fractions were collected and evaporated in vacuo. The residue was purified by reverse phase HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and the solvents evaporated in vacuo to yield compound 23ab (mixture 40/60 of diastereoisomers, 0.029 g, 18%), compound 24a (0.010 g, 6%), and compound 24b (0.035 g, 22%) as colorless oils.

### Preparation of Final Compounds 25ab and 25a

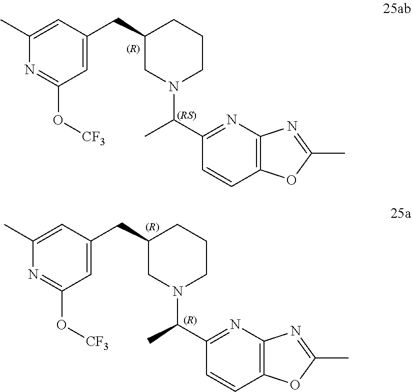

Compounds 25ab and 25a were prepared following an analogous procedure to the one described for the synthesis of compound 18 using intermediate 8d as starting material (0.10 g, 0.45 mmol). Compound 24ab was purified by flash column chromatography (SiO₂; 7N solution of NH₃ in MeOH in DCM in DCM, gradient from 0/100 to 10/90) and the desired fractions were collected and evaporated in vacuo. The residue was purified by reverse phase HPLC (stationary phase: C18 XBridge 30×100 mm 5 μm, mobile phase: gradient from 54% NH₄HCO₃ 0.25% solution in water, 46% CH₃CN to 36% NH₄HCO₃ 0.25% solution in water, 63% CH₃CN). The desired fractions were collected and the solvents were evaporated in vacuo to yield compound 25ab (0.022 g, 14%) and compound 25a (0.013 g, 8%) as yellow oils.

### Preparation of Final Compound 26

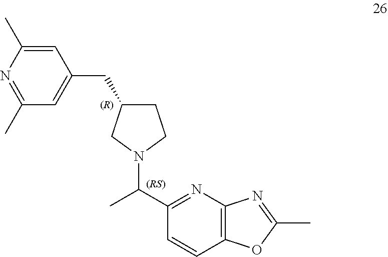

Intermediate 19a (0.170 g, 1.051 mmol) and titanium (IV) isopropoxide (CAS: 546-68-9; 0.467 mL, 1.58 mmol) were added to a stirred solution of intermediate 8e (0.10 g, 0.48 mmol) in DCM (4 mL) at rt under N₂. The mixture was stirred at rt for 16 h, cooled down to 0° C. and then methylmagnesium bromide (1.4M in THF/toluene 1.72 mL, 2.40 mmol) was added. The mixture was stirred at 0° C. for 1 h. Then a saturated NaHCO₃ solution and DCM was added and the mixture was filtered through a Celite® pad. The filtrate was extracted with DCM and the organic layer was separated, dried (MgSO₄), filtered and the solvents evaporated in vacuo. The residue was purified by flash column chromatography (SiO₂; 7N solution of NH₃ in MeOH in DCM, gradient from 0/100 to 10/90). The desired fractions were collected and concentrated in vacuo and the residue was purified by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected and evaporated in vacuo and the residue was purified by flash column chromatography (SiO₂; MeOH in DCM, gradient from 0/100 to 8/95). The desired fractions were collected and evaporated in vacuo to yield compound 26 as a colorless oil (0.022 g, 6%).

### Preparation of Final Compounds 27a and 27b

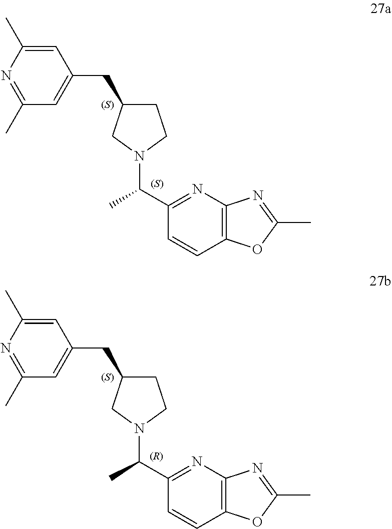

Compounds 27a and 27a were prepared following an analogous procedure to the one described for the synthesis of compound 26 using intermediate 8g as starting material (0.469 g, 2.47 mmol). The mixture of compounds 27a and 27b was purified by flash column chromatography (SiO₂; MeOH in EtOAc, gradient from 20/80 to 0/100) and the desired fractions were collected and evaporated in vacuo. The residue was purified by preparative LC (irregular bare silica; 0.8% NH₄OH and 8% MeOH in 92% DCM) and the desired fractions were collected and evaporated in vacuo. Compounds 27a and 27b were separated by chiral SFC (stationary phase: Chiralpak IC 5 μm 250×30 mm, mobile phase: 60% CO₂, 40% (EtOH (0.3% ᵢPrNH₂)). The desired fractions were collected and the solvents evaporated in vacuo to yield compound 27a (0.048 g, 6%) and compound 27b (0.051 g, 6%) as yellow films.

### Preparation of Final Compounds 28

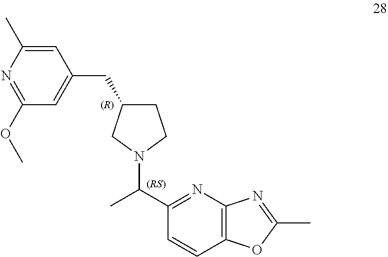

Compound 28 was prepared following an analogous procedure to the one described for the synthesis of compound 18 using intermediate 8f as starting material (0.275 g, 1.33 mmol). Compound 28 was purified by flash column chromatography (SiO₂; 7N solution of NH₃ in MeOH in DCM in DCM, gradient from 0/100 to 5/95) and the desired fractions were collected and evaporated in vacuo. The desired fractions were collected and concentrated in vacuo and the residue was purified by reverse phase HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and evaporated in vacuo and the residue was purified by flash column chromatography (SiO₂; 7N solution of NH₃ in MeOH in DCM in DCM, gradient from 0/100 to 2/98). The desired fractions were collected and evaporated in vacuo to yield compound 28 as a colorless oil (0.60 g, 15%)

### Preparation of Final Compound 29

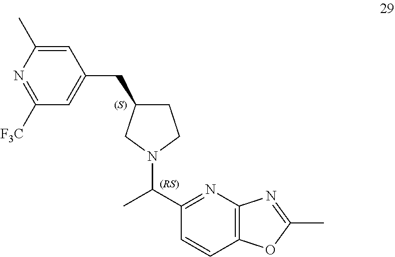

Compound 29 was prepared following an analogous procedure to the one described for the synthesis of compound 18 using intermediate 8h as starting material (0.10 g, 0.41 mmol). Compound 29 was purified by flash column chromatography (SiO₂; 7N solution of NH₃ in MeOH in DCM in DCM, gradient from 0/100 to 10/90) and the desired fractions were collected and evaporated in vacuo. The residue was purified by reverse phase HPLC (stationary phase: C18 XBridge 30×100 mm 5 μm, mobile phase: gradient from 67% NH₄HCO₃ 0.25% solution in water, 33% CH₃CN to 50% NH₄HCO₃ 0.25% solution in water, 50% CH₃CN). The desired fractions were collected and the solvents evaporated in vacuo to yield compound 29 as a white solid (0.050 g, 30%).

### Preparation of Final Compound 30

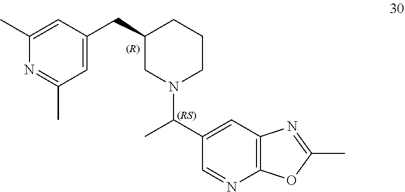

Compound 30 was prepared following an analogous procedure to the one described for the synthesis of compound 18 using intermediates 8a (0.09 g, 0.44 mmol) and 19a (0.10 g, 0.62 mmol) as starting materials. Compound 30 was purified by flash column chromatography (SiO₂; MeOH in DCM in DCM, gradient from 0/100 to 5/95) and the desired fractions were collected and evaporated in vacuo. The residue was purified by reverse phase HPLC (stationary phase: C18 XBridge 30×100 mm 5 μm, mobile phase: gradient from 90% NH₄HCO₃ 0.25% solution in water, 10% CH₃CN to 65% NH₄HCO₃ 0.25% solution in water, 45% CH₃CN). The desired fractions were collected and the solvents were evaporated in vacuo to yield compound 30 as a colorless sticky solid (0.091 g, 56%).

### Preparation of Final Compound 31

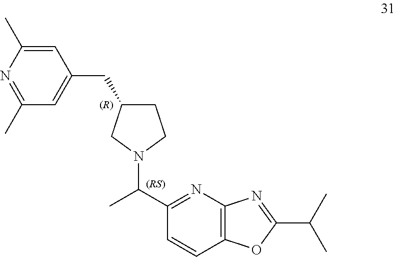

Compound 31 was prepared following an analogous procedure to the one described for the synthesis of compound 18 using intermediates 8e (0.10 g, 0.53 mmol) and 19b (0.120 g, 0.63 mmol) as starting materials. Compound 31 was purified by flash column chromatography (SiO₂; MeOH in DCM in DCM, gradient from 0/100 to 30/70) and the desired fractions were collected and evaporated in vacuo. The residue was purified by reverse phase HPLC (stationary phase: C18 XBridge 30×100 mm 5 μm, mobile phase: gradient from 67% NH₄HCO₃ 0.25% solution in water, 33% CH₃CN to 50% NH₄HCO₃ 0.25% solution in water, 50% CH₃CN). The desired fractions were collected and the solvents evaporated in vacuo to yield compound 31 as a yellow oil (0.080 g, 40%).

### Preparation of Final Compound 32

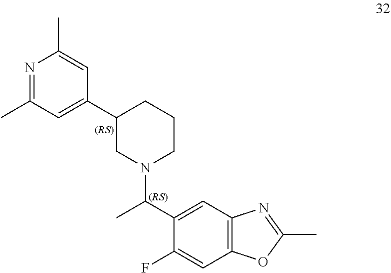

Compound 32 was prepared following an analogous procedure to the one described for the synthesis of compound 18 using intermediates 3a (0.10 g, 0.53 mmol) and 19c (0.123 g, 0.63 mmol) as starting materials. Compound 32 was purified by flash column chromatography (SiO₂; 7N solution of NH₃ in MeOH in DCM in DCM, gradient from 0/100 to 10/90) and the desired fractions were collected and evaporated in vacuo. The residue was purified by reverse phase HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and the solvents were evaporated in vacuo to yield compound 32 as a colorless oil (0.030 g, 16%).

### Preparation of Final Compound 33ab, 33a and 33b

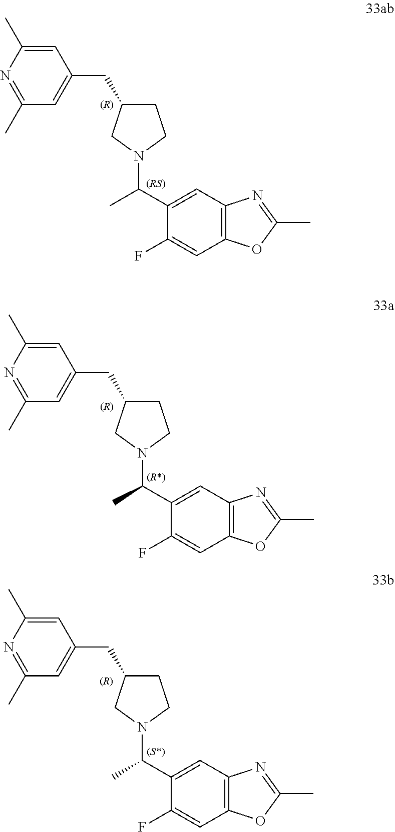

Compounds 33ab, 33a and 33b were prepared following an analogous procedure to the one described for the synthesis of compound 18 using intermediates 8e (0.067 g, 0.35 mmol) and 19c (0.060 g, 0.33 mmol) as starting materials. Compound 33ab was purified by flash column chromatography (SiO₂; 7N solution of NH₃ in MeOH in DCM in DCM, gradient from 0/100 to 10/90) and the desired fractions were collected and evaporated in vacuo. Compounds 33a and 33b were separated by reverse phase HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and the solvents evaporated in vacuo and the residues were dissolved in EtOAc and washed with a saturated NaHCO₃ solution. The organic layer was separated, dried (MgSO₄), filtered and the solvents evaporated in vacuo to yield compounds 33a (0.032 g, 26%) and compound 33b (0.014 g, 22%) as colorless oils.

### Preparation of Final Compound 34

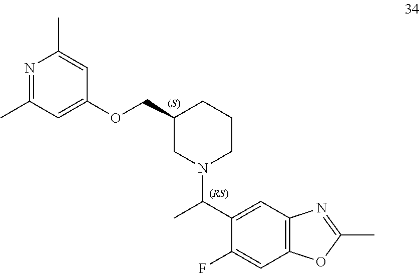

Compound 34 was prepared following an analogous procedure to the one described for the synthesis of compound 18 using intermediates 10a (0.10 g, 0.45 mmol) and 19c (0.085 g, 0.48 mmol) as starting materials. Compound 34 was purified by flash column chromatography (SiO₂; 7N solution of NH₃ in MeOH in DCM in DCM, gradient from 0/100 to 10/90) and the desired fractions were collected and evaporated in vacuo to yield compound 34 as a colorless oil (0.055 g, 30%).

### Preparation of Final Compound 35

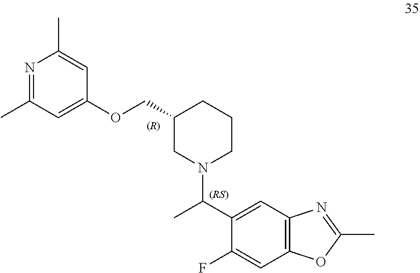

Compound 34 was prepared following an analogous procedure to the one described for the synthesis of compound 18 using intermediates 10b (0.10 g, 0.45 mmol) and 19c (0.085 g, 0.48 mmol) as starting materials. Compound 35 was purified by flash column chromatography (SiO₂; 7N solution of NH₃ in MeOH in DCM in DCM, gradient from 0/100 to 10/90) and the desired fractions were collected and evaporated in vacuo to yield compound 35 as a colorless oil (0.083 g, 46%).

### Preparation of Final Compound 36

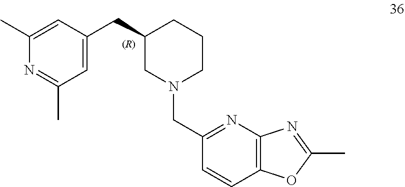

Intermediate 19d (0.085 g, 0.53 mmol) and sodium triacetoxyborohydride (CAS: 56553-60-7, 0.168 g, 0.79 mmol) were added to a stirred mixture of intermediate 8a (0.090 g, 0.44 mmol) in DCM (9.4 mL). The mixture was stirred at rt for 16 h and then a saturated NaHCO₃ solution was added. The organic layer was separated, dried (MgSO₄), filtered and the solvents were removed in vacuo. The residue was purified by flash column chromatography (SiO₂; MeOH in DCM, gradient from 0/100 to 10/90). The desired fractions were collected and concentrated in vacuo to yield compound 36 as a colorless oil (0.48 g, 31%).

### Preparation of Final Compound 37

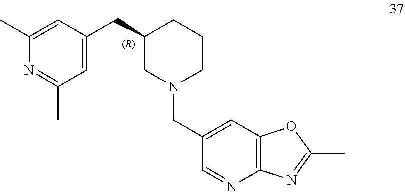

Intermediate 19e (0.041 g, 0.26 mmol) and titanium (IV) isopropoxide (CAS: 546-68-9; 0.108 mL, 0.365 mmol) were added to a stirred solution of intermediate 8a (0.05 g, 0.24 mmol) in DCM (0.79 mL). The mixture was stirred at rt for 16 h and then sodium cyanoborohydride (CAS: 25895-60-7; 0.018 g, 0.29 mmol) was added. The mixture was stirred at rt for a further 16 h and then a 10% NH₄Cl solution was added. The mixture was extracted with DCM and the organic layer was separated, dried (Na₂SO₄), filtered and the solvents were evaporated in vacuo. The residue was purified by reverse phase HPLC (stationary phase: C18 XBridge 30×100 mm 5 μm, mobile phase: gradient from 54% 10 mM NH₄HCO₃/NH₄OH pH=9 solution in water, 46% CH₃CN to 36% 10 mM NH₄HCO₃/NH₄OH pH=9 solution in water, 64% CH₃CN). The desired fractions were collected and evaporated in vacuo, and the residue was partitioned between a saturated NaHCO₃ solution and DCM. The organic layer was separated, dried (Na₂SO₄), filtered and the solvents were evaporated in vacuo to yield compound 37 as a white solid (0.018 g, 21%).

### Preparation of Final Compound 38

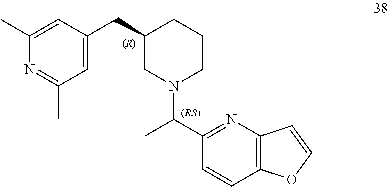

Titanium (IV) isopropoxide (CAS: 546-68-9; 0.170 mL, 0.57 mmol) and then sodium cyanoborohydride (CAS: 25895-60-7; 0.059 g, 0.94 mmol) were added to a stirred solution of intermediate 8a (0.139 g, 0.68 mmol) and intermediate 20a (0.104 g, 0.65 mmol) in 1,2-dichloroethane (2.2 mL) in a sealed tube under N₂. The mixture was stirred at 80° C. for 21 h and after cooling to rt a saturated NaHCO₃ solution and DCM were added and the mixture was filtered through a Celite® pad. The filtrate was extracted with DCM and the organic layer was separated, dried (MgSO₄), filtered and the solvents were evaporated in vacuo. The residue was purified by flash column chromatography (SiO₂; 7N solution of NH₃ in MeOH in DCM, gradient from 0/100 to 5/95). The desired fractions were collected and concentrated in vacuo and the residue was purified by reverse phase HPLC (stationary phase: C18 XBridge 30×100 mm 5 μm, mobile phase: gradient from 67% NH₄HCO₃ 0.25% solution in water, 33% CH₃CN to 50% NH₄HCO₃ 0.25% solution in water, 50% CH₃CN). The desired fractions were collected and extracted with EtOAc and the organic layer was separated, dried (MgSO₄), filtered and the solvents were evaporated in vacuo. and the residue was washed with a saturated NaHCO₃ solution to yield compounds 38 as a yellow oil (0.036 g, 14%).

### Preparation of Final Compound 39

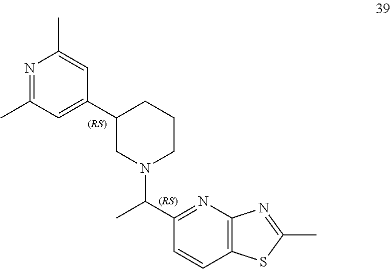

Intermediate 28a (0.121 g, 0.63 mmol) and titanium (IV) isopropoxide (CAS: 546-68-9; 0.231 mL, 0.79 mmol) were added to a stirred solution of intermediate 3a (0.10 g, 0.53 mmol) in DCM (2.2 mL) at rt under N₂. The mixture was stirred at rt for 20 h, cooled down to 0° C. and then methylmagnesium bromide (1.4M in THF/toluene, 1.73 mL, 2.42 mmol) was added. The mixture was stirred at 0° C. for 2 h and then a saturated NH₄Cl solution and DCM were added. The organic layer was separated, dried (MgSO₄), filtered and the solvents evaporated in vacuo. The residue was purified by flash column chromatography (SiO₂; 7N solution of NH₃ in MeOH in DCM, gradient from 0/100 to 10/90). The desired fractions were collected and concentrated in vacuo and the residue was purified by reverse phase HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and a saturated NaHCO₃ solution was added and the mixture extracted with DCM. The organic layer was separated, dried (MgSO₄), filtered and the solvents were evaporated in vacuo to yield compound 39 as a white solid (0.040 g, 21%).

### Preparation of Final Compound 40

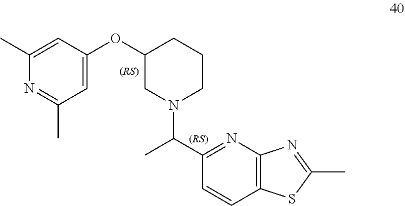

Compound 40 was prepared following an analogous procedure to the one described for the synthesis of compound 39 using intermediates 5a (0.100 g, 0.48 mmol) and 28a (0.103 g, 0.58 mmol) as starting materials. Compound 40 was purified by flash column chromatography (SiO₂; 7N solution of NH₃ in MeOH in DCM in DCM, gradient from 0/100 to 10/90) and the desired fractions were collected and evaporated in vacuo. The residue was purified by reverse phase HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and the solvents evaporated in vacuo to yield compound 40 as a colorless oil (0.009 g, 5%).

### Preparation of Final Compound 41

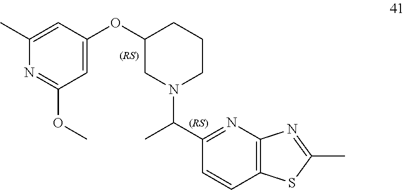

Compound 41 was prepared following an analogous procedure to the one described for the synthesis of compound 39 using intermediates 5d (0.100 g, 0.45 mmol) and 28a (0.84 g, 0.47 mmol) as starting materials. Compound 40 was purified by flash column chromatography (SiO₂; 7N solution of NH₃ in MeOH in DCM in DCM, gradient from 0/100 to 10/90) and the desired fractions were collected and evaporated in vacuo to compound 41 as a light-brown oil (0.124 g, 69%).

### Preparation of Final Compound 42a and 42b

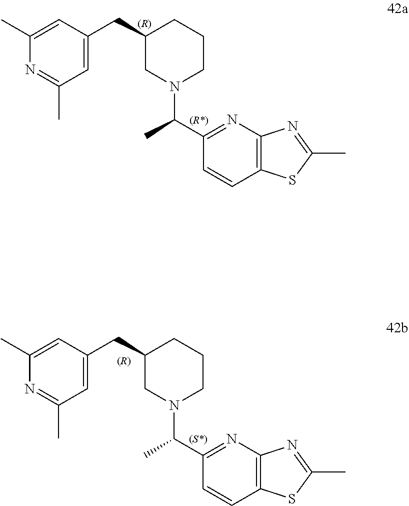

Compounds 42a and 42b were prepared following an analogous procedure to the one described for the synthesis of compound 39 using intermediates 8a (0.068 g, 0.33 mmol) and 28a (0.060 g, 0.33 mmol) as starting materials. The mixture of compounds 42a and 42b was purified by flash column chromatography (SiO₂; MeOH in DCM, gradient from 0/100 to 6/94) and the desired fractions were collected and evaporated in vacuo. Compounds 41a and 41b were separated by chiral SFC (stationary phase: Chiralpak IC 5 μm 250×30 mm, mobile phase: 60% CO₂, 40% (EtOH (0.3% ᵢPrNH₂)). The desired fractions were collected and the solvents evaporated in vacuo to yield compound 42a (0.013 g, 10%) and compound 42b (0.010 g, 8%) as yellow films.

### Preparation of Final Compound 43

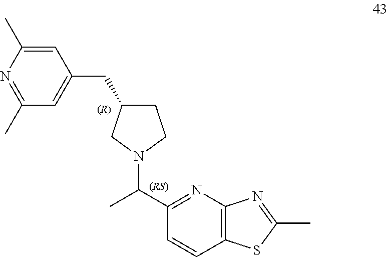

Intermediate 28a (0.178 g, 0.53 mmol) and titanium (IV) isopropoxide (CAS: 546-68-9; 0.233 mL, 0.79 mmol) were added to a stirred solution of intermediate 8e (0.10 g, 0.53 mmol) in DCM (2 mL) at rt under N₂. The mixture was stirred at rt for 16 h, cooled down to 0° C. and then methylmagnesium bromide (1.4M in THF/toluene, 1.72 mL, 2.40 mmol) was added. The mixture was stirred at 0° C. for 1 h. Then a saturated NaHCO₃ solution and DCM were added and the mixture was filtered through a Celite® pad. The filtrate was extracted with DCM and the organic layer was separated, dried (MgSO₄), filtered and the solvents were evaporated in vacuo. The residue was purified by flash column chromatography (SiO₂; 7N solution of NH₃ in MeOH in DCM, gradient from 0/100 to 10/90). The desired fractions were collected and concentrated in vacuo and the residue was purified by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected and evaporated in vacuo to yield compound 43 as a colorless oil (0.035 g, 18%).

### Preparation of Final Compound 44

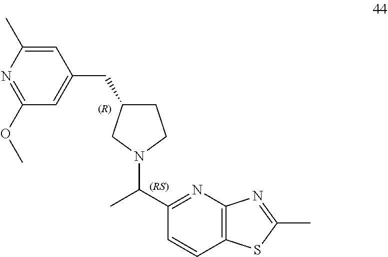

Compound 44 was prepared following an analogous procedure to the one described for the synthesis of compound 43 using intermediates 8g (0.139 g, 0.67 mmol) and 28a (0.100 g, 0.56 mmol) as starting materials. Compound 43 was purified by flash column chromatography (SiO₂; 7N solution of NH₃ in MeOH in DCM in DCM, gradient from 0/100 to 5/95) and the desired fractions were collected and evaporated in vacuo. The residue was purified by reverse phase HPLC (stationary phase: C18 XBridge 30×100 mm 5 μm, mobile phase: gradient from 67% NH₄HCO₃ 0.25% solution in water, 33% CH₃CN to 67% NH₄HCO₃ 0.25% solution in water, 33% CH₃CN). The desired fractions were collected and the solvents evaporated in vacuo yield compound 44 as an oil (0.140 g, 63%).

### Preparation of Final Compounds 45ab and 45a

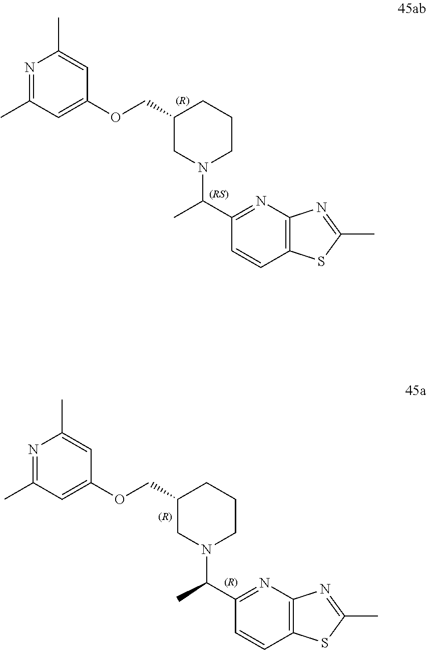

Compounds 45ab and 45a were prepared following an analogous procedure to the one described for the synthesis of compound 39 using intermediate 10b as starting material (0.10 g, 0.45 mmol). Compound 45ab was purified by flash column chromatography (SiO₂; 7N solution of NH₃ in MeOH in DCM in DCM, gradient from 0/100 to 10/90) and the desired fractions were collected and evaporated in vacuo. The residue was purified by reverse phase HPLC (stationary phase: C18 XBridge 30×100 mm 5 μm, mobile phase: gradient from 67% NH₄HCO₃ 0.25% solution in water, 33% CH₃CN to 50% NH₄HCO₃ 0.25% solution in water, 50% CH₃CN). The desired fractions were collected and the solvents evaporated in vacuo to yield compound 45ab (0.034 g, 19%) and compound 45a (0.029 g, 16%) as yellow oils.

### Preparation of Final Compounds 46Ab and 46a

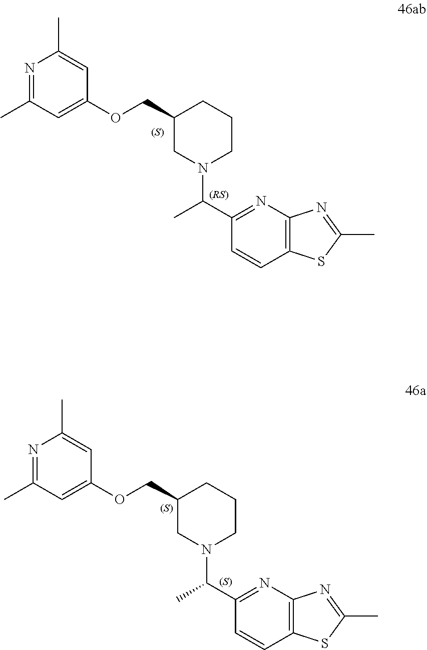

Compounds 46ab and 46a were prepared following an analogous procedure to the one described for the synthesis of compound 39 using intermediate 10a as starting material (0.10 g, 0.45 mmol). Compound 46ab was purified by flash column chromatography (SiO₂; 7N solution of NH₃ in MeOH in DCM in DCM, gradient from 0/100 to 10/90) and the desired fractions were collected and evaporated in vacuo. The residue was purified by reverse phase HPLC (stationary phase: C18 XBridge 30×100 mm 5 μm, mobile phase: gradient from 67% NH₄HCO₃ 0.25% solution in water, 33% CH₃CN to 50% NH₄HCO₃ 0.25% solution in water, 50% CH₃CN). The desired fractions were collected and the solvents evaporated in vacuo. The residue was dissolved in EtOAc and washed with a saturated NaHCO₃ solution. The organic layers were separated, dried (Na₂SO₄), filtered and the solvents evaporated in vacuo to yield compound 46ab (0.035 g, 20%) and compound 46a (0.043 g, 24%) as yellow oils.

### Preparation of Final Compound 47

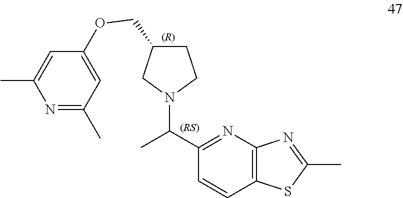

Intermediate 28a (0.118 g, 0.66 mmol) and titanium (IV) isopropoxide (CAS: 546-68-9; 0.294 mL, 0.99 mmol) were added to a stirred solution of intermediate 10d (0.150 g, 0.72 mmol) in DCM (2 mL) at rt under N₂. The mixture was stirred at rt for 16 h, cooled down to 0° C. and then methylmagnesium bromide (1.4M in THF/toluene, 1.72 mL, 2.40 mmol) was added. The mixture was stirred at 0° C. for 1 h. Then MeOH and DCM were added and the mixture was filtered through a Celite® pad. The filtrate was treated with a saturated NH₄Cl solution and extracted with DCM and the organic layer was separated, dried (MgSO₄), filtered and the solvents were evaporated in vacuo. The residue was purified by flash column chromatography (SiO₂; 7 N solution of NH₃ in MeOH in DCM, gradient from 0/100 to 08/92). The desired fractions were collected and concentrated in vacuo to yield compound 47 as an oil (0.160 g, 63%).

### Preparation of Final Compound 48

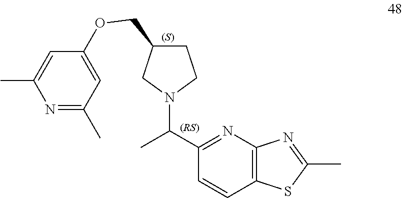

Compound 48 was prepared following an analogous procedure to the one described for the synthesis of compound 46 using intermediates 10e (0.150 g, 0.73 mmol) and intermediate 28a (0.117 g, 0.66 mol) as starting materials. Compound 48 was purified by flash column chromatography (SiO₂; 7N solution of NH₃ in MeOH in DCM in DCM, gradient from 0/100 to 08/92) and the desired fractions were collected and evaporated in vacuo to yield compound 48 (0.155 g, 61%) as an oil.

### Preparation of Final Compounds 49ab, 49a and 49b

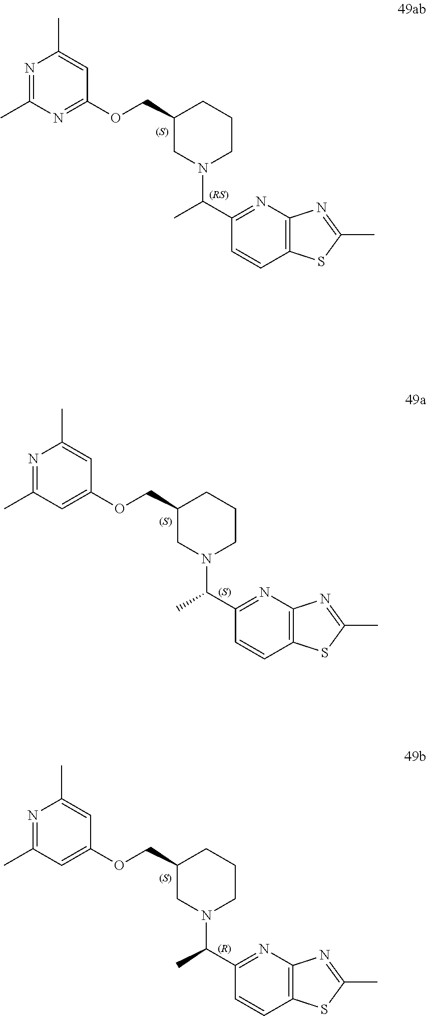

Compounds 49ab, 49a and 49b were prepared following an analogous procedure to the one described for the synthesis of compound 39 using intermediates 10c (0.150 g, 0.68 mmol) and 28a (0.127 g, 0.71 mmol) as starting materials. Compound 40ab was purified by flash column chromatography (SiO₂; 7N solution of NH₃ in MeOH in DCM in DCM, gradient from 0/100 to 10/90) and the desired fractions were collected and evaporated in vacuo. The residue was purified by reverse phase HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and the solvents evaporated in vacuo and the residues were dissolved in EtOAc and extracted with a saturated solution of NaHCO₃. The organic layers were separated, dried (Na₂SO₄), filtered and evaporated in vacuo to yield compound 49ab as a yellow oil (0.008 g, 3%), compound 49a as a grey oil (0.016 g, 6%) and compound 49b as a yellow oil (0.017, 6%).

### Preparation of Final Compound 50

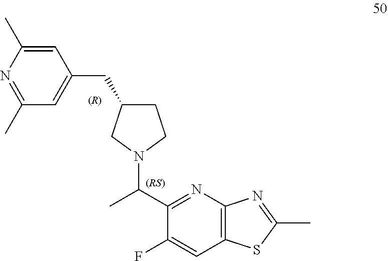

Intermediate 28b (0.015 g, 0.071 mmol) and titanium (IV) isopropoxide (CAS: 546-68-9; 0.030 mL, 0.11 mmol) were added to a stirred solution of intermediate 8e (0.013 g, 0.068 mmol) in THF (0.50 mL) at rt under N₂. The mixture was stirred at 70° C. for 16 h and then sodium cyanoborohydride (CAS: 25895-60-7; 0.018 g, 0.29 mmol) was added. The mixture was stirred at rt for a further 16 h and then water was added. The mixture was extracted with EtOAc and the organic layer was separated, dried (MgSO₄), filtered and the solvents were evaporated in vacuo. The residue was purified by reverse phase HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and evaporated in vacuo to yield compound 50 as a colorless oil (0.010 g, 38%).

### Preparation of Final Compound 51

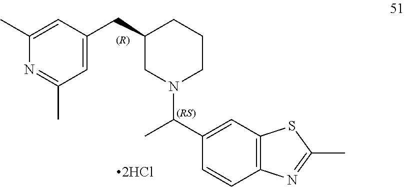

Sodium cyanoborohydride (CAS: 25895-60-7; 0.054 g, 0.87 mmol) was added to a stirred mixture of intermediate 8a (0.200 g, 0.72 mmol), intermediate 28c (0.138 g, 0.72 mmol), titanium (IV) isopropoxide (CAS: 546-68-9; 0.214 mL, 0.72 mmol) and Et₃N (0.300 mL, 2.16 mmol) in DCM (2.37 mL) at rt. The mixture was stirred at 80° C. for 16 h and then water was added. The mixture was extracted with DCM and the organic layer was separated, dried (Na₂SO₄), filtered and the solvents were evaporated in vacuo. The residue was purified by reverse phase HPLC (stationary phase: C18 XBridge 30×100 mm 5 μm, mobile phase: gradient from 54% NH₄HCO₃ 0.25% solution in water, 46% CH₃CN to 46% NH₄HCO₃ 0.25% solution in water, 54% CH₃CN). The desired fractions were collected and evaporated in vacuo and the residue dissolved in MeOH and treated with a 6M solution of HCl in iPrOH. The mixture was stirred at rt for 2 h and then the solvents were evaporated in vacuo to yield compound 51 as a white solid (0.105 g, 32%).

### Preparation of Final Compound 52

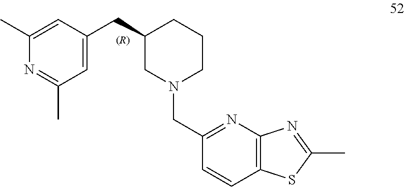

Et₃N (0.062 mL, 0.45 mmol) was added to a stirred solution of intermediate 8a (0.031 g, 0.11 mmol) in DCM (1.7 mL). The mixture was stirred at rt for 10 min and then intermediate 28a (0.020 g, 0.11 mmol) and sodium triacetoxyborohydride (CAS: 56553-60-7, 0.071 g, 0.34 mmol) were added. The mixture was stirred at rt for 18 h and then a saturated NaHCO₃ solution was added. The mixture was extracted with DCM and the organic layer was separated, dried (MgSO₄), filtered and the solvents were removed in vacuo. The residue was purified by reverse phase HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and concentrated in vacuo to yield compound 52 as a colorless oil (0.015 g, 36%).

### Preparation of Final Compound 53

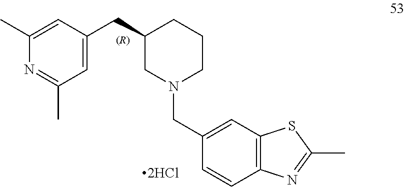

Sodium triacetoxyborohydride (CAS: 56553-60-7, 0.2151 g, 1.02 mmol) was added to a stirred solution of intermediate 8a (0.187 g, 0.68 mmol), intermediate 28c (0.120 g, 0.68 mmol) and Et₃N (0.282 mL, 2.03 mmol) in MeOH (2.19 mL). The mixture was stirred at rt for 16 h and then water was added. The mixture was extracted with EtOAc and the organic layer was separated, dried (Na₂SO₄), filtered and the solvents removed in vacuo. The residue was purified by flash column chromatography (SiO₂; 7N solution of NH₃ in MeOH in DCM in DCM, gradient from 0/100 to 05/95) and the desired fractions were collected and evaporated in vacuo. The residue was purified by reverse phase HPLC (stationary phase: C18 XBridge 30×100 mm 5 μm, mobile phase: gradient from 60% 10 mM NH₄HCO₃/NH₄OH pH 7.9 solution in water, 40% CH₃CN to 43% 10 mM NH₄HCO₃/NH₄OH pH 7.9 solution in water, 57% CH₃CN). The desired fractions were collected and concentrated in vacuo and the residue dissolved in MeOH and treated with a 6M solution of HCl in iPrOH. The mixture was stirred at rt for 2 h and then the solvents were evaporated in vacuo to yield compound 53 as a blue solid (0.055 g, 19%).

### Preparation of Final Compound 54

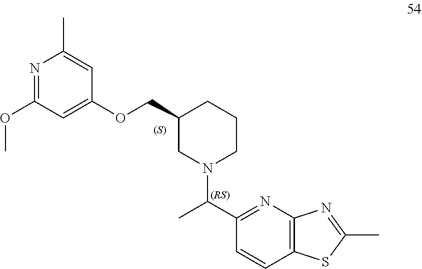

Compound 54 was prepared following an analogous procedure to the one described for the synthesis of compound 39 using intermediate 10f as starting material (0.10 g, 0.42 mmol). Compound 54 was purified by flash column chromatography (SiO₂; 7N solution of NH₃ in MeOH in DCM in DCM, gradient from 0/100 to 10/90) and the desired fractions were collected and evaporated in vacuo. The residue was purified by reverse phase HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and the solvents evaporated in vacuo. The residue was dissolved in DCM and washed with a saturated NaHCO₃ solution. The organic layers were separated, dried (MgSO₄), filtered and the solvents evaporated in vacuo to yield compound 54 as a colorless oil (0.050 g, 29%).

### Preparation of Compound 55

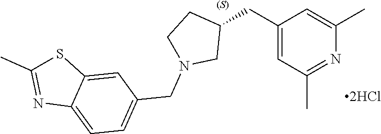

Sodium triacetoxyborohydride (CAS: 56553-60-7; 181 mg, 0.86 mmol) was added to a stirred mixture of intermediate I-8f.2HCl (150 mg, 0.57 mmol), intermediate I-28c (101 mg, 0.57 mmol) and Et₃N (0.24 mL. 1.71 mmol) in MeOH (1.85 mL) at room temperature. The reaction mixture was stirred for 16 h and concentrated in vacuo. The crude mixture was purified by flash column chromatography (SiO₂, NH₃ (7M in MeOH) in DCM, gradient from 0:100 to 10:90). The residue was purified by RP HPLC (stationary phase: C18 XBridge 30×100 mm 5 um), mobile phase: 10 mM NH₄CO₃H pH 7.9 solution in water/ACN, gradient from 60:40 to 43:57). The product was stirred in MeOH and treated with HCl (12M solution, 0.5 mL, 6.0 mmol) at room temperature for 10 min. The mixture was concentrated in vacuo and the product was dried under vacuum at 50° C. for 16 h to afford compound 55 (106 mg, 44%).

### Preparation of Compounds 56 and 57

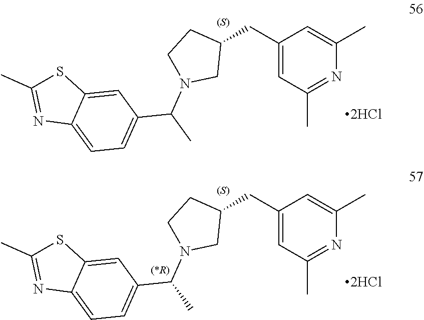

Ti(Oi-Pr)₄ (CAS: 546-68-9; 281 μL, 0.95 mmol) and sodium cyanoborohydride (CAS: 25895-60-7; 71.6 mg, 1.14 mmol) were added sequentially to a mixture of intermediate I-8F.2HCl (250 mg, 0.95 mmol), intermediate I-63 (182 mg, 0.95 mmol) and Et₃N (0.40 mL, 2.85 mL) in DCM (3.12 mL) at room temperature. The reaction mixture was stirred at 80° C. for 16 h in a sealed tube. The reaction was quenched with water and extracted with DCM (3 times). The combined organic layers were dried (MgSO₄), filtered and concentrated in vacuo. The crude mixture was purified by flash column chromatography (SiO₂, (10% 7N NH₃ in MeOH in DCM) in DCM, gradient from 0:100 to 50:50). The residue was purified again by RP HPLC (stationary phase: XBridge C18 50×100 mm, 5 □m), mobile phase: NH₄HCO₃ (0.25% solution in water)/ACN, gradient from 80:20 to 0:100) to afford fraction A (28 mg) and fraction B (100 mg). HCl (37% in H₂O, 91 μL, 1.09 mmol) was added to a stirred mixture of fraction B (100 mg, 0.27 mmol) in MeOH (0.67 mL) in a sealed tube. The reaction mixture was stirred at room temperature for 1 h and concentrated in vacuo to afford compound 56 (118 mg).

Product 56 was prepared following an analogous procedure using fraction A (28 mg) as starting material.

### Preparation of Compound 58

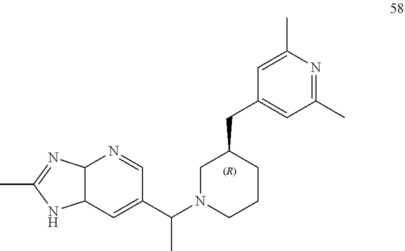

A solution of intermediate I-8a (75 mg, 0.37 mmol) in MeOH (2 mL) followed by Ti(Oi-Pr)₄ (CAS: 546-68-9; 180 μL, 0.61 mmol) and sodium cyanoborohydride (CAS: 25895-60-7 (44 mg, 0.7 mmol) were added to intermediate I-65 (57 mg, 0.33 mmol) in a sealed tube and under N₂ atmosphere. The reaction mixture was stirred at 80° C. for 60 h. The solvent was evaporated in vacuo and the crude mixture was purified by flash column chromatography (SiO₂, 7N solution of NH₃ in MeOH in DCM, gradient from 0:100 to 10:90). Another purification was performed by RP HPLC (stationary phase: C18 XBridge 30×100 mm 5 μm), mobile phase: NH₄HCO₃ (0.25% solution in water)/ACN, gradient from 80:20 to 60:40). The product was treated with water and extracted with DCM. The organic layer was dried (MgSO₄), filtered and the solvents evaporated in vacuo to afford compound 58 (22 mg, 19%).

### Preparation of Compounds 59 and 60

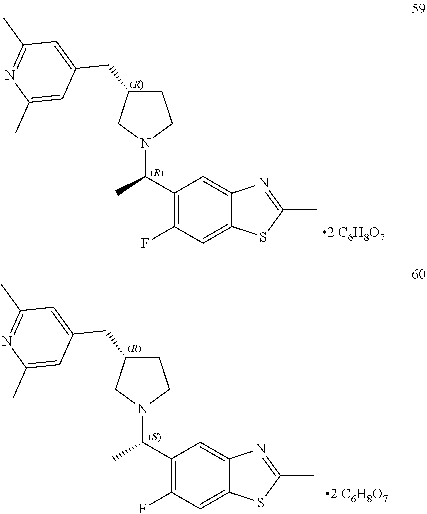

Sodium cyanoborohydride (CAS: 25895-60-7 (34.3 mg, 0.55 mmol) was added to a stirred mixture of intermediate I-71 (100 mg, 0.48 mmol), intermediate I-8e (86.6 mg, 0.46 mmol) and Ti(Oi-Pr)₄ (CAS: 546-68-9; 200 μL, 0.68 mmol) in THF (3.35 mL) at room temperature and under N₂ atmosphere. The reaction mixture was stirred at 70° C. for 16 h and diluted with water. The mixture was extracted with EtOAc. The organic layer was dried (MgSO₄), filtered and the solvents were evaporated in vacuo. The crude mixture was purified by flash column chromatography (SiO₂, MeOH in DCM, gradient from 0:100 to 30:70). A second purification was performed by flash column chromatography (SiO₂, NH₃ (7N in MeOH) in DCM, gradient from 0:100 to 5:95). The desired fractions were combined and concentrated in vacuo. The residue was purified by RP HPLC (stationary phase: XBridge C18 50×100 mm, 5 μm), mobile phase: NH₄HCO₃ (0.25% solution in water)/ACN, gradient from 60:40 to 43:57) to afford fraction A (38 mg) and fraction B (38 mg).

A solution of citric acid (37.1 mg, 0.19 mmol) in 1,4-dioxane (0.62 mL) was added to a solution of fraction B (37 mg, 96.5 μmol) in Et₂O (1.83 mL). The mixture was stirred at room temperature for 3 h. The precipitate was filtered off and washed with Et₂O. The solid was dissolved in MeOH, Et₂O was added and the mixture was concentrated in vacuo. The solid was dried in a desiccator at 50° C. for 16 h to afford compound 60 (47 mg) as a white solid.

Compound 59 was prepared following the same procedure using fraction A as starting material.

### Preparation of Compounds 61 and 62

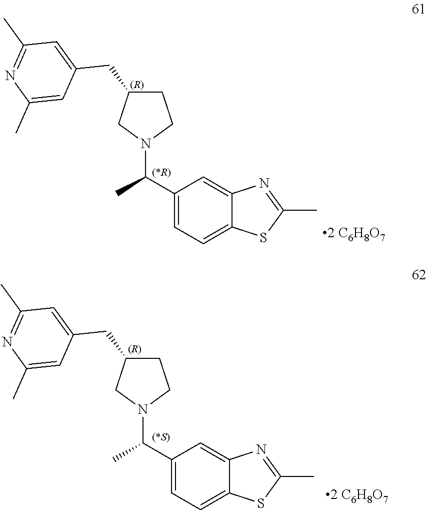

Compounds 61 and 62 were prepared following an analogous procedure to the one described for the synthesis of compounds 59 and 60 using intermediate I-74 and intermediate I-8e as starting materials.

The crude mixture was purified by flash column chromatography (SiO₂, DCM/MeOH, gradient from 100:0 to 70:30). A second purification was performed by flash column chromatography (SiO₂, DCM/NH₃ (7N in MeOH), gradient from 100:0 to 95:5). The desired fractions were concentrated in vacuo. The residue was purified by RP HPLC (stationary phase: XBridge C18 50×100 mm, 5 μm), mobile phase: NH₄HCO₃ (0.25% solution in water)/ACN, gradient from 69:31 to 52:48) to afford fraction A (42 mg) and fraction B (102 mg).

A solution of citric acid (41.9 mg, 0.22 mmol) in 1,4-dioxane (0.70 mL) was added to a solution of fraction A (40.0 mg, 0.11 mmol) in Et₂O (2.07 mL). The mixture was stirred at room temperature for 3 h. The precipitate was filtered off and washed with Et₂O. The solid was dissolved in MeOH, Et₂O was added and the mixture was concentrated in vacuo. The product was dried in a desiccator at 50° C. for 4 days to afford compound 61 (56 mg) as a white solid.

Compound 62 was prepared following an analogous procedure using fraction B as starting material.

### Preparation of Compound 63

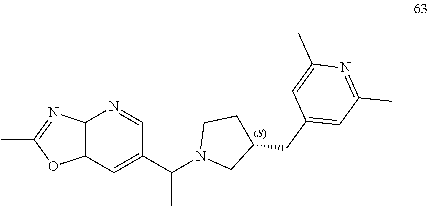

To a solution of intermediate I-8h (100 mg, 0.53 mmol) in DCM (31.5 mL) were added intermediate I-19a (93.7 mg, 0.58 mmol) and Ti(Oi-Pr)₄ (CAS: 546-68-9; 0.23 mL, 0.79 mmol). The reaction mixture was stirred at room temperature overnight. Then the reaction was cooled to 0° C. and methylmagnesium bromide (3M, 0.88 mL, 2.63 mmol) was added dropwise. The reaction mixture was stirred at 0° C. for 5 min and at room temperature for 1 h. NH₄Cl (sat., aq.) was added and the mixture was extracted with DCM. The organic layer was dried (MgSO₄), filtered and the solvents were evaporated in vacuo. The crude mixture was purified by flash column chromatography (SiO₂, NH₃ (7M in MeOH)/DCM, gradient from 0:100 to 3:97). The residue was purified by RP HPLC (stationary phase: C18 XBridge 30×100 mm 5 μm), mobile phase: NH₄HCO₃ (0.25% solution in water)/ACN, gradient from 67:33 to 50:50) to afford compound 63 (21 mg, 11%).

### Preparation of Compound 64

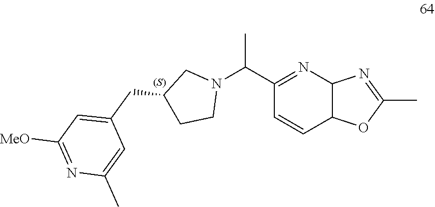

Compound 64 was prepared following an analogous procedure to the one described for the synthesis of compound 63 using intermediate I-19a and intermediate I-32 as starting materials.

The crude mixture was purified by flash column chromatography (SiO₂, NH₃ (7M in MeOH)/DCM, gradient from 0:100 to 3:97). The residue was purified by ion exchange chromatography using an Isolute SCX2 cartridge and eluting with MeOH, and then with NH₃ (7M in MeOH). The desired fractions were collected and concentrated in vacuo. The red oil was purified by RP HPLC (stationary phase: C18 XBridge 30×100 mm 5 μm), mobile phase: NH₄HCO₃ (0.25% solution in water)/ACN, gradient from 67:33 to 50:50). The desired fractions were collected and the solvents partially concentrated in vacuo. The aqueous phase was extracted with EtOAc. The organic phase was dried (Na₂SO₄), filtered and the solvent was evaporated in vacuo to afford compound 64 (40 mg, 32%).

### Preparation of Compound 65

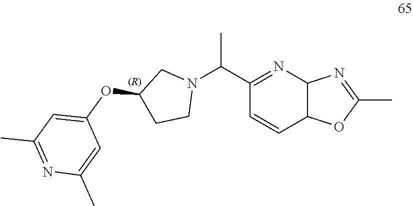

Compound 65 was prepared following an analogous procedure to the one described for the synthesis of compound 63 using intermediate I-28a and intermediate I-44 as starting materials.

The crude mixture was purified by flash column chromatography (SiO₂, NH₃ (7N in MeOH)/DCM, gradient from 0;100 to 10:90). The residue was purified by RP HPLC (stationary phase: C18 XBridge 30×100 mm 5 □m), mobile phase: (10 mM NH₄HCO₃/NH₄OH pH=9 solution in water)/ACN, gradient from 80:20 to 60:40). The product was dissolved in DCM and washed with NaHCO₃ (sat., aq.). The organic layer was dried (Na₂SO₄), filtered and evaporated in vacuo to afford compound 65 (124 mg, 43%).

### Preparation of Compounds 66 and 67

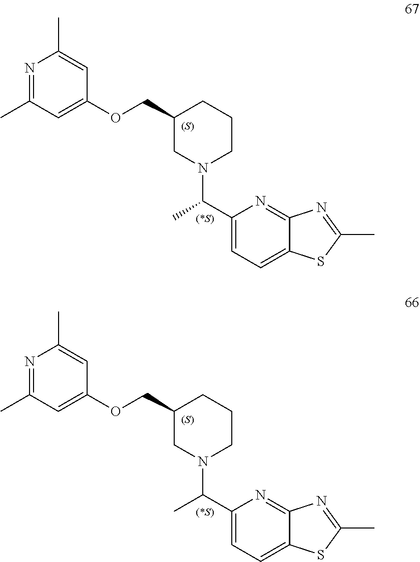

Compounds 66 and 67 were prepared following an analogous to that described for the synthesis of compound 63 using intermediate I-28a and intermediate I-46 as starting materials.

The crude mixture was purified by flash column chromatography (SiO₂, NH₃ (7N in MeOH)/DCM, gradient from 0;100 to 10:90). The residue was purified by RP HPLC (stationary phase: C18 XBridge 30×100 mm 5 μm), mobile phase: NH₄HCO₃ (0.25% solution in water)/ACN, gradient from 80:20 to 0:100). The residue was dissolved in EtOAc and washed with NaHCO₃ (sat., aq.). The organic layer was dried (Na₂SO₄), filtered and concentrated in vacuo to afford compound 67 (26.2 mg, 15%) and compound 66 (26.7 mg, 15%).

### Preparation of Compounds 68 and 69

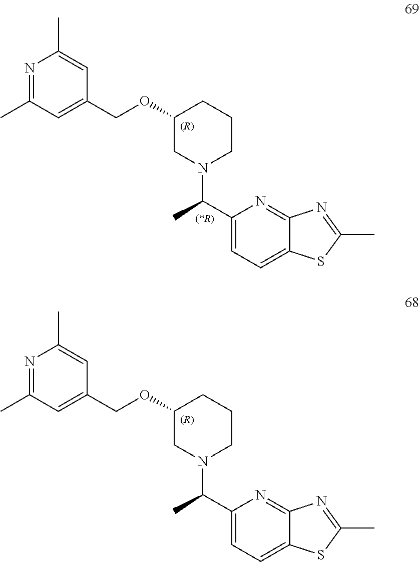

Compounds 68 and 69 were prepared following an analogous procedure to the one described for the synthesis of compound 63 using intermediate I-28a and intermediate I-48 as starting materials.

The crude mixture was purified by flash column chromatography (SiO₂, NH₃ (7N in MeOH)/DCM, gradient from 0;100 to 10:90). The residue was purified by RP HPLC (stationary phase: C18 XBridge 30×100 mm 5 μm), mobile phase: NH₄HCO₃ (0.25% solution in water)/ACN, gradient from 80:20 to 0:100). The residue was dissolved in EtOAc and washed with NaHCO₃ (sat., aq.). The organic layer was dried (Na₂SO₄), filtered and concentrated in vacuo to afford compound 69 (12 mg, 7%) and compound 68 (10 mg, 6%).

### Preparation of Compounds 70 and 71

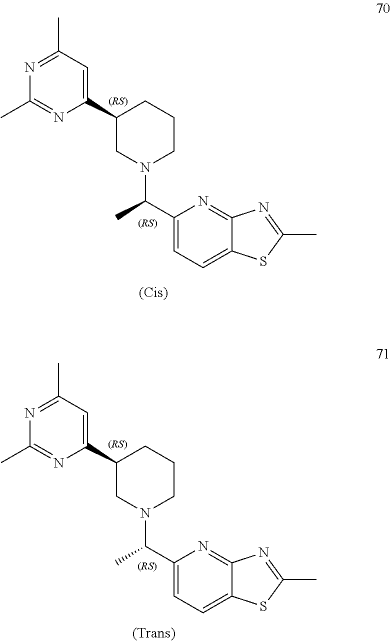

Compounds 70 and 71 were prepared following an analogous procedure to the one described for the synthesis of compound 63 using intermediate I-28a and intermediate I-35 as starting materials.

The crude product was purified by flash column chromatography (SiO₂, NH₃ (7N in MeOH)/DCM, gradient from 0:100 to 10:90). The residue was purified by RP HPLC (stationary phase: C18 XBridge 30×100 mm 5 μm), mobile phase: NH₄HCO₃ (0.25% solution in water)/ACN, gradient from 67:33 to 50:50) to afford compound 70 and compound 71. The compounds were separately dissolved in EtOAc and washed with NaHCO₃ (sat., aq.). The organic layer was dried (Na₂SO₄), filtered and concentrated in vacuo to afford compound 70 (65 mg, 34%) and compound 71 (18 mg, 9%).

### Preparation of Compound 72

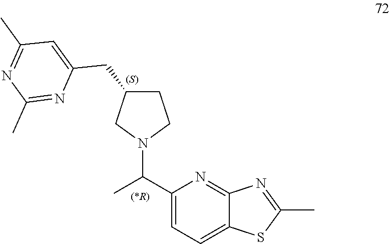

Compound 72 was prepared following an analogous procedure to the one described for the synthesis of compound 63 using intermediate I-28a and intermediate I-42 as starting materials.

The crude mixture was purified by flash column chromatography (SiO₂, NH₃ (7N in MeOH)/DCM, gradient from 0:100 to 10:90) to afford compound 72 (105 mg, 55%).

### Preparation of Compounds 73 and 74

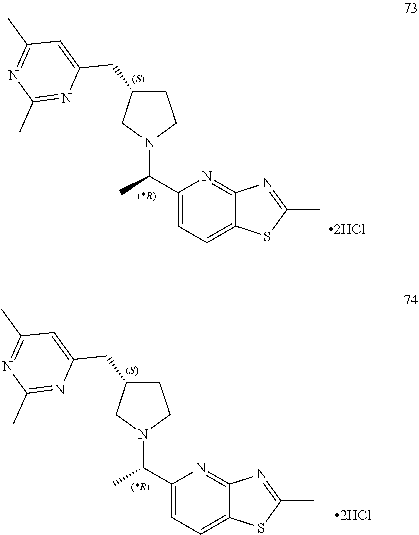

Compounds 73 and 74 were prepared following an analogous procedure to the one described for the synthesis of compound 63 using intermediate I-28a and intermediate 1-44 as starting materials.

The crude mixture was purified by flash column chromatography (SiO₂, NH₃ (7N in MeOH)/DCM, gradient from 0:100 to 10:90). A second purification was performed by RP HPLC (stationary phase: C18 XBridge 30×100 mm 5 □m), mobile phase: (10 mM NH₄HCO₃/NH₄OH pH=9 solution in water)/ACN, gradient from 80:20 to 60:40). The product was dissolved in DCM and washed with NaHCO₃ (sat., aq.). The organic layer was dried (Na₂SO₄), filtered and evaporated in vacuo to give a mixture of diastereoisomers (112 mg). A purification was performed via chiral SFC (stationary phase: Chiralpak IG 5 μm 250*20 mm, mobile phase: 55% CO₂, 45% MeOH (0.3% i-PrNH₂)) and delivered fraction A and fraction B.

Fraction A was taken up in diethyl ether and treated with HCl (6N solution in i-PrOH). The solvents were evaporated in vacuo to afford compound 73 (53 mg, 15%) as a white solid.

Fraction B was submitted to the same treatment to afford compound 74 (35 mg, 10%).

### Preparation of Compounds 75 and 76

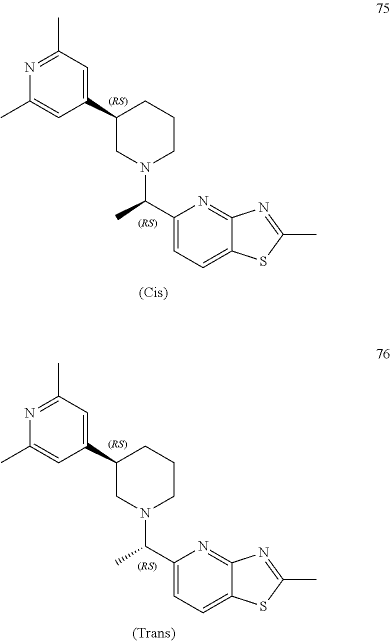

Compounds 75 and 76 were prepared following an analogous procedure to the one described for the synthesis of compound 63 using intermediate I-28a and intermediate I-3a as starting materials.

The crude mixture was purified by flash column chromatography (SiO₂, DCM/MeOH, gradient from 100:0 to 90:10). A second purification was performed by RP HPLC (stationary phase: C18 XBridge 30×100 mm 5 μm), mobile phase: NH₄HCO₃ (0.25% solution in water)/ACN, gradient from 75:25 to 57:43%). NaHCO₃ (sat., aq.) was added and the product extracted with DCM. The organic layer was dried (MgSO₄), filtered and the solvents were evaporated in vacuo to afford compound 75 (43.8 mg, 6%) and compound 76 (51.8 mg, 7%) as white solids.

### Preparation of Compounds 77 and 78

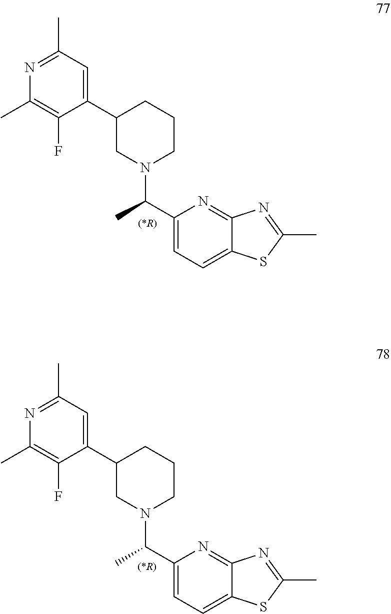

Compounds 77 and 78 were prepared following an analogous procedure to the one described for the synthesis of compound 63 using intermediate I-28a and intermediate I-38 as starting materials.

The crude mixture was purified by flash column chromatography (SiO₂, DCM/MeOH, gradient from 100:0 to 90:10). A second purification was performed by RP HPLC (stationary phase: C18 XBridge 30×100 mm 5 μm), mobile phase: NH₄HCO₃ (0.25% solution in water)/ACN, gradient from 90:10 to 60:40) to afford compound 77 (62 mg, 34%) and compound 78 (70 mg, 38%) as white solids.

### Preparation of Compound 79

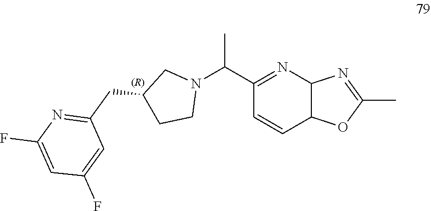

Intermediate I-40 (96.1 mg, 0.49 mmol), intermediate I-19a (94.3 mg, 0.58 mmol) and Ti(Oi-Pr)₄ (CAS: 546-68-9; 0.21 mL, 0.73 mmol) were dissolved in DCM (2.0 mL) at room temperature and under N₂ atmosphere. The reaction mixture was stirred for 16 h, cooled to 0° C. and methylmagnesium bromide (1.4M in THF, 1.73 mL, 2.42 mmol) was added dropwise. The reaction mixture was stirred at this temperature for 15 min and at room temperature for 1 h. The mixture was treated with NH₄Cl (sat., aq.), diluted with DCM and the mixture was filtered through a pad of diatomaceus earth. The organic layer was separated, dried (MgSO₄), filtered and the solvents were evaporated in vacuo. The crude mixture was purified by flash column chromatography (SiO₂, EtOAc/MeOH, gradient from 100:0 to 90:10). The residue was purified by RP HPLC (stationary phase: C18 XBridge 30×100 mm 5 μm), mobile phase: NH₄HCO₃ (0.25% solution in water)/ACN, gradient from 75:25 to 57:43) to afford compound 79 (74 mg, 43%).

### Preparation of Compound 80

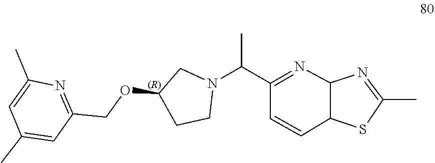

Intermediate I-28a (123 mg, 0.69 mmol) and Ti(Oi-Pr)₄ (CAS: 546-68-9; 280 μL, 0.95 mmol) were added to a solution of intermediate I-50 (130 mg, 0.63 mmol) in DCM (2.5 mL). The reaction mixture was stirred at room temperature for 16 h. The reaction was cooled to 0° C. and methylmagnesium bromide (1.4M, 2.25 mL, 3.15 mmol) was added and the reaction mixture was stirred for 2 h. The reaction was quenched with MeOH and diluted with DCM and water. The emulsion was filtered through a pad of Celite®. The filtrate was treated with NH₄Cl (sat., aq.) and extracted with DCM. The organic layer was dried (MgSO₄), filtered and the solvents were evaporated in vacuo. The crude mixture was purified by flash column chromatography (SiO₂, NH₃ (7M in MeOH)/DCM, gradient from 0:100 to 5:95). A second purification was performed by RP HPLC (stationary phase: C18 XBridge 30×100 mm 5 μm), mobile phase: NH₄HCO₃ (0.25% solution in water)/ACN, gradient from 80:20 to 60:40) to afford compound 80 (110 mg, 46%).

### Preparation of Compound 81

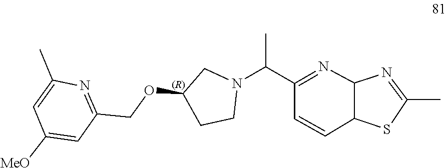

Intermediate I-28a (132 mg, 0.74 mmol) and Ti(Oi-Pr)₄ (CAS: 546-68-9; 300 μL, 1.01 mmol) were added to a solution of intermediate I-52 (150 mg, 0.68 mmol) in DCM (2.7 mL). The reaction mixture was stirred at 40° C. for 16 h. The reaction was cooled to 0° C. and methylmagnesium bromide (1.4M solution, 2.40 mL, 3.37 mmol) was added and the reaction mixture was stirred for 2 h. The reaction was quenched with MeOH and diluted with DCM and water. The emulsion was filtered through a pad of Celite®. The filtrate was treated with NH₄Cl (sat., aq.) and extracted with DCM. The organic layer was dried (MgSO₄), filtered and the solvents were evaporated in vacuo. The crude mixture was purified by flash column chromatography (SiO₂, NH₃ (7M in MeOH)/DCM, gradient from 0:100 to 5:95). A second purification was performed by RP HPLC (stationary phase: C18 XBridge 30×100 mm 5 μm), mobile phase: NH₄HCO₃ (0.25% solution in water)/ACN, gradient from 67:33 to 50:50) to afford compound 81 (120 mg, 45%).

### Preparation of Compound 82

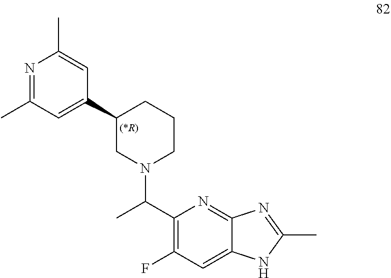

Intermediate I-3aR (50 mg, 0.26 mmol) was dissolved in ACN (2.1 mL). Intermediate I-80 (103 mg, 0.33 mmol) and K₂CO₃ (109 mg, 0.79 mmol) were added. The reaction mixture was stirred at 80° C. for 16 h. The solvent was evaporated in vacuo. The crude mixture was purified by RP HPLC (stationary phase: XBridge C18 50×100 mm, 5 μm), mobile phase: NH₄HCO₃ (0.25% solution in water)/ACN, gradient from 90:10 to 65:35). The residue was purified using an Isolute® SCX-2 cartridge which was washed with MeOH, and the product was eluted with NH₃ (7N in MeOH). The fraction was evaporated in vacuo and the residue was dried at 50° C. in a desiccator to afford compound 82 (20 mg, 21%) as a light yellow solid

### Preparation of Compound 83

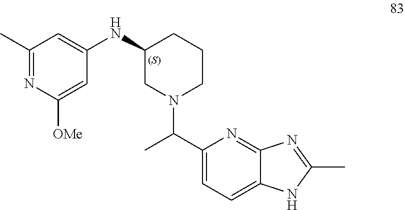

Compound 83 was prepared following an analogous procedure to the one described for the synthesis of compound 82 using intermediate I-56 and intermediate I-86 as starting materials.

The crude mixture was purified reverse phase ([65 mM NH₄OAc/ACN (90:10)]/[ACN/MeOH (1:1)], gradient from 91:19 to 45:55 to afford compound 83 (45 mg, 28%) as a white solid.

### Preparation of Compound 84

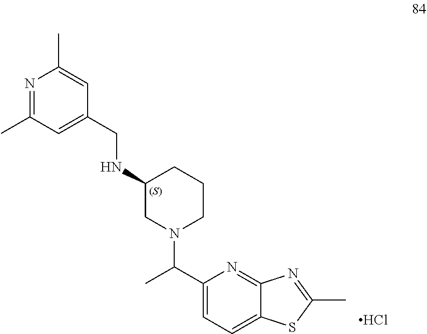

Compound 84 was prepared following an analogous procedure to the one described for the synthesis of compound 82 using intermediate I-86 and intermediate I-62 as starting materials.

The crude mixture was purified by flash column chromatography (SiO₂, DCM/MeOH, gradient from 100:0 to 96:4). The residue was triturated in Et₂O to afford a yellow oil (100 mg).

The residue was taken into DCM and treated with HCl (4N in 1,4-dioxane, 1 eq). The solvents were evaporated in vacuo and the product was triturated in DIPE to afford compound 84 (93 mg, 38%) as a slightly pink solid.

### Preparation of Compounds 85, 86 and 87

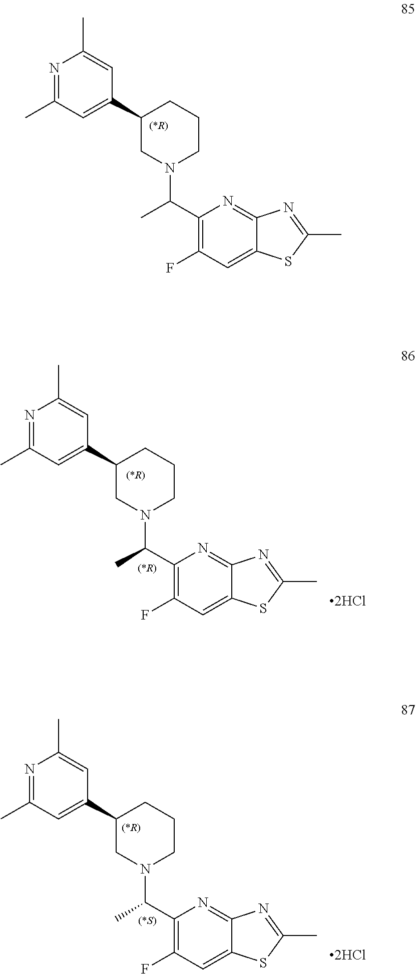

K₂CO₃ (545 mg, 3.94 mmol) was added to a solution of intermediate I-67 (33 mg, 1.45 mmol) and intermediate I-3aR (250 mg, 1.31 mmol) in ACN (8 mL). The reaction mixture was stirred for 20 h at 70° C. The reaction was diluted with EtOAc, filtered through Celite®, washed with EtOAc and the filtrate was concentrated in vacuo. The crude mixture was purified by flash column chromatography (SiO₂, NH₃ (7N in MeOH)/DCM, gradient from 0:100 to 5:95). A second purification was performed by RP HPLC (stationary phase: C18 XBridge 30×100 mm 5 μm), mobile phase: NH₄HCO₃ (0.25% solution in water)/ACN, gradient from 80:20 to 0:100) to afford compound 85 (95 mg, 19%). A purification via chiral SFC (stationary phase: Chiralcel OD-H 5 μm 250×21.2 mm, mobile phase: 75% CO₂, 25% i-PrOH (0.3% i-PrNH₂)) delivered fraction A (35 mg) and fraction B (36 mg, 7%).

Fraction A (35 mg) was dissolved in tert-butyl methyl ether (2 mL) and HCl (2M, 0.14 mL, 0.27 mmol) was added under stirring. The resulting precipitate was filtered and dried at 50° C. under vacuum to afford compound 86 (38 mg) as a dihydrochloride salt. Fraction B (saalonso_3593) was subjected to an analogous treatment than the one reported for fraction A to afford product 87.

### Preparation of Compound 88

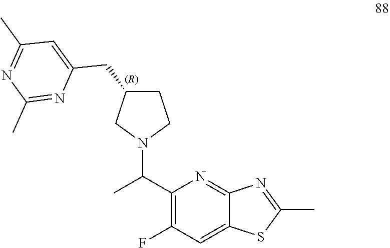

Compound 88 was prepared following an analogous procedure to the one described for the synthesis of compounds 85, 86 and 87 using intermediate I-73 and intermediate I-44 as starting materials.

The crude mixture was purified by flash column chromatography (SiO₂, DCM/NH₃ (7N in MeOH), gradient from 100:0 to 98:2). A second purification was performed by RP HPLC (stationary phase: C18 XBridge 30×100 mm 5 μm), mobile phase: NH₄HCO₃ (0.25% solution in water)/ACN, gradient from 67:33 to 50:50). The aqueous phase was extracted with EtOAc. The combined organic extracts were dried (Na₂SO₄), filtered and concentrated in vacuo to afford compound 88 (114 mg, 38%) as a yellow oil.

### Preparation of Compound 89

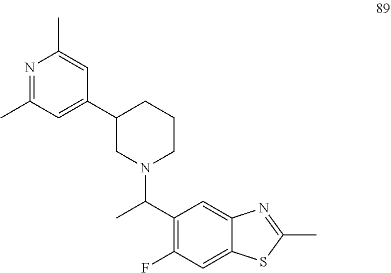

Intermediate I-3a (45.6 mg, 0.24 mmol) and K₂CO₃ (90.3 mg, 0.65 mmol) were added to a stirred solution of intermediate I-73 (50.0 mg, 0.22 mmol) in ACN (1.74 mL). The reaction mixture was stirred overnight at 80° C. Water was added and the mixture was extracted with DCM. The combined organic layers were dried (Na₂SO₄), filtered and evaporated in vacuo. The crude mixture was purified by flash column chromatography (SiO₂, NH₃ (7M in MeOH)/DCM, gradient from 0:100 to 10/90) to afford compound 89 (27 mg, 32%) as a light yellow oil.

### Preparation of Compound 90

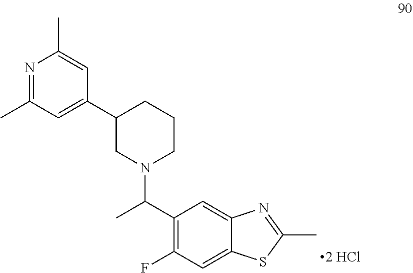

HCl (6M in i-PrOH, 0.16 mL, 1.0 mmol) was added to a stirred solution of compound 89 (14.0 mg, 36.5 μmol) in Et₂O (0.1 mL). The reaction mixture was stirred at room temperature for 4 h. The solvent was concentrated in vacuo. Tert-butyl methyl ether was added and the mixture was sonicated for 5 min. The solvent was evaporated in vacuo. The process was repeated until the obtention of a solid which was dried under vacuum to afford compound 90 (16.4 mg, 98%) as a yellow solid.

### Preparation of Compound 91

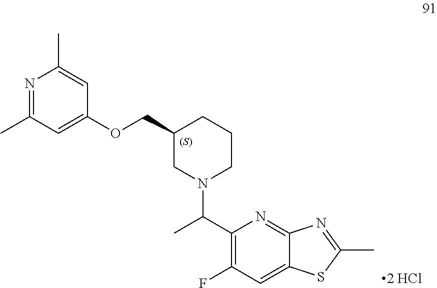

Compound 91 was prepared following an analogous procedure to the one described for the synthesis of compound 89 using intermediate I-67 and intermediate I-10a as starting materials.

The crude mixture was purified by flash column chromatography (SiO₂, DCM/MeOH, gradient from 100:0 to 95:5). A second purification was performed via RP HPLC (stationary phase: C18 XBridge 30×100 mm 5 μm), mobile phase: NH₄HCO₃ (0.25% solution in water)/ACN, gradient from 67:33 to 50:50).

The residue (65 mg) was dissolved in Et₂O (0.3 mL) and HCl (7N in i-PrOH) (0.3 mL) was added. The mixture was stirred at room temperature for 16 h. The solvent was concentrated in vacuo. Tert-butylmethylether was added and the mixture was sonicated for 10 min. The solvent was removed in vacuo. The process was repeated until the obtention of a solid, which was dried under vacuum at 50° C. The residue was dissolved in MeOH (1 mL) and the mixture was concentrated in vacuo. Tert-butylmethylether was added and the mixture was sonicated for 10 min. The solvent was evaporated in vacuo and the solid was dried at 50° C. in a desiccator to afford compound 91 (45 mg, 30%) as a white solid

### Preparation of Compound 92

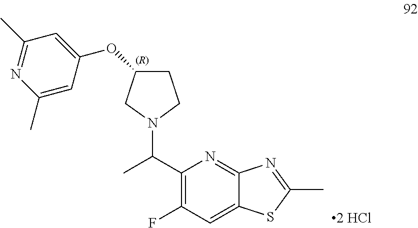

Compound 92 was prepared following an analogous procedure to the one described for the synthesis of compound 89 using intermediate I-44 and intermediate I-67 as starting materials.

The crude mixture was purified by flash column chromatography (SiO₂, DCM/NH₃ (7N in MeOH), gradient from 100:0 to 95:5). A second purification was performed via RP HPLC (stationary phase: C18 XBridge 30×100 mm 5 μm), mobile phase: NH₄HCO₃ (0.25% solution in water)/ACN, gradient from 75:25 to 57:43) to afford an oil (65 mg).

The residue (56 mg) was dissolved in tert-butylmethylether (2 mL) and HCl (2M in Et₂O, 0.29 mL, 0.58 mmol) was added under stirring. The precipitate was filtered off and the product was dried in the oven at 50° C. under vacuum to afford compound 92 (65 mg) as a white solid.

### Preparation of Compounds 93 and 94

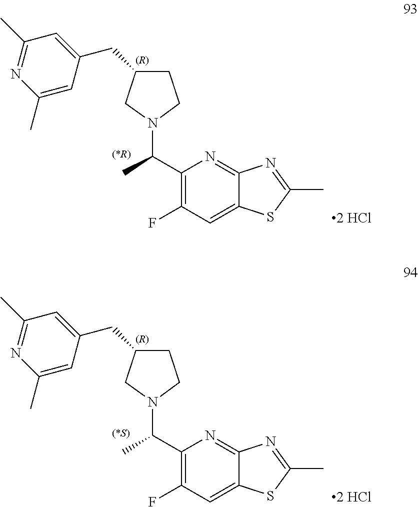

Compound 50 was purified via chiral SFC (stationary phase: CHIRACEL OJ-H 5 μm 250*30 mm, mobile phase: 82% CO₂, 18% i-PrOH (0.3% i-PrNH₂)) to afford fraction A (44 mg) and fraction B (42 mg).

Fraction A (44 mg, 0.11 mmol) was dissolved in Et₂O (2.38 mL) and HCl (2N in Et₂O, 0.17 mL, 0.34 mmol) was added. The precipitated was filtered to give compound 93 (38.4 mg, 73%) as a white solid.

Product 94 (39.2 mg, 79%) was obtained following an analogous procedure to the one described for the synthesis of product 93 using fraction B as starting material.

### Preparation of Compounds 95 and 96

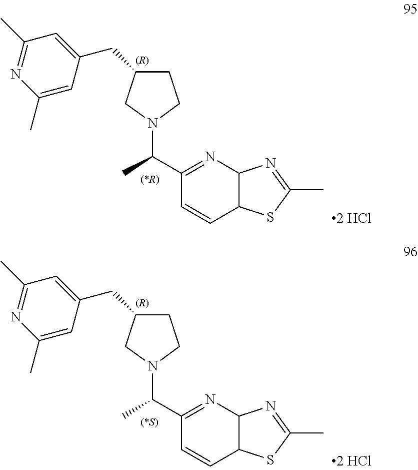

Compound 43 (364 mg) was purified via chiral SFC (stationary phase: CHIRALPAK AD-H 5 μm 250*30 mm, mobile phase: 80% CO₂, 20% EtOH (0.3% i-PrNH₂)) to afford fraction A (141 mg) and fraction B (149 mg).

Fraction A (130 mg, 0.36 mmol) was dissolved in tert-butyl methyl ether (2 mL) and HCl (2M in Et₂O, 2 mL, 4 mmol) was added under stirring. The precipitate was filtered and the compound was dried in the oven at 50° C. under vacuum. The crude product was purified by RP HPLC (stationary phase: C18 XBridge 30×100 mm 5 μm), mobile phase: NH₄HCO₃ (0.25% solution in water)/ACN, gradient from 80:20 to 0:100). The desired fractions were collected and concentrated in vacuo. The resulting product was dissolved in tert-butyl methyl ether (2 mL) and HCl (2M in Et₂O, 2 mL, 4 mmol) was added under stirring. The resulting precipitate was filtered and dried at 50° C. under vacuum to afford compound 95 (95 mg, 61%).

Fraction B (120 mg, 0.33 mmol) was dissolved in tert-butyl methyl ether (2 mL) and HCl (2M in Et₂O, 2 mL, 4 mmol) was added under stirring. The precipitate was filtered and the compound was dried in the oven at 50° C. under vacuum to afford compound 96 (90 mg, 63%).

### Preparation of Compound 97

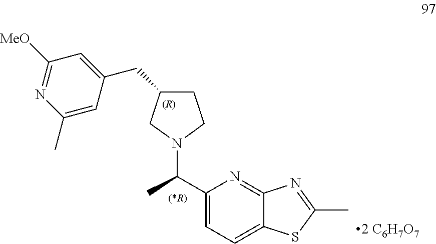

Compound 44 (140 mg) was purified via chiral SFC (stationary phase: CHIRALPAK AD-H 5 μm 250*30 mm, mobile phase: 80% CO₂, 20% MeOH (0.3% i-PrNH₂)) to afford fraction A (54 mg) and fraction B (49 mg).

Fraction B (49 mg, 0.13 mmol) was dissolved in tert-butyl methyl ether (2 mL) and citric acid (49.2 mg, 0.26 mmol) was added under stirring. The resulting precipitate was filtered and dried at 50° C. under vacuum for 48 h to afford compound 97 (55 mg, 56%).

### Preparation of Compound 98

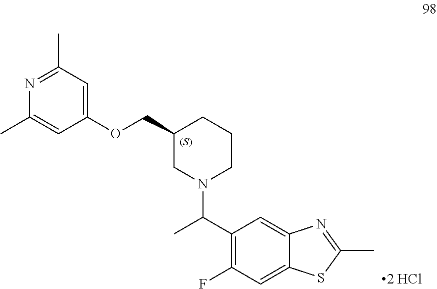

Intermediate I-73 (65.0 mg, 0.27 mmol) was dissolved in ACN (2.2 mL) and intermediate I-10a (65.8 mg, 0.30 mmol) and K₂CO₃ (113 mg, 0.82 mmol) were added. The reaction mixture was stirred for 16 h at 80° C. The mixture was diluted with water and extracted with DCM. The organic was dried (Na₂SO₄), filtered and evaporated in vacuo. The crude mixture was purified by flash column chromatography (SiO₂, NH₃ (7N in MeOH)/DCM, gradient from 0:100 to 10:90). The residue was purified by RP HPLC (stationary phase: C18 XBridge 30×100 mm 5 μm), mobile phase: NH₄HCO₃ (0.25% solution in water)/ACN, gradient from 53:46 to 36:64). The residue was dissolved in EtOAc and washed with a NaHCO₃ (sat., aq.). The organic layer was dried (Na₂SO₄), filtered and concentrated in vacuo.

The residue (25 mg) was dissolved in Et₂O (0.1 mL) and HCl (7N in i-PrOH) (0.1 mL) was added. The mixture was stirred at room temperature for 16 h and the solvent was evaporated in vacuo. Tert-butyl methyl ether was added and the mixture was sonicated for 10 min. The solvent was concentrated in vacuo. The process was repeated until the obtention of a solid which was dried under vacuum to afford compound 98 (35 mg, 26%) as a cream solid.

### Preparation of Compounds 99, 100 and 101

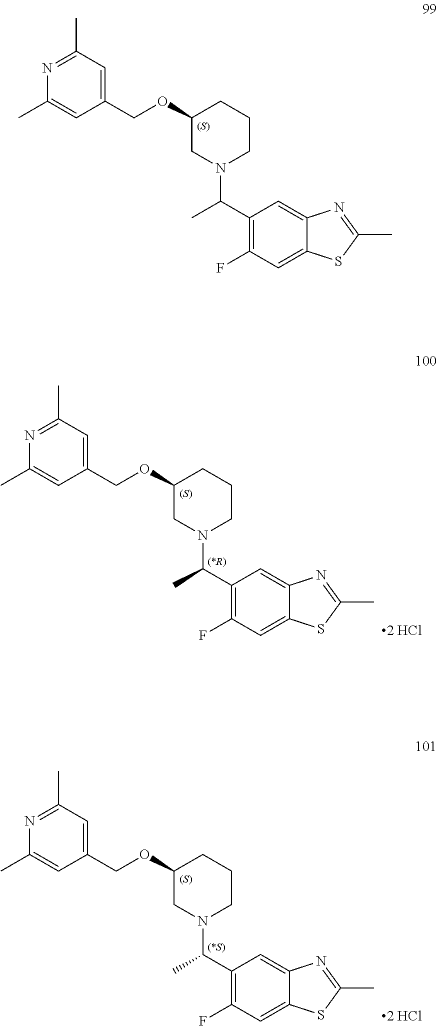

Compounds 99, 100 and 101 were prepared following an analogous procedure to the one reported for the synthesis of compound 98 using intermediate I-73 and intermediate 1-46 as starting materials.

The crude mixture was purified by flash column chromatography (SiO₂, NH₃ (7N in MeOH)/DCM, gradient from 0:100 to 10:90). The residue was purified by RP HPLC (stationary phase: C18 XBridge 30×100 mm 5 μm), mobile phase: NH₄HCO₃ (0.25% solution in water)/ACN, gradient from 80:20 to 0:100). The residue was dissolved in EtOAc, washed with NaHCO₃ (sat., aq.), dried (Na₂SO₄), filtered and concentrated in vacuo to afford compound 99 (92.6 mg, 38%) as a white solid.

A purification was performed via chiral SFC (stationary phase: CHIRALPAK AD-H 5 μm 250*30 mm, mobile phase: 80% CO₂, 20% i-PrOH (0.3% i-PrNH₂)) to afford fraction A (37 mg) and fraction B (37 mg).

The products were separately dissolved in Et₂O (0.2 mL) and HCl (7N in i-PrOH) (0.2 mL) was added. The mixtures were stirred at room temperature for 16 h. The solvents were evaporated in vacuo and tert-butyl methyl ether was added. The mixtures were sonicated for 10 min and the solvents were removed in vacuo. The process was repeated until the obtention of solids which were dried under vacuum at 50° C. for 5 h to afford compound 100 (42.3 mg, 15%) and compound 101 (44.3 mg, 15%) as solids.

### Preparation of Compound 102

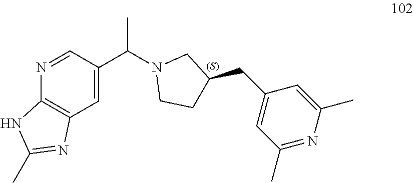

To a solution of intermediate 92 (123 mg, 0.26 mmol) in DCM (1 mL) was added TFA (0.35 mL, 4.62 mmol). The reaction mixture was stirred at room temperature for 18 h. The reaction was concentrated to dryness in vacuo. The residue was purified by ion exchange chromatography using an Isolute SCX2 cartridge and eluting with MeOH, and then with NH₃ (7M in MeOH). Fractions were collected and the solvents were evaporated in vacuo. The residue was purified by RP HPLC (stationary phase: C18 XBridge 30×100 mm 5 μm), mobile phase: NH₄HCO₃ (0.25% solution in water)/ACN, gradient from 80:20 to 60:40). The desired fractions were collected and Na₂CO₃ (sat., aq.) was added. The product extracted with DCM. The solvents were evaporated in vacuo to afford compound 102 (30 mg, 33%).

### Preparation of Compound 103

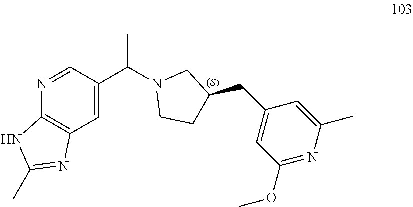

TFA (0.24 mL, 3.18 mmol) was added to a solution of intermediate 91 (84.0 mg, 0.17 mmol) in DCM (1.3 mL) and the reaction mixture was stirred at room temperature for 18 h. The reaction was concentrated in vacuo. The crude mixture was purified by RP HPLC (stationary phase: C18 XBridge 30×100 mm 5 μm), mobile phase: NH₄HCO₃ (0.25% solution in water)/ACN, gradient from 80:20 to 60:40). The residue was washed with water and NaHCO₃ (sat., aq.) and extracted with EtOAc. The organic layer was dried (Na₂SO₄), filtered and the solvent was evaporated in vacuo to afford compound 103 (40 mg, 65%).

### Preparation of Compound 104

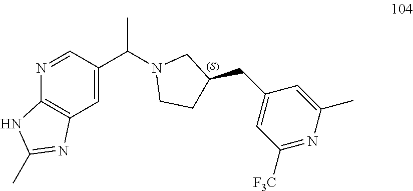

TFA (0.78 mL, 10.2 mmol) was added to a stirred solution of intermediate 93 (150 mg, 0.28 mmol) in DCM (1.55 mL). The reaction mixture was stirred at room temperature for 3 days. The solvent was evaporated in vacuo and the residue dissolved in TFA neat (1 mL). The mixture was stirred at room temperature for 16 h. The solvent was evaporated in vacuo. The residue was dissolved in DCM and washed with Na₂CO₃ (sat., aq.). The organic layer was dried (Na₂SO₄), filtered and the solvents were evaporated in vacuo. The crude mixture was purified by RP HPLC (stationary phase: C18 XBridge 30×100 mm 5 μm), mobile phase: NH₄HCO₃ (0.25% solution in water)/ACN, gradient from 80:20 to 60:40). The desired fractions were collected and concentrated in vacuo. The aqueous phase was extracted with EtOAc (3 times). The combined organic layers were dried (Na₂SO₄), filtered and concentrated in vacuo to afford compound 104 (58 mg, 51%).

### Preparation of Compounds 105 and 106

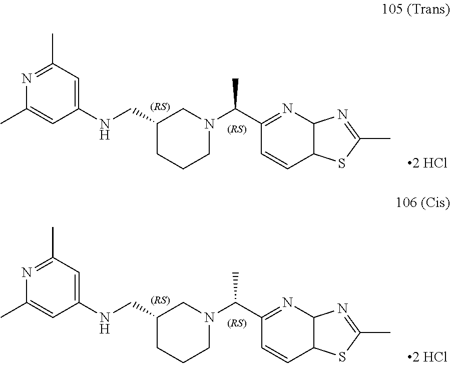

Intermediate I-28a (120 mg, 0.67 mmol) and Ti(Oi-Pr)₄ (CAS: 546-68-9; 0.70 mL, 2.36 mmol) were added to a solution of intermediate I-54 (155 mg, 0.71 mmol) in anhydrous THF (2.22 mL) at room temperature. The reaction mixture was stirred for 18 h. The mixture was distillated and dried in vacuo. Anhydrous THF (2.22 mL) was added and the mixture was cooled to 0° C. Methylmagnesium bromide (1.4M in THF, 2.41 mL, 3.37 mmol) was added dropwise and the reaction mixture was stirred at 0° C. for 15 min, and at room temperature for 15 h. NH₄Cl (sat., aq., 2 mL) was added and the mixture was extracted with DCM and MeOH (9:1) (3 times). The combined organics layers were dried (MgSO₄), filtered and concentrated in vacuo. The crude mixture was purified by flash column chromatography (SiO₂, NH₃ (7N in MeOH)/DCM, gradient from 0:100 to 10:90) to afford a mixture compounds (60 mg, 23%). The mixture (170 mg, 0.43 mmol) was purified by reverse phase ([65 mM NH₄OAc/ACN (90:10)]/[ACN/MeOH (1:1)], gradient from 95:5 to 63:37). The desired fractions were collected and concentrated in vacuo. Another purification was performed by reverse phase ([H₂O (25 mM NH₄HCO₃)/[MeCN/MeOH (1:1)], gradient from 81:19 to 45:55) to afford fraction A (52 mg, 87%) and fraction B (30 mg, 50%).

The products were separately taken into DCM and treated with HCl (4N in 1,4-dioxane, 2 eq). The solvents were evaporated in vacuo and the products were triturated in Et₂O to afford compound 106 (51 mg) and compound 105 (27 mg) as white solids.

### Preparation of Compound 107

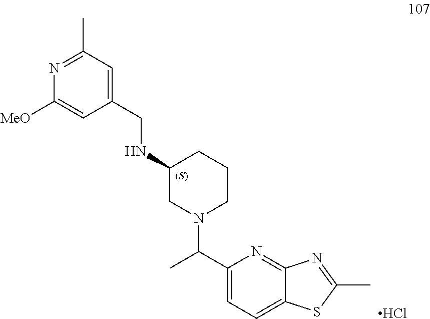

K₂CO₃ (44.7 mg, 0.32 mmol) was added to a stirred solution of intermediate 94 (54.7 mg, 0.11 mmol) in MeOH (0.29 mL) and H₂O (0.11 mL) at room temperature. The reaction mixture was stirred at 60° C. for 16 h and the organic solvent was evaporated in vacuo. The mixture was extracted with EtOAc. The organic layer was dried (MgSO₄), filtered and the solvents were evaporated in vacuo. The crude mixture was purified by flash column chromatography (SiO₂, DCM/MeOH, gradient from 100:0 to 90:10). to afford compound 107 (33.2 mg, 74%) as a yellow oil. The residue (33.2 mg) was taken into DCM and treated with HCl (4N in 1,4-dioxane, 1 eq). The solvents were evaporated in vacuo and the product was triturated in Et₂O to afford compound 107 (14 mg, 29%) as a white solid.

### Preparation of Compound 108

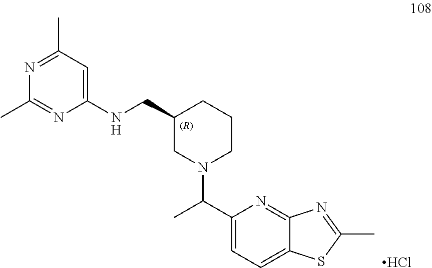

DIPEA (0.11 mL, 0.64 mmol) was added to a stirred solution of 4-chloro-2,6-dimethylpyrimidine [4472-45-1] (61.1 mg, 0.43 mmol), intermediate 90 (137 mg, 0.47 mmol) in 1-butanol (5 mL). The reaction mixture was stirred at 80° C. for 20 h and at 110° C. for 2 h. The mixture was diluted with DCM and NaHCO₃ (sat., aq.) was added. The organic phase was separated, dried (MgSO₄), filtered and the solvents were evaporated in vacuo. The crude mixture was purified by flash column chromatography (SiO₂, DCM:MeOH (10:1)/DCM, gradient from 0:100 to 80:20). The residue (97 mg) was dissolved in DCM (5 mL) and HCl (4M in 1,4-dioxane, 60.5 μL, 0.24 mmol) was added. The solvents were concentrated in vacuo and the product was triturated in Et₂O. The solid was collected by filtration and dried to afford compound 108 (90 mg, 48%) as a white solid.

### Preparation of Compound 109

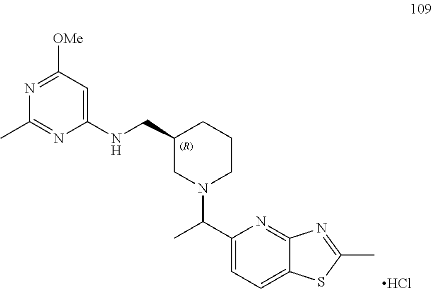

NaOt-Bu (31.0 mg, 0.32 mmol) was added to a stirred suspension of Pd₂dba₃ (5.91 mg, 6.46 μmol) and t-BuXPhos (8.22 mg, 19.4 μmop in 1,4-dioxane (15 mL) in a sealed tube and under N₂ atmosphere at room temperature. The reaction mixture was stirred at 95° C. for 5 min, then a mixture of intermediate I-90 (45.0 mg, 0.16 mmol) and 4-bromo-2-methoxy-6-methylpyridine (CAS: 1083169-00-9; 26.1 mg, 0.13 mmol) in 1,4dioxane (5 mL) was added to the reaction mixture under N₂ atmosphere at 95° C. The reaction mixture was stirred at 100° C. for 1.5 h. The mixture was diluted with NaHCO₃ (sat., aq.) and extracted with DCM. The organic layer was dried (MgSO₄), filtered and the solvents were evaporated in vacuo. The crude mixture was purified by reverse phase ([65 mM NH₄OAc/ACN (90:10)]/[ACN:MeOH (1:1)], gradient from 90:10 to 54:46). The residue (18 mg) was taken into DCM and treated with HCl (4N in 1,4-dioxane, 1 eq). The solvents were evaporated in vacuo to afford compound 109 (16 mg, 26%) as a white solid.

The following compounds were prepared following the methods exemplified in the Experimental Part. In case no salt form is indicated, the compound was obtained as a free base.

TABLE 1

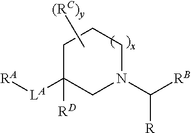

| Co. No. | Structure | Salt Form |
|---|---|---|
| 1 | 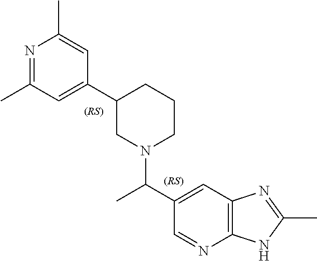 | |
| 2 | 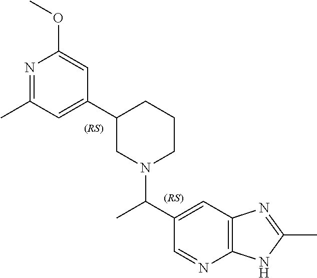 | |
| 3 | 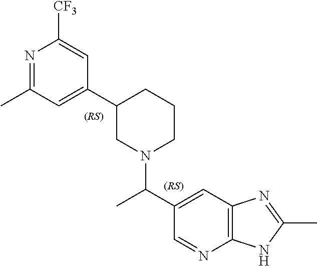 | |
| 4a | 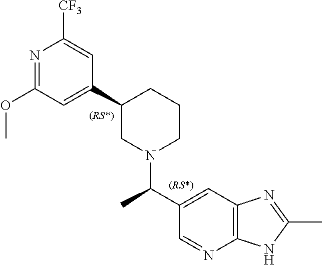 [SMILES: COc1cc([C@H]2CCCN([C@H](C)c3cnc4[nH]c(C)nc4c3)C2)cc(C(F)(F)F)n1] | |
| 4b | 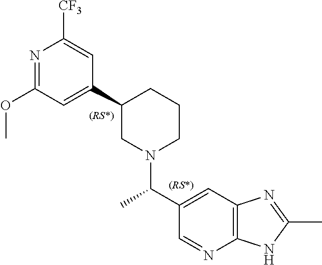 [SMILES: COc1cc([C@H]2CCCN([C@@H](C)c3cnc4[nH]c(C)nc4c3)C2)cc(C(F)(F)F)n1] | |
| 5 | 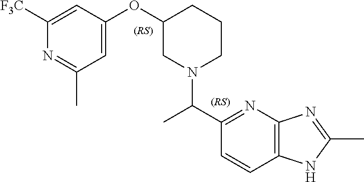 [SMILES: Cc1cc(OC2CCCN(C(C)c3ccc4[nH]c(C)nc4n3)C2)cc(C(F)(F)F)n1] | |
| 6a | 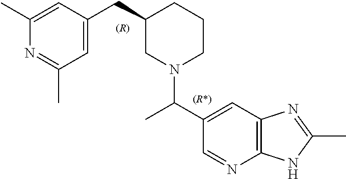 | |
| 6b | 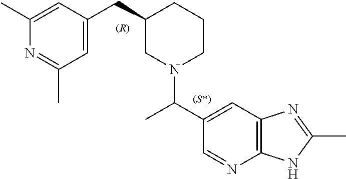 | |
| 7 | 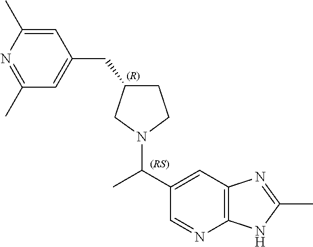 | |
| 8 | 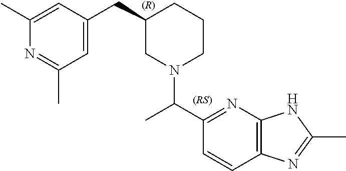 | |
| 9 | 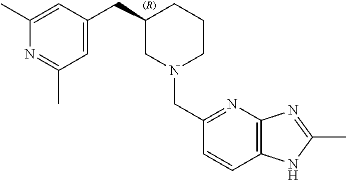 | |
| 10 | 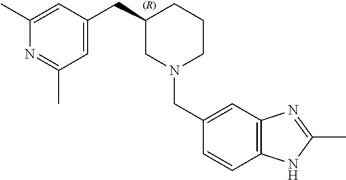 | |
| 11 | 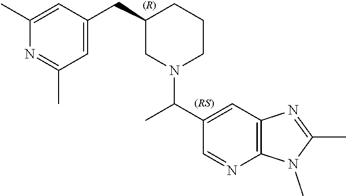 | |
| 12ab | 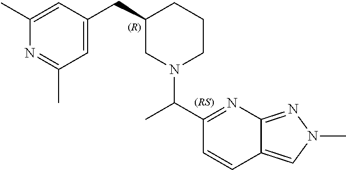 | |
| 12a | 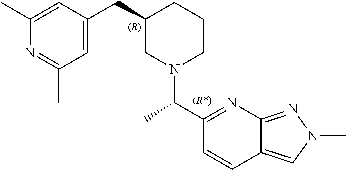 | |
| 12b | 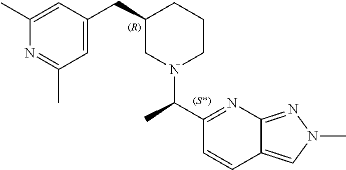 | |
| 13 | 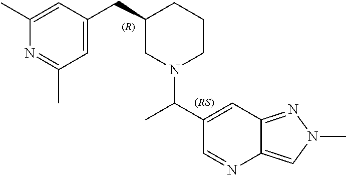 | |
| 14 | 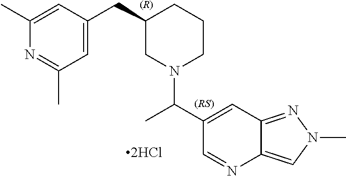 | •2 HCl |
| 15 | 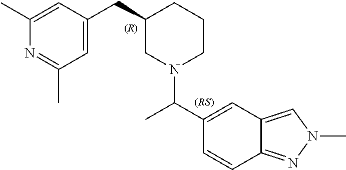 | |
| 16 | 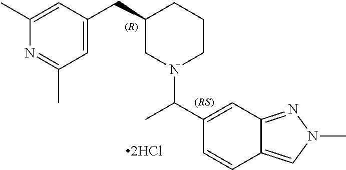 | •2 HCl |
| 17 | 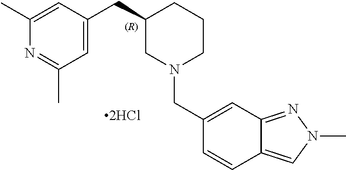 | •2 HCl |
| 18 | 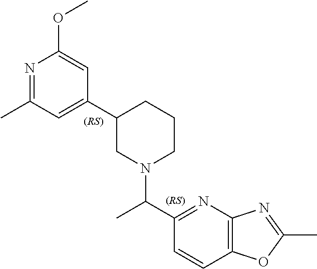 | |
| 19 | 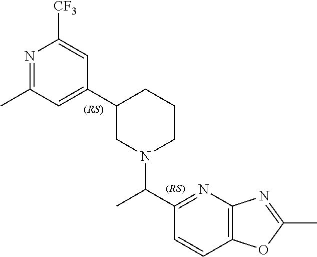 | |
| 20 | 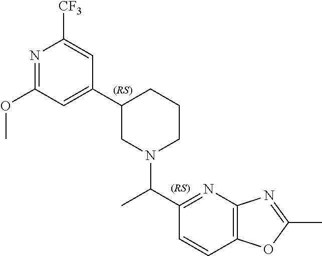 | |
| 21ab | 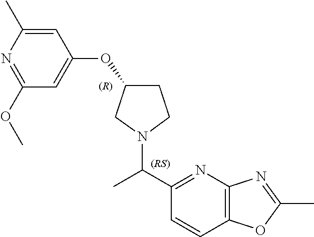 | |
| 21a | 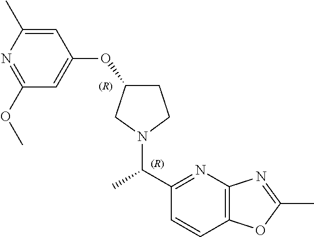 | |
| 22 | 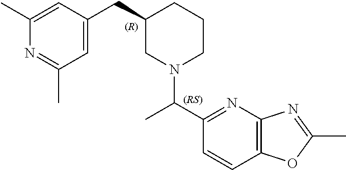 | |
| 23a | 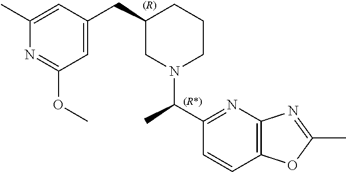 | |
| 23b | 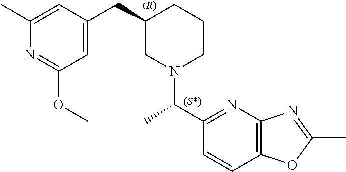 | |
| 24ab | 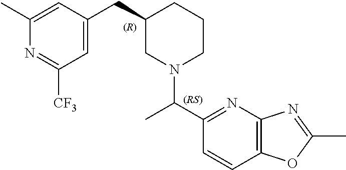 | |
| 24a | 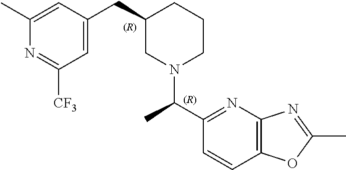 | |
| 24b | 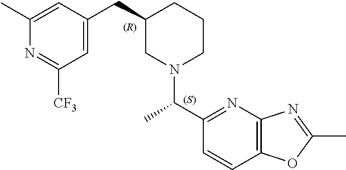 | |
| 25ab | 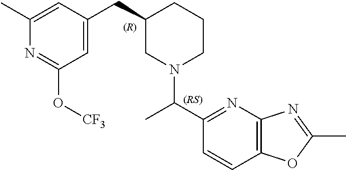 | |
| 25a | 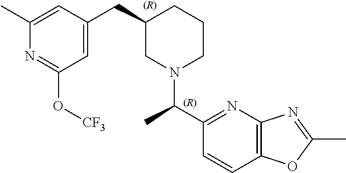 | |
| 26 | 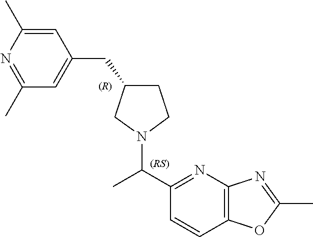 | |
| 27a | 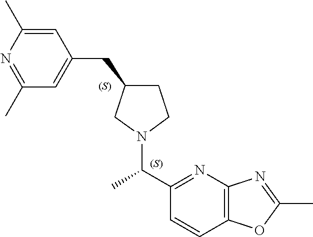 | |
| 27b | 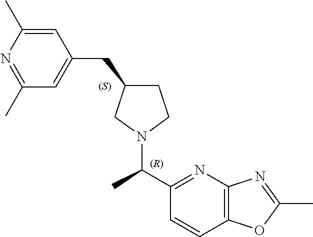 | |
| 28 | 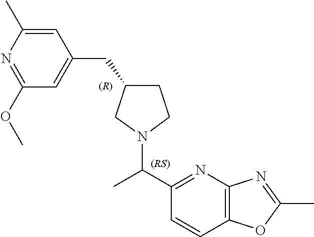 | |
| 29 | 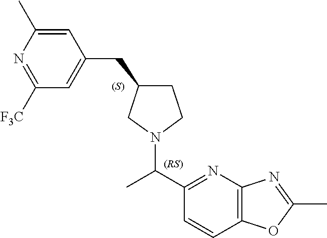 | |
| 30 | 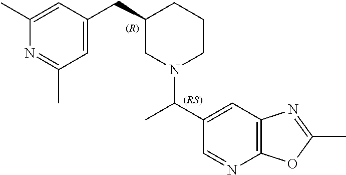 | |
| 31 | 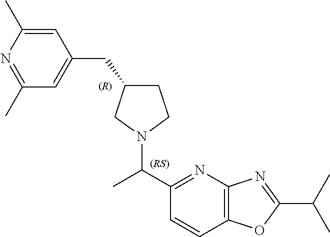 | |
| 32 | 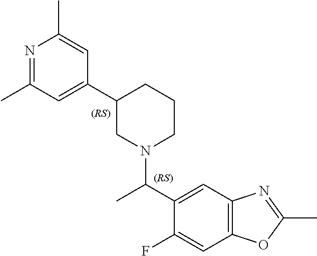 | |
| 33ab | 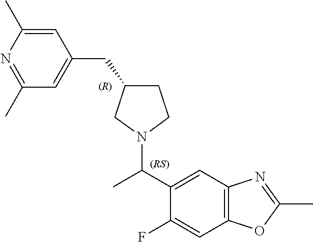 | |
| 33a | 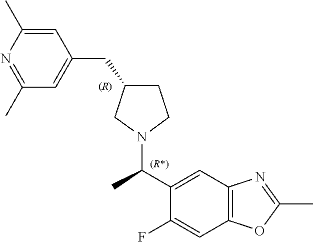 | |
| 33b | 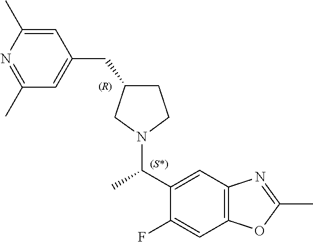 | |
| 34 | 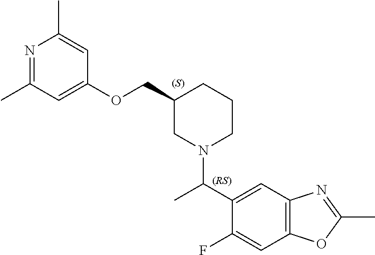 | |
| 35 | 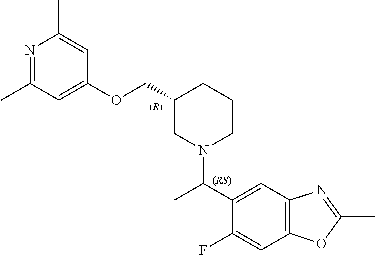 | |
| 36 | 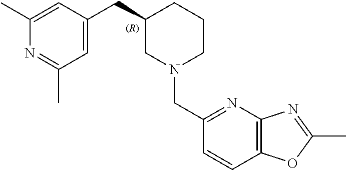 | |
| 37 | 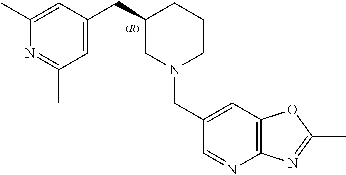 | |
| 38 | 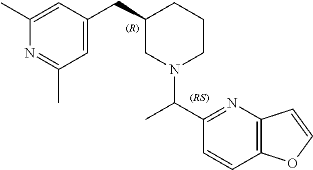 | |
| 39 | 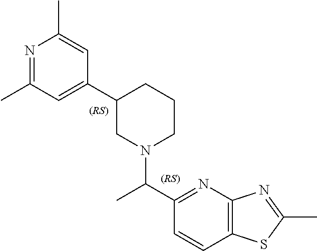 | |
| 40 | 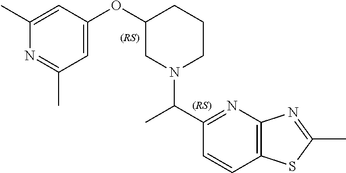 | |
| 41 | 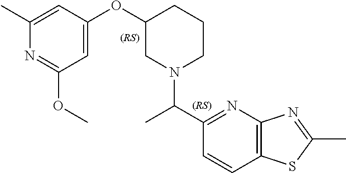 | |
| 42a | 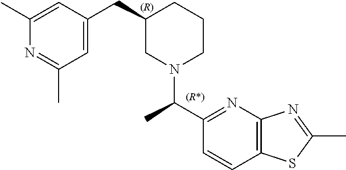 | |
| 42b | 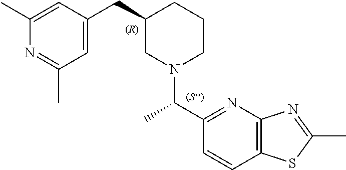 | |
| 43 | 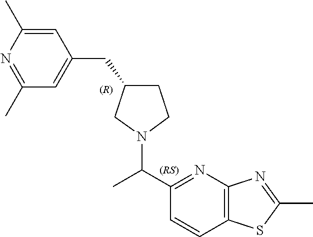 | |
| 44 | 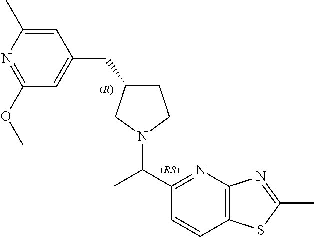 | |
| 45ab | 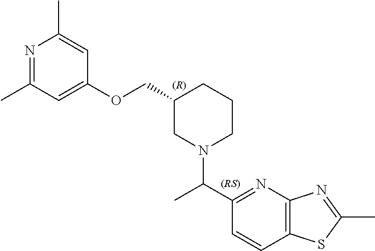 | |
| 45a | 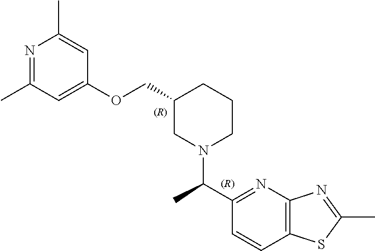 | |
| 46ab | 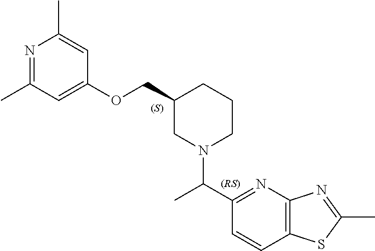 | |
| 46a | 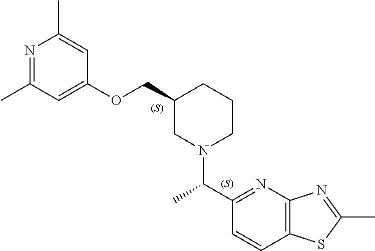 | |
| 47 | 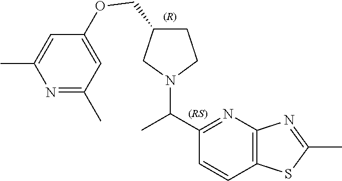 | |
| 48 | 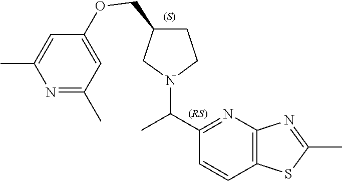 | |
| 49ab | 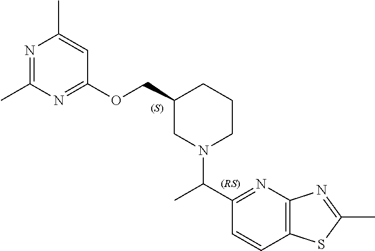 | |
| 49a | 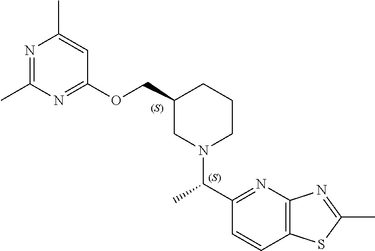 | |
| 49b | 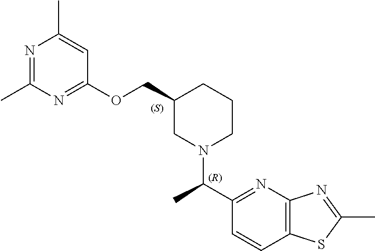 | |
| 50 | 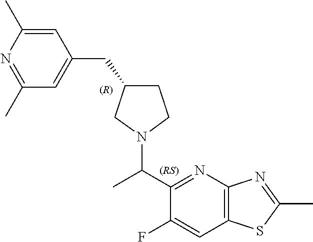 | |
| 51 | 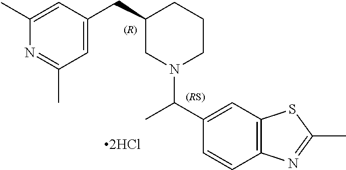 | •2 HCl |
| 52 | 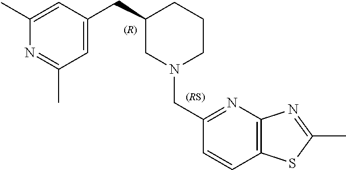 | |
| 53 | 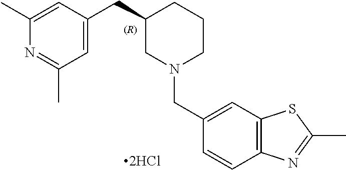 | •2 HCl |
| 54 | 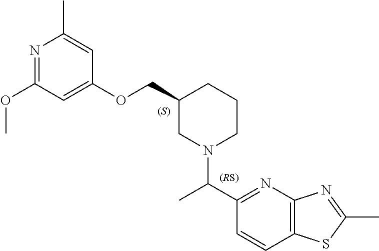 | |
| 55 | 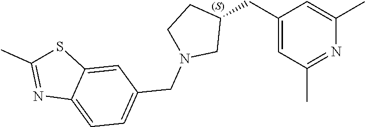 | 2 HCl |
| 56 | 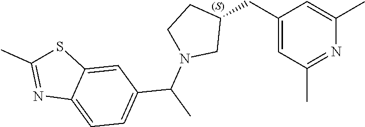 | 2 HCl |
| 57 | 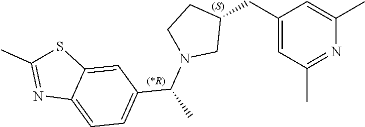 | 2 HCl |
| 58 | 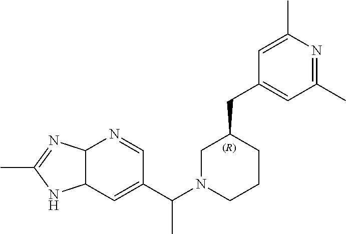 | |
| 59 | 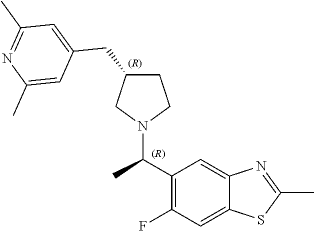 | 2 C₆H₈O₇ citric acid |
| 60 | 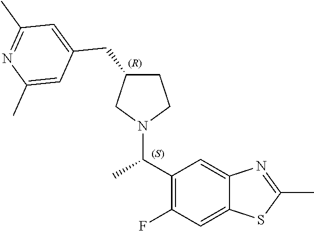 | 2 C₆H₈O₇ |
| 61 | 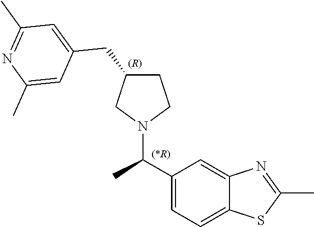 | 2 C₆H₈O₇ |
| 62 | 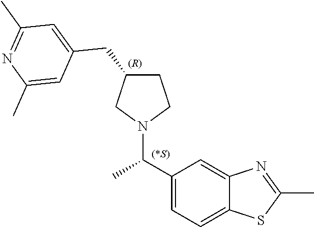 | 2 C₆H₈O₇ |
| 63 | 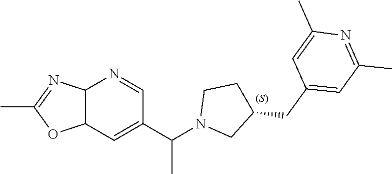 | |
| 64 | 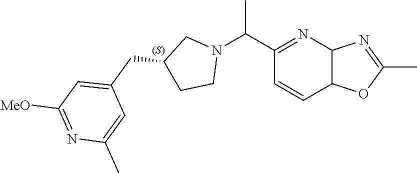 | |
| 65 | 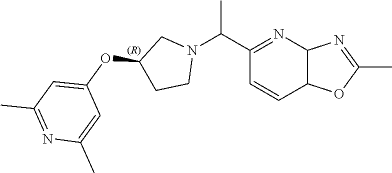 | |
| 66 | 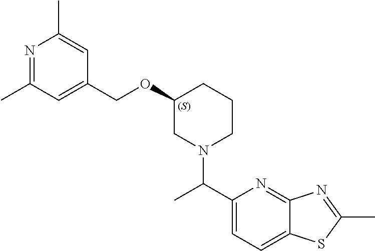 | |
| 67 | 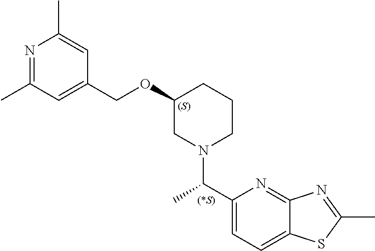 | |
| 68 | 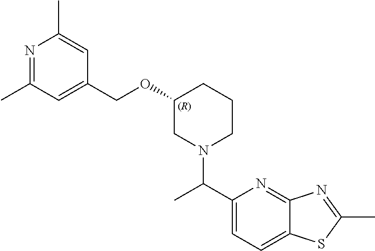 | |
| 69 | 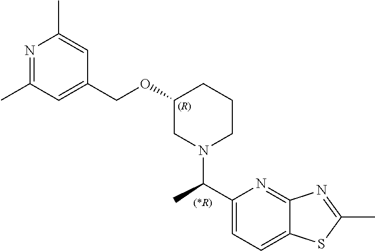 | |
| 70 | 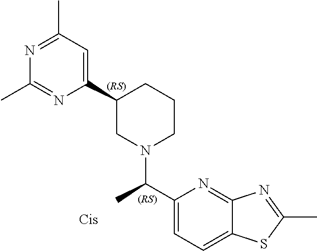 | |
| 71 | 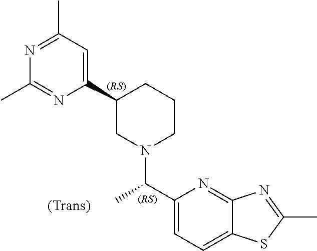 | |
| 72 | 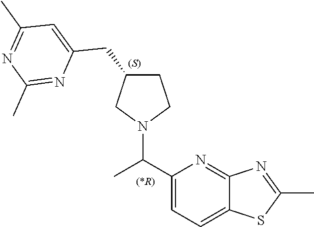 | |
| 73 | 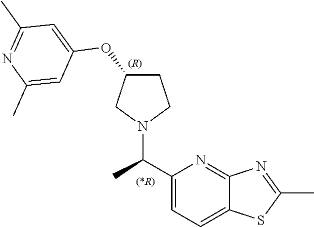 | 2 HCl |
| 74 | 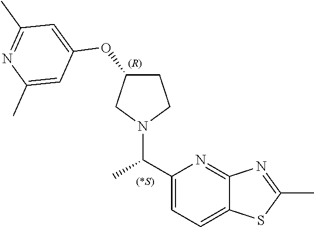 | 2 HCl |
| 75 | 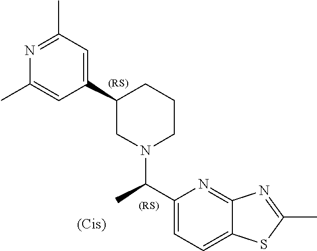 | |
| 76 | 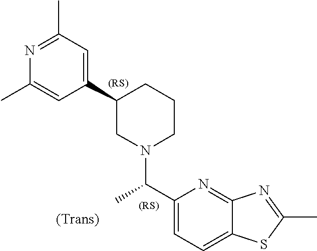 | |
| 77 | 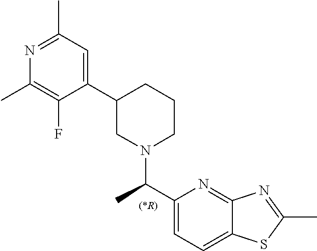 | |
| 78 | 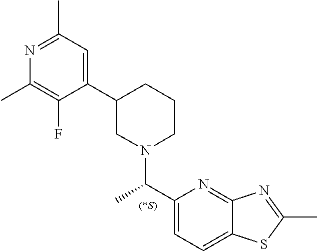 | |
| 79 | 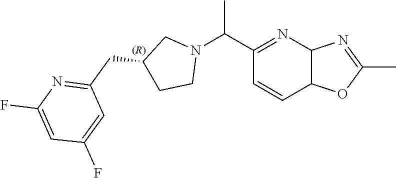 | |
| 80 | 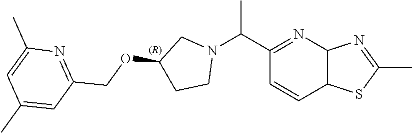 | |
| 81 | 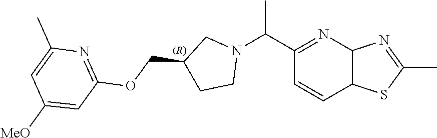 | |
| 82 | 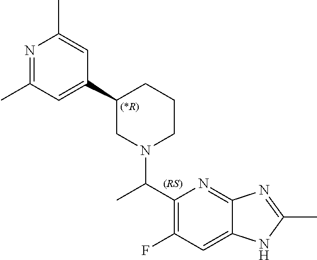 | |
| 83 | 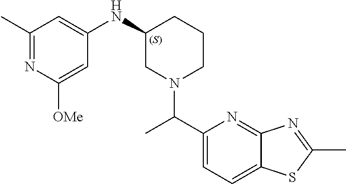 | |
| 84 | 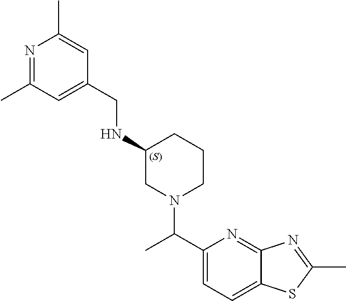 | HCl |
| 85 | 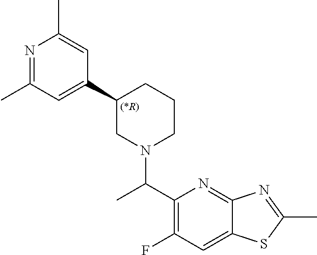 | |
| 86 | 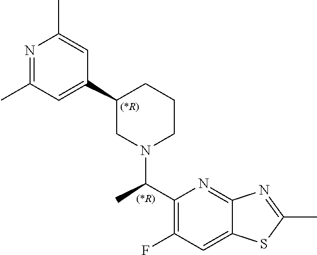 | 2 HCl |
| 87 | 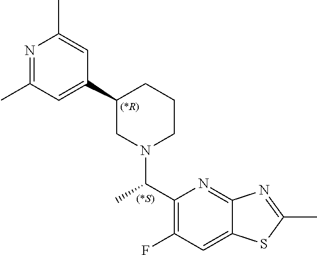 | 2 HCl |
| 88 | 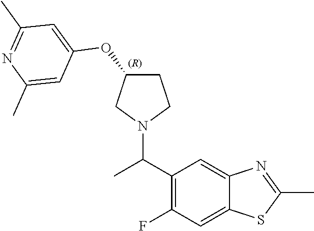 | |
| 89 | 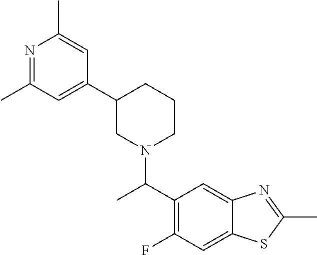 | |
| 90 | 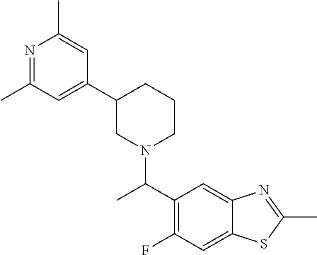 | 2 HCl |
| 91 | 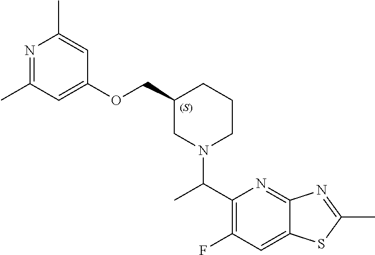 | 2 HCl |
| 92 | 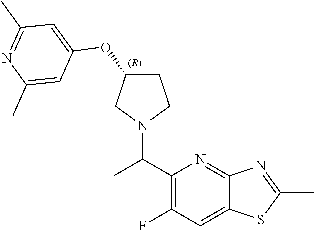 | 2 HCl |
| 93 | 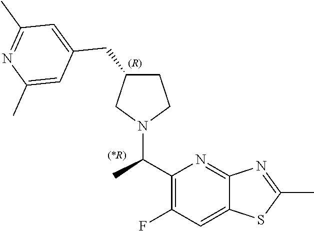 | 2 HCl |
| 94 | 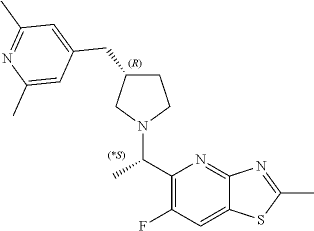 | 2 HCl |
| 95 | 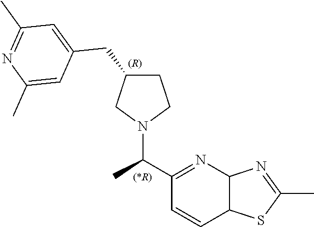 | 2 HCl |
| 96 | 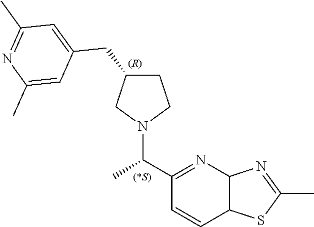 | 2 HCl |
| 97 | 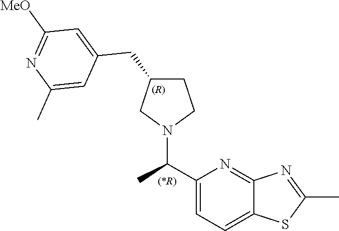 | 2 C₆H₈O₇ |
| 98 | 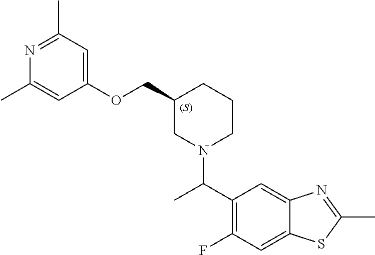 | 2 HCl |
| 99 | 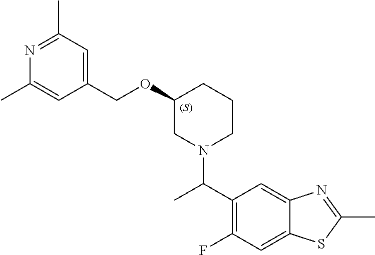 | |
| 100 | 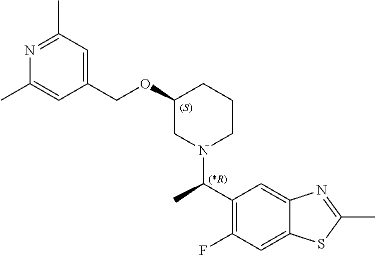 | 2 HCl |
| 101 | 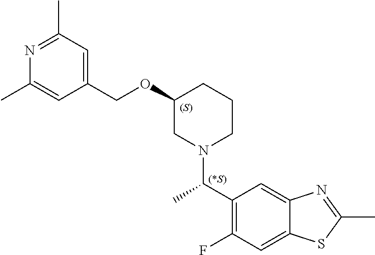 | 2 HCl |
| 102 | 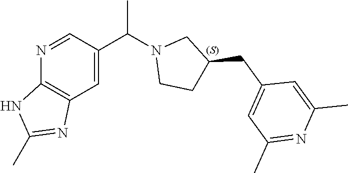 | |
| 103 | 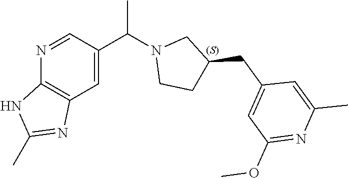 | |
| 104 | 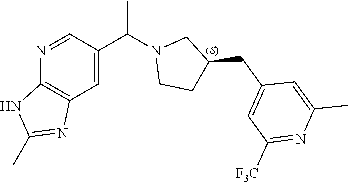 | |
| 105 | 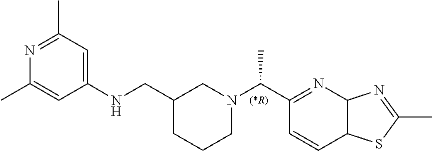 | 2 HCl |
| 106 | 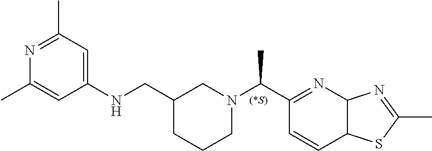 | 2 HCl |
| 107 | 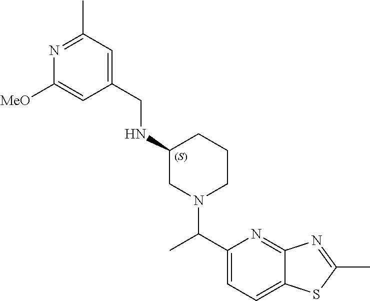 | HCl |
| 108 | 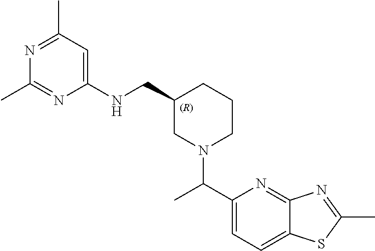 | HCl |
| 109 | 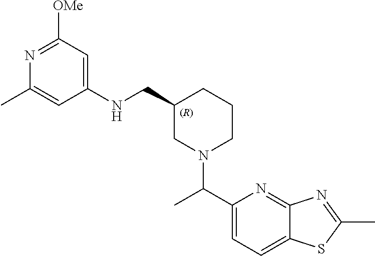 | HCl |

The values of salt stoichiometry or acid content in the compounds as provided herein, are those obtained experimentally. The content of hydrochloric acid reported herein was determined by ¹H NMR integration and/or elemental analysis.

### Analytical Part

### Melting Points

Values are peak values, and are obtained with experimental uncertainties that are commonly associated with this analytical method.

DSC823e (A): For a number of compounds, melting points were determined with a DSC823e (Mettler-Toledo) apparatus. Melting points were measured with a temperature gradient of 10° C./minute. Maximum temperature was 300° C. Values are peak values (A).

### LCMS

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, [M+CH₃COO]⁻ etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” Single Quadrupole Detector, “MSD” Mass Selective Detector, “QTOF” Quadrupole-Time of Flight, “rt” room temperature, “BEH” bridged ethylsiloxane/silica hybrid, HSS″ High Strength Silica, “CSH” charged surface hybrid, “UPLC” Ultra Performance Liquid Chromatography, “DAD” Diode Array Detector.

TABLE 2
LC-MS Methods (Flow expressed in mL/min;
column temperature (T) in ° C.; Run time in min).

| | | | | | Flow | Run |
|---|---|---|---|---|---|---|
| Method | Instrument | Column | Mobile Phase | Gradient | Col T | Time |
| 1 | Waters: | Waters: | A: 95% | From 95% A | 1 | 5 |
| | Acquity ® | BEH C18 | CH₃COONH₄ | to 5% A in | 50 | |
| | IClass | (1.7 μm, | 6.5 mM + 5% | 4.6 min, held | | |
| | UPLC ®- | 2.1 × 50 mm) | CH₃CN, B: | for 0.4 min | | |
| | DAD and | | CH₃CN | | | |
| | Xevo G2-S | | | | | |
| | QTOF | | | | | |
| 2 | Waters: | Waters: | A: 95% | From 95% A | 1 | 2 |
| | Acquity ® | BEH C18 | CH₃COONH₄ | to 40% A in | 50 | |
| | IClass | (1.7 μm, | 6.5 mm + 5% | 1.2 min, to | | |
| | UPLC ®- | 2.1 × 50 mm) | CH₃CN, B: | 5% A in | | |
| | DAD and | | CH₃CN | 0.6 min, held | | |
| | Xevo G2-S | | | for 0.2 min | | |
| | QTOF | | | | | |
| 3 | Waters: | Waters: | A: 95% | 84.2% A for | 0.343 | 6.2 |
| | Acquity | BEH C18 | CH₃COONH₄ | 0.49 min, to | 40 | |
| | UPLC ®- | (1.7 μm, | 7 mM/5% | 10.5% A in | | |
| | DAD and | 2.1 × 100 mm) | CH₃CN, B: | 2.18 min, | | |
| | Quattro | | CH₃CN | held for | | |
| | Micro ™ | | | 1.94 min, | | |
| | | | | back to | | |
| | | | | 84.2% A in | | |
| | | | | 0.73 min, | | |
| | | | | held for | | |
| | | | | 0.73 min. | | |
| 4 | Waters: | Agilent: | A: 95% | From 95% A | 0.8 | 5 |
| | Acquity ® | RRHD | CH₃COONH₄ | to 5% A in | 50 | |
| | IClass | (1.8 μm, | 6.5 mM +5% | 4.5 min, held | | |
| | UPLC ®- | 2.1 × 50 mm) | CH₃CN, B: | for 0.5 min | | |
| | DAD and | | CH₃CN | | | |
| | SQD | | | | | |
| 5 | Agilent | YMC-pack | A: 0.1% | From 95% A | 2.6 | 6.2 |
| | 1100 | ODS-AQ C18 | HCOOH in | to 5% A in | 35 | |
| | HPLC | (50 × 4.6 mm, | H2O | 4.8 min, | | |
| | DAD | 3 μm) | B: CH3CN | held for 1.0 | | |
| | LC/MS | | | min, to 95% | | |
| | G1956A | | | A in 0.2 min. | | |
| 6 | Waters: | Waters: | A: 95% | From 95% A | 0.8 | 2.5 |
| | Acquity ® | BEH C18 | CH₃COONH₄ | to 5% A in | 50 | |
| | UPLC ®- | (1.7 μm, | 6.5 mM +5% | 2.0 min, | | |
| | DAD and | 2.1 × 50 mm) | CH₃CN, B: | held for | | |
| | SQD | | CH₃CN | 0.5 min | | |
| 7 | Agilent | YMC-pack | A: 0.1% | From 95% A | 2.6 | 6.8 |
| | 1260 | ODS-AQ C18 | HCOOH in | to 5% A in | 35 | |
| | Infinity | (50 × 4.6 mm, | H2O | 4.8 min, | | |
| | DAD | 3 μm) | B: CH3CN | held for 1.0 | | |
| | TOF- | | | min, to 95% | | |
| | LC/MS | | | A in 0.2 min. | | |
| | G6224A | | | | | |
| 8 | Waters: | Waters: | A: 95% | From 95% A | 0.8 | 5.0 |
| | Acquity ® | BEH C18 | CH₃COONH₄ | to 5% A in | 50 | |
| | UPLC ®- | (1.7 μm, | 6.5 mm + 5% | 4.5 min, held | | |
| | DAD and | 2.1 × 50 mm) | CH₃CN, B: | for 0.5 min | | |
| | SQD | | CH₃CN | | | |

TABLE 3
Analytical data-melting point (M.p.) and LCMS: [M + H]⁺ means the
protonated mass of the free base of the compound, [M − H]⁻ means
the deprotonated mass of the free base of the compound or the type
of adduct specified [M + CH₃COO]⁻). Rₜ means retention time (in
min). For some compounds, exact mass was determined.

| Co. | | | | LCMS |
|---|---|---|---|---|
| No. | M.p. (° C.) | [M + H]⁺ | Rₜ | Method |
| 1 | n.d. | 350 | 1.04/1.06 | 1 |
| | | | (39%/58%) | |
| 2 | n.d. | 366 | 1.36 | 1 |
| 3 | n.d. | 404 | 1.60/1.62 | 1 |
| | | | (40%/56%) | |
| 4a | n.d. | 420 | 2.00 | 1 |
| 4b | n.d. | 420 | 2.02 | 1 |
| 5 | n.d. | 420 | 1.72/1.74 | 1 |
| | | | (50%/50%) | |
| 6a | 180.52 (A) | 364 | 1.01 | 1 |
| 6b | n.d. | 364 | 1.03 | 1 |
| 7 | n.d. | 350 | 0.82/0.84 | 1 |
| | | | (32%/68%) | |
| 8 | n.d. | 364 | 0.91 | 1 |
| 9 | Decomposition | 350 | 0.96 | 1 |
| | (A) | | | |
| 10 | n.d. | 349 | 0.91 | 1 |
| 11 | n.d. | 378 | 1.24 | 1 |
| 12ab | n.d. | 364 | 1.21/1.24 | 1 |
| | | | (24%/75%) | |
| 12a | n.d. | 364 | 2.07 | 3 |
| 12b | n.d. | 364 | 2.14 | 3 |
| 13 | Decomposition | 364 | 0.94 | 1 |
| | (A) | | | |
| 14 | n.d. | 364 | 1.40 | 1 |
| 15 | n.d. | 363 | 1.26 | 1 |
| 16 | Decomposition | 363 | 1.30/1.33 | 1 |
| | (A) | | (63%/37%) | |
| 17 | n.d. | 349 | 1.33 | 1 |
| 18 | n.d. | 367 | 1.91/1.94 | 1 |
| | | | (37%/58%) | |
| 19 | n.d. | 405 | 2.19/2.22 | 1 |
| | | | (55%/44%) | |
| 20 | n.d. | 421 | 2.63 | 1 |
| 21ab | n.d. | 369 | 1.67/1.68 | 1 |
| | | | (38%/62%) | |
| 21a | n.d. | 369 | 1.67 | 1 |
| 22 | n.d. | 365 | 1.44/1.47 | 1 |
| | | | (55%/43%) | |
| 23a | n.d. | 381 | 1.77 | 1 |
| 23b | n.d. | 381 | 1.85 | 1 |
| 24ab | n.d. | 419 | 2.27/2.31 | 4 |
| 24a | n.d. | 419 | 2.02 | 1 |
| 24b | n.d. | 419 | 1.97 | 1 |
| 25ab | n.d. | 435 | 2.24/2.30 | 1 |
| | | | (20%/79%) | |
| 25a | n.d. | 435 | 2.32 | 1 |
| 26 | n.d. | 351 | 1.07 | 1 |
| 27a | n.d. | 351 | 1.91 | 3 |
| 27b | n.d. | 351 | 1.92 | 3 |
| 28 | n.d. | 367 | 1.38 | 1 |
| 29 | n.d. | 403 [M − H]⁻ | 1.73 | 1 |
| 30 | n.d. | 365 | 1.82/1.88 | 1 |
| | | | (44%/52%) | |
| 31 | n.d. | 379 | 1.53 | 1 |
| 32 | n.d. | 368 | 2.20/2.24 | 1 |
| | | | (41%/57%) | |
| 33ab | n.d. | 368 | 1.79/1.87 | 1 |
| | | | (16%/82%) | |
| 33a | n.d. | 368 | 1.09 | 2 |
| 33b | n.d. | 368 | 1.79 | 1 |
| 34 | n.d. | 398 | 2.18/2.19 | 1 |
| | | | (63%/37%) | |
| 35 | n.d. | 398 | 2.20/2.21 | 1 |
| | | | (63%/37%) | |
| 36 | n.d. | 351 | 1.48 | 1 |
| 37 | n.d. | 351 | 1.51 | 1 |
| 38 | n.d. | 350 | 1.79 | 1 |
| 39 | n.d. | 367 | 1.61/1.70 | 1 |
| | | | (18%/81%) | |
| 40 | n.d. | 383 | 1.68/1.73 | 1 |
| | | | (28%/72%) | |
| 41 | n.d. | 399 | 2.16/2.19 | 1 |
| | | | (45%/55%) | |
| 42a | n.d. | 381 | 2.27 | 3 |
| 42b | n.d. | 381 | 2.35 | 3 |
| 43 | n.d. | 367 | 1.17 | 1 |
| 44 | n.d. | 383 | 1.51 | 1 |
| 45ab | n.d. | 397 | 1.58/1.61 | 1 |
| | | | (7%/93%) | |
| 45a | n.d. | 397 | 1.59 | 1 |
| 46ab | n.d. | 397 | 1.58/1.61 | 1 |
| | | | (7%/93%) | |
| 46a | n.d. | 397 | 1.58 | 1 |
| 47 | n.d. | 383 | 1.24/1.25 | 1 |
| | | | (28%/72%) | |
| 48 | n.d. | 383 | 1.28 | 1 |
| 49ab | n.d. | 398 | 1.54/1.58 | 1 |
| | | | (51%/48%) | |
| 49a | n.d. | 398 | 1.56 | 1 |
| 49b | n.d. | 398 | 1.58 | 1 |
| 50 | n.d. | 385 | 1.41/1.44 | 1 |
| | | | (50%/49%) | |
| 51 | n.d. | 380 | 1.94/1.95 | 1 |
| | | | (46%/54%) | |
| 52 | n.d. | 367 | 1.51 | 1 |
| 53 | Decomposition | 366 | 1.93 | 1 |
| 54 | n.d. | 413 | 2.03/2.15 | 1 |
| | | | (57%/44%) | |
| 55 | n.d. | 352.18 | 1.52 | 1 |
| 56 | n.d. | 366.2 | 1.63/1.72 | 1 |
| free | | | | |
| base | | | | |
| 56 | n.d. | 366.2018/ | 1.62/1.69 | 1 |
| | | 366.2002 | | |
| 57 | n.d. | 366.2 | 1.65 | 1 |
| free | | | | |
| base | | | | |
| 57 | n.d. | 366.2 | 1.63 | 1 |
| 58 | n.d. | 364.2 | 1.01 | 1 |
| 58 | n.d. | 364 | 1.84 | 3 |
| | | 362.1 | | |
| 58 | n.d. | 362.2 | 1 | 1 |
| 58 | n.d. | 364.3 | 1 | 1 |
| 59 | n.d. | 384.2 | 2.13 | 1 |
| 59 | n.d. | 384.2 | 2.13 | 1 |
| 59 | n.d. | 384.2 | 2.13 | 1 |
| free | | | | |
| base | | | | |
| 60 | n.d. | 384.2 | 2.05 | 1 |
| 60 | n.d. | 384.2 | 2.06 | 1 |
| free | | | | |
| base | | | | |
| 61 | n.d. | 367.2 | 1.6 | 1 |
| 61 | n.d. | 367.2 | 1.61 | 1 |
| free | | | | |
| base | | | | |
| 62 | n.d. | 367.2 | 1.65 | 1 |
| adc | | | | |
| 62 | n.d. | 367.2 | 1.67 | 1 |
| free | | | | |
| base | | | | |
| 63 | n.d. | 351.2 | 1.14 | 1 |
| 64 | n.d. | 367.21 | 1.46 | 1 |
| 65 | n.d. | 369.2 | 1.38 | 1 |
| 66 | n.d. | 397 | 1.68 | 1 |
| 67 | n.d. | 397 | 1.66 | 1 |
| 68 | n.d. | 397.2 | 1.65 | 1 |
| 69 | n.d. | 397.2 | 1.66 | 1 |
| 70 | n.d. | 368.19 | 1.38 | 1 |
| 71 | n.d. | 368.19 | 1.37 | 1 |
| 72 | n.d. | 368.2 | 0.93 | 1 |
| 73 | n.d. | 369.2 | 1.36 | 1 |
| 74 | n.d. | 369.2 | 1.38 | 1 |
| 75 | n.d. | 367.2 | 1.66 | 1 |
| 76 | n.d. | 367.2 | 1.66 | 1 |
| 77 | n.d. | 385.2 | 2.10/2.20 | 1 |
| 78 | n.d. | 385.2 | 2.08/2.14 | 1 |
| 79 | n.d. | 359.2 | 1.37 | 4 |
| 80 | n.d. | 381.2 | 1.32 | 1 |
| | | N.B. [M − H]− | | |
| 80 | n.d. | 383.2 | 1.32 | 1 |
| 81 | n.d. | 399.2 | 1.68 | 1 |
| 82 | n.d. | 368.2 | 1.35 | 1 |
| 83 | n.d. | 398.2 | 1.19 | 5 |
| 84 | n.d. | 396.3 | 1.14 | 5 |
| 85 | n.d. | 385.9 | 2.73 | 3 |
| | | 443.1 | | |
| | | [M + CH3COO]− | | |
| 85 | n.d. | 385.2 | 1.93/1.95 | 1 |
| 86 | n.d. | 385.2 | 1.93 | 1 |
| 86 | n.d. | 385.4 | 2.75 | 3 |
| free | | 443.3 | | |
| base | | [M + CH3COO]− | | |
| 87 | n.d. | 385.2 | 1.94 | 1 |
| 87 | n.d. | 385.4 | 2.75 | 3 |
| free | | 443.3 | | |
| base | | [M + CH3COO]− | | |
| 88 | n.d. | 386.17 | 2.14/2.18 | 1 |
| 90 | n.d. | 384.2 | 2.45 | 1 |
| 89 | n.d. | 384.2 | 2.44 | 1 |
| 91 | n.d. | 415.3 | 1.96 and | 8 |
| | | | 1.98 | |
| 92 | n.d. | 387.2 | 1.60 and | 1 |
| free | | | 1.61 | |
| base | | | | |
| 92 | n.d. | 387.16 | 1.60 and | 1 |
| | | | 1.61 | |
| 93 | n.d. | 385.2 | 1.53 | 1 |
| 94 | n.d. | 385.2 | 1.52 | 1 |
| 95 | n.d. | 367.2 | 1.21 | 1 |
| 95 | n.d. | 367.1 | 2.04 | 3 |
| free | | | | |
| base | | | | |
| 96 | n.d. | 367.2 | 1.2 | 1 |
| 96 | n.d. | 367.1 | 2.03 | 3 |
| free | | | | |
| base | | | | |
| 97 | n.d. | 383.2 | 1.5 | 1 |
| 97 | n.d. | 383 | 2.37 | 3 |
| free | | | | |
| base | | | | |
| 98 | n.d. | 414.2 | 2.43/2.47 | 1 |
| 98 | n.d. | 414.4 | 1.49/1.50 | 6 |
| free | | | | |
| base | | | | |
| 99 | n.d. | 414.5 | 3.24 | 3 |
| | | 472.4 | | |
| | | [M + CH3COO]− | | |
| 99 | n.d. | 414.2 | 2.46/2.47 | 1 |
| 100 | n.d. | 414.2 | 2.5 | 1 |
| 101 | n.d. | 414.2 | 2.46 | 1 |
| 102 | n.d. | 350.2 | 0.82-0.84 | 1 |
| 103 | n.d. | 364 | 1.12/1.15 | 1 |
| 104 | n.d. | 404.2058/ | 1.33/1.36 | 1 |
| | | 404.2058 | | |
| 105 | n.d. | 396 | 1.25 | 7 |
| 106 | n.d. | 396 | 1.32 | 7 |
| 107 | n.d. | 412.2 | 1.44 | 5 |
| 108 | n.d. | 397 | 1.11/1.15 | 5 |
| 109 | n.d. | 412.1 | 1.17/1.22 | 5 |

n.d. means not determined.

### Optical Rotations

Optical rotations were measured on a Perkin-Elmer 341 polarimeter with a sodium lamp and reported as follows: [α]° (λ, c g/100 ml, solvent, T ° C.).

[α]_{λ}^{T}=(100α)/(l×c): where l is the path length in dm and c is the concentration in g/100 ml for a sample at a temperature T (° C.) and a wavelength λ (in nm). If the wavelength of light used is 589 nm (the sodium D line), then the symbol D might be used instead. The sign of the rotation (+ or −) should always be given. When using this equation, the concentration and solvent are always provided in parentheses after the rotation. The rotation is reported using degrees and no units of concentration are given (it is assumed to be g/100 mL).

TABLE 4
Optical Rotation data.

| | | Wavelength | Concentration | | Temp. |
|---|---|---|---|---|---|
| Co. No. | α_{D} (°) | (nm) | w/v % | Solvent | (° C.) |
| 9 | −28.6 | 589 | 0.56 | DMF | 20 |
| 10 | −12.0 | 589 | 0.53 | DMF | 20 |
| 17 | −9.8 | 589 | 0.41 | MeOH | 20 |
| 36 | −16.4 | 589 | 0.63 | DMF | 20 |
| 52 | −14.8 | 589 | 0.51 | DMF | 20 |
| 53 | −3.6 | 589 | 0.72 | MeOH | 20 |
| 55 | −3.2 | 589 | 0.53 | MeOH | 20 |
| 56 | −6.2 | 589 | 0.51 | MeOH | 20 |
| 57 | +11.9 | 589 | 0.46 | MeOH | 20 |

### SFCMS-Methods

### General Procedure for SFC-MS Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 5
Analytical SFC-MS Methods (Flow expressed in mL/min; column
temperature (T) in ° C.; Run time in minutes, Backpressure (BPR) in bars).

| Method | | | | Flow | Run time |
|---|---|---|---|---|---|
| code | Column | Mobile phase | gradient | Col T | BPR |
| 1 | Daicel Chiralcel ® | A: CO₂ | 25% B | 3.5 | 3 |
| | OD-3 column (3 | B: EtOH | hold 3 | 35 | 103 |
| | μm, 100 × 4.6 mm) | (+0.3% iPrNH₂) | min | | |
| 2 | Daicel Chiralpak ® | A: CO₂ | 20% B | 3.5 | 3 |
| | AD-3 column (3 μm, | B: EtOH | hold 3 | 35 | 103 |
| | 100 × 4.6 mm) | (+0.3% iPrNH₂) | min | | |
| 3 | Daicel Chiralpak ® | A: CO₂ | 40% B | 3.5 | 3 |
| | IC-3 column (3 μm, | B: EtOH | hold 3 | 35 | 103 |
| | 100 × 4.6 mm) | (+0.3% iPrNH₂) | min | | |
| 4 | Daicel Chiralpak ® | A: CO₂ | 45% B | 3.5 | 3 |
| | IC-3 column (3 μm, | B: IPOH | hold 3 | 35 | 103 |
| | 100 × 4.6 mm) | (+0.3% iPrNH₂) | min | | |
| 5 | Daicel Chiralpak ® | A: CO₂ | 45% B | 3.5 | 3 |
| | IG-3 column (3 μm, | B: MeOH | hold 3 | 35 | 103 |
| | 100 × 4.6 mm) | (+0.3% iPrNH₂) | min | | |
| 6 | Daicel Chiralcel ® | A: CO₂ | 25% B | 3.5 | 3 |
| | OD-3 column (3 | B: IPOH | hold 3 | 35 | 103 |
| | μm, 100 × 4.6 mm) | (+0.3% iPrNH₂) | min | | |
| 7 | Daicel Chiralpak ® | A: CO₂ | 20% B | 3.5 | 3 |
| | AD-3 column (3 | B: MeOH | hold 3 | 35 | 103 |
| | μm, 100 × 4.6 mm) | (+0.3% iPrNH₂) | min | | |
| 8 | Daicel Chiralpak ® | A: CO₂ | 20% B | 3.5 | 3 |
| | AD-3 column (3 μm, | B: IPOH | hold 3 | 35 | 103 |
| | 100 × 4.6 mm) | (+0.3% iPrNH₂) | min | | |
| 9 | Daicel Chiralcel ® | A: CO₂ | 25% B | 3.5 | 3 |
| | OD-3 column (3 μm, | B: IPOH | hold 3 | 35 | 103 |
| | 100 × 4.6 mm) | (+0.3% iPrNH₂) | min | | |

TABLE 6
Analytical SFC data-Rₜ means retention time (in minutes), [M + H]⁺
means the protonated mass of the compound, method refers to the
method used for (SFC)MS analysis of enantiomerically pure
compounds.

| | | | | | Isomer Elution |
|---|---|---|---|---|---|
| Co. No. | Rₜ | [M + H]⁺ | UV Area % | Method | Order |
| 12a | 0.97 | 364 | 100 | 1 | A |
| 12b | 1.27 | 364 | 100 | 1 | B |
| 27a | 0.84 | 351 | 100 | 2 | A |
| 27b | 1.07 | 351 | 99.48 | 2 | B |
| 42a | 1.61 | 381 | 100 | 3 | A |
| 42b | 2.06 | 381 | 100 | 3 | B |
| 58 | 1.25, | 364 | 50.43, | 4 | |
| | 1.63 | | 49.57 | | |
| 65 | 1.34, | 369 | 59.65, | 5 | |
| | 1.83 | | 40.35 | | |
| 85 | 1.09, | 385 | 50.52, | 6 | |
| | 1.49 | | 49.48 | | |
| 97 free | 1.51 | 383 | 100.00 | 7 | B |
| base | | | | | |
| 96 free | 1.05 | 367 | 100.00 | 2 | A |
| base | | | | | |
| 95 free | 1.34 | 367 | 98.38 | 2 | B |
| base | | | | | |
| 99 | 0.90, | 414 | 49.82, | 8 | |
| | 1.29 | | 50.18 | | |
| 86 free | 1.09 | 385 | 100.00 | 9 | A |
| base | | | | | |
| 87 free | 1.49 | 385 | 100.00 | 9 | B |
| base | | | | | |

### NMR

For a number of compounds, ¹H NMR spectra were recorded on a Bruker DPX-400 spectrometer operating at 400 MHz, on a Bruker Avance I operating at 500 MHz, using CHLOROFORM-d (deuterated chloroform, CDCl₃) or DMSO-d₆ (deuterated DMSO, dimethyl-d6 sulfoxide) as solvent. Chemical shifts (6) are reported in parts per million (ppm) relative to tetramethylsilane (TMS), which was used as internal standard.

TABLE 7
¹H NMR results

| Co. | |
|---|---|
| No. | ¹H NMR result |
| 4a | ¹H NMR (500 MHz, CHLOROFORM-d) δ ppm 1.32-1.43 (m, |
| | 1 H), 1.44-1.52 (m, 3 H), 1.63-1.95 (m, 2 H), 2.01-2.16 (m, 2 |
| | H), 2.70 (s, 3 H), 2.72-2.84 (m, 1 H), 2.88 (br d, J = 9.83 Hz, |
| | 1 H), 2.93-2.95 (m, 1 H), 3.04 (br d, J = 10.69 Hz, 1 H), 3.71 |
| | (q, J = 6.65 Hz, 1 H), 3.92 (s, 3 H), 6.66 (s, 1 H), 7.06 (s, 1 |
| | H), 7.93 (br s, 1 H), 8.26 (br s, 1 H), one H exchanged (NH). |
| 4b | ¹H NMR (500 MHz, CHLOROFORM-d) δ ppm 1.30-1.51 (m, |
| | 5 H), 1.67-1.79 (m, 1 H), 1.84-1.93 (m, 1 H), 1.96-2.15 (m, 2 |
| | H), 2.70 (s, 3 H), 2.80-2.93 (m, 2 H), 3.05 (br d, J = 9.54 Hz, |
| | 1 H), 3.64-3.77 (m, 1 H), 3.95 (s, 3 H), 6.74 (s, 1 H), 7.11 (s, |
| | 1 H), 7.93 (br s, 1 H), 8.25 (br s, 1 H), 10.83 (br s, 1 H). |
| 6a | ¹H NMR (500 MHz, CHLOROFORM-d) δ ppm 0.84-0.96 (m, |
| | 1 H), 1.44 (d, J = 6.94 Hz, 3 H), 1.48-1.58 (m, 1 H), 1.59- |
| | 1.71 (m, 2 H), 1.73 (br d, J = 10.40 Hz, 1 H), 1.76-1.85 (m, |
| | 1 H), 1.96-2.05 (m, 1 H), 2.25-2.34 (m, 1 H), 2.36-2.42 (m, |
| | 1 H), 2.43 (s, 6 H), 2.68 (br d, J = 10.40 Hz, 1 H), 2.72 (s, 3 |
| | H), 2.90 (br d, J = 10.69 Hz, 1 H), 3.63 (q, J = 6.65 Hz, 1 H), |
| | 6.68 (s, 2 H), 7.95 (d, J = 1.45 Hz, 1 H), 8.24 (d, J = 1.44 Hz, |
| | 1 H), 12.27 (br s, 1 H). |
| 6b | ¹H NMR (400 MHz, CHLOROFORM-d) δ ppm 0.81-0.97 (m, |
| | 1 H), 1.37-1.53 (m, 1 H), 1.44 (d, J = 6.70 Hz, 3 H,) 1.61 (br |
| | d, J = 10.87 Hz, 2 H), 1.70-1.82 (m, 1 H), 1.83-2.03 (m, 2 H), |
| | 2.31-2.42 (m, 1 H), 2.42-2.51 (m, 1 H), 2.47 (s, 6 H), 2.66- |
| | 2.80 (m, 1 H), 2.72 (s, 3 H), 2.86 (br d, J = 9.71 Hz, 1 H), |
| | 3.64 (q, J = 6.86 Hz, 1 H), 6.74 (s, 2 H), 7.94 (d, J = 1.62 Hz, |
| | 1 H), 8.23 (d, J = 1.62 Hz, 1 H), 12.07 (br s, 1 H). |
| 9 | ¹H NMR (400 MHz, CHLOROFORM-d) δ ppm 0.95-1.06 (m, |
| | 1 H), 1.55-1.74 (m, 3 H), 1.92 (br dd, J = 19.19, 9.94 Hz, 2 |
| | H), 2.10 (br d, J = 9.48 Hz, 1 H), 2.35-2.51 (m, 2 H), 2.46 (s, |
| | 6 H), 2.69 (s, 3 H), 2.86 (br d, J = 6.94 Hz, 2 H), 3.62-3.78 |
| | (m, 2 H), 6.72 (s, 2 H), 7.90 (d, J = 1.62 Hz, 1 H), 8.24 (d, |
| | J = 1.39 Hz, 1 H), one H exchanged (NH). |
| 12a | ¹H NMR (400 MHz, CHLOROFORM-d) δ ppm 0.79-1.01 (m, |
| | 1 H), 1.44 (d, J = 6.7 Hz, 3 H), 1.48-1.72 (m, 3 H), 1.88-1.99 |
| | (m, 2 H), 2.08 (br t, J = 10.3 Hz, 1 H), 2.22-2.38 (m d, 2 H), |
| | 2.41 (s, 6 H), 2.70 (br d, J = 10.2 Hz, 1 H), 2.83-3.05 (m, 1 |
| | H), 3.74 (br d, J = 6.0 Hz, 1 H), 4.23 (s, 3 H), 6.67 (s, 2 H), |
| | 7.25 (s, 1 H), 7.84 (s, 1 H), 7.92 (d, J = 8.6 Hz, 1 H). |
| 12b | ¹H NMR (400 MHz, CHLOROFORM-d) δ ppm 0.81-1.01 (m, |
| | 1 H), 1.43 (d, J = 6.7 Hz, 3 H), 1.46-1.67 (m, 3 H), 1.77-1.96 |
| | (m, 2 H), 2.18 (br t, J = 10.5 Hz, 1 H), 2.27-2.39 (m, 1 H), |
| | 2.45 (s, 6 H), 2.48 (s, 1 H), 2.73 (br d, J = 10.9 Hz, 1 H), |
| | 2.88 (br d, J = 7.4 Hz, 1 H), 3.74 (q, J = 6.5 Hz, 1 H), 4.22 (s, |
| | 3 H), 6.72 (s, 2 H) 7.24 (d, J = 8.6 Hz, 1 H), 7.84 (s, 1 H), |
| | 7.94 (d, J = 8.6 Hz, 1 H). |
| 17 | ¹H NMR (400 MHz, CHLOROFORM-d) δ ppm 1.03-1.28 (m, |
| | 1 H), 1.41-1.71 (m, 1 H), 1.74-1.97 (m, 2 H), 2.31-2.44 (m, 1 |
| | H), 2.56 (s, 1 H), 2.61-2.85 (m, 9 H), 3.20-3.33 (m, 2 H), |
| | 4.14-4.25 (m, 3 H), 4.26-4.51 (m, 2 H), 7.26-7.41 (m, 1 H) |
| | 7.49-7.59 (m, 2 H), 7.68-7.81 (m, 1 H), 7.86 (s, 1 H), 8.36- |
| | 8.43 (m, 1 H), 11.01-11.30 (m, 1 H), 15.85 (br s, 1 H). |
| 23a | ¹H NMR (400 MHz, CHLOROFORM-d) δ ppm 0.78-1.02 (m, |
| | 1 H), 1.42 (d, J = 6.70 Hz, 3 H), 1.45-1.55 (m, 1 H), 1.61- |
| | 1.67 (m, 2 H), 1.75-1.90 (m, 2 H), 1.99-2.09 (m, 1 H), 2.21- |
| | 2.33 (m, 1 H), 2.36 (s, 3 H), 2.37-2.43 (m, 1 H), 2.69-2.74 |
| | (m, 1 H), 2.69 (s, 3 H), 2.81-2.93 (m, 1 H), 3.76 (q, J = 6.86 |
| | Hz, 1 H), 3.87 (s, 3 H), 6.26 (s, 1 H), 6.47 (s, 1 H), 7.39 (d, |
| | J = 8.32 Hz, 1 H), 7.68 (d, J = 8.32 Hz, 1 H). |
| 23b | ¹H NMR (400 MHz, CHLOROFORM-d) δ ppm 0.82-0.95 (m, |
| | 1 H), 1.42 (d, J = 6.94 Hz, 3 H), 1.40-1.51 (m, 1 H), 1.55- |
| | 1.65 (m, 2 H), 1.75-1.93 (m, 2 H), 2.07 (br t, J = 10.69 Hz, |
| | 1 H), 2.34 (dd, J = 13.58, 7.51 Hz, 1 H), 2.39 (s, 3 H), 2.41- |
| | 2.48 (m, 1 H), 2.68 (s, 3 H), 2.70-2.75 (m, 1 H), 2.87 (br d, |
| | J = 9.83 Hz, 1 H), 3.75 (q, J = 6.65 Hz, 1 H), 3.89 (s, 3 H), |
| | 6.30 (s, 1 H), 6.51 (s, 1 H), 7.36 (d, J = 8.38 Hz, 1 H), 7.69 |
| | (d, J = 8.38 Hz, 1 H). |
| 24a | ¹H NMR (400 MHz, CHLOROFORM-d) δ ppm 0.89-1.01 (m, |
| | 1 H), 1.42 (d, J = 6.94 Hz, 3 H), 1.42-1.54 (m, 1 H), 1.56- |
| | 1.68 (m, 2 H), 1.82-1.96 (m, 2 H), 2.13 (br t, J = 10.11 Hz, |
| | 1 H), 2.48 (dd, J = 13.73, 7.37 Hz, 1 H), 2.57 (s, 3 H), 2.57- |
| | 2.62 (m, 1 H), 2.69 (s, 3 H), 2.74 (br d, J = 12.4 Hz, 1H), 2.82 |
| | (br d, J = 8.67 Hz, 1H), 3.79 (q, J = 6.84 Hz, 1 H), 7.09 (s, 1 |
| | H), 7.25 (s, 1 H), 7.34 (d, J = 8.38 Hz, 1 H), 7.69 (d, J = 8.38 |
| | Hz, 1H). |
| 24b | ¹H NMR (400 MHz, CHLOROFORM-d) δ ppm 0.89-1.00 (m, |
| | 1 H), 1.42 (d, J = 6.65 Hz, 3H), 1.48-1.72 (m, 3 H), 1.79-1.90 |
| | (m, 2 H), 2.06 (td, J = 11.13 2.31 Hz, 1H), 2.41-2.47 (m, |
| | 1 H), 2.49-2.55 (m, 1 H), 2.54 (s, 3 H), 2.67 (br d, J = 8.09 |
| | Hz, 2H), 2.69 (s, 2 H), 2.91 (br d, J = 10.98 Hz, 1 H), 3.74 (q, |
| | J = 6.94 Hz, 1 H), 7.04 (s, 1 H), 7.20 (s, 1 H), 7.35 (d, J = |
| | 8.38 Hz, 1 H), 7.67 (d, J = 8.38 Hz, 1 H). |
| 27a | ¹H NMR (500 MHz, CHLOROFORM-d) δ ppm 1.42 (d, J = |
| | 6.65 Hz, 3 H), 1.42-1.50 (m, 1 H), 1.88-1.96 (m, 1 H), 2.14 |
| | (dd, J = 9.25, 6.65 Hz, 1 H), 2.44-2.52 (m, 1H), 2.41-2.47 (m, |
| | 2 H), 2.46 (s, 6 H), 2.56-2.60 (m, 2 H), 2.65-2.72 (m, 2H), |
| | 2.68 (s, 2 H), 2.80 (dd, J = 9.25, 7.51 Hz, 1 H), 3.60 (q, J = |
| | 6.65 Hz, 1 H), 6.74 (s, 2 H), 7.41 (d, J = 8.38 Hz, 1 H), 7.71 |
| | (d, J = 8.38 Hz, 1H). |
| 27b | ¹H NMR (500 MHz, CHLOROFORM-d) δ ppm 1.42 (d, J = |
| | 6.65 Hz, 3 H), 1.41-1.50 (m, 1 H), 1.91-2.01 (m, 1 H), 2.23 |
| | (br dd, J = 8.81, 7.37 Hz, 1 H), 2.39-2.51 (m, 2H), 2.45 (s, 6 |
| | H), 2.56 (d, J = 7.51 Hz, 2 H), 2.57-2.63 (m, 1H), 2.68 (s, 3 |
| | H), 2.79-2.87 (m, 1 H), 3.60 (q, J = 6.65 Hz, 1 H), 6.73 (s, 2 |
| | H), 7.40 (d, J = 8.38 Hz, 1 H), 7.70 (d, J = 8.38 Hz, 1 H). |
| 42a | ¹H NMR (400 MHz, CHLOROFORM-d) δ ppm 0.83-1.00 (m, |
| | 1 H), 1.49 (d, J = 6.70 Hz, 3 H), 1.53-1.74 (m, 3 H), 1.81- |
| | 2.03 (m, 2 H), 2.11 (br t, J = 10.75 Hz, 1 H), 2.26-2.34 (m, |
| | 1 H), 2.38-2.42 (m, 1 H), 2.43 (s, 6 H), 2.74-2.84 (m, 1 H), |
| | 2.89 (s, 3 H), 2.90-2.98 (m, 1 H), 3.79-3.93 (m, 1 H), 6.69 (s, |
| | 2 H), 7.47 (d, J = 8.32 Hz, 1 H), 8.11 (d, J = 8.32 Hz, 1 H). |
| 42b | ¹H NMR (400 MHz, CHLOROFORM-d) δ ppm 0.82-1.01 (m, |
| | 1 H), 1.48 (d, J = 6.94 Hz, 3 H), 1.51-1.56 (m, 1 H), 1.57- |
| | 1.69 (m, 2 H), 1.79-2.01 (m, 2 H), 2.16-2.26 (m, 1 H), 2.33- |
| | 2.38 (m, 1 H), 2.39-2.48 (m, 1 H), 2.46 (s, 6 H), 2.77-2.86 (m, |
| | 1 H), 2.89 (s, 3 H), 2.89-2.96 (m, 1 H), 3.81-3.96 (m, 1 H), |
| | 6.73 (s, 2 H), 7.44 (d, J = 8.32 Hz, 1 H), 8.12 (d, J = 8.32 Hz, |
| | 1 H). |
| 49a | ¹H NMR (500 MHz, CHLOROFORM-d) δ ppm 1.04-1.16 (m, |
| | 1 H), 1.48 (d, J = 6.65 Hz, 3 H), 1.54-1.65 (m, 1 H), 1.66- |
| | 1.72 (m, 1 H), 1.73-1.80 (m, 1 H), 2.03-2.12 (m, 2 H), 2.17 |
| | (td, J = 10.84, 2.60 Hz, 1 H), 2.37 (s, 3 H), 2.55 (s, 3 H), |
| | 2.81-2.88 (m, 1 H), 2.88 (s, 3 H), 2.92 (br d, J = 7.80 Hz, 1 |
| | H), 3.89 (q, J = 6.94 Hz, 1 H), 4.14 (d, J = 6.07 Hz, 2 H), 6.28 |
| | (s, 1 H), 7.48 (d, J = 8.38 Hz, 1 H), 8.06 (d, J = 8.38 Hz, 1 H). |
| 49b | ¹H NMR (500 MHz, CHLOROFORM-d) δ ppm 1.06-1.16 (m, |
| | 1 H), 1.48 (d, J = 6.94 Hz, 3 H), 1.51-1.61 (m, 1 H), 1.64- |
| | 1.71 (m, 1 H), 1.71-1.78 (m, 1 H), 2.00-2.16 (m, 1 H), 2.06- |
| | 2.15 (m, 1 H), 2.22 (td, J = 10.76, 2.17 Hz, 1 H), 2.38 (s, 3 |
| | H), 2.56 (s, 3 H), 2.73-2.79 (m, 1 H), 2.88 (s, 3 H), 3.00 (br d, |
| | J = 10.40 Hz, 1 H), 3.83 (q, J = 6.94 Hz, 1 H), 4.14-4.23 (m, |
| | 2 H), 6.32 (s, 1 H), 7.44 (d, J = 8.38 Hz, 1 H), 8.06 (d, J = |
| | 8.38 Hz, 1 H). |
| 60 | ¹H NMR (400 MHz, METHANOL-d) δ ppm 1.79 (d, J = 6.70 |
| | Hz, 4 H) 2.14 (br dd, J = 12.72, 5.32 Hz, 1 H) 2.50 (s, 6 H) |
| | 2.84 (s, 6 H) 3.00-3.12 (m, 1 H) 3.33-3.39 (m, 1 H) 3.42-3.56 |
| | (m, 1 H) 4.76 (q, J = 6.94, 1 H) 7.11 (s, 2 H) 7.87 (d, J = 9.94 |
| | Hz, 1 H) 8.15 (d, J = 6.24 Hz, 1 H) |
| 98 | ¹H NMR (400 MHz, DMSO-d6) δ ppm 1.67-2.00 (m, 5 H) |
| | 2.57-2.71 (m, 8 H) 2.71-2.87 (m, 5 H) 3.63-3.89 (m, 1 H) |
| | 4.12-4.48 (m, 2 H) 4.83-5.05 (m, 1 H) 7.13-7.51 (m, 1 H) |
| | 8.12 (dd, J = 10.06, 1.97 Hz, 1 H) 8.48 8s, 1 H) 11.26-11.61 |
| | (m, 1 H), 14.87 (br d, J = 1.39 Hz, 1 H) |
| 93 | ¹H NMR (400 MHz, DMSO-d6) δ ppm 1.61-1.70 (m, 3 H) |
| | 1.71-1.83 (m, 1 H) 2.05 (br dd, J = 12.83, 7.51 Hz, 1 H) 2.70 |
| | (d, J = 12.25 Hz, 7 H) 2.74-2.85 (m, 1 H) 2.90 (s, 5 H) 3.02- |
| | 3.21 (m, 1 H) 3.33-3.43 (m, 2 H) 3.61-3.76 (m, 1 H) 4.90- |
| | 5.15 (m, 1 H) 7.64 (s, 1 H) 8.71 (dd, J = 9.25, 5.32 Hz, 1 H) |
| | 11.11-11.42 (M , 1 H) 15.99 (br s, 1 H) |
| 88 | ¹H NMR (400 MHz, CHLOROFORM-d) δ ppm 1.46 (d, J = |
| | 6.82 Hz, 3 H) 1.94 (dtd, J = 10.81, 5.12, 5.12, 2.77 Hz, 1 H) |
| | 2.27 (ddd, J = 13.70, 6.76, 2.43 Hz, 1 H) 2.45 (s, 6 H) 2.66- |
| | 2.75 (m, 2 H) 2.82 (s, 3 H) 3.00 (ddd, J = 17.22, 10.63, 6.36 |
| | Hz, 1 H) 3.83-3.93 (m, 1 H) 4.74-4.87 (m, 1 H) 6.41 (d, J = |
| | 3.70 Hz, 2 H) 7.46 (dd, J = 9.48, 0.92 Hz, 1 H) 8.08 (dd, J = |
| | 13.64, 6.24 Hz, 1 H) |
| 91 | ¹H NMR (400 MHz, DMSO-d6) δ ppm 1.66-1.94 (m, 6 H) |
| | 2.08 (br d, J = 12.48 Hz, 1 H) 2.63 (d, J = 4.39 Hz, 6 H) 2.89 |
| | (d, J = 3.01 Hz, 5 H) 3.45-3.82 (m, 2 H) 4.00-4.38 (m, 2 H) |
| | 4.84-5.10 (m, 1 H) 7.29 (d, J = 5.09 Hz, 2 H) 8.70 (d, J = |
| | 9.25, 1 H) 11.12-11.50 (m, 1 H) 15.17 (br s, 1 H) |
| 101 | ¹H NMR (400 MHz, DMSO-d6) δ ppm 1.33-1.74 (m, 2 H) |
| | 1.76-2.03 (m, 4 H) 2.09-2.31 (m, 1 H) 2.72 (d, J = 5.32 Hz, 6 |
| | H) 2.81 (s, 3 H) 2.94-3.30 (m, 1 H) 3.46-3.70 (m, 2 H) 3.79- |
| | 4.27 (m, 1 H) 4.64-5.00 (m, 3 H) 7.61 (br s, 1 H) 7.99 (br s, 1 |
| | H) 8.09-8.19 (m, 1 H) 8.52 (br d, J = 6.01 Hz, 1 H) 10.27- |
| | 10.67 (m, 1 H) 11.59-11.82 (m, 1 H) |
| 76 | ¹H NMR (400 MHz, CHLOROFORM-d) δ ppm 1.39 (qd, J = |
| | 12.33, 4.05 Hz, 1 H) 1.50 (d, J = 6.94 Hz, 3 H) 1.59-1.69 (m, |
| | 1 H) 1.70-1.76 (m, 1 H) 1.82-1.89 (m, 1 H), 2.10-2.23 (m, 2 |
| | H) 2.47 (s, 6 H) 2.70-2.79 (m, 1 H) 2.88 (s, 3 H) 2.94 (br d, |
| | J = 11.27 Hz, 1 H) 3.01-3.06 (m, 1 H) 3.90 (q, J = 6.74 Hz, 1 |
| | H) 6.78 (s, 2 H) 7.46 (d, J = 8.38, 1 H) 8.12 (d, J = 8.09 Hz, |
| | 1 H) |

### Pharmacological Examples

### 1) OGA—Biochemical Assay

The assay is based on the inhibition of the hydrolysis of fluorescein mono-β-D-N-Acetyl-Glucosamine (FM-GlcNAc) (Mariappa et al. 2015, Biochem J 470:255) by the recombinant human Meningioma Expressed Antigen 5 (MGEA5), also referred to as O-GlcNAcase (OGA). The hydrolysis FM-GlcNAc (Marker Gene technologies, cat #M1485) results in the formation of β-D-N-glucosamineacetate and fluorescein. The fluorescence of the latter can be measured at excitation wavelength 485 nm and emission wavelength 538 nm. An increase in enzyme activity results in an increase in fluorescence signal. Full length OGA enzyme was purchased at OriGene (cat #TP322411). The enzyme was stored in 25 mM Tris.HCl, pH 7.3, 100 mM glycine, 10% glycerol at −20° C. Thiamet G and GlcNAcStatin were tested as reference compounds (Yuzwa et al. 2008 Nature Chemical Biology 4:483; Yuzwa et al. 2012 Nature Chemical Biology 8:393). The assay was performed in 200 mM Citrate/phosphate buffer supplemented with 0.005% Tween-20. 35.6 g Na₂HPO₄ 2H₂O (Sigma, #C0759) were dissolved in 1 L water to obtain a 200 mM solution. 19.2 g citric acid (Merck, #1.06580) was dissolved in 1 L water to obtain a 100 mM solution. pH of the sodiumphosphate solution was adjusted with the citric acid solution to 7.2. The buffer to stop the reaction consists of a 500 mM Carbonate buffer, pH 11.0. 734 mg FM-GlcNAc were dissolved in 5.48 mL DMSO to obtain a 250 mM solution and was stored at −20° C. OGA was used at a 2 nM concentration and FM-GlcNAc at a 100 uM final concentration. Dilutions were prepared in assay buffer.

50 nl of a compound dissolved in DMSO was dispensed on Black Proxiplate™ 384 Plus Assay plates (Perkin Elmer, #6008269) and 3 μl fl-OGA enzyme mix added subsequently. Plates were pre-incubated for 60 min at room temperature and then 2 μl FM-GlcNAc substrate mix added. Final DMSO concentrations did not exceed 1%. Plates were briefly centrifuged for 1 min at 1000 rpm and incubate at room temperature for 6 h. To stop the reaction 5 μl STOP buffer were added and plates centrifuge again 1 min at 1000 rpm. Fluorescence was quantified in the Thermo Scientific Fluoroskan Ascent or the PerkinElmer EnVision with excitation wavelength 485 nm and emission wavelength 538 nm.

For analysis a best-fit curve is fitted by a minimum sum of squares method. From this an IC₅₀ value and Hill coefficient was obtained. High control (no inhibitor) and low control (saturating concentrations of standard inhibitor) were used to define the minimum and maximum values.

### 2) OGA—Cellular Assay

HEK293 cells inducible for P301L mutant human Tau (isoform 2N4R) were established at Janssen. Thiamet-G was used for both plate validation (high control) and as reference compound (reference EC₅₀ assay validation). OGA inhibition is evaluated through the immunocytochemical (ICC) detection of O-GlcNAcylated proteins by the use of a monoclonal antibody (CTD110.6; Cell Signaling, #9875) detecting 0-GlcNAcylated residues as previously described (Dorfmueller et al. 2010 Chemistry & biology, 17:1250). Inhibition of OGA will result in an increase of O-GlcNAcylated protein levels resulting in an increased signal in the experiment. Cell nuclei are stained with Hoechst to give a cell culture quality control and a rough estimate of immediate compounds toxicity, if any. ICC pictures are imaged with a Perkin Elmer Opera Phenix plate microscope and quantified with the provided software Perkin Elmer Harmony 4.1.

Cells were propagated in DMEM high Glucose (Sigma, #D5796) following standard procedures. 2 days before the cell assay cells are split, counted and seeded in Poly-D-Lysine (PDL) coated 96-wells (Greiner, #655946) plate at a cell density of 12,000 cells per cm² (4,000 cells per well) in 100 μl of Assay Medium (Low Glucose medium is used to reduce basal levels of GlcNAcylation) (Park et al. 2014 The Journal of biological chemistry 289:13519). At the day of compound test medium from assay plates was removed and replenished with 90 μl of fresh Assay Medium. 10 μl of compounds at a 10fold final concentration were added to the wells. Plates were centrifuged shortly before incubation in the cell incubator for 6 hours. DMSO concentration was set to 0.2%. Medium is discarded by applying vacuum. For staining of cells medium was removed and cells washed once with 100 μl D-PBS (Sigma, #D8537). From next step onwards unless other stated assay volume was always 50 μl and incubation was performed without agitation and at room temperature. Cells were fixed in 50 μl of a 4% paraformaldehyde (PFA, Alpha aesar, #043368) PBS solution for 15 minutes at room temperature. The PFA PBS solution was then discarded and cells washed once in 10 mM Tris Buffer (LifeTechnologies, #15567-027), 150 mM NaCl (LifeTechnologies, #24740-0110, 0.1% Triton X (Alpha aesar, #A16046), pH 7.5 (ICC buffer) before being permeabilized in same buffer for 10 minutes. Samples are subsequently blocked in ICC containing 5% goat serum (Sigma, #G9023) for 45-60 minutes at room temperature. Samples were then incubated with primary antibody (1/1000 from commercial provider, see above) at 4° C. overnight and subsequently washed 3 times for 5 minutes in ICC buffer. Samples were incubated with secondary fluorescent antibody (1/500 dilution, Lifetechnologies, #A-21042) and nuclei stained with Hoechst 33342 at a final concentration of 1 μg/ml in ICC (Lifetechnologies, #H3570) for 1 hour. Before analysis samples were washed 2 times manually for 5 minutes in ICC base buffer.

Imaging is performed using Perkin Elmer Phenix Opera using a water 20× objective and recording 9 fields per well. Intensity readout at 488 nm is used as a measure of O-GlcNAcylation level of total proteins in wells. To assess potential toxicity of compounds nuclei were counted using the Hoechst staining. IC₅₀-values are calculated using parametric non-linear regression model fitting. As a maximum inhibition Thiamet G at a 200 uM concentration is present on each plate. In addition, a concentration response of Thiamet G is calculated on each plate.

TABLE 8
Results in the biochemical and cellular assays.

| | | | Cellular | |
|---|---|---|---|---|
| | Enzymatic | Enzymatic | hOGA; | Cellular |
| Co. No. | hOGA; pIC₅₀ | Eₘₐₓ (%) | pEC₅₀ | Eₘₐₓ (%) |
| 1 | 6.5 | 98 | | |
| 2 | 6.7 | 97 | | |
| 3 | 6.5 | 96 | | |
| 4a | 5.0 | 50 | | |
| 4b | 6.3 | 96 | | |
| 5 | 6.1 | 94 | | |
| 6a | 5.8 | 89 | | |
| 6b | 8.1 | 100 | | |
| 7 | 7.3 | 100 | | |
| 8 | 7.0 | 99 | <6 | 23 |
| 9 | 6.7 | 96 | 6.2 | 59 |
| 10 | 6.0 | 89 | | |
| 11 | 7.5 | 100 | 6.2 | 64 |
| 12ab | 7.6 | 100 | 6.6 | 71 |
| 12a | 5.8 | 88 | | |
| 12b | 7.8 | 100 | | |
| 13 | 6.6 | 99 | | |
| 14 | 7.6 | 103 | <6 | 45 |
| 15 | 6.0 | 92 | | |
| 16 | 6.9 | 97 | <6 | 14 |
| 17 | 6.1 | 90 | | |
| 18 | 7.7 | 100 | 6.8 | 76 |
| 19 | 7.7 | 99 | 6.3 | 68 |
| 20 | 7.7 | 101 | 6.2 | 60 |
| 21ab | 6.6 | 97 | | |
| 21a | <5 | 22 | | |
| 22 | 8.5 | 101 | 7.9 | 91 |
| 23a | 8.1 | 101 | 7.6 | 79 |
| 23b | 6.7 | 101 | | |
| 24ab | 7.9 | 103 | 7.3 | 91 |
| 24a | 8.1 | 99 | 7.4 | 81 |
| 24b | 5.3 | 69 | | |
| 25ab | 7.8 | 101 | 6.7 | 86 |
| 25a | 8.4 | 101 | 7.3 | 85 |
| 26 | 7.4 | 101 | | |
| 27a | 6.7 | 99 | | |
| 27b | <5 | 47 | | |
| 28 | 7.0 | 99 | | |
| 29 | 6.9 | 103 | | |
| 30 | 7.9 | 102 | 6.7 | 79 |
| 31 | <5 | 30 | <6 | −7 |
| 32 | 8.1 | 99 | | |
| 33ab | 7.7 | 99 | | |
| 33a | 8.6 | 100 | | |
| 33b | 7.2 | 101 | | |
| 34 | 8.0 | 101 | | |
| 35 | 7.0 | 101 | | |
| 36 | 7.2 | 101 | | |
| 37 | 5.1 | 55 | <6 | 2 |
| 38 | 6.9 | 100 | | |
| 39 | 7.9 | 99 | 7.2 | 79 |
| 40 | 7.9 | 99 | 6.1 | 49 |
| 41 | 6.9 | 101 | | |
| 42a | 6.3 | 98 | | |
| 42b | 8.6 | 101 | 8.0 | 79 |
| 43 | 7.27 | 103 | 6.2 | 60 |
| 44 | 6.9 | 99 | | |
| 45ab | 6.5 | 98 | | |
| 45a | 6.3 | 95 | | |
| 46ab | 8.3 | 101 | 7.8 | 85 |
| 46a | 6.2 | 96 | | |
| 47 | 6.4 | 99 | <6 | 11 |
| 48 | 6.1 | 93 | <6 | 16 |
| 49ab | 7.9 | 100 | 7.0 | 68 |
| 49a | 5.7 | 84 | <6 | −2 |
| 49b | 8.2 | 99 | 7.5 | 70 |
| 50 | 7.7 | 102 | 6.7 | 77 |
| 51 | 6.3 | 99 | <6 | |
| 52 | 7.2 | 103 | 6.3 | 70 |
| 53 | 5.4 | 77 | | |
| 54 | 8.0 | 100 | 6.8 | 67 |
| 55 | <5 | 18 | | |
| 56 | <5 | 41 | | |
| 57 | <5 | 28 | | |
| 58 | 7.9 | 100 | 6.5 | 69 |
| 59 | 6.7 | 98 | <6 | 11 |
| 60 | 8.7 | 99 | 7.2 | 66 |
| 61 | 7.8 | 100 | 6.1 | 53 |
| 62 | 6.2 | 93 | <6 | −2 |
| 63 | 7.0 | 103 | 6.8 | 77 |
| 64 | 6.8 | 100 | 6.1 | 56 |
| 65 | 6.5 | 97 | <6 | 3 |
| 66 | 7.4 | 100 | 6.3 | 55 |
| 67 | 6.3 | 94 | <6 | 5 |
| 68 | 6.9 | 98 | <6 | 27 |
| 69 | 6.3 | 96 | <6 | −6 |
| 70 | 7.2 | 98 | 6.1 | 55 |
| 71 | 5.1 | 51 | <6 | −5 |
| 72 | 6.5 | 95 | <6 | 18 |
| 73 | <5 | 8 | <6 | −9 |
| 74 | 6.8 | 97 | <6 | −4 |
| 75 | 5.7 | 86 | <6 | −1 |
| 76 | 8.3 | 98 | 7.5 | 67 |
| 77 | 5.6 | 75 | <6 | −10 |
| 78 | 8.0 | 93 | 7.1 | 80 |
| 79 | 6.8 | 99 | | |
| 80 | 5.7 | 85 | <6 | 5 |
| 81 | 6.0 | 93 | <6 | 2 |
| 82 | 6.9 | 97 | <6 | 39 |
| 83 | 8.0 | 96 | 6.99 | 78 |
| 84 | 7.6 | 98 | 6.3 | 66 |
| 85 | 8.4 | 100 | 8.0 | 75 |
| 86 | 5.8 | 95 | <6 | 12 |
| 87 | 8.6 | 99 | 8.5 | 79 |
| 88 | 8.5 | 99 | 6.6 | 66 |
| 89 | 8.2 | 102 | 7.8 | 64 |
| 90 | 8.2 | 101 | 7.5 | 84 |
| 91 | 8.4 | 101 | 8.1 | 66 |
| 92 | 7.4 | 99 | <6 | 28 |
| 93 | 8.5 | 92 | 7.4 | 91 |
| 94 | 5.8 | 76 | <6 | 2 |
| 95 | 5.6 | 81 | <6 | −5 |
| 96 | 7.2 | 100 | 6.2 | 60 |
| 97 | 5.2 | 56 | <6 | −2 |
| 98 | 8.6 | 102 | 7.9 | 60 |
| 99 | 8.5 | 99 | 7.2 | 66 |
| 100 | 5.9 | 92 | <6 | −6 |
| 101 | 8.4 | 98 | 7.3 | 73 |
| 102 | 6.4 | 96 | <6 | 32 |
| 103 | 6.6 | 99 | | |
| 104 | 6.3 | 94 | <6 | 20 |
| 105 | 6.3 | 94 | <6 | 14 |
| 106 | 8.3 | 99 | 8.2 | 92 |
| 107 | 7.2 | 93 | 6.1 | 60 |
| 108 | 8.1 | 101 | 7.9 | 84 |
| 109 | 8.2 | 100 | 8.0 | 89 |

### Ex Vivo Oga Occupancy Assay Using [³H]-Ligand

### Drug Treatment and Tissue Preparation

Male NMRI or C57Bl6j mice were treated by oral (p.o.) administration of vehicle or compound. Animals were sacrificed 24 hours after administration. Brains were immediately removed from the skull, hemispheres were separated and the right hemisphere, for ex vivo OGA occupancy assay, was rapidly frozen in dry-ice cooled 2-methylbutane (−40° C.). Twenty □m-thick sagittal sections were cut using a Leica CM 3050 cryostat-microtome (Leica, Belgium), thaw-mounted on microscope slides (SuperFrost Plus Slides, Thermo Fisher Scientific) and stored at −20° C. until use. After thawing, sections were dried under a cold stream of air. The sections were not washed prior to incubation. The 10 minutes incubation with 3 nM [³H]-ligand was rigorously controlled. All brain sections (from compound-treated and vehicle-treated animals) were incubated in parallel. After incubation, the excess of [³H]-ligand was washed off in ice-cold buffer (PBS 1× and 1% BSA) 2 times 10 minutes, followed by a quick dip in distilled water. The sections were then dried under a stream of cold air.

### Quantitative Autoradiography and Data Analysis

Radioactivity in the forebrain area of brain slices was measured using a β-imager with M3 vision analysis software (Biospace Lab, Paris). Specific binding was calculated as the difference between total binding and non-specific binding measured in Thiamet-G (10 μM) treated sections. Specific binding in sections from drug treated animals was normalised to binding in sections from vehicle treated mice to calculate percentage of OGA occupancy by the drug.

| | | | Occupancy |
|---|---|---|---|
| Co. No. | Time (h) | Dose (mg/kg) | (% +/− sd) |
| 39 | 24 | 25 | 3.33 +/− 6.81 |

1. A compound of Formula (I) 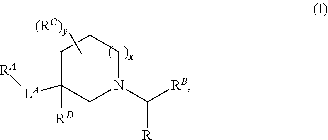
or a tautomer or a stereoisomeric form thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents;
C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;
L^{A} is selected from the group consisting of a covalent bond, —CH₂—, —O—, —OCH₂—, —CH₂O—, —NH—, —N(CH₃)—, —NHCH₂— and —CH₂NH—;
x represents 0 or 1;
R is H or CH₃; and
R^{B} is an aromatic heterobicyclic radical selected from the group consisting of (b-1) to (b-12) 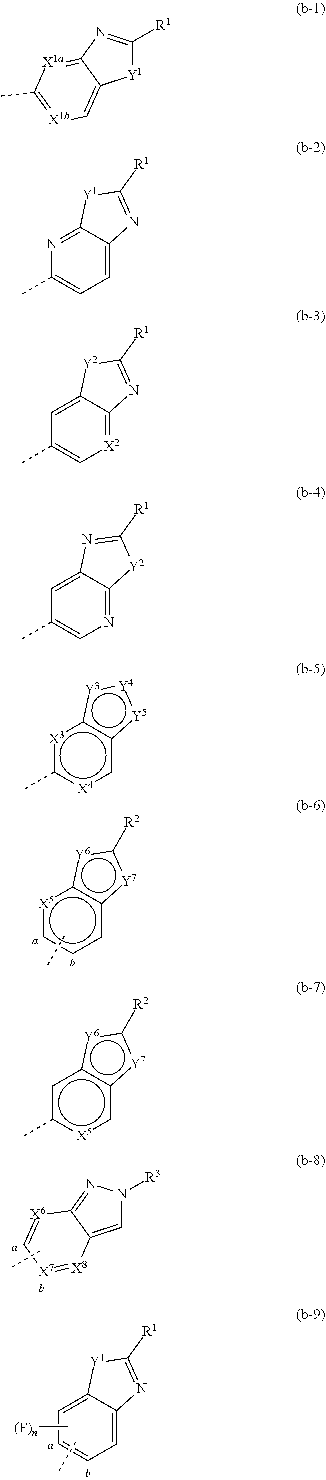 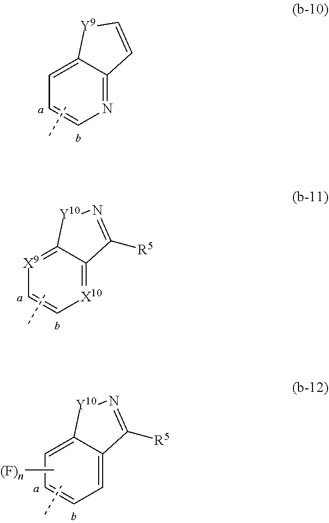
wherein
X^{1a} and X^{1b} each independently represents CH or N; and Y¹ represents O or S, with the proviso that at least one of X^{1a} and X^{1b} is CH, and when Y¹ is S, X^{1a} or X^{1b} is N;
X² represents CH or N; and Y² represents O or S;
X³ and X⁴ are each independently selected from N and CF; with the proviso that when X³ is N, X⁴ is CF and when X³ is CF, X⁴ is N;
one or two of Y³-Y⁵ is a heteroatom each independently selected from the group consisting of ═N—, >NH, >N(C₁₋₄ alkyl), S and O, with the proviso that up to one of Y³—Y⁵ may be O or S when present; and the remaining Y³-Y⁵ are each independently selected from the group consisting of CH and C(C₁₋₄alkyl);
X⁵ represents CH or N;
one of Y⁶ or Y⁷ is ═N— and the other is >NH or >NCH₃;
X⁶, X⁷ and X⁸ each independently represent CH or N, with the proviso that up to one of them can be N and with the proviso that X⁷ is C when b is the point of attachment to CHR;
Y⁸ and Y⁹ are each independently selected from the group consisting of O, S, NH and NCH₃;
X⁹ and X¹⁰ each independently represent CH or N, with the proviso that at least one of them is CH;
a and b, when present, represent the point of attachment of the aromatic heterobicyclic radical R^{B} to CHR;
R¹, R², and R³ are each selected from C₁₋₄alkyl;
R⁴ and R⁵ are each selected from the group consisting of H and C₁₋₄alkyl;
Y represents O or S;
n represents 1 or 2;
R^{C} is selected from the group consisting of fluoro, methyl, hydroxy, methoxy, trifluoromethyl, and difluoromethyl;
R^{D} is selected from the group consisting of hydrogen, fluoro, methyl, hydroxy, methoxy, trifluoromethyl, and difluoromethyl; and
y represents 0, 1 or 2;
with the provisos that
a) R^{C} is not hydroxy or methoxy when present at the carbon atom adjacent to the nitrogen atom of the piperidinediyl or pyrrolidinediyl ring;
b) R^{C} or R^{D} cannot be selected simultaneously from hydroxy or methoxy when R^{C} is present at the carbon atom adjacent to C—R^{D};
c) R^{D} is not hydroxy or methoxy when L^{A} is —O—, —OCH₂—, —CH₂O—, —NH—, —N(CH₃)—, —NH(CH₂)— or —(CH₂)NH—;
or a pharmaceutically acceptable addition salt or a solvate thereof.

2. The compound according to claim 1, wherein
R^{A} is a heteroaryl radical selected from the group consisting of pyridin-2-yl, pyridin-4-yl, and pyrimidin-4-yl, each of which may be optionally substituted with 1, 2 or 3 substituents each independently selected from the group consisting of halo; cyano, C₁₋₄alkyl optionally substituted with 1, 2, or 3 independently selected halo substituents;
C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents.

3. The compound according to claim 1, wherein L^{A} is selected from the group consisting of —CH₂—, —O—, —OCH₂—, —CH₂O—, —NH—, —N(CH₃)—, —NHCH₂— and —CH₂NH—.

4. The compound according to claim 1, wherein L^{A} is selected from the group consisting of a covalent bond, —CH₂—, —O—, —OCH₂— —CH₂O—, —NH—, —NHCH₂— and —CH₂NH—.

5. The compound of claim 1, wherein y is 0.

6. The compound of claim 1, wherein R^{B} is selected from the group consisting of (b-1), (b-2), (b-3), (b-4), (b-5), (b-6), (b-8), (b-9) and (b-10).

7. The compound of claim 1, wherein R^{B} is selected from the group consisting of (b-1), (b-2), (b-5), and (b-9).

8. The compound of claim 1, wherein R^{B} is selected from the group consisting of

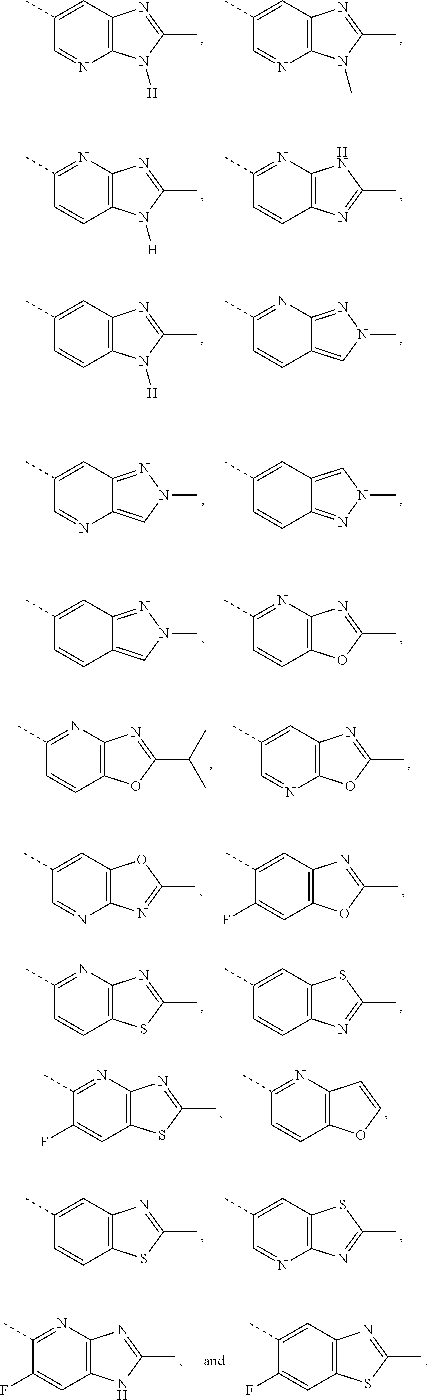

9. A pharmaceutical composition comprising a prophylactically or a therapeutically effective amount of a compound of claim 1 and a pharmaceutically acceptable carrier.

10. (canceled)

11. (canceled)

12. (canceled)

13. A method of preventing or treating a disorder selected from the group consisting of tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of a compound according of claim 1.

14. (canceled)
